SEQUENCE LISTING (123 sequences)

SEQ ID NO: 1 - RNA, 13 nt, Artificial Sequence
  source: /note=“Description of Artificial Sequence Synthetic oligonucleotide”
guuuuagagcuag

SEQ ID NO: 2 - RNA, 15 nt, Artificial Sequence
  source: /note=“Description of Artificial Sequence Synthetic oligonucleotide”
auagcaaguuaaaau

SEQ ID NO: 3 - RNA, 11 nt, Artificial Sequence
  source: /note=“Description of Artificial Sequence Synthetic oligonucleotide”
guuuuagagcu

SEQ ID NO: 4 - RNA, 13 nt, Artificial Sequence
  source: /note=“Description of Artificial Sequence Synthetic oligonucleotide”
agcaaguuaaaau

SEQ ID NO: 5 - RNA, 13 nt, Artificial Sequence
  source: /note=“Description of Artificial Sequence Synthetic oligonucleotide”
guuuuagagcuag

SEQ ID NO: 6 - RNA, 15 nt, Artificial Sequence
  source: /note=“Description of Artificial Sequence Synthetic oligonucleotide”
cuagcaaguuaaaau

SEQ ID NO: 7 - RNA, 14 nt, Artificial Sequence
  source: /note=“Description of Artificial Sequence Synthetic oligonucleotide”
guuuuagagcuaug

SEQ ID NO: 8 - RNA, 16 nt, Artificial Sequence
  source: /note=“Description of Artificial Sequence Synthetic oligonucleotide”
cauagcaaguuaaaau

SEQ ID NO: 9 - RNA, 21 nt, Artificial Sequence
  source: /note=“Description of Artificial Sequence Synthetic oligonucleotide”
guauuagagcuaugcuguuuu

SEQ ID NO: 10 - RNA, 23 nt, Artificial Sequence
  source: /note=“Description of Artificial Sequence Synthetic oligonucleotide”
aaaacagcauagcaaguuaauau

SEQ ID NO: 11 - RNA, 16 nt, Artificial Sequence
  source: /note=“Description of Artificial Sequence Synthetic oligonucleotide”
guauuagagcuaugcu

SEQ ID NO: 12 - RNA, 18 nt, Artificial Sequence
  source: /note=“Description of Artificial Sequence Synthetic oligonucleotide”
agcauagcaaguuaauau

SEQ ID NO: 13 - RNA, 21 nt, Artificial Sequence
  source: /note=“Description of Artificial Sequence Synthetic oligonucleotide”
guuuuagagcuaugcuguuuu

SEQ ID NO: 14 - RNA, 23 nt, Artificial Sequence
  source: /note=“Description of Artificial Sequence Synthetic oligonucleotide”
aaaacagcauagcaaguuaaaau

SEQ ID NO: 15 - RNA, 16 nt, Artificial Sequence
  source: /note=“Description of Artificial Sequence Synthetic oligonucleotide”
guuuuagagcuaugcu

SEQ ID NO: 16 - RNA, 18 nt, Artificial Sequence
  source: /note=“Description of Artificial Sequence Synthetic oligonucleotide”
agcauagcaaguuaaaaa

SEQ ID NO: 17 - RNA, 15 nt, Artificial Sequence
  source: /note=“Description of Artificial Sequence Synthetic oligonucleotide”
guuuuagagcuaaag

SEQ ID NO: 18 - RNA, 17 nt, Artificial Sequence
  source: /note=“Description of Artificial Sequence Synthetic oligonucleotide”
auuuagcaaguuaaaau

SEQ ID NO: 19 - RNA, 13 nt, Artificial Sequence
  source: /note=“Description of Artificial Sequence Synthetic oligonucleotide”
guuuuagagcuaa

SEQ ID NO: 20 - RNA, 15 nt, Artificial Sequence
  source: /note=“Description of Artificial Sequence Synthetic oligonucleotide”
uuagcaaguuaaaau

SEQ ID NO: 21 - RNA, 17 nt, Artificial Sequence
  source: /note=“Description of Artificial Sequence Synthetic oligonucleotide”
guuuuagagcuaaaggg

SEQ ID NO: 22 - RNA, 19 nt, Artificial Sequence
  source: /note=“Description of Artificial Sequence Synthetic oligonucleotide”
accuuuagcaaguuaaaau

SEQ ID NO: 23 - RNA, 13 nt, Artificial Sequence
  source: /note=“Description of Artificial Sequence Synthetic oligonucleotide”
guuuuagagcuag

SEQ ID NO: 24 - RNA, 13 nt, Artificial Sequence
  source: /note=“Description of Artificial Sequence Synthetic oligonucleotide”
guuuuaguacucu

SEQ ID NO: 25 - RNA, 15 nt, Artificial Sequence
  source: /note=“Description of Artificial Sequence Synthetic oligonucleotide”
agaaucuacuaaaac

SEQ ID NO: 26 - RNA, 16 nt, Artificial Sequence
  source: /note=“Description of Artificial Sequence Synthetic oligonucleotide”
guuuuaguacucugua

SEQ ID NO: 27 - RNA, 18 nt, Artificial Sequence
  source: /note=“Description of Artificial Sequence Synthetic oligonucleotide”
uacagaaucuacuaaaac

SEQ ID NO: 28 - RNA, 24 nt, Artificial Sequence
  source: /note=“Description of Artificial Sequence Synthetic oligonucleotide”
guuuuaguacucuguaauuuuagg

SEQ ID NO: 29 - RNA, 26 nt, Artificial Sequence
  source: /note=“Description of Artificial Sequence Synthetic oligonucleotide”
ccuaaaauuacagaaucuacuaaaac

SEQ ID NO: 30 - RNA, 29 nt, Artificial Sequence
  source: /note=“Description of Artificial Sequence Synthetic oligonucleotide”
guuuuaguacucuguaauuuuagguauga

SEQ ID NO: 31 - RNA, 31 nt, Artificial Sequence
  source: /note=“Description of Artificial Sequence Synthetic oligonucleotide”
ucauaccuaaaauuacagaaucuacuaaaac

SEQ ID NO: 32 - RNA, 100 nt, Artificial Sequence
  source: /note=“Description of Artificial Sequence Synthetic polynucleotide”
  modified_base (1)..(20): a, c, u, g, unknown or other
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 33 - RNA, 34 nt, Artificial Sequence
  source: /note=“Description of Artificial Sequence Synthetic oligonucleotide”
  modified_base (1)..(20): a, c, u, g, unknown or other
nnnnnnnnnnnnnnnnnnnnguuuuagagcuaga

SEQ ID NO: 34 - RNA, 66 nt, Artificial Sequence
  source: /note=“Description of Artificial Sequence Synthetic oligonucleotide”
aauagcaaguuaaaauaaggcuaguccguuaucaacuugaaaaaguggcaccgagucggugcuuuu

SEQ ID NO: 35 - RNA, 34 nt, Artificial Sequence; identical to SEQ ID NO: 33
  source: /note=“Description of Artificial Sequence Synthetic oligonucleotide”
  modified_base (1)..(20): a, c, u, g, unknown or other

SEQ ID NO: 36 - RNA, 34 nt, Artificial Sequence
  source: /note=“Description of Artificial Sequence Synthetic oligonucleotide”
guaacggcagacuucuccucguuuuagagcuaga

SEQ ID NO: 37 - RNA, 32 nt, Artificial Sequence
  source: /note=“Description of Artificial Sequence Synthetic oligonucleotide”
guaacggcagacuucuccucguuuuagagcua

SEQ ID NO: 38 - RNA, 34 nt, Artificial Sequence
  source: /note=“Description of Artificial Sequence Synthetic oligonucleotide”
guaacggcagacuucuccucguuuuagagcuagg

SEQ ID NO: 39 - RNA, 35 nt, Artificial Sequence
  source: /note=“Description of Artificial Sequence Synthetic oligonucleotide”
guaacggcagacuucuccucguuuuagagcuaugc

SEQ ID NO: 40 - RNA, 42 nt, Artificial Sequence
  source: /note=“Description of Artificial Sequence Synthetic oligonucleotide”
guaacggcagacuucuccucguauuagagcuaugcuguuuug

SEQ ID NO: 41 - RNA, 37 nt, Artificial Sequence
  source: /note=“Description of Artificial Sequence Synthetic oligonucleotide”
guaacggcagacuucuccucguauuagagcuaugcug

SEQ ID NO: 42 - RNA, 42 nt, Artificial Sequence
  source: /note=“Description of Artificial Sequence Synthetic oligonucleotide”
guaacggcagacuucuccucguuuuagagcuaugcuguuuug

SEQ ID NO: 43 - RNA, 37 nt, Artificial Sequence
  source: /note=“Description of Artificial Sequence Synthetic oligonucleotide”
guaacggcagacuucuccucguuuuagagcuaugcug

SEQ ID NO: 44 - RNA, 36 nt, Artificial Sequence
  source: /note=“Description of Artificial Sequence Synthetic oligonucleotide”
guaacggcagacuucuccucguuuuagagcuaaaga

SEQ ID NO: 45 - RNA, 34 nt, Artificial Sequence
  source: /note=“Description of Artificial Sequence Synthetic oligonucleotide”
guaacggcagacuucuccucguuuuagagcuaaa

SEQ ID NO: 46 - RNA, 38 nt, Artificial Sequence
  source: /note=“Description of Artificial Sequence Synthetic oligonucleotide”
guaacggcagacuucuccucguuuuagagcuaaaggga

SEQ ID NO: 47 - RNA, 35 nt, Artificial Sequence
  source: /note=“Description of Artificial Sequence Synthetic oligonucleotide”
guaacggcagacuucuccucguuuuagagcuagda

SEQ ID NO: 48 - RNA, 35 nt, Artificial Sequence
  source: /note=“Description of Artificial Sequence Synthetic oligonucleotide”
cuaacaguugcuuuuaucacguuuuagagcuaugc

SEQ ID NO: 49 - RNA, 37 nt, Artificial Sequence
  source: /note=“Description of Artificial Sequence Synthetic oligonucleotide”
cuaacaguugcuuuuaucacguuuuagagcuaugcug

SEQ ID NO: 50 - RNA, 42 nt, Artificial Sequence
  source: /note=“Description of Artificial Sequence Synthetic oligonucleotide”
cuaacaguugcuuuuaucacguuuuagagcuaugcuguuuug

SEQ ID NO: 51 - RNA, 42 nt, Artificial Sequence
  source: /note=“Description of Artificial Sequence Synthetic oligonucleotide”
cuaacaguugcuuuuaucacguauuagagcuaugcuguuuug

SEQ ID NO: 52 - RNA, 34 nt, Artificial Sequence
  source: /note=“Description of Artificial Sequence Synthetic oligonucleotide”
cuaacaguugcuuuuaucacguuuuagagcuaga

SEQ ID NO: 53 - RNA, 34 nt, Artificial Sequence
  source: /note=“Description of Artificial Sequence Synthetic oligonucleotide”
guaacggcagacuucuccucguuuuaguacucug

SEQ ID NO: 54 - RNA, 37 nt, Artificial Sequence
  source: /note=“Description of Artificial Sequence Synthetic oligonucleotide”
guaacggcagacuucuccucguuuuaguacucuguaa

SEQ ID NO: 55 - RNA, 45 nt, Artificial Sequence
  source: /note=“Description of Artificial Sequence Synthetic oligonucleotide”
guaacggcagacuucuccucguuuuaguacucuguaauuuuaggu

SEQ ID NO: 56 - RNA, 50 nt, Artificial Sequence
  source: /note=“Description of Artificial Sequence Synthetic oligonucleotide”
guaacggcagacuucuccucguuuuaguacucuguaauuuuagguaugag

SEQ ID NO: 57 - RNA, 66 nt, Artificial Sequence; identical to SEQ ID NO: 34
  source: /note=“Description of Artificial Sequence Synthetic oligonucleotide”

SEQ ID NO: 58 - RNA, 66 nt, Artificial Sequence; identical to SEQ ID NO: 34
  source: /note=“Description of Artificial Sequence Synthetic oligonucleotide”

SEQ ID NO: 59 - RNA, 64 nt, Artificial Sequence
  source: /note=“Description of Artificial Sequence Synthetic oligonucleotide”
uagcaaguuaaaauaaggcuaguccguuaucaacuugaaaaaguggcaccgagucggugcuuuu

SEQ ID NO: 60 - RNA, 66 nt, Artificial Sequence
  source: /note=“Description of Artificial Sequence Synthetic oligonucleotide”
ccuagcaaguuaaaauaaggcuaguccguuaucaacuugaaaaaguggcaccgagucggugcuuuu

SEQ ID NO: 61 - RNA, 67 nt, Artificial Sequence
  source: /note=“Description of Artificial Sequence Synthetic oligonucleotide”
gcauagcaaguuaaaauaaggcuaguccguuaucaacuugaaaaaguggcaccgagucggugcuuuu

SEQ ID NO: 62 - RNA, 74 nt, Artificial Sequence
  source: /note=“Description of Artificial Sequence Synthetic oligonucleotide”
caaaacagcauagcaaguuaauauaaggcuaguccguuaucaacuugaaaaaguggcaccgagucggugcuuuu

SEQ ID NO: 63 - RNA, 69 nt, Artificial Sequence
  source: /note=“Description of Artificial Sequence Synthetic oligonucleotide”
cagcauagcaaguuaauauaaggcuaguccguuaucaacuugaaaaaguggcaccgagucggugcuuuu

SEQ ID NO: 64 - RNA, 74 nt, Artificial Sequence
  source: /note=“Description of Artificial Sequence Synthetic oligonucleotide”
caaaacagcauagcaaguuaaaauaaggcuaguccguuaucaacuugaaaaaguggcaccgagucggugcuuuu

SEQ ID NO: 65 - RNA, 69 nt, Artificial Sequence
  source: /note=“Description of Artificial Sequence Synthetic oligonucleotide”
cagcauagcaaguuaaaaaaaggcuaguccguuaucaacuugaaaaaguggcaccgagucggugcuuuu

SEQ ID NO: 66 - RNA, 68 nt, Artificial Sequence
  source: /note=“Description of Artificial Sequence Synthetic oligonucleotide”
aauuuagcaaguuaaaauaaggcuaguccguuaucaacuugaaaaaguggcaccgagucggugcuuuu

SEQ ID NO: 67 - RNA, 66 nt, Artificial Sequence
  source: /note=“Description of Artificial Sequence Synthetic oligonucleotide”
uuuagcaaguuaaaauaaggcuaguccguuaucaacuugaaaaaguggcaccgagucggugcuuuu

SEQ ID NO: 68 - RNA, 70 nt, Artificial Sequence
  source: /note=“Description of Artificial Sequence Synthetic oligonucleotide”
aaccuuuagcaaguuaaaauaaggcuaguccguuaucaacuugaaaaaguggcaccgagucggugcuuuu

SEQ ID NO: 69 - RNA, 66 nt, Artificial Sequence; identical to SEQ ID NO: 34
  source: /note=“Description of Artificial Sequence Synthetic oligonucleotide”

SEQ ID NO: 70 - RNA, 67 nt, Artificial Sequence; identical to SEQ ID NO: 61
  source: /note=“Description of Artificial Sequence Synthetic oligonucleotide”

SEQ ID NO: 71 - RNA, 69 nt, Artificial Sequence
  source: /note=“Description of Artificial Sequence Synthetic oligonucleotide”
cagcauagcaaguuaaaauaaggcuaguccguuaucaacuugaaaaaguggcaccgagucggugcuuuu

SEQ ID NO: 72 - RNA, 74 nt, Artificial Sequence; identical to SEQ ID NO: 64
  source: /note=“Description of Artificial Sequence Synthetic oligonucleotide”

SEQ ID NO: 73 - RNA, 74 nt, Artificial Sequence; identical to SEQ ID NO: 62
  source: /note=“Description of Artificial Sequence Synthetic oligonucleotide”

SEQ ID NO: 74 - RNA, 66 nt, Artificial Sequence; identical to SEQ ID NO: 34
  source: /note=“Description of Artificial Sequence Synthetic oligonucleotide”

SEQ ID NO: 75 - RNA, 62 nt, Artificial Sequence
  source: /note=“Description of Artificial Sequence Synthetic oligonucleotide”
cagaaucuacuaaaacaaggcaaaaugccguguuuaucucgucaacuuguuggcgagauuuu

SEQ ID NO: 76 - RNA, 65 nt, Artificial Sequence
  source: /note=“Description of Artificial Sequence Synthetic oligonucleotide”
uuacagaaucuacuaaaacaaggcaaaaugccguguuuaucucgucaacuuguuggcgagauuuu

SEQ ID NO: 77 - RNA, 73 nt, Artificial Sequence
  source: /note=“Description of Artificial Sequence Synthetic oligonucleotide”
accuaaaauuacagaaucuacuaaaacaaggcaaaaugccguguuuaucucgucaacuuguuggcgagauuuu

SEQ ID NO: 78 - RNA, 78 nt, Artificial Sequence
  source: /note=“Description of Artificial Sequence Synthetic oligonucleotide”
cucauaccuaaaauuacagaaucuacuaaaacaaggcaaaaugccguguuuaucucgucaacuuguuggcgagauuuu

SEQ ID NO: 79 - RNA, 54 nt, Artificial Sequence
  source: /note=“Description of Artificial Sequence Synthetic oligonucleotide”
  modified_base (1)..(15): a, c, u, g, unknown or other
  modified_base (27)..(48): a, c, u, g, unknown or other
  misc_feature (27)..(48): /note=“This region may encompass 2-22 nucleotides”
  modified_base (49)..(54): a, c, u, g, unknown or other
  misc_feature (49)..(54): /note=“This region may encompass 0-6 nucleotides”
  source: /note=“See specification as filed for detailed description of substitutions and preferred embodiments”
nnnnnnnnnnnnnnnuuuuagagcuannnnnnnnnnnnnnnnnnnnnnnnnnnn

SEQ ID NO: 80 - RNA, 71 nt, Artificial Sequence
  source: /note=“Description of Artificial Sequence Synthetic oligonucleotide”
  modified_base (1)..(6): a, c, u, g, unknown or other
  misc_feature (1)..(6): /note=“This region may encompass 0-6 nucleotides”
  modified_base (7)..(28): a, c, u, g, unknown or other
  misc_feature (7)..(28): /note=“This region may encompass 2-22 nucleotides”
  modified_base (42)..(71): a, c, u, g, unknown or other
  source: /note=“See specification as filed for detailed description of substitutions and preferred embodiments”
nnnnnnnnnnnnnnnnnnnnnnnnnnnnuagcaaguuaaaannnnnnnnnnnnnnnnnnnnnnnnnnnnnn

SEQ ID NO: 81 - RNA, 54 nt, Artificial Sequence
  source: /note=“Description of Artificial Sequence Synthetic oligonucleotide”
  modified_base (1)..(15): a, c, u, g, unknown or other
  modified_base (27)..(48): a, c, u, g, unknown or other
  misc_feature (27)..(48): /note=“This region may encompass 2-22 nucleotides”
  modified_base (49)..(54): a, c, u, g, unknown or other
  misc_feature (49)..(54): /note=“This region may encompass 0-6 nucleotides”
  source: /note=“See specification as filed for detailed description of substitutions and preferred embodiments”
nnnnnnnnnnnnnnnuauuagagcuannnnnnnnnnnnnnnnnnnnnnnnnnnn

SEQ ID NO: 82 - RNA, 71 nt, Artificial Sequence
  source: /note=“Description of Artificial Sequence Synthetic oligonucleotide”
  modified_base (1)..(6): a, c, u, g, unknown or other
  misc_feature (1)..(6): /note=“This region may encompass 0-6 nucleotides”
  modified_base (7)..(28): a, c, u, g, unknown or other
  misc_feature (7)..(28): /note=“This region may encompass 2-22 nucleotides”
  modified_base (42)..(71): a, c, u, g, unknown or other
  source: /note=“See specification as filed for detailed description of substitutions and preferred embodiments”
nnnnnnnnnnnnnnnnnnnnnnnnnnnnuagcaaguuaauannnnnnnnnnnnnnnnnnnnnnnnnnnnnn

SEQ ID NO: 83 - RNA, 57 nt, Artificial Sequence
  source: /note=“Description of Artificial Sequence Synthetic oligonucleotide”
  modified_base (1)..(15): a, c, u, g, unknown or other
  modified_base (30)..(51): a, c, u, g, unknown or other
  misc_feature (30)..(51): /note=“This region may encompass 2-22 nucleotides”
  modified_base (52)..(57): a, c, u, g, unknown or other
  misc_feature (52)..(57): /note=“This region may encompass 0-6 nucleotides”
  source: /note=“See specification as filed for detailed description of substitutions and preferred embodiments”
nnnnnnnnnnnnnnnguuuuaguacucugnnnnnnnnnnnnnnnnnnnnnnnnnnnn

SEQ ID NO: 84 - RNA, 74 nt, Artificial Sequence
  source: /note=“Description of Artificial Sequence Synthetic oligonucleotide”
  modified_base (1)..(6): a, c, u, g, unknown or other
  misc_feature (1)..(6): /note=“This region may encompass 0-6 nucleotides”
  modified_base (7)..(28): a, c, u, g, unknown or other
  misc_feature (7)..(28): /note=“This region may encompass 2-22 nucleotides”
  modified_base (45)..(74): a, c, u, g, unknown or other
  source: /note=“See specification as filed for detailed description of substitutions and preferred embodiments”
nnnnnnnnnnnnnnnnnnnnnnnnnnnncagaaucuacuaaaacnnnnnnnnnnnnnnnnnnnnnnnnnnnnnn

SEQ ID NO: 85 - RNA, 57 nt, Artificial Sequence
  source: /note=“Description of Artificial Sequence Synthetic oligonucleotide”
  modified_base (1)..(15): a, c, u, g, unknown or other
  modified_base (30)..(51): a, c, u, g, unknown or other
  misc_feature (30)..(51): /note=“This region may encompass 2-22 nucleotides”
  modified_base (52)..(57): a, c, u, g, unknown or other
  misc_feature (52)..(57): /note=“This region may encompass 0-6 nucleotides”
  source: /note=“See specification as filed for detailed description of substitutions and preferred embodiments”
nnnnnnnnnnnnnnnguauuaguacucugnnnnnnnnnnnnnnnnnnnnnnnnnnnn

SEQ ID NO: 86 - RNA, 74 nt, Artificial Sequence
  source: /note=“Description of Artificial Sequence Synthetic oligonucleotide”
  modified_base (1)..(6): a, c, u, g, unknown or other
  misc_feature (1)..(6): /note=“This region may encompass 0-6 nucleotides”
  modified_base (7)..(28): a, c, u, g, unknown or other
  misc_feature (7)..(28): /note=“This region may encompass 2-22 nucleotides”
  modified_base (45)..(74): a, c, u, g, unknown or other
  source: /note=“See specification as filed for detailed description of substitutions and preferred embodiments”
nnnnnnnnnnnnnnnnnnnnnnnnnnnncagaaucuacuaauacnnnnnnnnnnnnnnnnnnnnnnnnnnnnnn

SEQ ID NO: 87 - RNA, 10 nt, Artificial Sequence
  source: /note=“Description of Artificial Sequence Synthetic oligonucleotide”
uaauuuuagg

SEQ ID NO: 88 - RNA, 11 nt, Artificial Sequence
  source: /note=“Description of Artificial Sequence Synthetic oligonucleotide”
uaauuuuaggu

SEQ ID NO: 89 - RNA, 12 nt, Artificial Sequence
  source: /note=“Description of Artificial Sequence Synthetic oligonucleotide”
uaauuuuaggua

SEQ ID NO: 90 - RNA, 13 nt, Artificial Sequence
  source: /note=“Description of Artificial Sequence Synthetic oligonucleotide”
uaauuuuagguau

SEQ ID NO: 91 - RNA, 14 nt, Artificial Sequence
  source: /note=“Description of Artificial Sequence Synthetic oligonucleotide”
uaauuuuagguaug

SEQ ID NO: 92 - RNA, 15 nt, Artificial Sequence
  source: /note=“Description of Artificial Sequence Synthetic oligonucleotide”
uaauuuuagguauga

SEQ ID NO: 93 - RNA, 101 nt, Artificial Sequence
  source: /note=“Description of Artificial Sequence Synthetic polynucleotide”
  modified_base (1)..(21): a, c, u, g, unknown or other
nnnnnnnnnnnnnnnnnnnnnguuuuagagcuagaaauagcaaguuaaaauaaggcuaguccguuaucaacuugaaaaaguggcaccgagucggugcuuuu

SEQ ID NO: 94 - RNA, 104 nt, Artificial Sequence
  source: /note=“Description of Artificial Sequence Synthetic polynucleotide”
  modified_base (1)..(21): a, c, u, g, unknown or other
nnnnnnnnnnnnnnnnnnnnnguuuuaguacucuggaaacagaaucuacuaaaacaaggcaaaaugccguguuuaucucgucaacuuguuggcgagauuuuuuu

SEQ ID NO: 95 - RNA, 34 nt, Artificial Sequence; identical to SEQ ID NO: 33
  source: /note=“Description of Artificial Sequence Synthetic oligonucleotide”
  modified_base (1)..(20): a, c, u, g, unknown or other

SEQ ID NO: 96 - RNA, 66 nt, Artificial Sequence; identical to SEQ ID NO: 34
  source: /note=“Description of Artificial Sequence Synthetic oligonucleotide”

SEQ ID NO: 97 - RNA, 32 nt, Artificial Sequence
  source: /note=“Description of Artificial Sequence Synthetic oligonucleotide”
  modified_base (1)..(20): a, c, u, g, unknown or other
nnnnnnnnnnnnnnnnnnnnguuuuagagcua

SEQ ID NO: 98 - RNA, 64 nt, Artificial Sequence; identical to SEQ ID NO: 59
  source: /note=“Description of Artificial Sequence Synthetic oligonucleotide”

SEQ ID NO: 99 - RNA, 34 nt, Artificial Sequence
  source: /note=“Description of Artificial Sequence Synthetic oligonucleotide”
  modified_base (1)..(20): a, c, u, g, unknown or other
nnnnnnnnnnnnnnnnnnnnguuuuagagcuagg

SEQ ID NO: 100 - RNA, 66 nt, Artificial Sequence; identical to SEQ ID NO: 60
  source: /note=“Description of Artificial Sequence Synthetic oligonucleotide”

SEQ ID NO: 101 - RNA, 35 nt, Artificial Sequence
  source: /note=“Description of Artificial Sequence Synthetic oligonucleotide”
  modified_base (1)..(20): a, c, u, g, unknown or other
nnnnnnnnnnnnnnnnnnnnguuuuagagcuaugc

SEQ ID NO: 102 - RNA, 67 nt, Artificial Sequence; identical to SEQ ID NO: 61
  source: /note=“Description of Artificial Sequence Synthetic oligonucleotide”

SEQ ID NO: 103 - RNA, 42 nt, Artificial Sequence
  source: /note=“Description of Artificial Sequence Synthetic oligonucleotide”
  modified_base (1)..(20): a, c, u, g, unknown or other
nnnnnnnnnnnnnnnnnnnnguauuagagcuaugcuguuuug

SEQ ID NO: 104 - RNA, 74 nt, Artificial Sequence; identical to SEQ ID NO: 62
  source: /note=“Description of Artificial Sequence Synthetic oligonucleotide”

SEQ ID NO: 105 - RNA, 37 nt, Artificial Sequence
  source: /note=“Description of Artificial Sequence Synthetic oligonucleotide”
  modified_base (1)..(20): a, c, u, g, unknown or other
nnnnnnnnnnnnnnnnnnnnguauuagagcuaugcug

SEQ ID NO: 106 - RNA, 69 nt, Artificial Sequence; identical to SEQ ID NO: 63
  source: /note=“Description of Artificial Sequence Synthetic oligonucleotide”

SEQ ID NO: 107 - RNA, 42 nt, Artificial Sequence
  source: /note=“Description of Artificial Sequence Synthetic oligonucleotide”
  modified_base (1)..(20): a, c, u, g, unknown or other
nnnnnnnnnnnnnnnnnnnnguuuuagagcuaugcuguuuug

SEQ ID NO: 108 - RNA, 74 nt, Artificial Sequence; identical to SEQ ID NO: 64
  source: /note=“Description of Artificial Sequence Synthetic oligonucleotide”

SEQ ID NO: 109 - RNA, 37 nt, Artificial Sequence
  source: /note=“Description of Artificial Sequence Synthetic oligonucleotide”
  modified_base (1)..(20): a, c, u, g, unknown or other
nnnnnnnnnnnnnnnnnnnnguuuuagagcuaugcug

SEQ ID NO: 110 - RNA, 69 nt, Artificial Sequence; identical to SEQ ID NO: 65
  source: /note=“Description of Artificial Sequence Synthetic oligonucleotide”

SEQ ID NO: 111 - RNA, 37 nt, Artificial Sequence; identical to SEQ ID NO: 109
  source: /note=“Description of Artificial Sequence Synthetic oligonucleotide”
  modified_base (1)..(20): a, c, u, g, unknown or other

SEQ ID NO: 112 - RNA, 69 nt, Artificial Sequence; identical to SEQ ID NO: 71
  source: /note=“Description of Artificial Sequence Synthetic oligonucleotide”

SEQ ID NO: 113 - RNA, 34 nt, Artificial Sequence
  source: /note=“Description of Artificial Sequence Synthetic oligonucleotide”
  modified_base (1)..(20): a, c, u, g, unknown or other
nnnnnnnnnnnnnnnnnnnnguuuuaguacucug

SEQ ID NO: 114 - RNA, 62 nt, Artificial Sequence; identical to SEQ ID NO: 75
  source: /note=“Description of Artificial Sequence Synthetic oligonucleotide”

SEQ ID NO: 115 - RNA, 37 nt, Artificial Sequence
  source: /note=“Description of Artificial Sequence Synthetic oligonucleotide”
  modified_base (1)..(20): a, c, u, g, unknown or other
nnnnnnnnnnnnnnnnnnnnguuuuaguacucuguaa

SEQ ID NO: 116 - RNA, 65 nt, Artificial Sequence; identical to SEQ ID NO: 76
  source: /note=“Description of Artificial Sequence Synthetic oligonucleotide”

SEQ ID NO: 117 - RNA, 45 nt, Artificial Sequence
  source: /note=“Description of Artificial Sequence Synthetic oligonucleotide”
  modified_base (1)..(20): a, c, u, g, unknown or other
nnnnnnnnnnnnnnnnnnnnguuuuaguacucuguaauuuuaggu

SEQ ID NO: 118 - RNA, 73 nt, Artificial Sequence; identical to SEQ ID NO: 77
  source: /note=“Description of Artificial Sequence Synthetic oligonucleotide”

SEQ ID NO: 119 - RNA, 50 nt, Artificial Sequence
  source: /note=“Description of Artificial Sequence Synthetic oligonucleotide”
  modified_base (1)..(20): a, c, u, g, unknown or other
nnnnnnnnnnnnnnnnnnnnguuuuaguacucuguaauuuuagguaugag

SEQ ID NO: 120 - RNA, 78 nt, Artificial Sequence; identical to SEQ ID NO: 78
  source: /note=“Description of Artificial Sequence Synthetic oligonucleotide”

SEQ ID NO: 121 - DNA, 10 bp, Artificial Sequence
  source: /note=“Description of Artificial Sequence Synthetic oligonucleotide”
  misc_feature (1)..(10): /note=“This sequence may encompass 1-10 nucleotides”
aaaaaaaaaa

SEQ ID NO: 122 - DNA, 20 bp, Artificial Sequence
  source: /note=“Description of Artificial Sequence Synthetic oligonucleotide”
  misc_feature (1)..(20): /note=“This sequence may encompass 10-20 nucleotides”
aaaaaaaaaaaaaaaaaaaa

SEQ ID NO: 123 - DNA, 200 bp, Artificial Sequence
  source: /note=“Description of Artificial Sequence Synthetic polynucleotide”
  misc_feature (1)..(200): /note=“This sequence may encompass 25-200 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2023/0111575 A1
(43) Pub. Date: Apr. 13, 2023

(54) SYNTHETIC GUIDE MOLECULES, COMPOSITIONS AND METHODS RELATING THERETO

(71) Applicant: Editas Medicine, Inc., Cambridge, MA (US)

(72) Inventors: Jim Heil, Boulder, CO (US); Stephanie King, Boulder, CO (US); Sam Saccomano, Boulder, CO (US); Stacy Capehart, Boulder, CO (US); Cecilia Fernandez, Cambridge, MA (US); Hariharan Jayaram, San Mateo, CA (US); Bruce Eaton, Boulder, CO (US); Karin Zemski Berry, Boulder, CO (US)

(21) Appl. No.: 16/474,198

(22) PCT Filed: Dec. 29, 2017
(86) PCT No.: PCT/US17/69019

§ 371 Date: Jun. 27, 2019

Related U.S. Application Data
(60) Provisional application No. 62/492,001, filed on Apr. 28, 2017.
(60) Provisional application No. 62/441,046, filed on Dec. 30, 2016.

(51) Int. Cl. C12N 15/113; C07H 21/04; C07H 21/02; A61K 31/712

(52) U.S. Cl. CPC C12N 15/113; C07H 21/04; C07H 21/02; A61K 31/712; C12N 2310/20; C12N 2330/30

(57) ABSTRACT

Chemical syntheses of guide molecules are disclosed, along with compositions and methods relating thereto.

### CROSS REFERENCE TO RELATED APPLICATIONS

This application claims the benefit of U.S. Application No. 62/441,046, filed on Dec. 30, 2016, and U.S. Application No. 62/492,001, filed on Apr. 28, 2017, the disclosure of each of which is hereby incorporated by reference in its entirety.

### FIELD

The present disclosure relates to CRISPR/Cas-related methods and components for editing a target nucleic acid sequence, or modulating expression of a target nucleic acid sequence. More particularly, this disclosure relates to synthetic guide molecules and related systems, methods and compositions.

### BACKGROUND

CRISPRs (Clustered Regularly Interspaced Short Palindromic Repeats) evolved in bacteria and archaea as an adaptive immune system to defend against viral attack. Upon exposure to a virus, short segments of viral DNA are integrated into the CRISPR locus. RNA is transcribed from a portion of the CRISPR locus that includes the viral sequence. That RNA, which contains sequence complementary to the viral genome, mediates targeting of an RNA-guided nuclease protein such as Cas9 or Cpf1 to a target sequence in the viral genome. The RNA-guided nuclease, in turn, cleaves and thereby silences the viral target.

Recently, CRISPR systems have been adapted for genome editing in eukaryotic cells. These systems generally include a protein component (the RNA-guided nuclease) and a nucleic acid component (generally referred to as a guide molecule, guide RNA or “gRNA”). These two components form a complex that interacts with specific target DNA sequences recognized by, or complementary to, the two components of the system and optionally edits or alters the target sequence, for example by means of site-specific DNA cleavage. The editing or alteration of the target sequence may also involve the recruitment of cellular DNA repair mechanisms such as non-homologous end-joining (NHEJ) or homology-directed repair (HDR).

The value of CRISPR systems as a means of treating genetic diseases has been widely appreciated, but certain technical challenges must be addressed for therapeutics based on these systems to achieve broad clinical application. Among other things, a need exists for cost-effective and straightforward commercial-scale synthesis of high-quality CRISPR system components.

For instance, most guide molecules are currently synthesized by one of two methods: in-vitro transcription (IVT) and chemical synthesis. IVT typically involves the transcription of RNA from a DNA template by means of a bacterial RNA polymerase such as T7 polymerase. At present, IVT manufacturing of guide molecules in accordance with good manufacturing practice (GMP) standards required by regulators in the US and abroad may be costly and limited in scale. In addition, IVT synthesis may not be suitable for all guide RNA sequences: the T7 polymerase tends to transcribe sequences which initiate with a 5′ guanine more efficiently than those initiated with another 5′ base, and may recognize stem-loop structures followed by poly-uracil tracts, which structures are present in certain guide molecules, as a signal to terminate transcription, resulting in truncated guide molecule transcripts.

Chemical synthesis, on the other hand, is inexpensive and GMP-production for shorter oligonucleotides (e.g., less than 100 nucleotides in length) is readily available. Chemical synthesis methods are described throughout the literature, for instance by Beaucage and Carruthers, Curr Protoc Nucleic Acid Chem. 2001 May; Chapter 3: Unit 3.3 (Beaucage & Carruthers), which is incorporated by reference in its entirety and for all purposes herein. These methods typically involve the stepwise addition of reactive nucleotide monomers until an oligonucleotide sequence of a desired length is reached. In the most commonly used synthesis regimes (such as the phosphoramidite method) monomers are added to the 5′ end of the oligonucleotide. These monomers are often 3′ functionalized (e.g. with a phosphoramidite) and include a 5′ protective group (such as a 4,4′ dimethoxytrityl), for example according to Formula I, below:

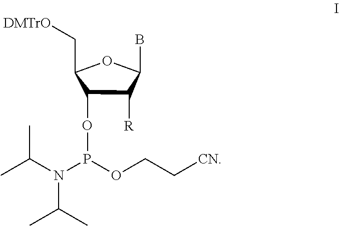

In Formula I, DMTr is 4,4′-dimethoxytrityl, R is a group which is compatible with the oligonucleotide synthesis conditions, non-limiting examples of which include H, F, O-alkyl, or a protected hydroxyl group, and B is any suitable nucleobase. (Beaucage & Carruthers). The use of 5′ protected monomers necessitates a deprotection step following each round of addition in which the 5′ protective group is removed to leave a hydroxyl group.

Whatever chemistry is utilized, the stepwise addition of 5′ residues does not occur quantitatively; some oligonucleotides will “miss” the addition of some residues. This results in a synthesis product that includes the desired oligonucleotide, but is contaminated with shorter oligonucleotides missing various residues (referred to as “n−1 species,” though they may include n−2, n−3, etc. as well as other truncation or deletion species). To minimize contamination by n−1 species, many chemical synthesis schemes include a “capping” reaction between the stepwise addition step and the deprotection step. In the capping reaction, a non-reactive moiety is added to the 5′ terminus of those oligonucleotides that are not terminated by a 5′ protective group; this non-reactive moiety prevents the further addition of monomers to the oligonucleotide, and is effective in reducing n−1 contamination to acceptably low levels during the synthesis of oligonucleotides of around 60 or 70 bases in length. However, the capping reaction is not quantitative either, and may be ineffective in preventing n−1 contamination in longer oligonucleotides such as unimolecular guide RNAs. On the other hand, there are occasions where DMT protection is lost during the coupling reaction, which result in longer oligonucleotides (referred to as “n+1 species,” though they may include n+2, n+3, etc.). Unimolecular guide RNAs contaminated with n−1 species and/or n+1 species may not behave in the same ways as full-length guide RNAs prepared by other means, potentially complicating the use of synthesized guide RNAs in therapeutics.

### SUMMARY

This disclosure addresses the need for a cost-effective and straightforward chemical synthesis of high-purity unimolecular guide molecules with minimal n−1 and/or n+1 species, truncation species, and other contaminants by providing, among other things, methods for synthesizing unimolecular guide molecules that involve cross-linking two or more pre-annealed guide fragments. In some embodiments, a unimolecular guide molecule provided herein has improved sequence fidelity at the 5′ end, reducing undesired off-target editing. Also provided herein are compositions comprising, or consisting essentially of, the full length unimolecular guide molecules, which are substantially free of n−1 and/or n+1 contamination.

Certain aspects of this disclosure encompass the realization that pre-annealing of guide fragments may be particularly useful when the guide fragments are homomultifunctional (e.g., homobifunctional), such as the amine-functionalized fragments used in the urea-based cross-linking methods described herein. Indeed, pre-annealing homomultifunctional guide fragments into heterodimers can reduce the formation of undesirable homodimers. This disclosure therefore also provides compositions comprising, or consisting essentially of, the full length unimolecular guide molecules, which are substantially free of side products (for example, homodimers).

In one aspect, the present disclosure relates to a method of synthesizing a unimolecular guide molecule for a CRISPR system, the method comprising the steps of:

annealing a first oligonucleotide and a second oligonucleotide to form a duplex between a 3′ region of the first oligonucleotide and a 5′ region of the second oligonucleotide, wherein the first oligonucleotide comprises a first reactive group which is at least one of a 2′ reactive group and a 3′ reactive group, and wherein the second oligonucleotide comprises a second reactive group which is a 5′ reactive group; and

conjugating the annealed first and second oligonucleotides via the first and second reactive groups to form a unimolecular guide RNA molecule that includes a covalent bond linking the first and second oligonucleotides.

In one aspect, the present disclosure relates to unimolecular guide molecules for a CRISPR system. In some embodiments, a unimolecular guide molecule provided herein is for a Type II CRISPR system.

In some embodiments, a 5′ region of the first oligonucleotide comprises a targeting domain that is fully or partially complementary to a target domain within a target sequence (e.g., a target sequence within a eukaryotic gene).

In some embodiments, a 3′ region of the second oligonucleotide comprises one or more stem-loop structures.

In some embodiments, a unimolecular guide molecule provided herein is capable of interacting with a Cas9 molecule and mediating the formation of a Cas9/guide molecule complex.

In some embodiments, a unimolecular guide molecule provided herein is in a complex with a Cas9 or an RNA-guided nuclease.

In some embodiments, a unimolecular guide molecule provided herein comprises, from 5′ to 3′:
a first guide molecule fragment, comprising:
   a targeting domain sequence;
   a first lower stem sequence;
   a first bulge sequence;
   a first upper stem sequence;
a non-nucleotide chemical linkage; and
a second guide molecule fragment, comprising
   a second upper stem sequence;
   a second bulge sequence; and
   a second lower stem sequence,

wherein (a) at least one nucleotide in the first lower stem sequence is base paired with a nucleotide in the second lower stem sequence, and (b) at least one nucleotide in the first upper stem sequence is base paired with a nucleotide in the second upper stem sequence.

In some embodiments, the unimolecular guide molecule does not include a tetraloop sequence between the first and second upper stem sequences. In some embodiments, the first and/or second upper stem sequence comprises nucleotides that number from 4 to 22, inclusive.

In some embodiments, the unimolecular guide molecule is of formula:

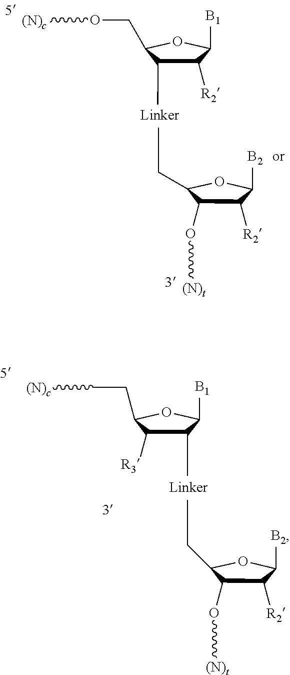

wherein each N in (N)_{c} and (N)ₜ is independently a nucleotide residue, optionally a modified nucleotide residue, each independently linked to its adjacent nucleotide(s) via a phosphodiester linkage, a phosphorothioate linkage, a phosphonoacetate linkage, a thiophosphonoacetate linkage, or a phosphoroamidate linkage;

(N)_{c} includes a 3′ region that is complementary or partially complementary to, and forms a duplex with, a 5′ region of (N)ₜ;

c is an integer 20 or greater;

t is an integer 20 or greater;

Linker is a non-nucleotide chemical linkage;

B₁ and B₂ are each independently a nucleobase;

each of R₂′ and R₃′ is independently H, OH, fluoro, chloro, bromo, NH₂, SH, S—R′, or O—R′ wherein each R′ is independently a protection group or an alkyl group, wherein the alkyl group may be optionally substituted; and

each [symbol] represents independently a phosphodiester linkage, a phosphorothioate linkage, a phosphonoacetate linkage, a thiophosphonoacetate linkage, or a phosphoroamidate linkage.

In some embodiments, (N)_{c} comprises a 3′ region that comprises at least a portion of a repeat from a Type II CRISPR system. In some embodiments, (N)_{c} comprises a 3′ region that comprises a targeting domain that is fully or partially complementary to a target domain within a target sequence. In some embodiments, (N)ₜ comprises a 3′ region that comprises one or more stem-loop structures.

In some embodiments, the unimolecular guide molecule is of formula:

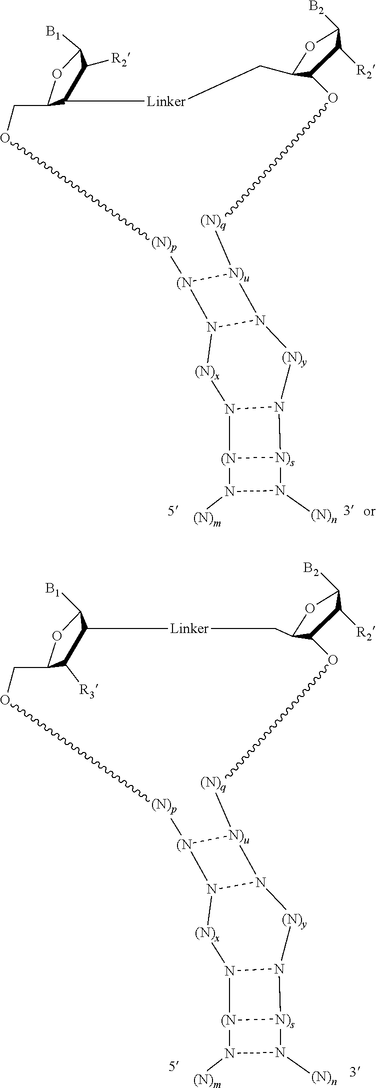

wherein:

each N is independently a nucleotide residue, optionally a modified nucleotide residue, each independently linked to its adjacent nucleotide(s) via a phosphodiester linkage, a phosphorothioate linkage, a phosphonoacetate linkage, a thiophosphonoacetate linkage, or a phosphoroamidate linkage; and

each [symbol] independently represents two complementary nucleotides, optionally two complementary nucleotides that are hydrogen bonding base-paired;

p and q are each 0;

u is an integer between 2 and 22, inclusive;

s is an integer between 1 and 10, inclusive;

x is an integer between 1 and 3, inclusive;

y is >x and an integer between 3 and 5, inclusive;

m is an integer 15 or greater; and

n is an integer 30 or greater.

In some embodiments, u is an integer between 2 and 22, inclusive;

s is an integer between 1 and 8, inclusive;

x is an integer between 1 and 3, inclusive;

y is >x and an integer between 3 and 5, inclusive;

m is an integer between 15 and 50, inclusive; and

n is an integer between 30 and 70, inclusive.

In some embodiments, the guide molecule does not comprise a tetraloop (p and q are each 0). In some embodiments, the lower stem sequence and the upper stem sequence do not comprise an identical sequence of more than 3 nucleotides. In some embodiments, u is an integer between 3 and 22, inclusive.

In some embodiments, a first reactive group and a second reactive group are each an amino group, and the step of conjugating comprises crosslinking the amine moieties of the first and second reactive groups with a carbonate-containing bifunctional crosslinking reagent to form a urea linkage. In some embodiments, a first reactive group and a second reactive group are a bromoacetyl group and a sulfhydryl group. In some embodiments, a first reactive group and a second reactive group are a phosphate group and a hydroxyl group.

In some embodiments, the unimolecular guide molecule comprises a chemical linkage of

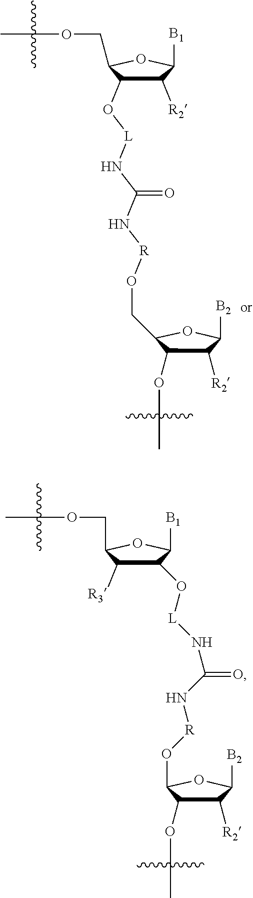

or a pharmaceutically acceptable salt thereof, wherein L and R are each independently a non-nucleotide chemical linker.

In some embodiments, the unimolecular guide molecule comprises a chemical linkage of formula:

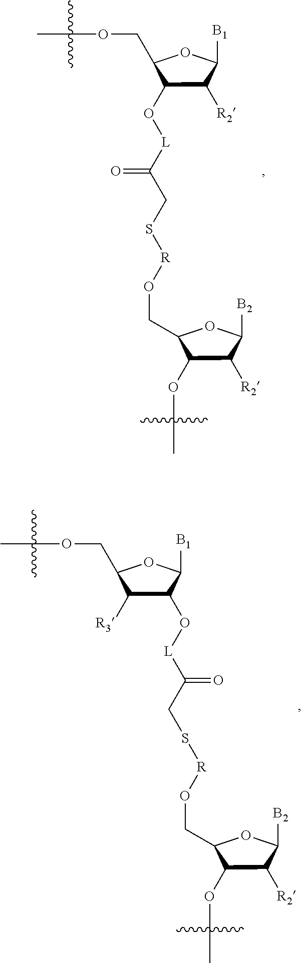

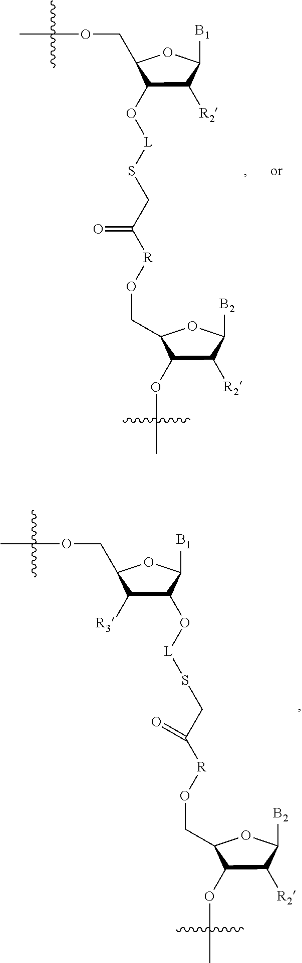

or a pharmaceutically acceptable salt thereof, wherein L and R are each independently a non-nucleotide chemical linker.

In some embodiments, the unimolecular guide molecule is of formula:

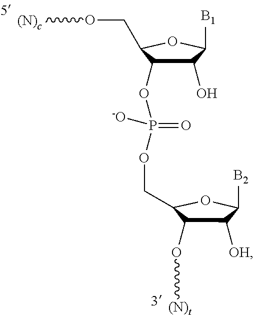

or a pharmaceutically acceptable salt thereof,
and is prepared by a process comprising a reaction between

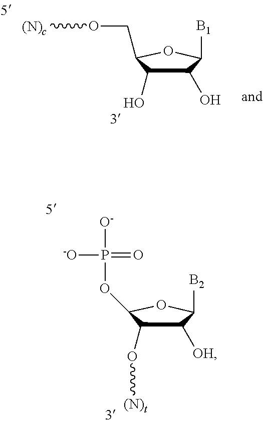

or salts thereof, in the presence of an activating agent to form a phosphodiester linkage.

In one aspect, the present disclosure relates to a composition of guide molecules for a CRISPR system, comprising, or consisting essentially of, unimolecular guide molecules of formula:

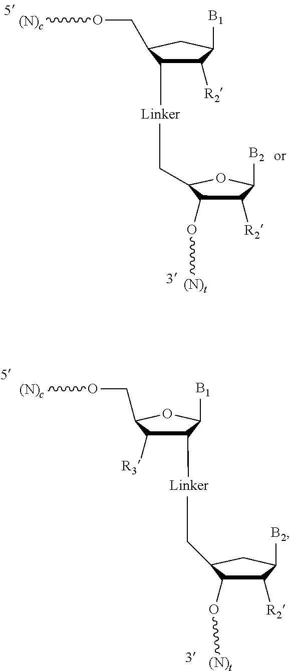

or a pharmaceutically acceptable salt thereof. In some embodiments, less than about 10% of the guide molecules comprise a truncation at a 5′ end, relative to a reference guide molecule sequence. In some embodiments, at least about 99% of the guide molecules comprise a 5′ sequence comprising nucleotides 1-20 of the guide molecule that is 100% identical to a corresponding 5′ sequence of the reference guide molecule sequence.

In some embodiments, the composition of guide molecules comprises, or consists essentially of, guide molecules of formula:

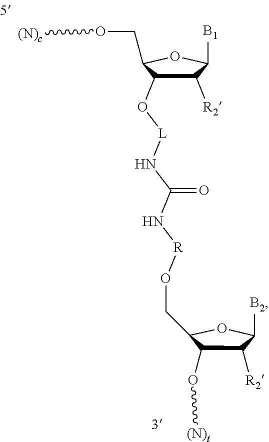

or a pharmaceutically acceptable salt thereof,

wherein the composition is substantially free of molecules of formula:

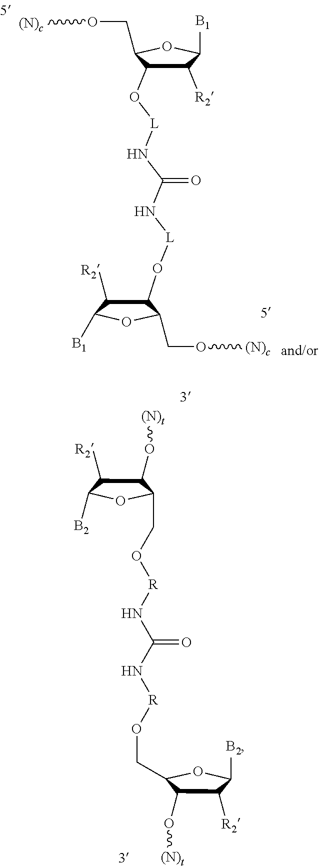

or a pharmaceutically acceptable salt thereof.

In some embodiments, the composition comprises, or consists essentially of, guide molecules of

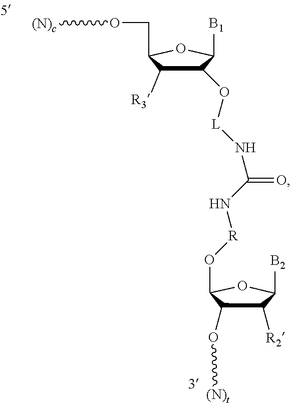

or a pharmaceutically acceptable salt thereof,

wherein the composition is substantially free of molecules of formula:

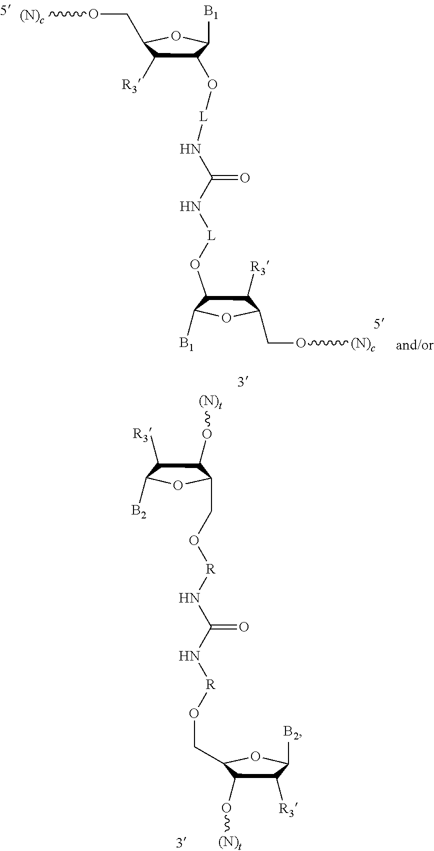

or a pharmaceutically acceptable salt thereof.

In some embodiments, the composition comprises, or consists essentially of, guide molecules of

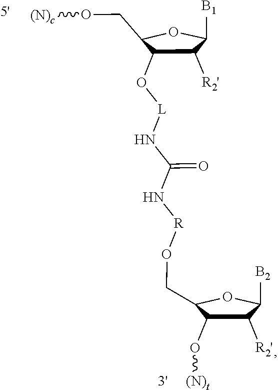

or a pharmaceutically acceptable salt thereof,
wherein the composition is substantially free of molecules of formula:

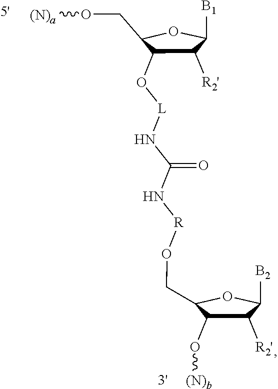

or a pharmaceutically acceptable salt thereof,
wherein:

a is not equal to c; and/or

b is not equal to t.

In some embodiments, the composition comprises, or consists essentially of, guide molecules of

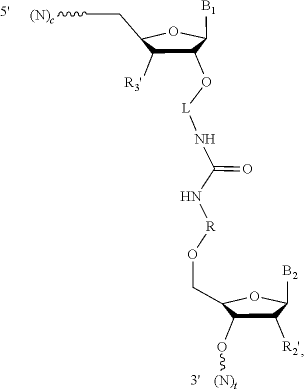

or a pharmaceutically acceptable salt thereof,
wherein the composition is substantially free of molecules of formula:

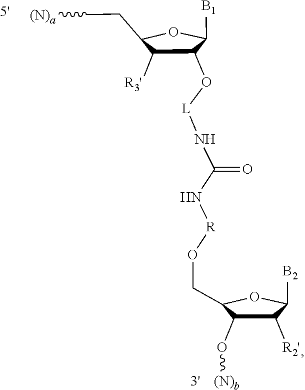

or a pharmaceutically acceptable salt thereof,
wherein:

a is not equal to c; and/or

b is not equal to t.

In some embodiments, the composition comprises, or consists essentially of, guide molecules of

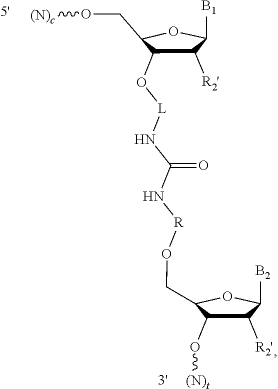

or a pharmaceutically acceptable salt thereof,
wherein the composition is substantially free of molecules of formula:

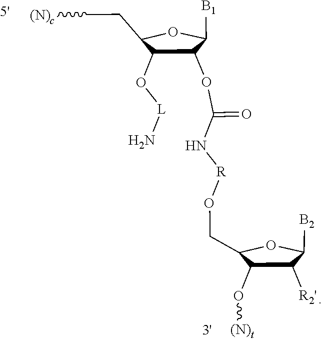

In some embodiments, the composition comprises, or consists essentially of, guide molecules of

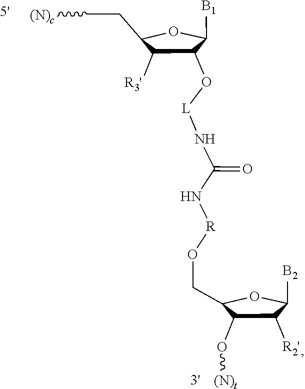

or a pharmaceutically acceptable salt thereof,

wherein the composition is substantially free of molecules of formula:

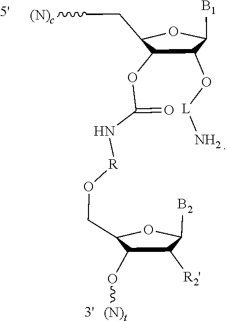

In some embodiments, the composition comprises, or consists essentially of, guide molecules of

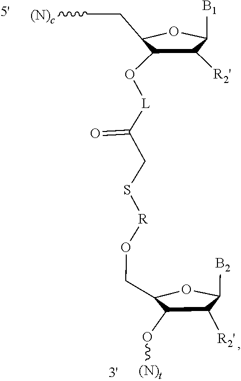

or a pharmaceutically acceptable salt thereof,
wherein the composition is substantially free of molecules of formula:

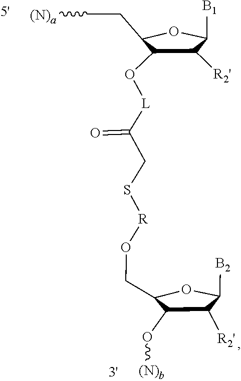

or a pharmaceutically acceptable salt thereof,
wherein:

a is not equal to c; and/or

b is not equal to t.

In some embodiments, the composition comprises, or consists essentially of, guide molecules of

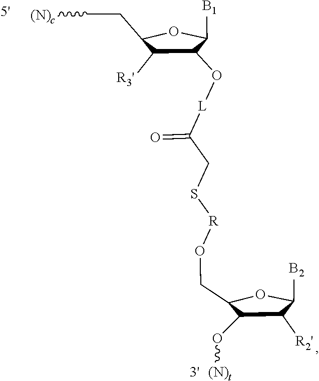

or a pharmaceutically acceptable salt thereof,
wherein the composition is substantially free of molecules of formula:

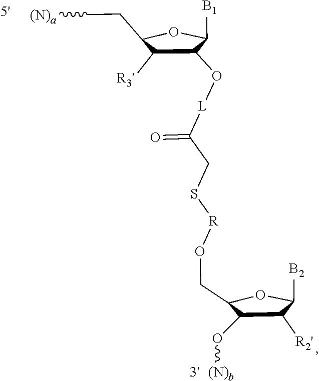

or a pharmaceutically acceptable salt thereof,
wherein:

a is not equal to c; and/or

b is not equal to t.

In some embodiments, the composition comprises, or consists essentially of, guide molecules of

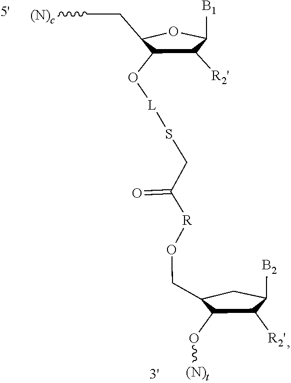

or a pharmaceutically acceptable salt thereof,
wherein the composition is substantially free of molecules of formula:

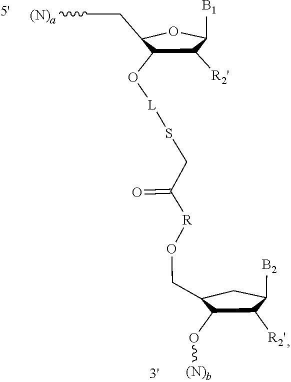

or a pharmaceutically acceptable salt thereof,
wherein:

a is not equal to c; and/or

b is not equal to t.

In some embodiments, the composition comprises, or consists essentially of, guide molecules of

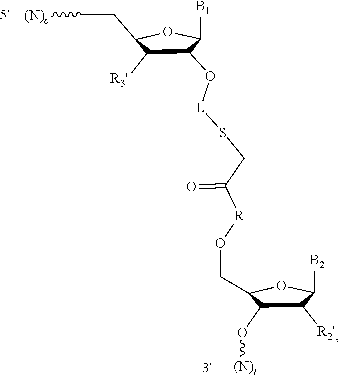

or a pharmaceutically acceptable salt thereof,
wherein the composition is substantially free of molecules of formula:

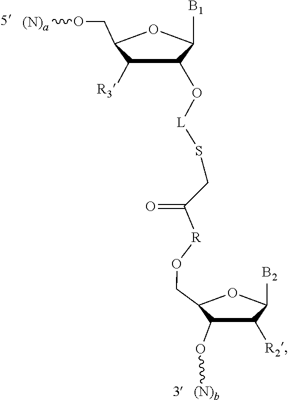

or a pharmaceutically acceptable salt thereof,
wherein:

a is not equal to c; and/or

b is not equal to t.

In some embodiments, the composition comprises

(a) a synthetic unimolecular guide molecule for a CRISPR system, wherein the guide molecule is of formula:

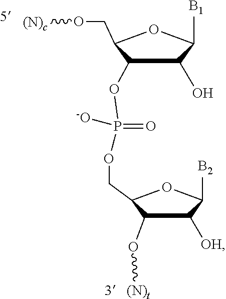

or a pharmaceutically acceptable salt thereof; and

(b) one or more of:
(i) a carbodiimide, or a salt thereof;
(ii) imidazole, cyanoimidazole, pyridine, and dimethylaminopyridine, or a salt thereof; and
(iii) a compound of formula:

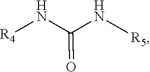

or a salt thereof, wherein R₄ and R₅ are each independently substituted or unsubstituted alkyl, or substituted or unsubstituted carbocyclic.

In some embodiments, the composition comprises

a synthetic unimolecular guide molecule for a CRISPR system, wherein the guide molecule is of formula:

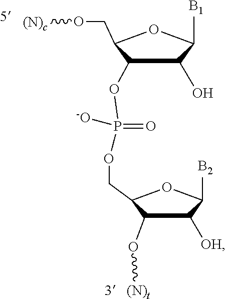

or a pharmaceutically acceptable salt thereof,
wherein the composition is substantially free of molecules of formula:

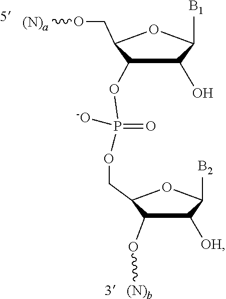

or a pharmaceutically acceptable salt thereof,
wherein:

a+b is c+t−k, wherein k is 1-10.

In some embodiments, the composition comprises, or consists essentially of, a synthetic unimolecular guide molecule for a CRISPR system, wherein the guide molecule is of formula:

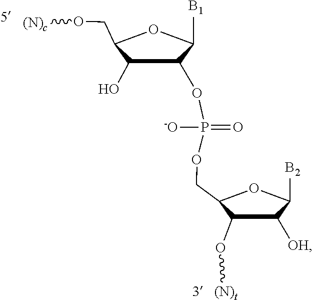

or a pharmaceutically acceptable salt thereof,
wherein the 2′-5′ phosphodiester linkage depicted in the formula is between two nucleotides in a duplex formed between a 3′ region of (N)_{c} and a 5′ region of (N)ₜ.

In one aspect, the present disclosure relates to oligonucleotides for synthesizing a unimolecular guide molecule provided herein and/or for synthesizing a unimolecular guide molecule by a method provided herein. In some embodiments, the oligonucleotide is of formula:

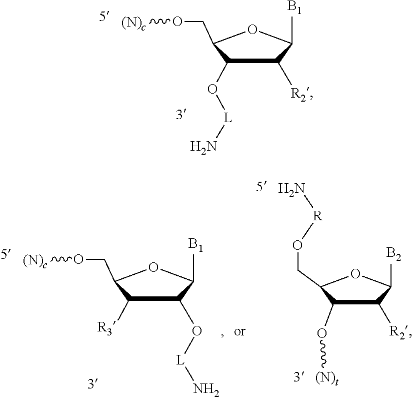

or salt thereof.

In some embodiments, the oligonucleotide is of formula:

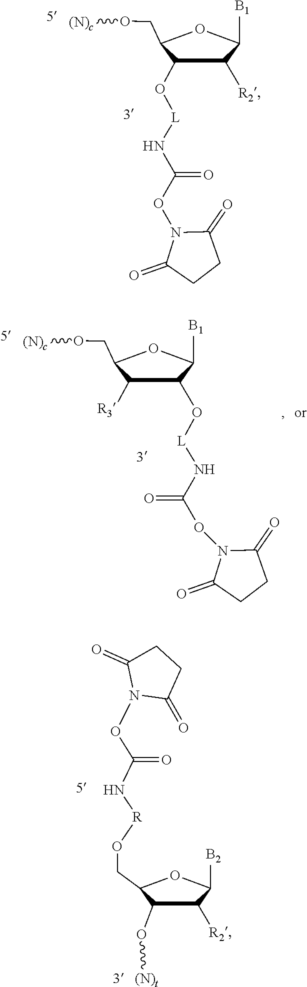

or salt thereof.

In some embodiments, the oligonucleotide is of formula:

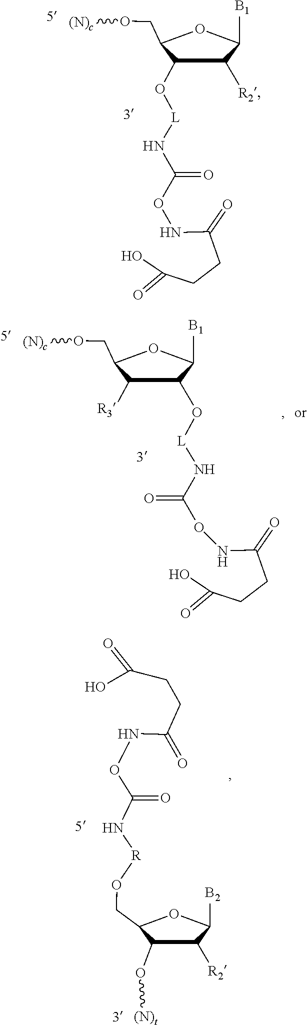

or salt thereof.

In some embodiments, a composition comprises oligonucleotides with an annealed duplex of formula:

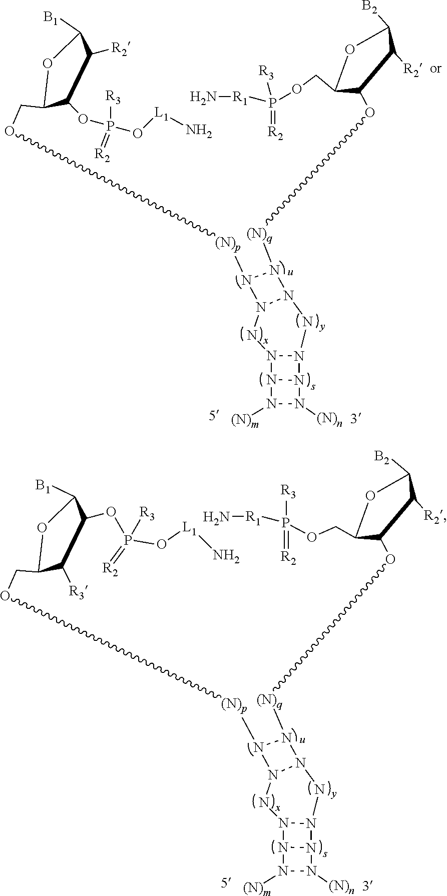

or salt thereof.

In some embodiments, the oligonucleotide is of formula:

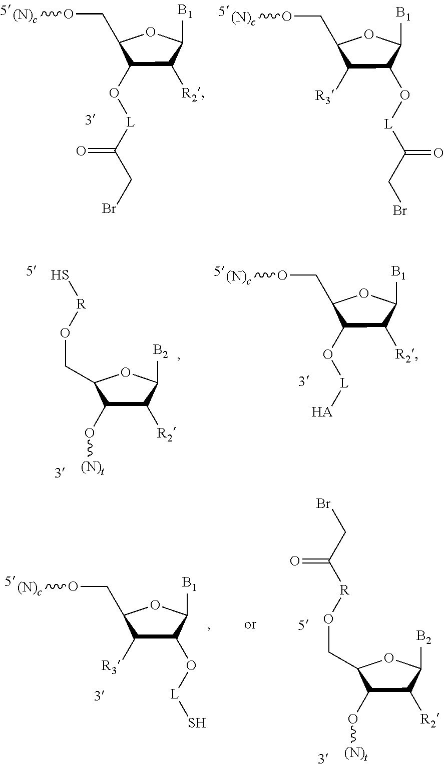

or a salt thereof.

In some embodiments, the oligonucleotide is of formula:

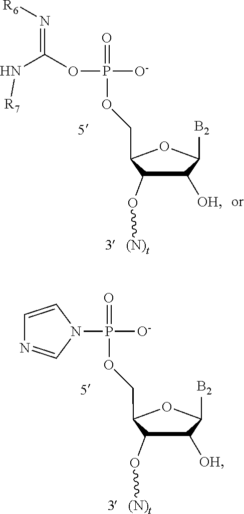

or a salt thereof.

In some embodiments, a composition comprises oligonucleotides with an annealed duplex of

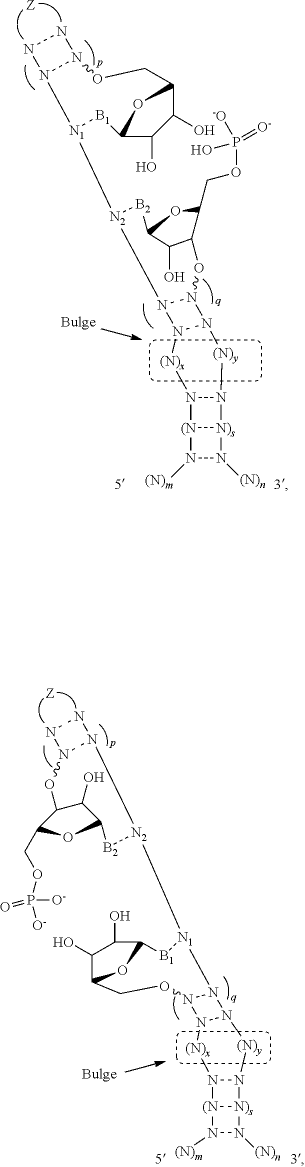

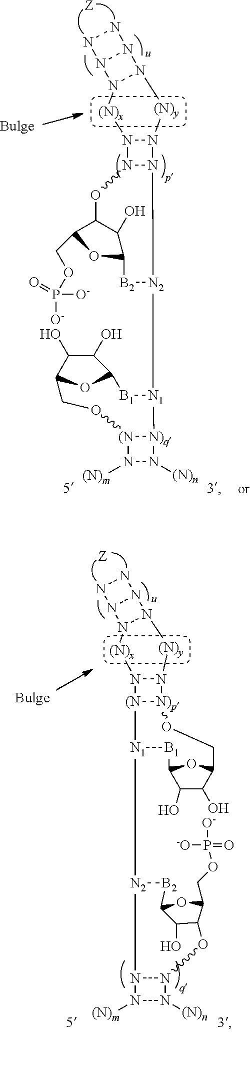

or a salt thereof.

In one aspect, the present disclosure relates to a compound of formula:

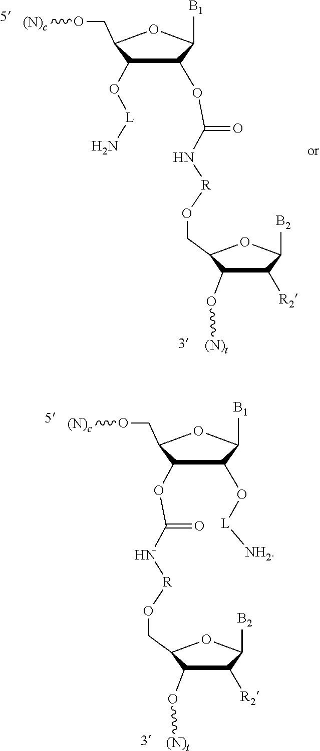

In one aspect, the present disclosure relates to a method of altering a nucleic acid in a cell or subject comprising administering to the subject a guide molecule or a composition provided herein.

In some embodiments, a composition provided herein has not been subjected to any purification steps.

In some embodiments, a composition provided herein comprises a unimolecular guide RNA molecule suspended in solution or in a pharmaceutically acceptable carrier.

In one aspect, the present disclosure relates to a genome editing system comprising a guide molecule provided herein. In some embodiments, the genome editing system and/or the guide molecule is for use in therapy. In some embodiments, the genome editing system and/or the guide molecule is for use in the production of a medicament.

### BRIEF DESCRIPTION OF THE DRAWINGS

The accompanying drawings are intended to provide illustrative, and schematic rather than comprehensive, examples of certain aspects and embodiments of the present disclosure. The drawings are not intended to be limiting or binding to any particular theory or model, and are not necessarily to scale. Without limiting the foregoing, nucleic acids and polypeptides may be depicted as linear sequences, or as schematic two- or three-dimensional structures; these depictions are intended to be illustrative rather than limiting or binding to any particular model or theory regarding their structure.

FIG. 1A depicts an exemplary cross-linking reaction process according to certain embodiments of this disclosure.

FIG. 1B depicts, in two-dimensional schematic form, an exemplary S. pyogenes guide molecule highlighting positions (with a star) at which first and second guide molecule fragments are cross-linked together according to various embodiments of this disclosure.

FIG. 1C depicts, in two-dimensional schematic form, an exemplary S. aureus guide molecule highlighting positions (with a star) at which first and second guide molecule fragments are cross-linked together according to various embodiments of this disclosure.

FIG. 2A depicts a step in an exemplary cross-linking reaction process according to certain embodiments of this disclosure.

FIG. 2B depicts a step in an exemplary cross-linking reaction process according to certain embodiments of this disclosure.

FIG. 2C depicts an additional step in the exemplary cross-linking reaction process using the reaction products from FIGS. 2A and 2B.

FIG. 3A depicts an exemplary cross-linking reaction process according to certain embodiments of this disclosure.

FIG. 3B depicts steps in an exemplary cross-linking reaction process according to certain embodiments of this disclosure.

FIG. 3C depicts, in two-dimensional schematic form, an exemplary S. pyogenes guide molecule highlighting positions at which first and second guide molecule fragments are cross-linked together according to various embodiments of this disclosure.

FIG. 3D depicts, in two-dimensional schematic form, an exemplary S. aureus guide molecule highlighting positions at which first and second guide molecule fragments are cross-linked together according to various embodiments of this disclosure.

FIG. 4 shows DNA cleavage dose-response curves for synthetic unimolecular guide molecules according to certain embodiments of this disclosure as compared to unligated, annealed guide molecule fragments and guide molecules prepared by IVT obtained from a commercial vendor. DNA cleavage was assayed by T7E1 assays as described herein. As the graph shows, the conjugated guide molecule supported cleavage in HEK293 cells in a dose-dependent manner that was consistent with that observed with the unimolecular guide molecule generated by IVT or the synthetic unimolecular guide molecule. It should be noted that unconjugated annealed guide molecule fragments supported a lower level of cleavage, though in a similar dose-dependent manner.

FIG. 5A shows a representative ion chromatograph and FIG. 5B shows a deconvoluted mass spectrum of an ion-exchange purified guide molecule conjugated with a urea linker according to the process of Example 1. FIG. 5C shows a representative ion chromatograph and FIG. 5D shows a deconvoluted mass spectrum of a commercially prepared synthetic unimolecular guide molecule. Mass spectra were assessed for the highlighted peaks in the ion chromatographs. FIG. 5E shows expanded versions of the mass spectra. The mass spectrum for the commercially prepared synthetic unimolecular guide molecule is on the left side (34% purity by total mass) while the mass spectrum for the guide molecule conjugated with a urea linker according to the process of Example 1 is on the right side (72% purity by total mass).

FIG. 6A shows a plot depicting the frequency with which individual bases and length variances occurred at each position from the 5′ end of complementary DNAs (cDNAs) generated from synthetic unimolecular guide molecules that included a urea linkage, and FIG. 6B shows a plot depicting the frequency with which individual bases and length variances occurred at each position from the 5′ end of cDNAs generated from commercially prepared synthetic unimolecular guide molecules (i.e., prepared without conjugation). Boxes surround the 20 bp targeting domain of the guide molecule. FIG. 6C shows a plot depicting the frequency with which individual bases and length variances occurred at each position from the 5′ end of cDNAs generated from synthetic unimolecular guide molecules that included the thioether linkage.

FIG. 7A and FIG. 7B are graphs depicting internal sequence length variances (+5 to −5) at the first 41 positions from the 5′ ends of cDNAs generated from various synthetic unimolecular guide molecules that included the urea linkage (FIG. 7A), and from commercially prepared synthetic unimolecular guide molecules (i.e., prepared without conjugation) (FIG. 7B).

FIGS. 8A-8H depict, in two-dimensional schematic form, the structures of certain exemplary guide molecules according to various embodiments of this disclosure. Complementary bases capable of base pairing are denoted by one (A-U or A-T pairing) or two (G-C) horizontal lines between bases. Bases capable of non-Watson-Crick pairing are denoted by a single horizontal line with a circle.

FIGS. 9A-9D depict, in two-dimensional schematic form, the structures of certain exemplary guide molecules according to various embodiments of this disclosure. Complementary bases capable of base pairing are denoted by one (A-U or A-T pairing) or two (G-C) horizontal lines between bases. Bases capable of non-Watson-Crick pairing are denoted by a single horizontal line with a circle.

FIGS. 10A-10D depict, in two-dimensional schematic form, the structures of certain exemplary guide molecules according to various embodiments of this disclosure. Complementary bases capable of base pairing are denoted by one (A-U or A-T pairing) or two (G-C) horizontal lines between bases. Bases capable of non-Watson-Crick pairing are denoted by a single horizontal line with a circle.

FIG. 11 shows a graph of DNA cleavage in CD34+ cells with a series of ribonucleoprotein complexes comprising conjugated guide molecules from Table 10. Cleavage was assessed using next generation sequencing techniques to quantify % insertions and deletions (indels) relative to a wild-type human reference sequence. Ligated guide molecules generated according to Example 1 support DNA cleavage in CD34+ cells. % indels were found to increase with increasing stemloop length, but incorporation of a U-A swap adjacent to the stemloop sequence (see gRNAs 1E, IF, and 2D) mitigates the effect.

FIG. 12A shows a liquid chromatography-mass spectrometry (LC-MS) trace after T1 endonuclease digestion of gRNA 1A, and FIG. 12B shows a mass spectrum of the peak with a retention time of 4.50 min (A34:G39). In particular, the fragment containing the urea linkage, A-[UR]-AAUAG (A34:G39), was detected at a retention time of 4.50 min with m/z=1190.7.

FIG. 13A shows LC-MS data for an unpurified composition of urea-linked guide molecules with both a major product (A-2, retention time of 3.25 min) and a minor product (A-1, retention time of 3.14 min) present. We note that the minor product (A-1) in FIG. 13A was enriched for purposes of illustration and is typically detected in up to 10% yield in the synthesis of guide molecules in accordance with the process of Example 1. FIG. 13B shows a deconvoluted mass spectrum of peak A-2 (retention time of 3.25 min), and FIG. 13C shows a deconvoluted mass spectrum of peak A-1 (retention time of 3.14 min). Analysis of each peak by mass spectrometry indicated that both products have the same molecular weight.

FIG. 14A shows LC-MS data for the guide molecule composition after chemical modification as described in Example 10. The major product (B-1, urea) has the same retention time as in the original analysis (3.26 min), while the retention time of minor product (B-2, carbamate) has shifted to 3.86 min, consistent with chemical functionalization of the free amine moiety. FIG. 14B shows a mass spectrum of peak B-2 (retention time of 3.86 min). Analysis of the peak at 3.86 min (M+134) indicates the predicted functionalization has occurred.

FIG. 15A shows the LC-MS trace of the fragment mixture after digestion with T1 endonuclease of a reaction mixture containing both major product (urea) and chemically modified minor product (carbamate). Both the urea linkage (G35-[UR]-C36) and the chemically modified carbamate linkage (G35-[CA+PAA]-C36) were detected at retention times of 4.31 min and 5.77 min, respectively. FIG. 15B shows the mass spectrum of the peak at 4.31 min, where m/z=532.13 is assigned to [M−2H]²⁻, and FIG. 15C shows the mass spectrum of the peak at 5.77 min, where m/z=599.15 is assigned to [M−2H]²⁻. FIG. 15D and FIG. 15E show LC/MS-MS collision-induced dissociation (CID) experiments of m/z=532.1 from FIG. 15B and of m/z=599.1 from FIG. 15C. In FIG. 15D, the typical a-d and x-z ions were observed, and MS/MS fragment ions on either side of the UR linkage from the 5′-end (m/z=487.1 and 461.1) and the 3′-end (m/z=603.1 and 577.1) were observed. In FIG. 15E, only two product ions were observed, including a MS/MS fragment ion from the 5′-end of the carbamate linkage (m/z=595.2) and the 3′-end of the CA linkage (m/z=603.1).

FIG. 16A shows LC-MS data of the crude reaction mixture for a reaction with a 2′-H modified 5′ guide molecule fragment (upper spectrum), compared to a crude reaction mixture for a reaction with an unmodified version of the same 5′ guide molecule (lower spectrum). There is no carbamate side product formation observed with the 2′-H modified 5′ guide molecule fragment (upper spectrum). In contrast, the crude reaction mixture for a reaction with an unmodified version of the same 5′ guide molecule fragment (lower spectrum) included a mixture of the major urea-linked product (A-2) and the minor carbamate side product (A-1). We note that, unlike in Example 10, the carbamate side product was not enriched and was therefore detected at much lower levels than in FIG. 13A of Example 10. FIG. 16B shows a deconvoluted mass spectrum of peak B (retention time of 3.14 min, upper spectrum of FIG. 16A), and FIG. 16C shows a deconvoluted mass spectrum of peak A-2 (retention time of 3.45 min, lower spectrum of FIG. 16A). Analysis of the product of the reaction with the 2′-H modified 5′ guide molecule fragment (B) gave M-16 (compared to A-2, the major unmodified urea-linked product), as expected for a molecule where a 2′-OH has been replaced with a 2′-H (see FIG. 16B and FIG. 16C).

FIG. 17A shows a LC-MS trace after T1 endonuclease digestion of gRNA 1L, and FIG. 17B shows a mass spectrum of the peak with a retention time of 4.65 min (A34:G39). In particular, the fragment containing the urea linkage, A-[UR]-AAUAG (A34:G39), was detected at a retention time of 4.65 min with m/z=1182.7.

### DETAILED DESCRIPTION

### Definitions and Abbreviations

Unless otherwise specified, each of the following terms has the meaning associated with it in this section.

The indefinite articles “a” and “an” refer to at least one of the associated noun, and are used interchangeably with the terms “at least one” and “one or more.” For example, “a module” means at least one module, or one or more modules.

The conjunctions “or” and “and/or” are used interchangeably as non-exclusive disjunctions.

The phrase “consisting essentially of” means that the species recited are the predominant species, but that other species may be present in trace amounts or amounts that do not affect structure, function or behavior of the subject composition. For instance, a composition that consists essentially of a particular species will generally comprise 90%, 95%, 96%, or more (by mass or molarity) of that species.

The phrase “substantially free of molecules” means that the molecules are not major components in the recited composition. For example, a composition substantially free of a molecule means that the molecule is less than 5%, 4%, 3%, 2%, 1%, 0.5%, or 0.1% (by mass or molarity) in the composition. The amount of a molecule can be determined by various analytical techniques, e.g., as described in the Examples. In some embodiments, compositions provided herein are substantially free of certain molecules, wherein the molecules are less than 5%, 4%, 3%, 2%, 1%, 0.5%, or 0.1% (by mass or molarity) as determined by gel electrophoresis. In some embodiments, compositions provided herein are substantially free of certain molecules, wherein the molecules are less than 5%, 4%, 3%, 2%, 1%, 0.5%, or 0.1% (by mass or molarity) as determined by mass spectrometry.

“Domain” is used to describe a segment of a protein or nucleic acid. Unless otherwise indicated, a domain is not required to have any specific functional property.

The term “complementary” refers to pairs of nucleotides that are capable of forming a stable base pair through hydrogen bonding. For example, U is complementary to A and G is complementary to C. It will be appreciated by those skilled in the art that whether a particular pair of complementary nucleotides are associated through hydrogen bond base pairing (e.g., within a guide molecule duplex) may depend on the context (e.g., surrounding nucleotides and chemical linkage) and external conditions (e.g., temperature and pH). It is therefore to be understood that complementary nucleotides are not necessarily associated through hydrogen bond base pairing.

A “covariant” sequence differs from a reference sequence by substitution of one or more nucleotides in the reference sequence with a complementary nucleotide (e.g., one or more Us are replaced with As, one or more Gs are replaced with Cs, etc.). When used with reference to a region that includes two complementary sequences that form a duplex (e.g., the upper stem of a guide molecule), the term “covariant” encompasses duplexes with one or more nucleotide swaps between the two complementary sequences of the reference duplex (i.e., one or more A-U swaps and/or one or more G-C swaps) as illustrated in Table 1 below:

TABLE 1
Covariant sequences of a sequence of three nucleotides.

| | |
|---|---|
| A----U | U----A |
| G----C | G----C |
| C----G | C----G |
| A----U | A----U |
| C----G | G----C |
| C----G | G----C |
| U----A | U----A |
| C----G | G----C |
| C----G | G----C |
| A----U | U----A |
| C----G | C----G |
| G----C | G----C |

In some embodiments, a covariant sequence may exhibit substantially the same energetic favorability of a particular annealing reaction as the reference sequence (e.g., formation of a duplex in the context of a guide molecule of the present disclosure). As described elsewhere in the present disclosure, the energetic favorability of a particular annealing reaction may be measured empirically or predicted using computational models.

An “indel” is an insertion and/or deletion in a nucleic acid sequence. An indel may be the product of the repair of a DNA double strand break, such as a double strand break formed by a genome editing system of the present disclosure. An indel is most commonly formed when a break is repaired by an “error prone” repair pathway such as the NHEJ pathway described below.

“Gene conversion” refers to the alteration of a DNA sequence by incorporation of an endogenous homologous sequence (e.g., a homologous sequence within a gene array). “Gene correction” refers to the alteration of a DNA sequence by incorporation of an exogenous homologous sequence, such as an exogenous single- or double stranded donor template DNA. Gene conversion and gene correction are products of the repair of DNA double-strand breaks by HDR pathways such as those described below.

Indels, gene conversion, gene correction, and other genome editing outcomes are typically assessed by sequencing (most commonly by “next-gen” or “sequencing-by-synthesis” methods, though Sanger sequencing may still be used) and are quantified by the relative frequency of numerical changes (e.g., ±1, ±2 or more bases) at a site of interest among all sequencing reads. DNA samples for sequencing may be prepared by a variety of methods known in the art, and may involve the amplification of sites of interest by polymerase chain reaction (PCR), the capture of DNA ends generated by double strand breaks, as in the GUIDEseq process described in Tsai et al. (Nat. Biotechnol. 34(5): 483 (2016), incorporated by reference herein) or by other means well known in the art. Genome editing outcomes may also be assessed by in situ hybridization methods such as the FiberComb™ system commercialized by Genomic Vision (Bagneux, France), and by any other suitable methods known in the art.

“Alt-HDR,” “alternative homology-directed repair,” or “alternative HDR” are used interchangeably to refer to the process of repairing DNA damage using a homologous nucleic acid (e.g., an endogenous homologous sequence, e.g., a sister chromatid, or an exogenous nucleic acid, e.g., a template nucleic acid). Alt-HDR is distinct from canonical HDR in that the process utilizes different pathways from canonical HDR, and can be inhibited by the canonical HDR mediators, RAD51 and BRCA2. Alt-HDR is also distinguished by the involvement of a single-stranded or nicked homologous nucleic acid template, whereas canonical HDR generally involves a double-stranded homologous template.

“Canonical HDR,” “canonical homology-directed repair” or “cHDR” refer to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA. In a normal cell, cHDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded.

Unless indicated otherwise, the term “HDR” as used herein encompasses both canonical HDR and alt-HDR.

“Non-homologous end joining” or “NHEJ” refers to ligation mediated repair and/or non-template mediated repair including canonical NHEJ (cNHEJ) and alternative NHEJ (altNHEJ), which in turn includes microhomology-mediated end joining (MMEJ), single-strand annealing (SSA), and synthesis-dependent microhomology-mediated end joining (SD-MMEJ).

“Replacement” or “replaced,” when used with reference to a modification of a molecule (e.g., a nucleic acid or protein), does not require a process limitation but merely indicates that the replacement entity is present.

“Subject” means a human or non-human animal. A human subject can be any age (e.g., an infant, child, young adult, or adult), and may suffer from a disease, or may be in need of alteration of a gene. Alternatively, the subject may be an animal, which term includes, but is not limited to, mammals, birds, fish, reptiles, amphibians, and more particularly non-human primates, rodents (such as mice, rats, hamsters, etc.), rabbits, guinea pigs, dogs, cats, and so on. In certain embodiments of this disclosure, the subject is livestock, e.g., a cow, a horse, a sheep, or a goat. In certain embodiments, the subject is poultry.

“Treat,” “treating,” and “treatment” mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

“Prevent,” “preventing,” and “prevention” refer to the prevention of a disease in a mammal, e.g., in a human, including (a) avoiding or precluding the disease; (b) affecting the predisposition toward the disease; or (c) preventing or delaying the onset of at least one symptom of the disease.

A “Kit” refers to any collection of two or more components that together constitute a functional unit that can be employed for a specific purpose. By way of illustration (and not limitation), one kit according to this disclosure can include a guide RNA complexed or able to complex with an RNA-guided nuclease, and accompanied by (e.g., suspended in, or suspendable in) a pharmaceutically acceptable carrier. The kit can be used to introduce the complex into, for example, a cell or a subject, for the purpose of causing a desired genomic alteration in such cell or subject. The components of a kit can be packaged together, or they may be separately packaged. Kits according to this disclosure also optionally include directions for use (DFU) that describe the use of the kit, e.g., according to a method of this disclosure. The DFU can be physically packaged with the kit, or it can be made available to a user of the kit, for instance by electronic means.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides, nucleotide sequences, nucleic acids, etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

Conventional IUPAC notation is used in nucleotide sequences presented herein, as shown in Table 2, below (see also Cornish-Bowden A, Nucleic Acids Res. 1985 May 10; 13(9):3021-30, incorporated by reference herein). It should be noted, however, that “T” denotes “Thymine or Uracil” in those instances where a sequence may be encoded by either DNA or RNA, for example in guide molecule targeting domains.

TABLE 2
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

The term “variant” refers to an entity such as a polypeptide, polynucleotide or small molecule that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity.

### Overview

Certain embodiments of this disclosure relate, in general, to methods for synthesizing guide molecules in which two or more guide fragments are (a) annealed to one another, and then (b) cross-linked using an appropriate cross-linking chemistry. The inventors have found that methods comprising a step of pre-annealing guide fragments prior to cross-linking them improves the efficiency of cross-linking and tends to favor the formation of a desired heterodimeric product, even when a homomultifunctional cross-linker is used. While not wishing to be bound by any theory, the improvements in cross-linking efficiency and, consequently, in the yield of the desired reaction product, are thought to be due to the increased stability of an annealed heterodimer as a cross-linking substrate as compared with non-annealed homodimers, and/or the reduction in the fraction of free RNA fragments available to form homodimers, etc. achieved by pre-annealing.

The methods of this disclosure, which include pre-annealing of guide fragments, have a number of advantages, including without limitation: they allow for high yields to be achieved even when the fragments are homomultifunctional (e.g., homobifunctional), such as the amine-functionalized fragments used in the urea-based cross-linking methods described herein; the reduction or absence of undesirable homodimers and other reaction products may in turn simplify downstream purification; and because the fragments used for cross-linking tend to be shorter than full-length guide molecules, they may exhibit a lower level of contamination by n−1 species, truncation species, n+1 species, and other contaminants than observed in full-length synthetic guide molecules.

With respect to pre-annealing, those of skill in the art will appreciate that longer tracts of annealed bases may be more stable than shorter tracts, and that between two tracts of similar length, a greater degree of annealing will generally be associated with greater stability. Accordingly, in certain embodiments of this disclosure, fragments are designed so as to maximize the degree of annealing between fragments, and/or to position functionalized 3′ or 5′ ends in close proximity to annealed bases and/or to each other.

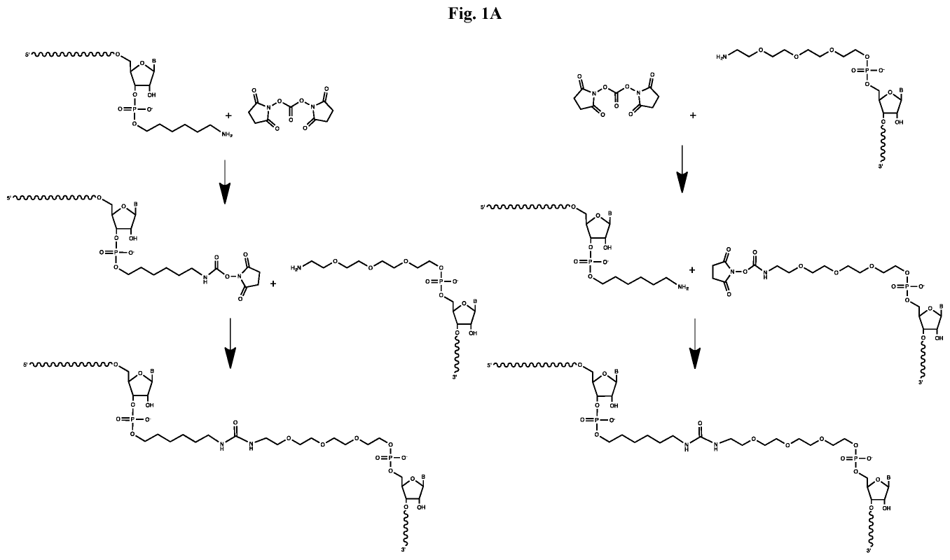

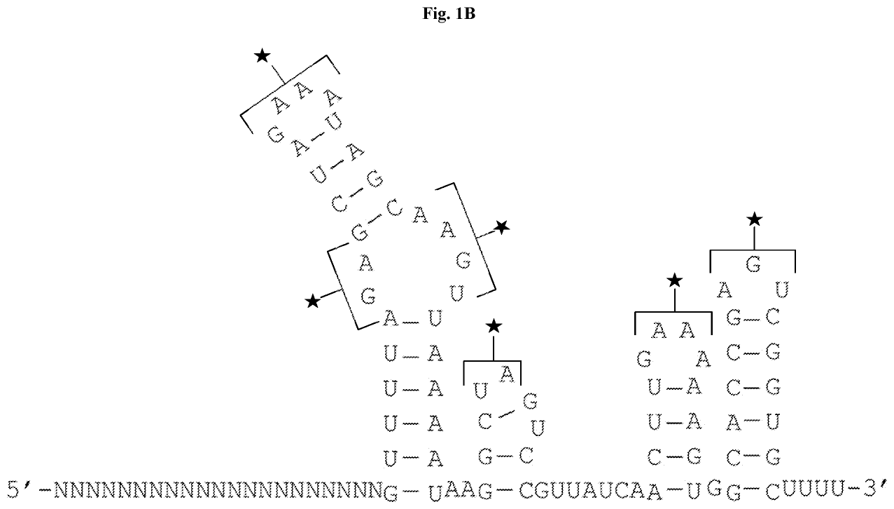

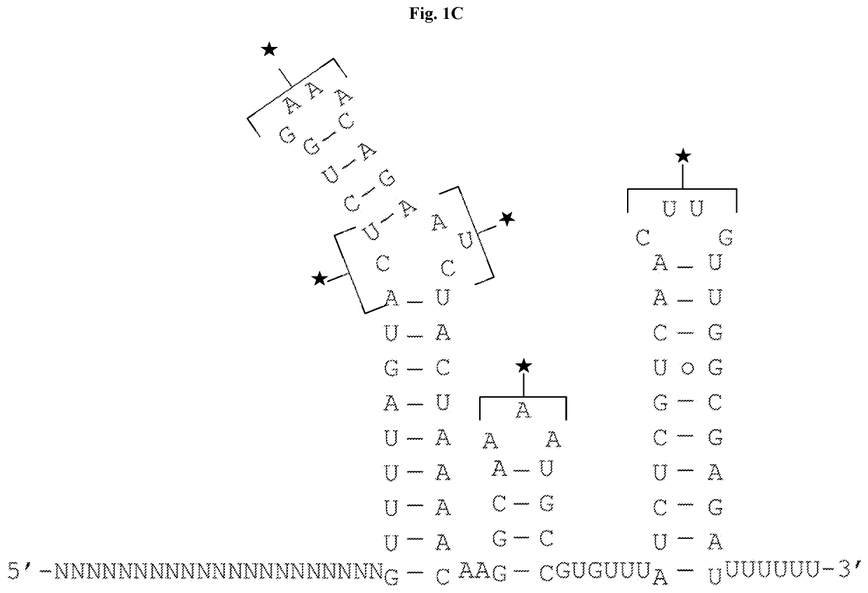

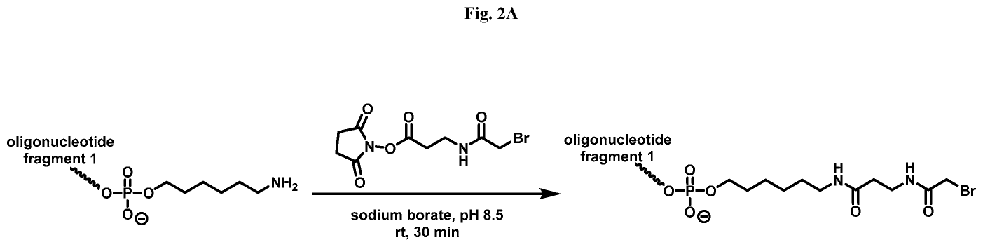

As is discussed in greater detail below, certain unimolecular guide molecules, particularly unimolecular Cas9 guide molecules, are characterized by comparatively large stem-loop structures. For example, FIGS. 1B and 1C depict the two-dimensional structures of unimolecular S. pyogenes and S. aureus gRNAs, and it will be evident from the figures that both gRNAs generally include a relatively long stem-loop structure with a “bulge.” In certain embodiments, synthetic guide molecules include a cross-link between fragments within this stem loop structure. This is achieved, in some cases, by cross-linking first and second fragments having complementary regions at or near their 3′ and 5′ ends, respectively; the 3′ and 5′ ends of these fragments are functionalized to facilitate the cross-linking reaction, as shown for example in Formulae II and III, below:

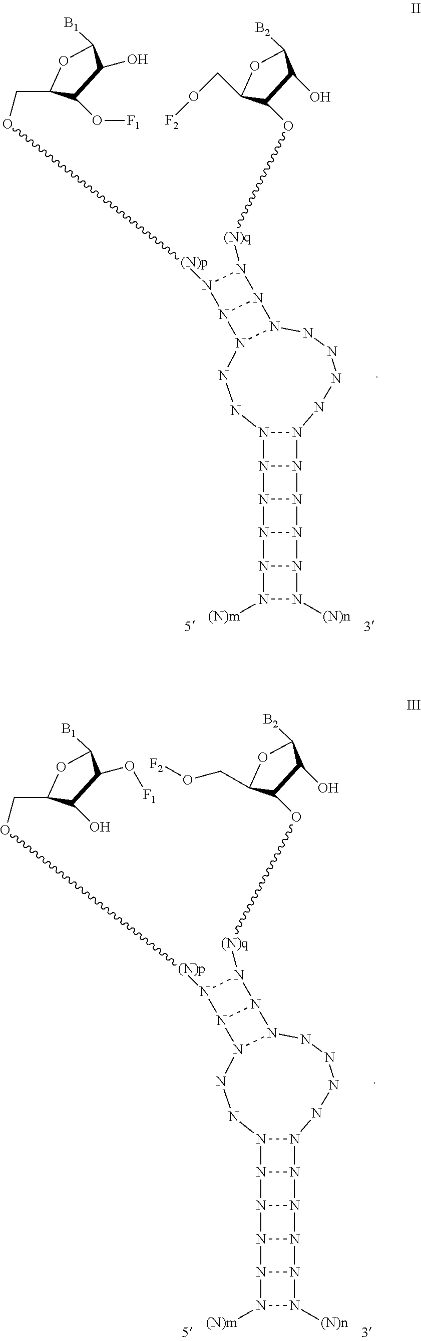

In these formulas, p and q are each independently 0-6, and p+q is 0-6; m is 20-40; n is 30-70; each [symbol] independently represents hydrogen bonding between corresponding nucleotides; each [symbol] represents a phosphodiester linkage, a phosphorothioate linkage, a phosphonoacetate linkage, a thiophosphonoacetate linkage, or a phosphoroamidate linkage; [symbol] independently represents two complementary nucleotides, optionally two complementary nucleotides that are hydrogen bonding base-paired; and F₁ and F₂ each comprise a functional group such that they can undergo a cross-linking reaction to cross-link the two guide fragments. Exemplary cross-linking chemistries are set forth in Table 3 below.

TABLE 3
Exemplary cross-linking chemistries

| Reaction | |
|---|---|
| Type | Reaction Summary |
| Thiol-yne | 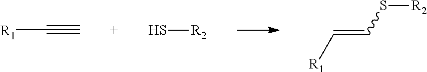 |
| NHS esters | 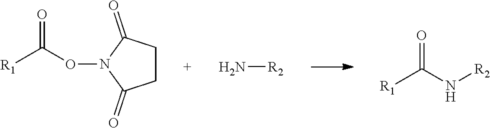 |
| Thiol-ene | 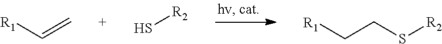 |
| Isocyanates | 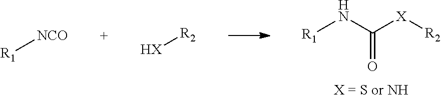 |
| Epoxide or aziridine | 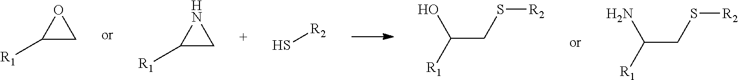 |
| Aldehyde- aminoxy | 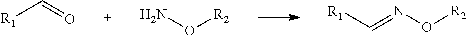 |
| Cu-catalyzed- azide-alkyne cycloaddition | 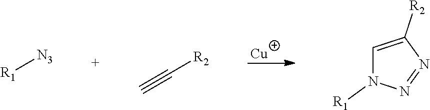 |
| Strain- promoted cycloaddition | Cyclooctyne cycloaddition (with azide or nitrile oxide or nitrone) 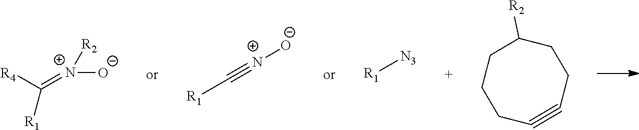 |
| | 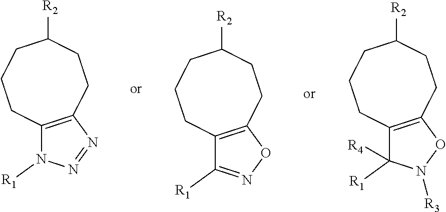 |
| | Norbornene cycloaddition (with azide or nitrile oxide or nitrone) 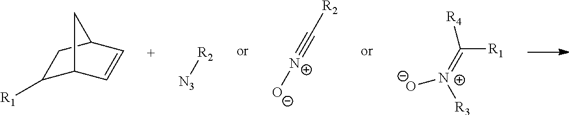 |
| | 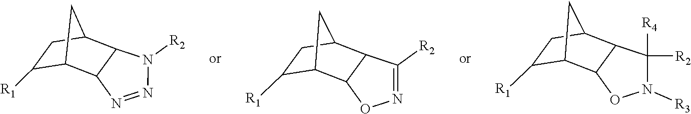 |
| | Oxanorbornadiene cycloaddition 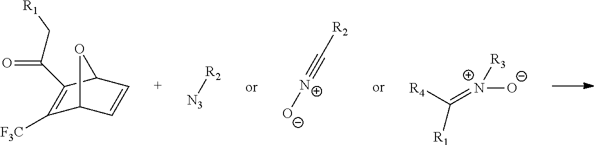 |
| | 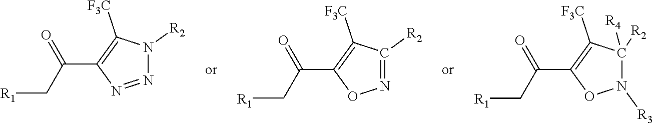 |
| Staudinger ligation | 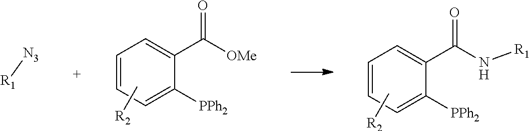 |
| Tetrazine ligation | 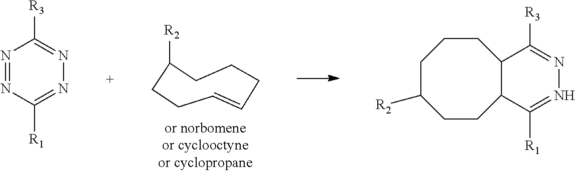 |
| Photo- induced tetrazole- alkene cycloaddition | 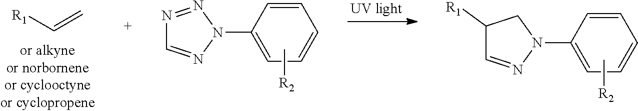 |
| [4+1] cycloaddition | 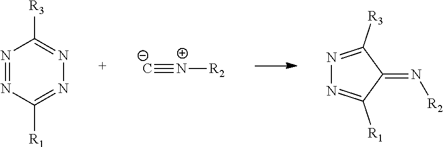 |
| Quadricyclane ligation | 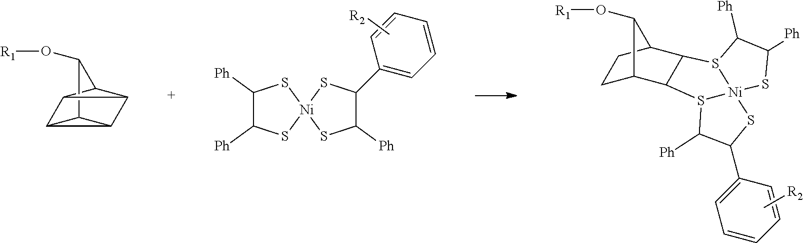 |

While Formulae II and III depict a cross-linker positioned within a “tetraloop” structure (or a cross-linker replacing the “tetraloop” structure) in the guide molecule repeat-antirepeat duplex, it will be appreciated that cross-linkers may be positioned anywhere in the molecules, for example, in any stem loop structure occurring within a guide molecule, including naturally-occurring stem loops and engineered stem loops. In particular, certain embodiments of this disclosure relate to guide molecules lacking a tetraloop structure and comprising a cross-linker positioned at the terminus of first and second complementary regions (for instance, at the 3′ terminus of a first upper stem region and the 5′ terminus of a second upper stem region).

Formulae II and III depict guide molecules that may (p>0 and q>0) or may not (p=0 and q=0) contain a “tetraloop” structure in the repeat-antirepeat duplex. One aspect of this invention is the recognition that guide molecules lacking a “tetraloop” may exhibit enhanced ligation efficiency as a result of having the functionalized 3′ and 5′ ends in close proximity and in a suitable orientation.

Alternatively, or additionally, a cross-linking reaction according to this disclosure can include a “splint” or a single stranded oligonucleotide that hybridizes to a sequence at or near the functionalized 3′ and 5′ ends in order to stably bring those functionalized ends into proximity with one-another.

Another aspect of this invention is the recognition that guide molecules with longer duplexes (e.g., with extended upper stems) may exhibit enhanced ligation efficiency as compared to guide molecules with shorter duplexes. These longer duplex structures are referred to in this disclosure interchangeably as “extended duplexes,” and are generally (but not necessarily) positioned in proximity to a functionalized nucleotide in a guide fragment. Thus, in some embodiments, the present disclosure provides guide molecules of Formulae VIII and IX, below:

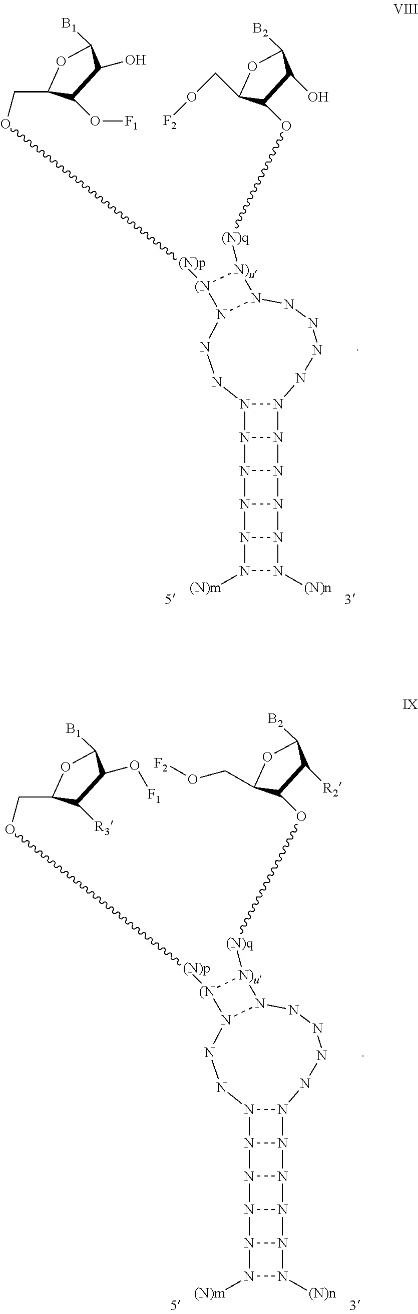

In Formulae VIII and IX, p′ and q′ are each independently an integer between 0 and 4, inclusive, p′+q′ is an integer between 0 and 4, inclusive, u′ is an integer between 2 and 22, inclusive, and other variables are defined as in Formulae II and III. Formulae VIII and IX depict a duplex with an optionally extended upper stem, as well as an optional tetraloop (i.e., when p and q are 0). Guide molecules of Formula VIII and IX may be advantageous due to increased ligation efficiency resulting from a longer upper stem. Furthermore, the combination of a longer upper stem and the absence of a tetraloop may be beneficial for achieving an appropriate orientation of reactive groups F₁ and F₂ for the ligation reaction.

Another aspect of this invention relates to the recognition that guide fragments may include multiple regions of complementary within a single guide fragment and/or between different guide fragments. For example, in certain embodiments of this disclosure, first and second guide fragments are designed with complementary upper and lower stem regions that, when fully annealed, result in a heterodimer in which (a) first and second functional groups are positioned at the terminus of a duplexed upper stem region in suitable proximity for a cross-linking reaction and/or (b) a duplexed structure is formed between the first and second guide fragments that is capable of supporting the formation of a complex between the guide molecule and the RNA-guided nuclease. However, it may be possible for the first and second guide fragments to anneal incompletely with one another, or to form internal duplexes or homodimers, whereby (a) and/or (b) does not occur. As one example, in S. pyogenes guide molecules based on the wild-type crRNA and tracrRNA sequences, there may be multiple highly complementary sequences such as poly-U or poly-A tracts in the lower and upper stem that may lead to improper “staggered” heterodimers involving annealing between upper and lower stem regions, rather than the desired annealing of upper stem regions with one another. Similarly, undesirable duplexes may form between the targeting domain sequence of a guide fragment and another region of the same guide fragment or a different fragment, and mispairing may occur between otherwise complementary regions of first and second guide fragments, potentially resulting in incomplete duplexation, bulges and/or unpaired segments.

While it is not practical to predict all possible undesirable internal or intermolecular duplex structures that may form between guide fragments, the inventors have found that, in some cases, a modification made to reduce or prevent the formation of a specific mis-pairing or undesirable duplex may have a significant effect on the yield of a desired guide molecule product in a cross-linking reaction, and/or result in a reduction of one or more contaminant species from the same reaction. Thus, in some embodiments, the present disclosure provides guide molecules and methods where the primary sequence of the guide fragments has been designed to avoid two or a particular mispairing or undesirable duplex (e.g., by swapping two complementary nucleotides between the first and second guide fragments). For example, an A-U swap in the upper stem of the wild-type S. pyogenes guide fragments mentioned above would produce a first guide fragment that includes non-identical UUUU and UAUU sequences and a second guide fragment that includes sequences complementary to the modified sequences of the first fragment, namely AAAA and AUAA sequences. More broadly, guides may incorporate sequence changes, such as a nucleotide swap between two duplexed portions of an upper or lower stem, an insertion, deletion or replacement of a sequence in an upper or lower stem, or structural changes such as the incorporation of locked nucleic acids (LNA)s in positions selected to reduce or eliminate the formation of a secondary structure.

While not wishing to be bound by any theory, it is believed that the duplex extensions, sequence modifications and structural modifications described herein promote the formation of desirable duplexes and reduce mis-pairing and the formation of undesirable duplexes by increasing the energetic favorability of the formation of a desirable duplex relative to the formation of a mis-paired or undesirable duplex. The energetic favorability of a particular annealing reaction may be represented by the Gibbs free energy (ΔG); negative ΔG values are associated with spontaneous reactions, and a first annealing reaction is more energetically favorable than a second reaction if the ΔG of the first reaction is less than (i.e., more negative than) the ΔG of the second reaction. ΔG may be assessed empirically, based on the thermal stability (melting behavior) of particular duplexes, for example using NMR, fluorescence quenching, UV absorbance, calorimetry, etc. as described by You, Tatourov and Owczarzy, “Measuring Thermodynamic Details of DNA Hybridization Using Fluorescence” Biopolymers Vol. 95, No. 7, pp. 472-486 (2011), which is incorporated by reference herein for all purposes. (See, e.g., “Introduction” at pp. 472-73 and “Materials and Methods” at pp. 473-475.) However, it may be more practical when designing guide fragments and annealing reactions to employ computational models to evaluate the free energy of correct duplexation and of selected mis-pairing or undesirable duplexation reactions, and a number of tools are available to perform such modeling, including the biophysics.idtdna.com tool hosted by Integrated DNA Technologies (Coralville, Iowa). Alternatively or additionally, a number of algorithms utilizing thermodynamic nearest neighbor models (TNN) are described in the literature. See, e.g., Tulpan, Andronescu and Leger, “Free energy estimation of short DNA duplex hybridizations,” BMC Bioinformatics, Vol. 11, No. 105 (2010). (See “Background” on pp. 1-2 describing TNN models and the MultiRNAFold package, the Vienna package and the UNAFold package). Other algorithms have also been described in the literature, e.g., by Kim et al. “An evolutionary Monte Carlo algorithm for predicting DNA hybridization,” J. Biosystems Vol. 7, No. 5 (2007). (See section 2 on pp. 71-2 describing the model.) Each of the foregoing references is incorporated by reference in its entirety and for all purposes.

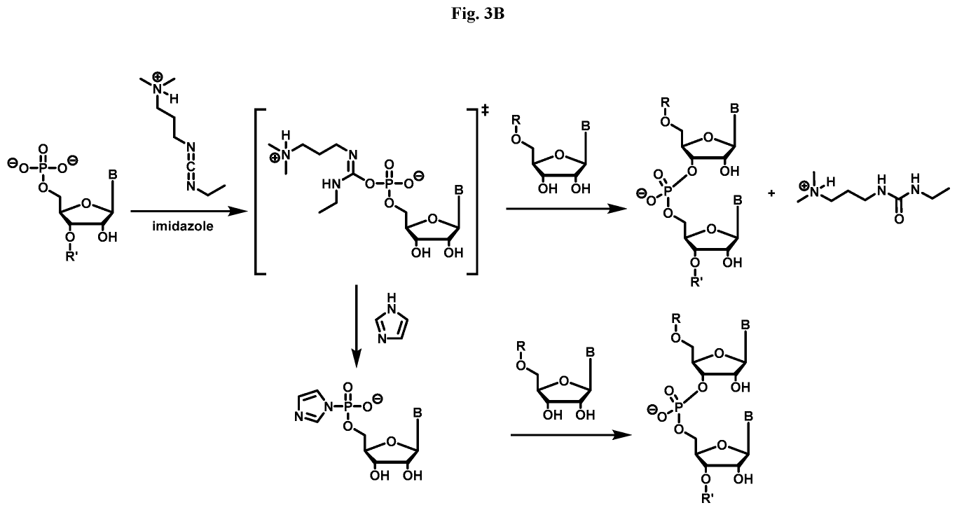

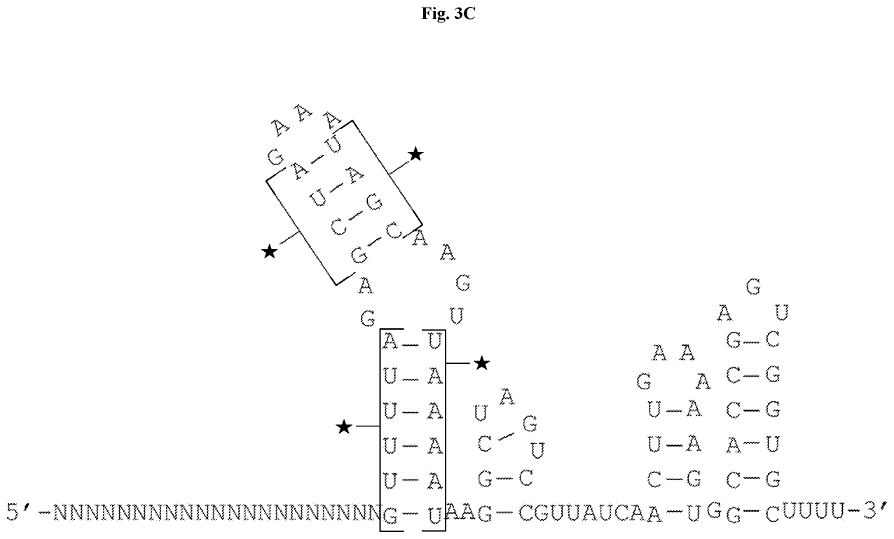

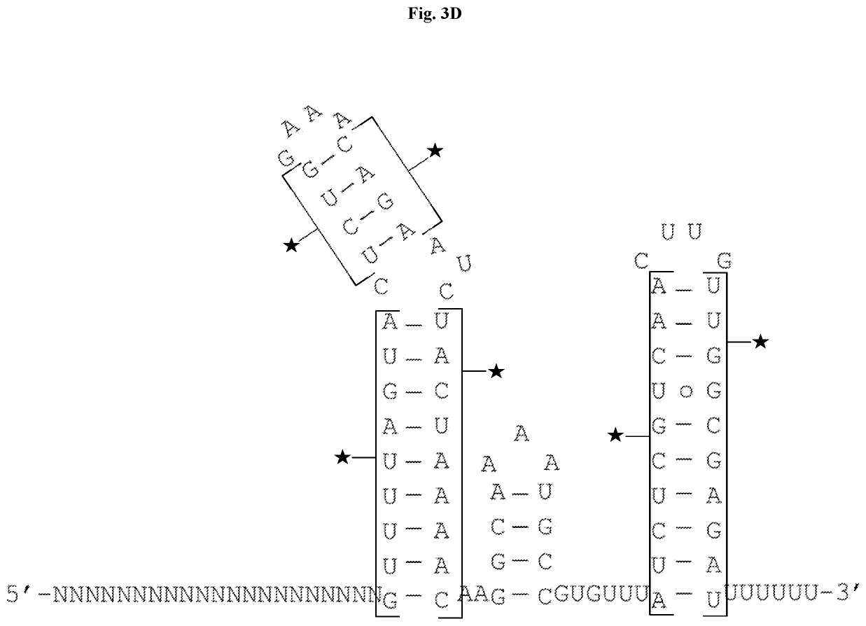

The arrangement depicted in Formulae II and III may be particularly advantageous where the functional groups are positioned on linking groups comprising multiple carbons. For less bulky cross-linkers, it may be desirable to achieve close apposition between functionalized 3′ and 5′ ends. FIGS. 3C and 3D identify duplexed portions of S. pyogenes and S. aureus gRNAs suitable for the use of shorter linkers, including without limitation phosphodiester bonds. These positions are generally selected to permit annealing between fragments, and to position functionalized 3′ and 5′ ends such that they are immediately adjacent to one another prior to cross-linking. Exemplary 3′ and 5′ positions located within (rather than adjacent to) a tract of annealed residues are shown in Formulae IV, V, VI and VII below:

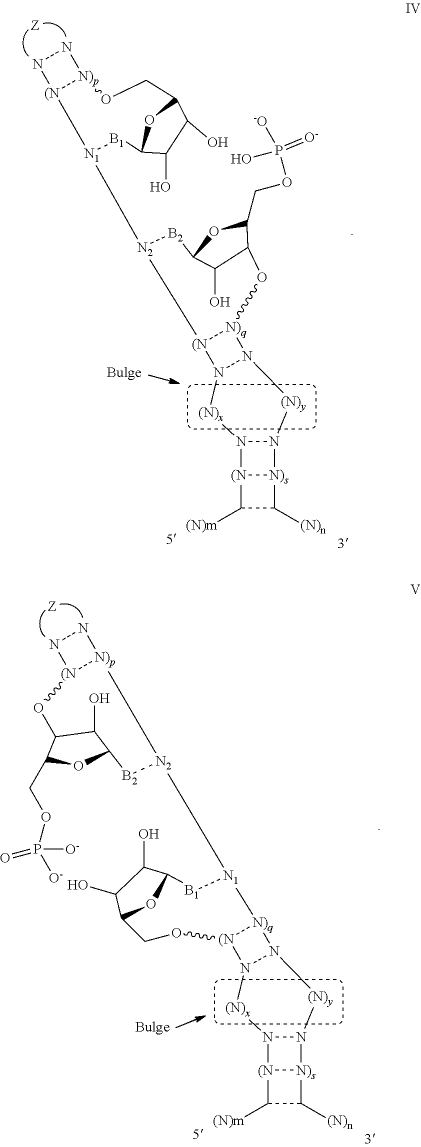

Z represents a nucleotide loop which is 4-6 nucleotides long, optionally 4 or 6 nucleotides long; p and q are each independently an integer between 0-2, inclusive, optionally 0; p′ is an integer between 0-4, inclusive, optionally 0; q′ is an integer between 2-4, inclusive, optionally 2; x is an integer between 0-6, inclusive optionally 2; y is an integer between 0-6, inclusive, optionally 4; u is an integer between 0-4, inclusive, optionally 2; s is an integer between 2-6, inclusive, optionally 4; m is an integer between 20-40, inclusive; n is an integer between 30-70, inclusive; B₁ and B₂ are each independently a nucleobase; each N in (N)ₘ and (N)ₙ is independently a nucleotide residue; N₁ and N₂ are each independently a nucleotide residue; [symbol] independently represents two complementary nucleotides, optionally two complementary nucleotides that are hydrogen bonding base-paired; and each [symbol] represents a phosphodiester linkage, a phosphorothioate linkage, a phosphonoacetate linkage, a thiophosphonoacetate linkage, or a phosphoroamidate linkage.

Another aspect of the invention is the recognition that the arrangement depicted in any of Formulae II, III, IV, V, VI, or VII may be advantageous for avoiding side products in cross-linking reactions, as well as allowing for homobifunctional reactions to occur without homodimerization. Pre-annealing of the two heterodimeric strands orients the reactive groups toward the desired coupling and disfavors reaction with other potential reactive groups in the guide molecule.

Another aspect of the invention is the recognition that hydroxyl groups in proximity to the reactive groups (e.g., the 2′-OH on the 3′ end of the first fragment) are preferably modified to avoid the formation of certain side products. In particular, as illustrated below, the inventors discovered that a carbamate side product may form when amine-functionalized fragments are used in the urea-based cross-linking methods described herein:

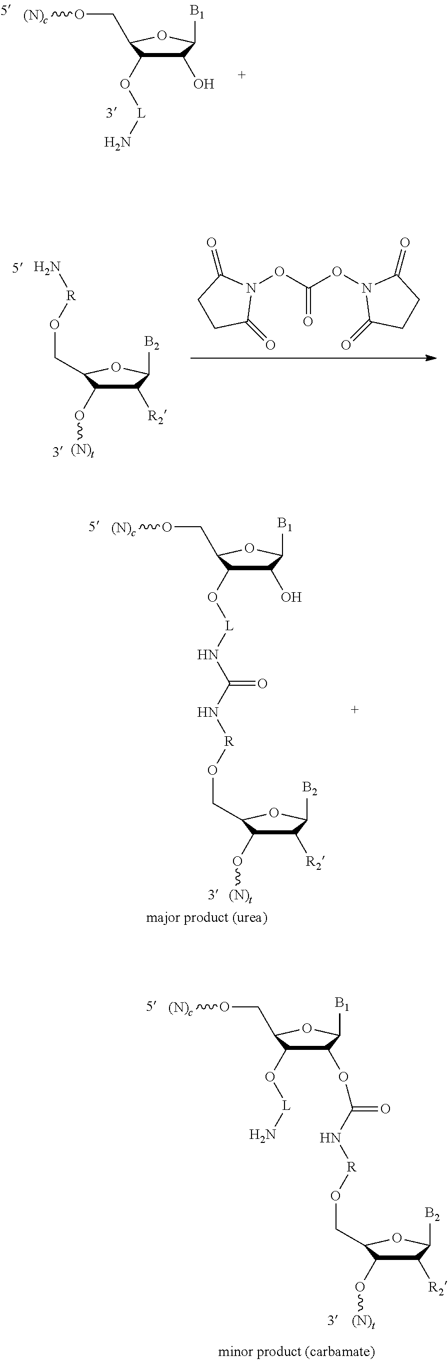

Thus, in certain embodiments, the 2′-OH on the 3′ end of the first fragment is modified (e.g., to H, halogen, O-Me, etc.) in order to prevent formation of the carbamate side product. For example, the 2′-OH is modified to a 2′-H:

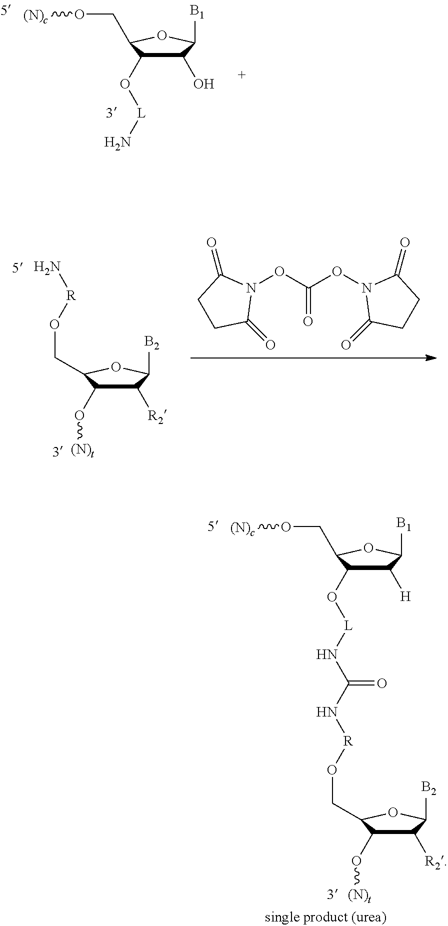

Turning next to cross-linking, several considerations are relevant in selection of cross-linker linking moieties, functional groups and reactive groups. Among these are linker size, solubility in aqueous solution and biocompatibility, as well as the functional group reactivity, optimal reaction conditions for cross-linking, and any necessary reagents, catalyst, etc. required for cross-linking.

In general, linker size and solubility are selected to preserve or achieve a desired RNA secondary structure, and to avoid disruption or destabilization of the complex between guide molecule and RNA-guided nuclease. These two factors are somewhat related, insofar as organic linkers above a certain length may be poorly soluble in aqueous solution and may interfere sterically with surrounding nucleotides within the guide molecule and/or with amino acids in an RNA-guided nuclease complexed with the guide molecule.

A variety of linkers are suitable for use in the various embodiments of this disclosure. Certain embodiments make use of common linking moieties including, without limitation, polyvinylether, polyethylene, polypropylene, polyethylene glycol (PEG), polypropylene glycol (PEG), polyvinyl alcohol (PVA), polyglycolide (PGA), polylactide (PLA), polycaprolactone (PCL), and copolymers thereof. In some embodiments, no linker is used.

As to functional groups, in embodiments in which a bifunctional cross-linker is used to link 5′ and 3′ ends of guide fragments, the 3′ or 5′ ends of the guide fragments to be linked are modified with functional groups that react with the reactive groups of the cross-linker. In general, these modifications comprise one or more of amine, sulfhydryl, carboxyl, hydroxyl, alkene (e.g., a terminal alkene), azide and/or another suitable functional group. Multifunctional (e.g., bifunctional) cross-linkers are also generally known in the art, and may be either heterofunctional or homofunctional, and may include any suitable functional group, including without limitation isothiocyanate, isocyanate, acyl azide, an NHS ester, sulfonyl chloride, tosyl ester, tresyl ester, aldehyde, amine, epoxide, carbonate (e.g., Bis(p-nitrophenyl) carbonate), aryl halide, alkyl halide, imido ester, carboxylate, alkyl phosphate, anhydride, fluorophenyl ester, HOBt ester, hydroxymethyl phosphine, O-methylisourea, DSC, NHS carbamate, glutaraldehyde, activated double bond, cyclic hemiacetal, NHS carbonate, imidazole carbamate, acyl imidazole, methylpyridinium ether, azlactone, cyanate ester, cyclic imidocarbonate, chlorotriazine, dehydroazepine, 6-sulfo-cytosine derivatives, maleimide, aziridine, TNB thiol, Ellman's reagent, peroxide, vinylsulfone, phenylthioester, diazoalkanes, diazoacetyl, epoxide, diazonium, benzophenone, anthraquinone, diazo derivatives, diazirine derivatives, psoralen derivatives, alkene, phenyl boronic acid, etc.

These and other cross-linking chemistries are known in the art, and are summarized in the literature, including by Greg T. Hermanson, Bioconjugate Techniques, 3^{rd} Ed. 2013, published by Academic Press, which is incorporated by reference herein in its entirety and for all purposes.

Compositions comprising guide molecules synthesized by the methods provided by this disclosure are, in certain embodiments, characterized by high purity of the desired guide molecule reaction product, with low levels of contamination with undesirable species, including n−1 species, truncations, n+1 species, guide fragment homodimers, unreacted functionalized guide fragments, etc. In certain embodiments of this disclosure, a purified composition comprising synthetic guide molecules can comprise a plurality of species within the composition (i.e., the guide molecule is the most common species within the composition, by mass or molarity). Alternatively, or additionally, compositions according to the embodiments of this disclosure can comprise ≥70%, ≥75%, ≥80%, ≥85%, ≥90%, ≥95%, ≥96%, ≥97%, ≥98%, and/or ≥99%, of a guide molecule having a desired length (e.g., lacking a truncation at a 5′ end, relative to a reference guide molecule sequence) and a desired sequence (e.g., comprising a 5′ sequence of a reference guide molecule sequence).

For example, in some embodiments, a composition comprising guide molecules according to the disclosure (e.g., guide molecules comprising fragments cross-linked using an appropriate cross-linking chemistry described herein) includes less than about 20%, 15%, 10%, 9%, 8%, 7%, 6%, 5%, 4%, 3%, 2%, 1%, or less, of guide molecules that comprise a truncation at a 5′ end, relative to a reference guide molecule sequence. Additionally or alternatively, a composition comprising guide molecules according to the disclosure (e.g., guide molecules comprising fragments cross-linked using an appropriate cross-linking chemistry described herein) includes at least about 90%, 95%, 96%, 97%, 98%, 99%, or 100% of guide molecules with a 5′ sequence (e.g., a 5′ sequence comprising or consisting of nucleotides 1-30, 1-25, or 1-20 of the guide molecule) that is 100% identical to a corresponding 5′ sequence of a reference guide molecule sequence. In some embodiments, if the composition comprises guide molecules with a 5′ sequence that is less than 100% identical to a corresponding 5′ sequence of the reference guide molecule sequence, and such guide molecules are present at a level greater than or equal to 0.1%, such guide molecule does not comprise a targeting domain for a potential off-target site. In some embodiments, a composition comprising guide molecules according to the disclosure includes at least about 80%, 85%, 90%, 91%, 92%, 93%, 94%, 95%, 96%, 97%, 98%, 99% or more, guide molecules that do not comprise a truncation at a 5′ end (relative to a reference guide molecule sequence), and at least about 90%, 95%, 96%, 97%, 98%, 99%, or 100% of such guide molecules (i.e., such guide molecule not comprising a truncation at a 5′ end) have a 5′ sequence (e.g., a 5′ sequence comprising or consisting of nucleotides 1-30, 1-25, or 1-20 of the guide molecule) that is 100% identical to the corresponding 5′ sequence of the reference guide molecule sequence, and if the composition comprises guide molecules with a 5′ sequence that is less than 100% identical to a corresponding 5′ sequence of the reference guide molecule sequence, and such guide molecules are present at a level greater than or equal to 0.1%, such guide molecule does not comprise a targeting domain for a potential off-target site. In some embodiments, compositions comprising guide molecules according to the disclosure include less than about 10%, of guide molecules that comprise a truncation at a 5′ end, relative to a reference guide molecule sequence and exhibit an acceptable level of activity/efficacy. In some embodiments, compositions comprising guide molecules according to the disclosure include (i) at least about 99% of guide molecules having a 5′ sequence (e.g., a 5′ sequence comprising or consisting of nucleotides 1-30, 1-25, or 1-20 of the guide molecule) that is 100% identical to the corresponding 5′ sequence of the reference guide molecule sequence, and (ii) if the composition comprises guide molecules with a 5′ sequence that is less than 100% identical to a corresponding 5′ sequence of the reference guide molecule sequence, and such guide molecules are present at a level greater than or equal to 0.1%, such guide molecule does not comprise a targeting domain for a potential off-target site, and compositions exhibit an acceptable level of specificity/safety. The purity of the composition may be expressed as a fraction of total guide molecule (by mass or molarity) within the composition, as a fraction of all RNA or all nucleic acid (by mass or molarity) within the composition, as a fraction of all solutes within the composition (by mass), and/or as a fraction of the total mass of the composition.

The purity of a composition comprising a guide molecule according to this disclosure is assessed by any suitable means known in the art. For example, the relative abundance of the desired guide molecule species can be assessed qualitatively or semi-quantitatively by means of gel electrophoresis. Alternatively or additionally, the purity of a desired guide molecule species is assessed by chromatography (e.g., liquid chromatography, HPLC, FPLC, gas chromatography), spectrometry (e.g., mass spectrometry, whether based on time-of-flight, sector field, quadrupole mass, ion trap, orbitrap, Fourier transform ion cyclotron resonance, or other technology), nuclear magnetic resonance (NMR) spectroscopy (e.g., visible, infrared or ultraviolet), thermal stability methods (e.g., differential scanning calorimetry, etc.), sequencing methods (e.g., using a template switching oligonucleotide) and combinations thereof (e.g., chromatography-spectrometry, etc.).

The synthetic guide molecules provided herein operate in substantially the same manner as any other guide molecules (e.g., gRNA), and generally operate by (a) forming a complex with an RNA-guided nuclease such as Cas9, (b) interacting with a target sequence including a region complementary to a targeting sequence of the guide molecule and a protospacer adjacent motif (PAM) recognized by the RNA-guided nuclease, and optionally (c) modifying DNA within or adjacent to the target sequence, for instance by forming a DNA double strand break, single strand break, etc. that may be repaired by DNA repair pathways operating within a cell containing the guide molecule and RNA-guided nuclease.

In some embodiments, a guide molecule described herein, e.g., a guide molecule produced using a method described herein, can act as a substrate for an enzyme (e.g., a reverse transcriptase) that acts on RNA. Without wishing to be bound by theory, cross-linkers present within guide molecules described herein may be compatible with such processive enzymes due to close apposition of reactive ends promoted by pre-annealing according to methods of the disclosure.

The exemplary embodiments described above have focused on the application of the synthesis and cross-linking methods described herein to the assembly of guide molecules from two guide fragments. However, the methods described herein have a variety of applications, many of which will be evident to skilled artisans. These applications are within the scope of the present disclosure. As one example, the methods of this disclosure may be employed in the linking of heterologous sequences to guide molecules. Heterologous sequences may include, without limitation, DNA donor templates as described in WO 2017/180711 by Cotta-Ramusino, et al., which is incorporated by reference herein for all purposes. (See, e.g., Section I, “gRNA Fusion Molecules” at p. 23, describing covalently linked template nucleic acids, and the use of splint oligos to facilitate ligation of the template to the 3′ end of the guide molecule.) Heterologous sequences can also include nucleic acid sequences that are recognized by peptide DNA or RNA binding domains, such as MS2 loops, also described in Section I of WO 2017/180711 above.

This overview has focused on a handful of exemplary embodiments that illustrate certain principles relating to the synthesis of guide molecules, and compositions comprising such guide molecules. For clarity, however, this disclosure encompasses modifications and variations that have not been described but that will be evident to those of skill in the art. With that in mind, the following disclosure is intended to illustrate the operating principles of genome editing systems more generally. What follows should not be understood as limiting, but rather illustrative of certain principles of genome editing systems, which, in combination with the instant disclosure, will inform those of skill in the art about additional implementations of and modifications that are within the scope of this disclosure.

### Genome Editing Systems

The term “genome editing system” refers to any system having RNA-guided DNA editing activity. Genome editing systems of the present disclosure include at least two components adapted from naturally occurring CRISPR systems: a guide molecule (e.g., guide RNA or gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

Naturally occurring CRISPR systems are organized evolutionarily into two classes and five types (Makarova et al. Nat Rev Microbiol. 2011 June; 9(6): 467-477 (Makarova), incorporated by reference herein), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e. target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide molecules described herein do not occur in nature, and both guide molecules and RNA-guided nucleases according to this disclosure may incorporate any number of non-naturally occurring modifications.

Genome editing systems can be implemented (e.g., administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as a lipid or polymer micro- or nano-particle, micelle, liposome, etc. In certain embodiments, a genome editing system is implemented as one or more nucleic acids encoding the RNA-guided nuclease and guide molecule components described above (optionally with one or more additional components); in certain embodiments, the genome editing system is implemented as one or more vectors comprising such nucleic acids, for instance a viral vector such as an adeno-associated virus; and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel—two or more specific nucleotide sequences through the use of two or more guide molecules. The use of multiple guide molecules is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (Maeder), which is incorporated by reference herein, describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e., flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino, et al. (“Cotta-Ramusino”), incorporated by reference herein, describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al. (“Palestrant”, incorporated by reference herein) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111(10): E924-932, Mar. 11, 2014 (Davis) (describing Alt-HDR); Frit et al. DNA Repair 17(2014) 81-97 (Frit) (describing Alt-NHEJ); and Iyama and Wilson III, DNA Repair (Amst.) 2013-August; 12(8): 620-636 (Iyama) (describing canonical HDR and NHEJ pathways generally).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a gene in or near the target sequence, without causing single- or double-strand breaks. For example, a genome editing system may include an RNA-guided nuclease fused to a functional domain that acts on DNA, thereby modifying the target sequence or its expression. As one example, an RNA-guided nuclease can be connected to (e.g., fused to) a cytidine deaminase functional domain, and may operate by generating targeted C-to-A substitutions. Exemplary nuclease/deaminase fusions are described in Komor et al. Nature 533, 420-424 (19 May 2016) (“Komor”), which is incorporated by reference. Alternatively, a genome editing system may utilize a cleavage-inactivated (i.e., a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc.

### Guide Molecules

The term “guide molecule” is used herein refer to any nucleic acid that promotes the specific association (or “targeting”) of an RNA-guided nuclease such as a Cas9 or a Cpf1 to a target sequence such as a genomic or episomal sequence in a cell. A guide molecule may be an RNA molecule or a hybrid RNA/DNA molecule. Guide molecules can be unimolecular (comprising a single molecule, and referred to alternatively as chimeric), or modular (comprising more than one, and typically two, separate molecules, such as a crRNA and a tracrRNA, which are usually associated with one another, for instance by duplexing). Guide molecules and their component parts are described throughout the literature, for instance in Briner et al. (Molecular Cell 56(2), 333-339, Oct. 23, 2014 (Briner), which is incorporated by reference), and in Cotta-Ramusino.

In bacteria and archaea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/guide molecule complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end). (Mali et al., Science. 2013 Feb. 15; 339(6121): 823-826 (“Mali”); Jiang et al., Nat Biotechnol. 2013 March; 31(3): 233-239 (“Jiang”); and Jinek et al., 2012 Science Aug. 17; 337(6096): 816-821 (“Jinek”), all of which are incorporated by reference herein.)

Guide molecules, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31(9): 827-832, (“Hsu”), incorporated by reference herein), “complementarity regions” (Cotta-Ramusino), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 guide molecule, and at or near the 3′ terminus in the case of a Cpf1 guide molecule.

In addition to the targeting domains, guide molecules typically (but not necessarily, as discussed below) include a plurality of domains that may influence the formation or activity of guide molecule/Cas9 complexes. For instance, as mentioned above, the duplexed structure formed by first and secondary complementarity domains of a guide molecule (also referred to as a repeat:anti-repeat duplex) interacts with the recognition (REC) lobe of Cas9 and can mediate the formation of Cas9/guide molecule complexes. (Nishimasu et al., Cell 156, 935-949, Feb. 27, 2014 (Nishimasu 2014) and Nishimasu et al., Cell 162, 1113-1126, Aug. 27, 2015 (Nishimasu 2015), both incorporated by reference herein).

Along with the first and second complementarity domains, Cas9 guide molecules typically include two or more additional duplexed regions that are involved in nuclease activity in vivo but not necessarily in vitro. (Nishimasu 2015). A first stem-loop near the 3′ portion of the second complementarity domain is referred to variously as the “proximal domain,” (Cotta-Ramusino) “stem loop 1” (Nishimasu 2014 and 2015) and the “nexus” (Briner). One or more additional stem loop structures are generally present near the 3′ end of the guide molecule, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and guide molecule structures more generally) organized by species is provided in Briner.

While the foregoing description has focused on guide molecules for use with Cas9, it should be appreciated that other RNA-guided nucleases have been (or may in the future be) discovered or invented which utilize guide molecules that differ in some ways from those described to this point. For instance, Cpf1 (“CRISPR from Prevotella and Franciscella 1”) is a recently discovered RNA-guided nuclease that does not require a tracrRNA to function. (Zetsche et al., 2015, Cell 163, 759-771 Oct. 22, 2015 (Zetsche I), incorporated by reference herein). A guide molecule for use in a Cpf1 genome editing system generally includes a targeting domain and a complementarity domain (alternately referred to as a “handle”). It should also be noted that, in guide molecules for use with Cpf1, the targeting domain is usually present at or near the 3′ end, rather than the 5′ end as described above in connection with Cas9 guide molecules (the handle is at or near the 5′ end of a Cpf1 guide molecule).

Those of skill in the art will appreciate, however, that although structural differences may exist between guide molecules from different prokaryotic species, or between Cpf1 and Cas9 guide molecules, the principles by which guide molecules operate are generally consistent. Because of this consistency of operation, guide molecules can be defined, in broad terms, by their targeting domain sequences, and skilled artisans will appreciate that a given targeting domain sequence can be incorporated in any suitable guide molecule, including a unimolecular or chimeric guide molecules, or a guide molecule that includes one or more chemical modifications and/or sequential modifications (substitutions, additional nucleotides, truncations, etc.). Thus, for economy of presentation in this disclosure, guide molecules may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term guide molecule should be understood to encompass any suitable guide molecule (e.g., gRNA) that can be used with any RNA-guided nuclease, and not only those guide molecules that are compatible with a particular species of Cas9 or Cpf1. By way of illustration, the term guide molecule can, in certain embodiments, include a guide molecule for use with any RNA-guided nuclease occurring in a Class 2 CRISPR system, such as a type II or type V or CRISPR system, or an RNA-guided nuclease derived or adapted therefrom.

Cross Linked Guide Molecules

Certain embodiments of this disclosure related to guide molecules that are cross linked through, for example, a non-nucleotide chemical linkage. As described above, the position of the linkage may be in the stem loop structure of a guide molecule. In some embodiments, the guide molecule comprises

In some embodiments, the unimolecular guide molecule comprises, from 5′ to 3′:
a first guide molecule fragment, comprising:
   a targeting domain sequence;
   a first lower stem sequence;
   a first bulge sequence;
   a first upper stem sequence;
a non-nucleotide chemical linkage; and
a second guide molecule fragment, comprising
   a second upper stem sequence;
   a second bulge sequence; and
   a second lower stem sequence,

wherein (a) at least one nucleotide in the first lower stem sequence is base paired with a nucleotide in the second lower stem sequence, and (b) at least one nucleotide in the first upper stem sequence is base paired with a nucleotide in the second upper stem sequence.

In some embodiments, the guide molecule does not include a tetraloop sequence between the first and second upper stem sequences. In some embodiments, the first and/or second upper stem sequence comprises nucleotides that number from 4 to 22 inclusive. In some embodiments, the first and/or second upper stem sequences comprise nucleotides that number from 1 to 22, inclusive. In some embodiments, the first and/or second upper stem sequences comprise nucleotides that number from 4 to 22, inclusive. In some embodiments, the first and second upper stem sequences comprise nucleotides that number from 8 to 22, inclusive. In some embodiments, the first and second upper stem sequences comprise nucleotides that number from 12 to 22, inclusive.

In some embodiments, the guide molecule is characterized in that a Gibbs free energy (ΔG) for the formation of a duplex between the first and second guide molecule fragments is less than a ΔG for the formation of a duplex between two first guide molecule fragments. In some embodiments, a ΔG for the formation of a duplex between the first and second guide molecule fragments is characterized by greater than 50%, 60%, 70%, 80%, 90% or 95% base pairing between each of (i) the first and second upper stem sequences and (ii) the first and second lower stem sequences is less than a ΔG for the formation of a duplex characterized by less than 50%, 60%, 70%, 80%, 90% or 95% base pairing between (i) and (ii).

In some embodiments, the synthetic guide molecule is of formula:

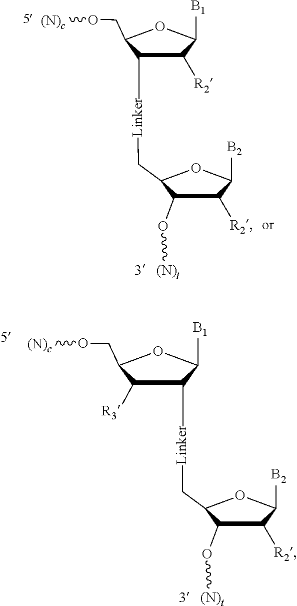

wherein each N in (N)_{c} and (N)ₜ is independently a nucleotide residue, optionally a modified nucleotide residue, each independently linked to its adjacent nucleotide(s) via a phosphodiester linkage, a phosphorothioate linkage, a phosphonoacetate linkage, a thiophosphonoacetate linkage, or a phosphoroamidate linkage;

(N)_{c} includes a 3′ region that is complementary or partially complementary to, and forms a duplex with, a 5′ region of (N)ₜ;

c is an integer 20 or greater;

t is an integer 20 or greater;

Linker is a non-nucleotide chemical linkage;

B₁ and B₂ are each independently a nucleobase;

each of R₂′ and R₃′ is independently H, OH, fluoro, chloro, bromo, NH₂, SH, S—R′, or O—R′ wherein each R′ is independently a protection group or an alkyl group, wherein the alkyl group may be optionally substituted; and

each [symbol] represents independently a phosphodiester linkage, a phosphorothioate linkage, a phosphonoacetate linkage, a thiophosphonoacetate linkage, or a phosphoroamidate linkage.

In some embodiments, the duplex regions of (N)ₜ and (N)_{c} comprise a sequence listed in Table 4.

TABLE 4
Exemplary sequences of (N)ₜ and (N)_{c}.

| SEQ ID NO. | Sequence |
|---|---|
| 1 | GUUUUAGAGCUAG |
| 2 | AUAGCAAGUUAAAAU |
| 3 | GUUUUAGAGCU |
| 4 | AGCAAGUUAAAAU |
| 5 | GUUUUAGAGCUAG |
| 6 | CUAGCAAGUUAAAAU |
| 7 | GUUUUAGAGCUAUG |
| 8 | CAUAGCAAGUUAAAAU |
| 9 | GUAUUAGAGCUAUGCUGUUUU |
| 10 | AAAACAGCAUAGCAAGUUAAUAU |
| 11 | GUAUUAGAGCUAUGCU |
| 12 | AGCAUAGCAAGUUAAUAU |
| 13 | GUUUUAGAGCUAUGCUGUUUU |
| 14 | AAAACAGCAUAGCAAGUUAAAAU |
| 15 | GUUUUAGAGCUAUGCU |
| 16 | AGCAUAGCAAGUUAAAAA |
| 17 | GUUUUAGAGCUAAAG |
| 18 | AUUUAGCAAGUUAAAAU |
| 19 | GUUUUAGAGCUAA |
| 20 | UUAGCAAGUUAAAAU |
| 21 | GUUUUAGAGCUAAAGGG |
| 22 | ACCUUUAGCAAGUUAAAAU |
| 23 | GUUUUAGAGCUAG |
| 24 | GUUUUAGUACUCU |
| 25 | AGAAUCUACUAAAAC |
| 26 | GUUUUAGUACUCUGUA |
| 27 | UACAGAAUCUACUAAAAC |
| 28 | GUUUUAGUACUCUGUAAUUUUAGG |
| 29 | CCUAAAAUUACAGAAUCUACUAAAAC |
| 30 | GUUUUAGUACUCUGUAAUUUUAGGUAUGA |
| 31 | UCAUACCUAAAAUUACAGAAUCUACUAAAAC |

In some embodiments, the guide molecule is of formula:

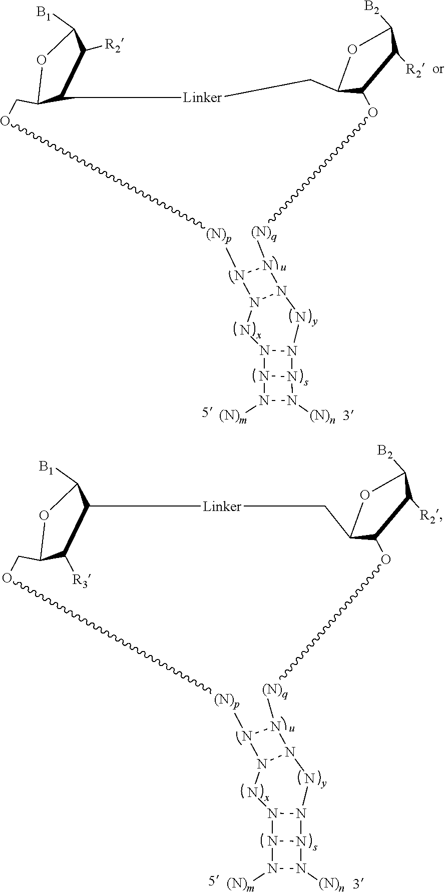

wherein N, B₁, B₂, R_{2′}, R₃′, Linker, and [symbol] are defined as above; and

[symbol] independently represents two complementary nucleotides, optionally two complementary nucleotides that are hydrogen bonding base-paired;

p and q are each independently an integer between 0 and 6, inclusive and p+q is an integer between 0 and 6, inclusive;

u is an integer between 2 and 22 inclusive;

s is an integer between 1 and 10, inclusive;

x is an integer between 1 and 3, inclusive;

y is >x and an integer between 3 and 5, inclusive;

m is an integer 15 or greater; and

n is an integer 30 or greater.

In some embodiments, ( [symbol] )ᵤ and ( [symbol] )ₛ do not comprise an identical sequence of 3 or more nucleotides. In some embodiments, ( [symbol] )ᵤ and ( [symbol] )ₛ do not comprise an identical sequence of 4 or more nucleotides. In some embodiments, ( [symbol] )ₛ comprises a N′UUU, UN′UU, UUN′U or UUUN′ sequence and ( [symbol] )ᵤ comprises a UUUU sequence, wherein N′ is A, G or C. In some embodiments, ( [symbol] )ₛ comprises a UUUU sequence and ( [symbol] )ᵤ comprises a N′UUU, UN′UU, UUN′U or UUUN′ sequence, wherein N′ is A, G or C. In some embodiments, N′ is A. In some embodiments, N′ is G. In some embodiments, N′ is C.

In some embodiments, the guide molecule is based on gRNAs used in S. pyogenes or S. aureus Cas9 systems. In some embodiments, the guide molecule is of formula:

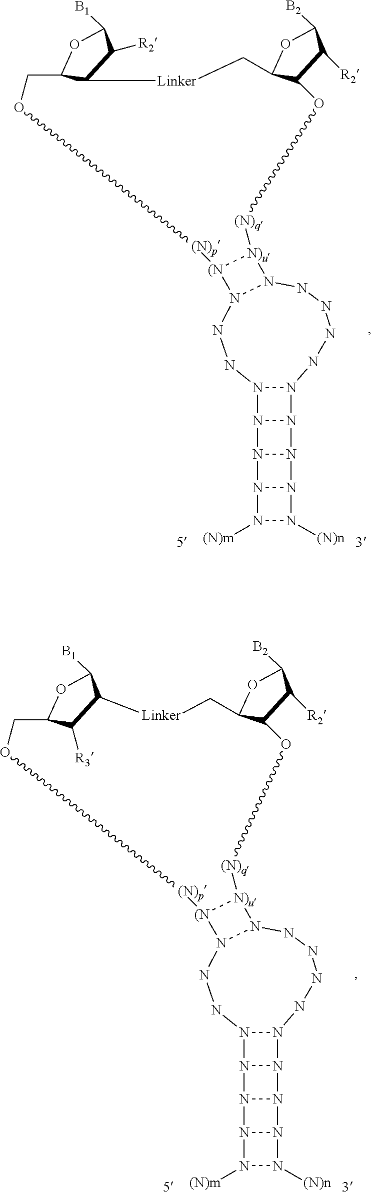

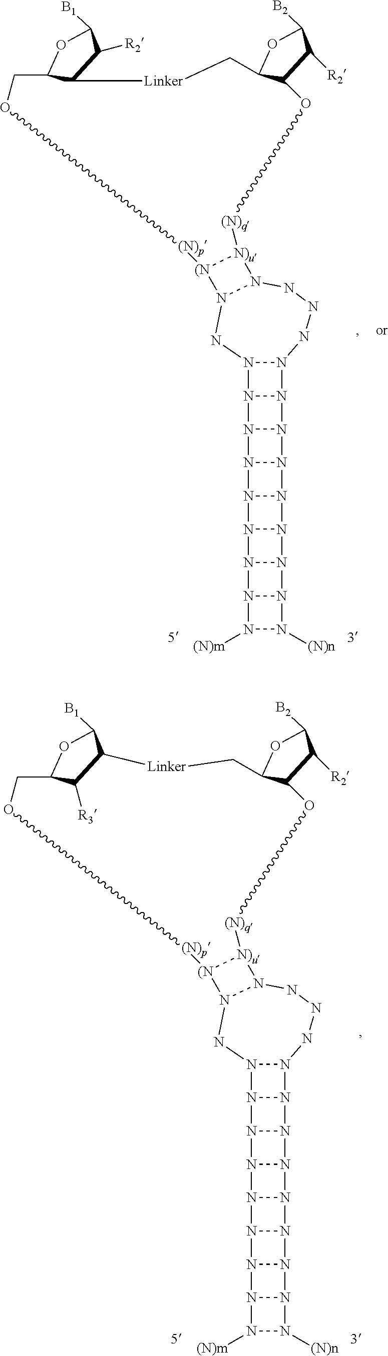

wherein: u′ is an integer between 2 and 22, inclusive; and p′ and q′ are each independently an integer between 0 and 6, inclusive, and p′+q′ is an integer between 0 and 6, inclusive.

In some embodiments, the guide molecule is of formula:

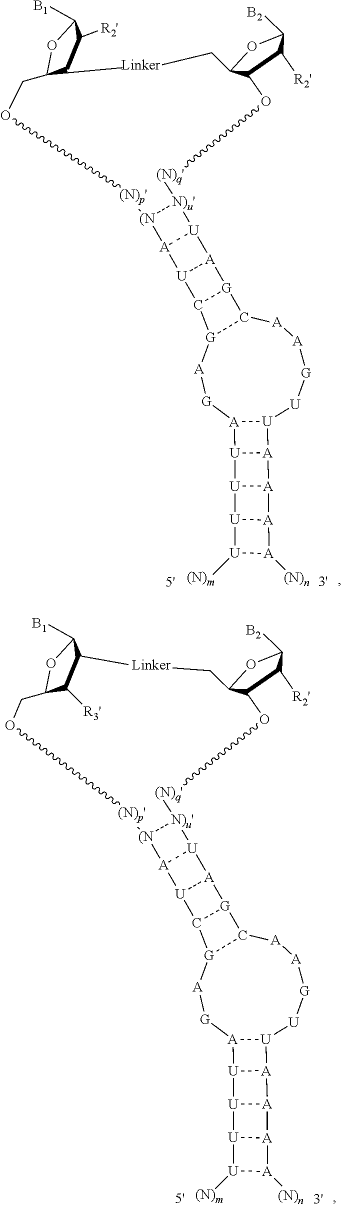

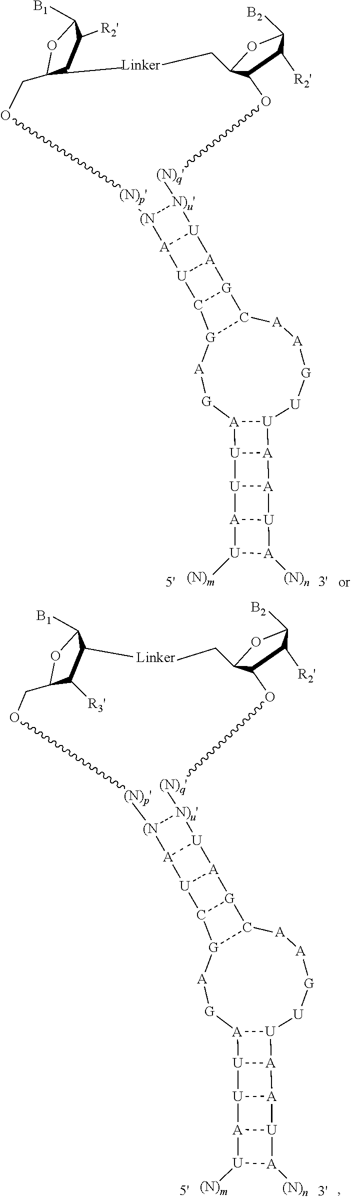

or covariants thereof. In some embodiments, ( [symbol] )_{u′} is of formula:

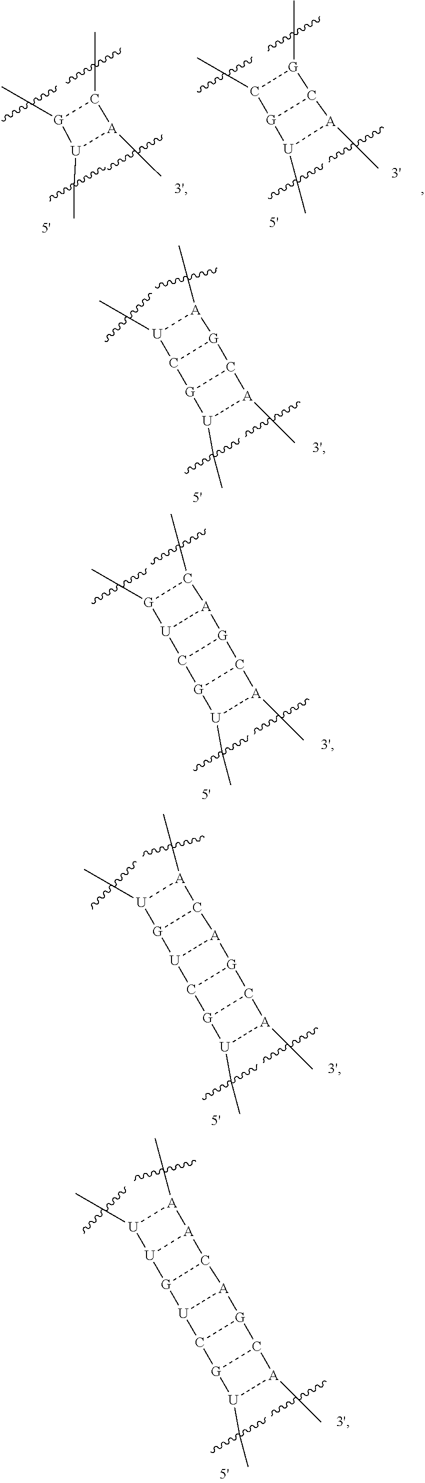

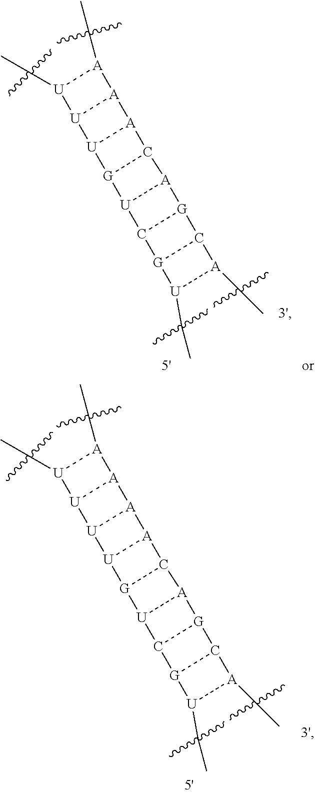

or covariants thereof. In some embodiments, ( [symbol] )_{u′} is of formula:

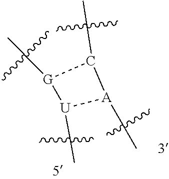

and B₁ is a cytosine residue and B₂ is a guanine residue, or a covariant thereof. In some embodiments, ( [symbol] )_{u′} is of formula:

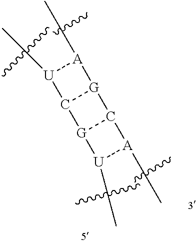

and B₁ is a guanine residue and B₂ is a cytosine residue, or a covariant thereof. In some embodiments, ( [symbol] )_{u′} is of formula:

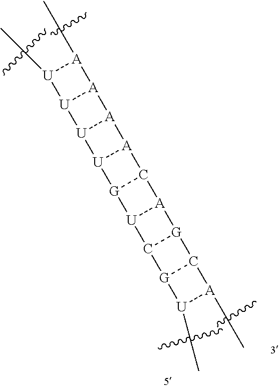

and B₁ is a guanine residue and B₂ is a cytosine residue, or a covariant thereof.

In some embodiments, the guide molecule is of formula:

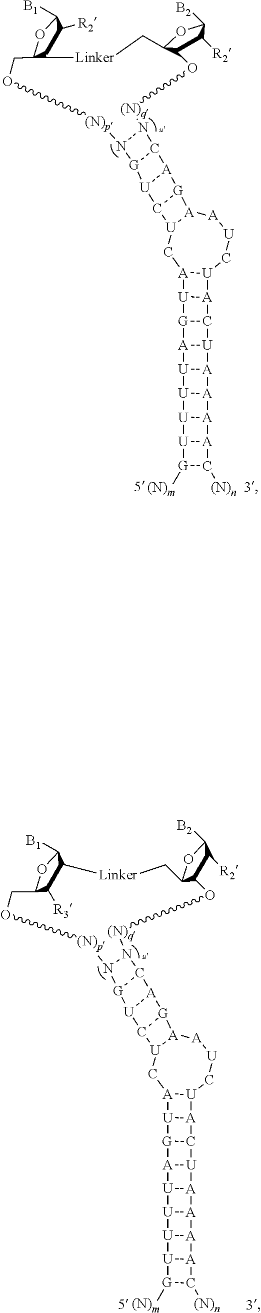

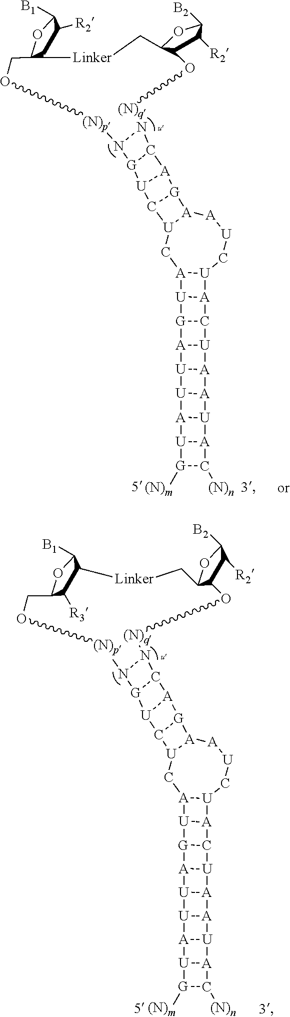

or covariants thereof.
In some embodiments, ( [symbol] )_{u′} is of formula:

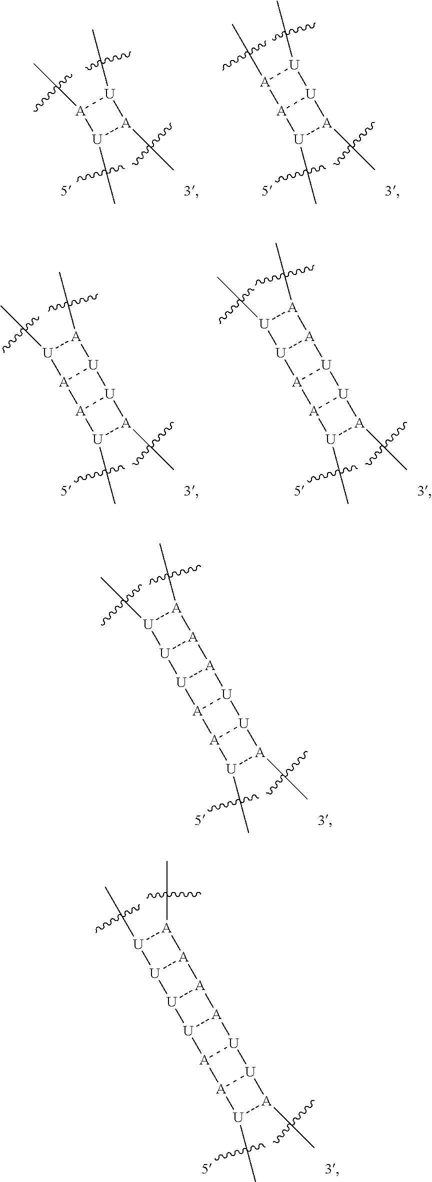

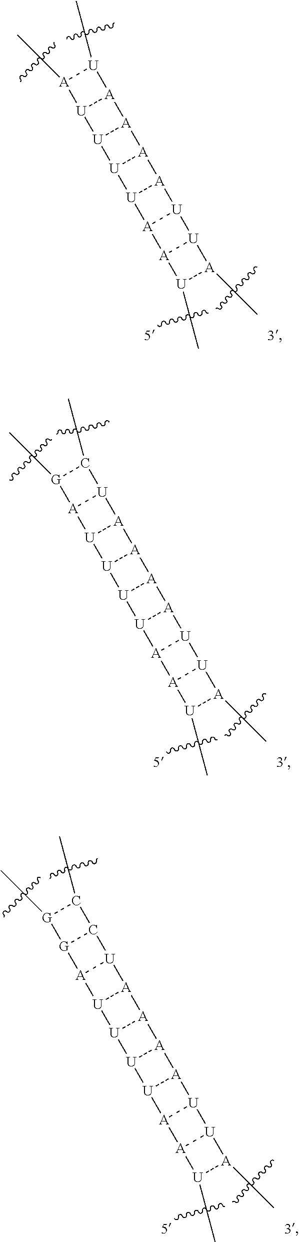

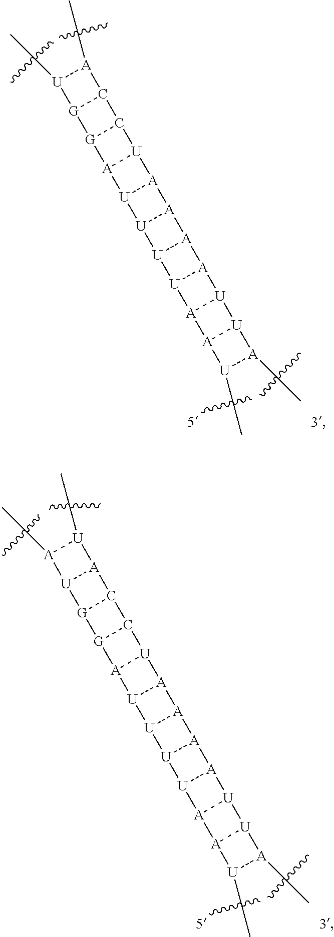

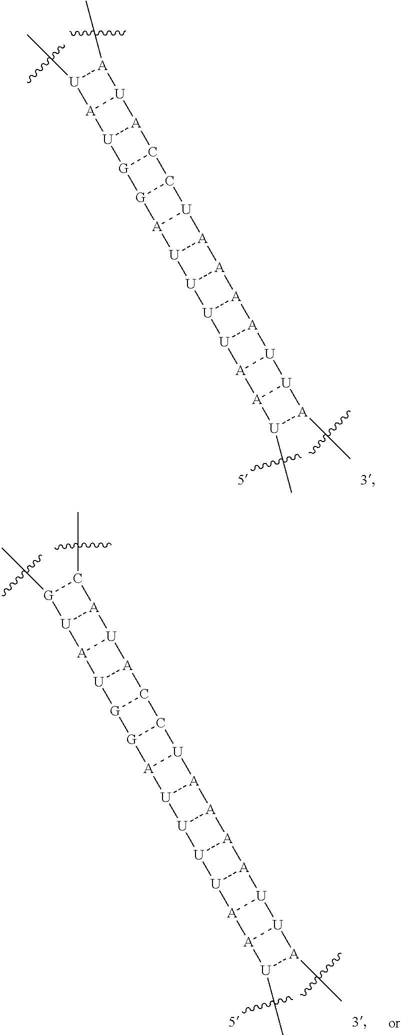

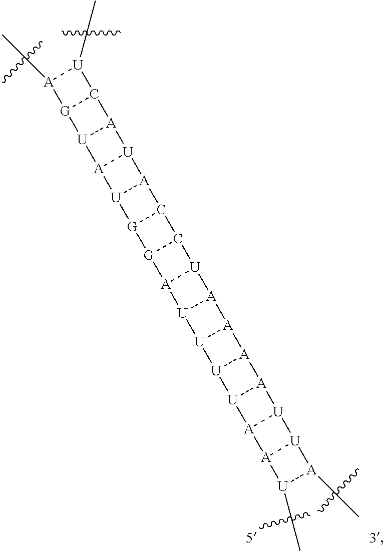

or covariants thereof. In some embodiments, ( [symbol] )_{u′} is of formula:

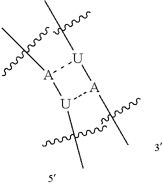

and B₁ is a adenine residue and B₂ is a uracil residue, or a covariant thereof. In some embodiments, ( [symbol] )_{u′} is of formula:

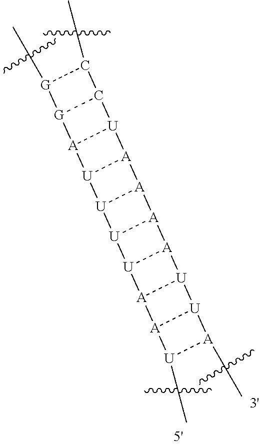

and B₁ is a uracil residue and B₂ is a adenine residue, or a covariant thereof. In some embodiments, ( [symbol] )_{u′} is of formula:

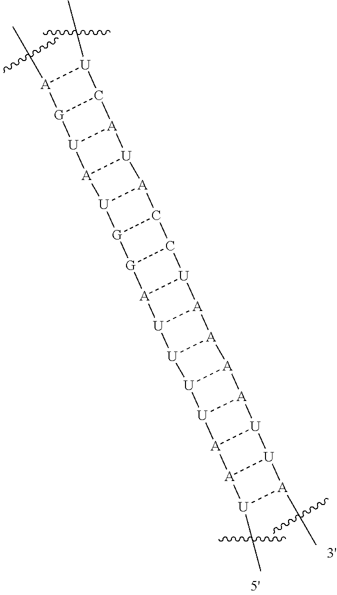

and B₁ is a guanine residue and B₂ is a cytosine residue, or a covariant thereof.

In some embodiments, Linker is of formula:

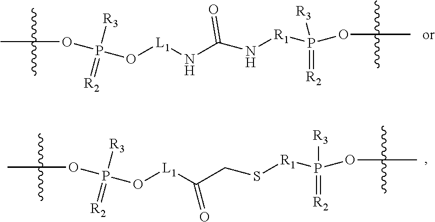

wherein:

each R₂ is independently O or S;

each R₃ is independently O⁻ or COO⁻; and

L₁ and R₁ are each a non-nucleotide chemical linker.

In some embodiments, the chemical linkage of a cross-linked guide molecule comprises a urea. In some embodiments, the guide molecule comprising a urea is of formula:

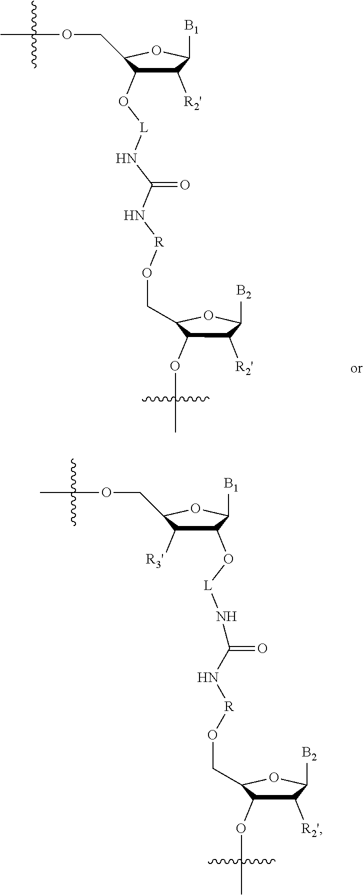

wherein L and R are each independently a non-nucleotide linker.

In some embodiments, the guide molecule comprising a urea is of formula:

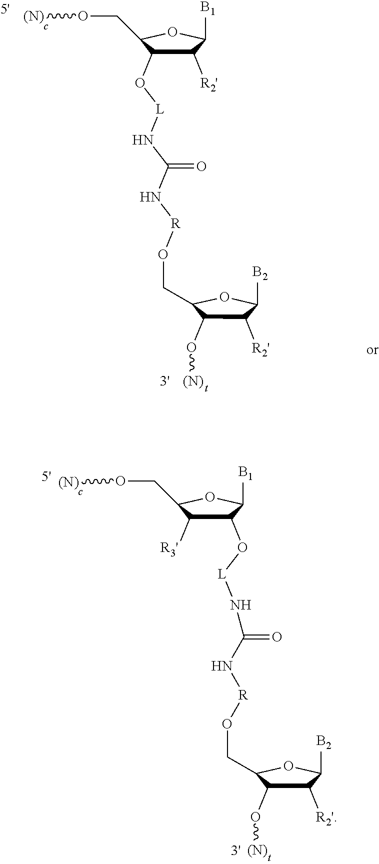

In some embodiments, the guide molecule comprising a urea is of formula:

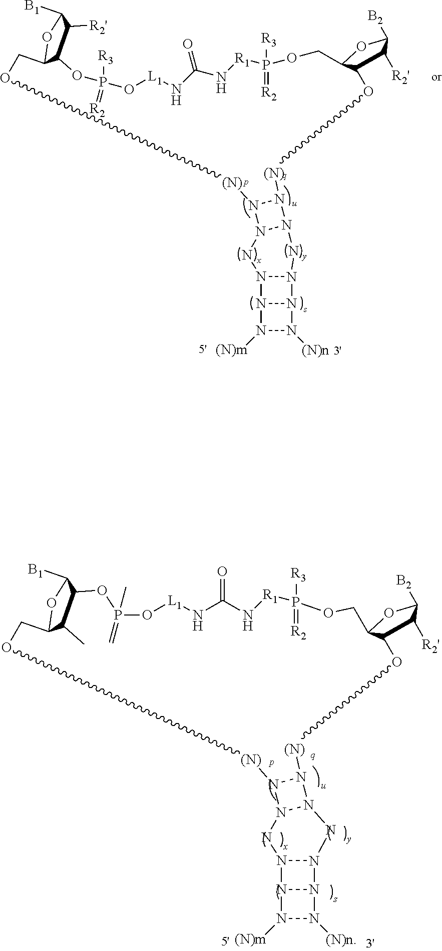

In some embodiments, the guide molecule comprising a urea is of formula:

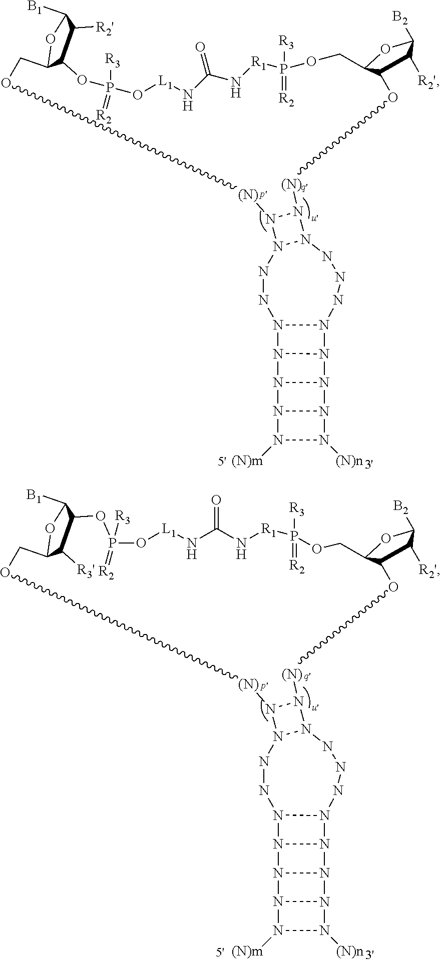

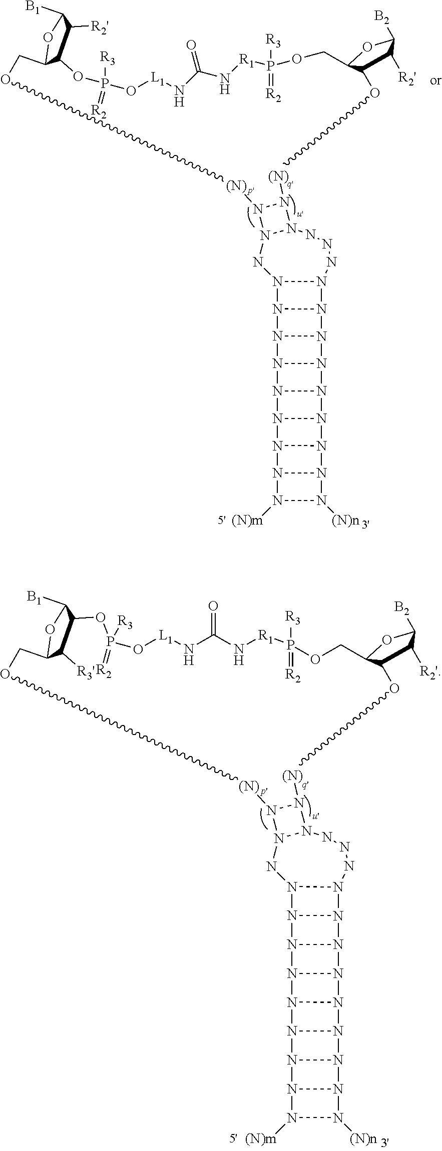

In some embodiments, the guide molecule comprising a urea is of formula:

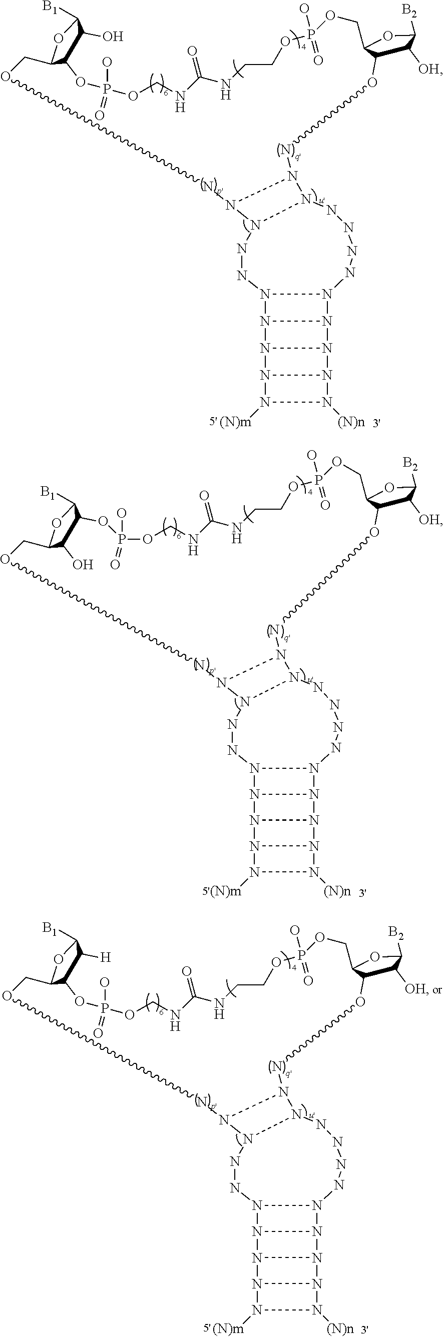

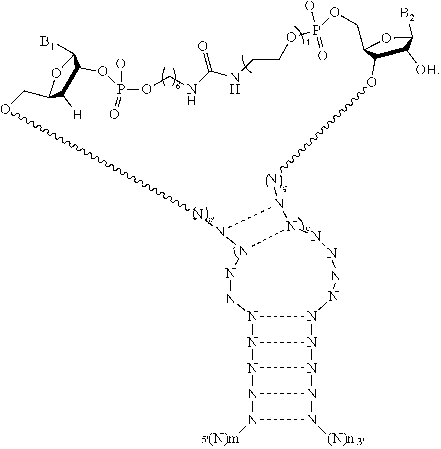

In some embodiments, the guide molecule comprising a urea is of sequence listed in Table 10 from the Examples section, wherein [UR] is a non-nucleotide linkage comprising a urea. In some embodiments, [UR] indicates the following linkage between two nucleotides with nucleobases B₁ and B₂:

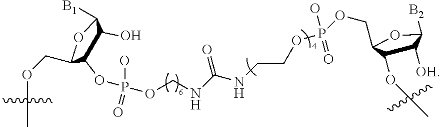

In some embodiments, the chemical linkage of a cross-linked guide molecule comprises a thioether. In some embodiments, the guide molecule comprising a thioether is of formula:

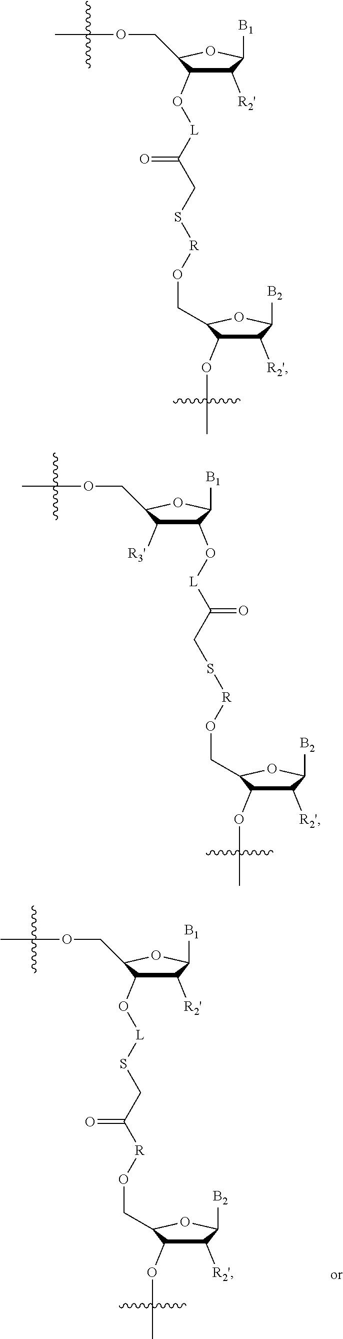

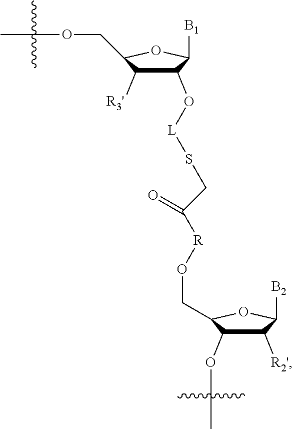

wherein L and R are each independently a non-nucleotide linker.

In some embodiments, the guide molecule comprising a thioether is of formula:

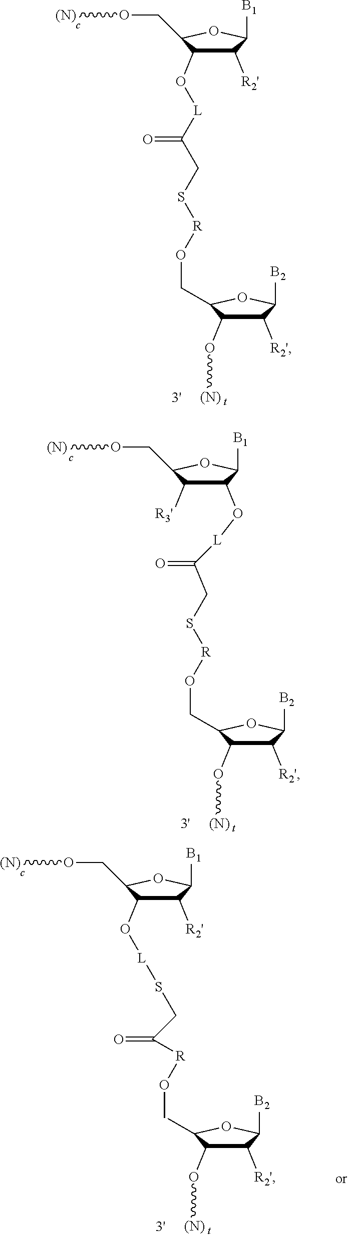

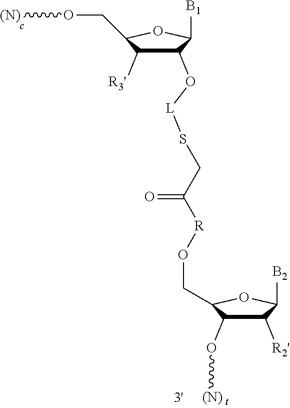

In some embodiments, the guide molecule comprising a thioether is of formula:

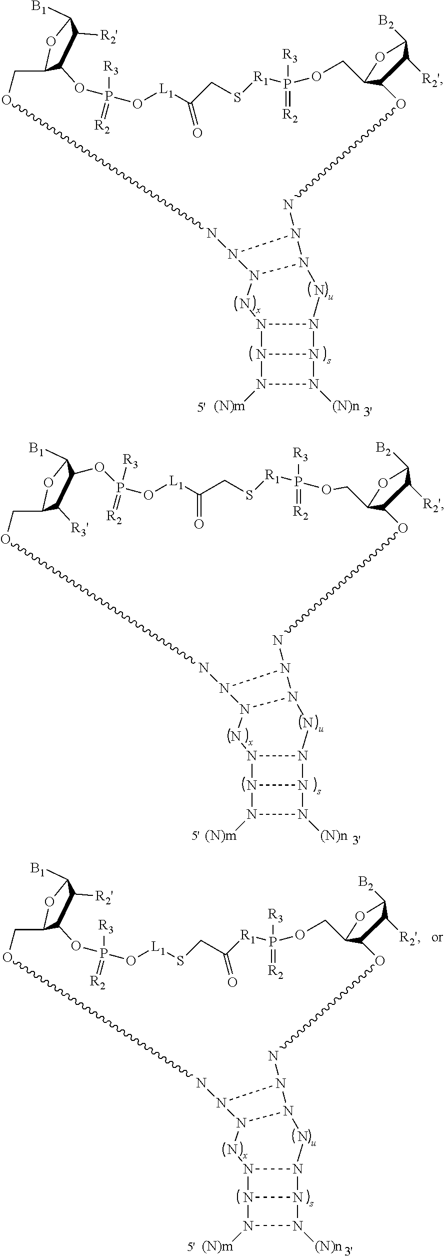

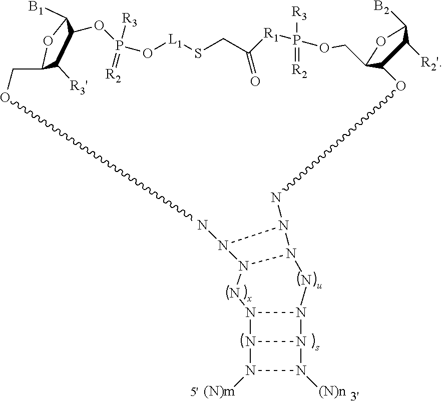

In some embodiments, the guide molecule comprising a thioether is of formula:

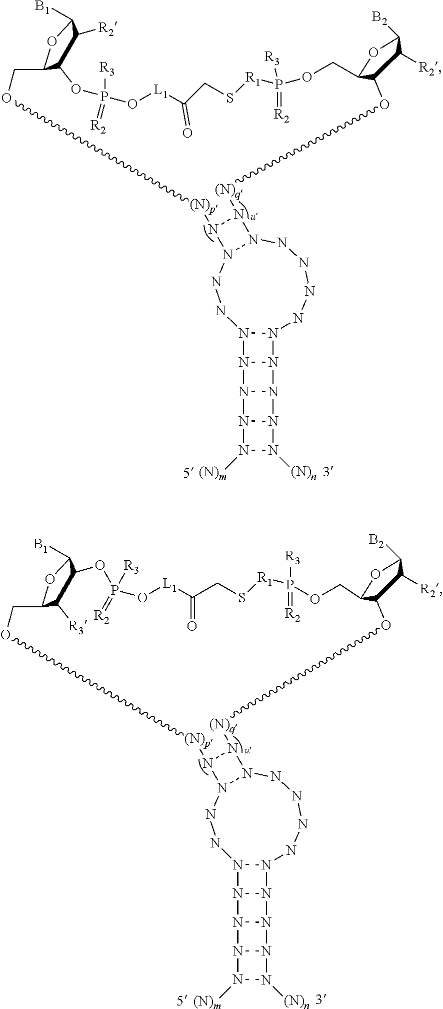

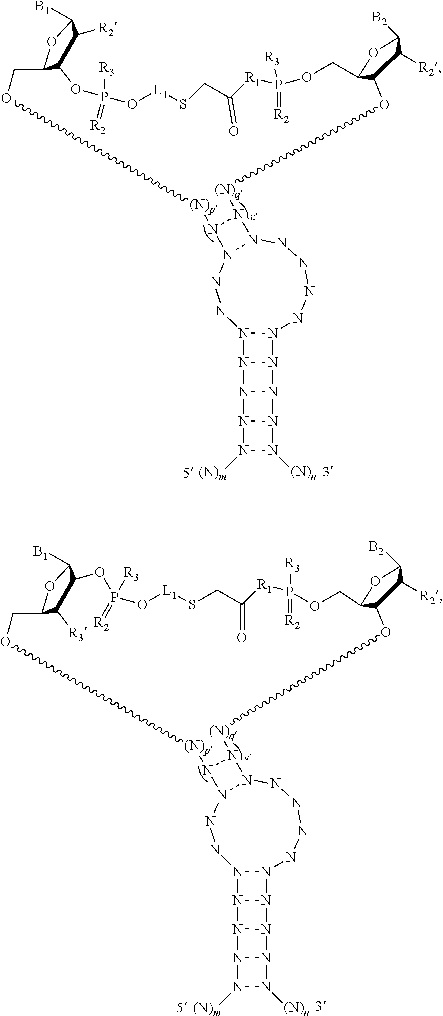

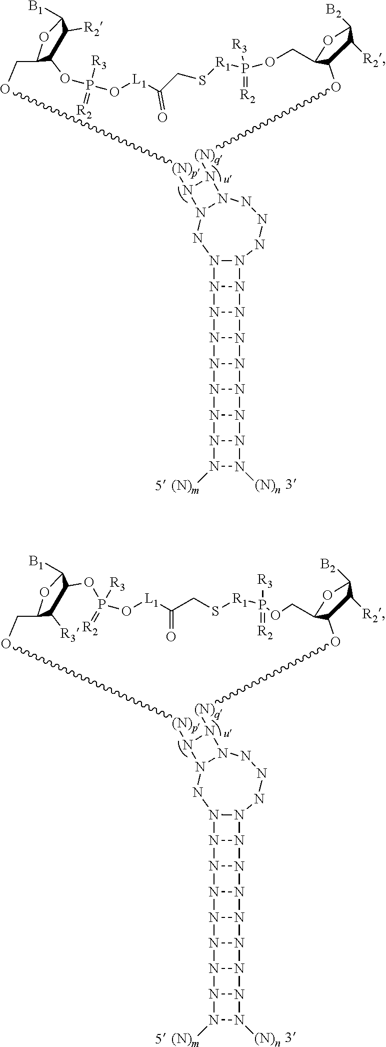

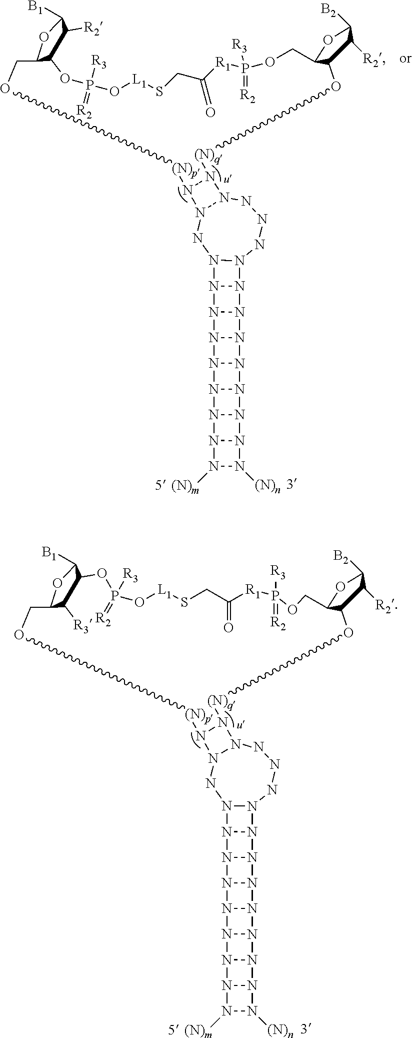

In some embodiments, the guide molecule comprising a thioether is of formula:

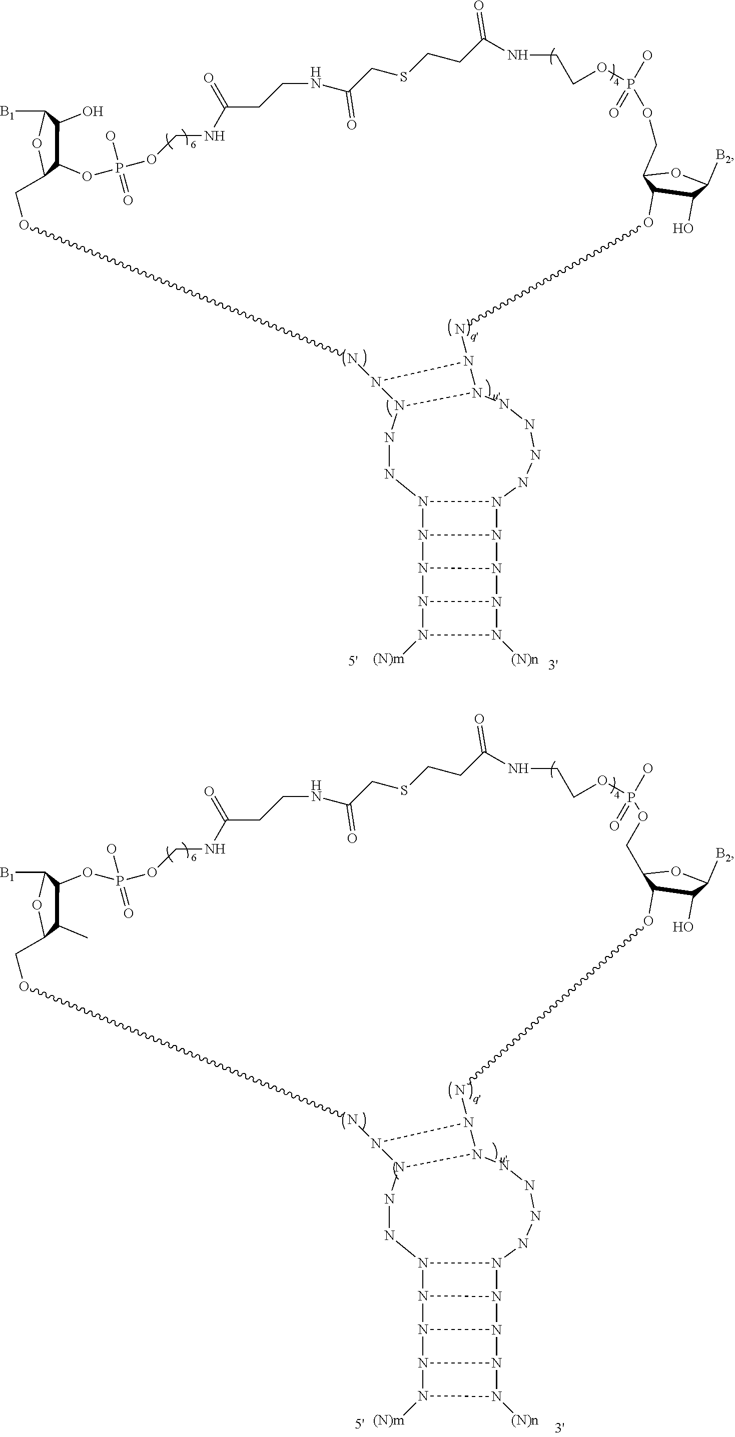

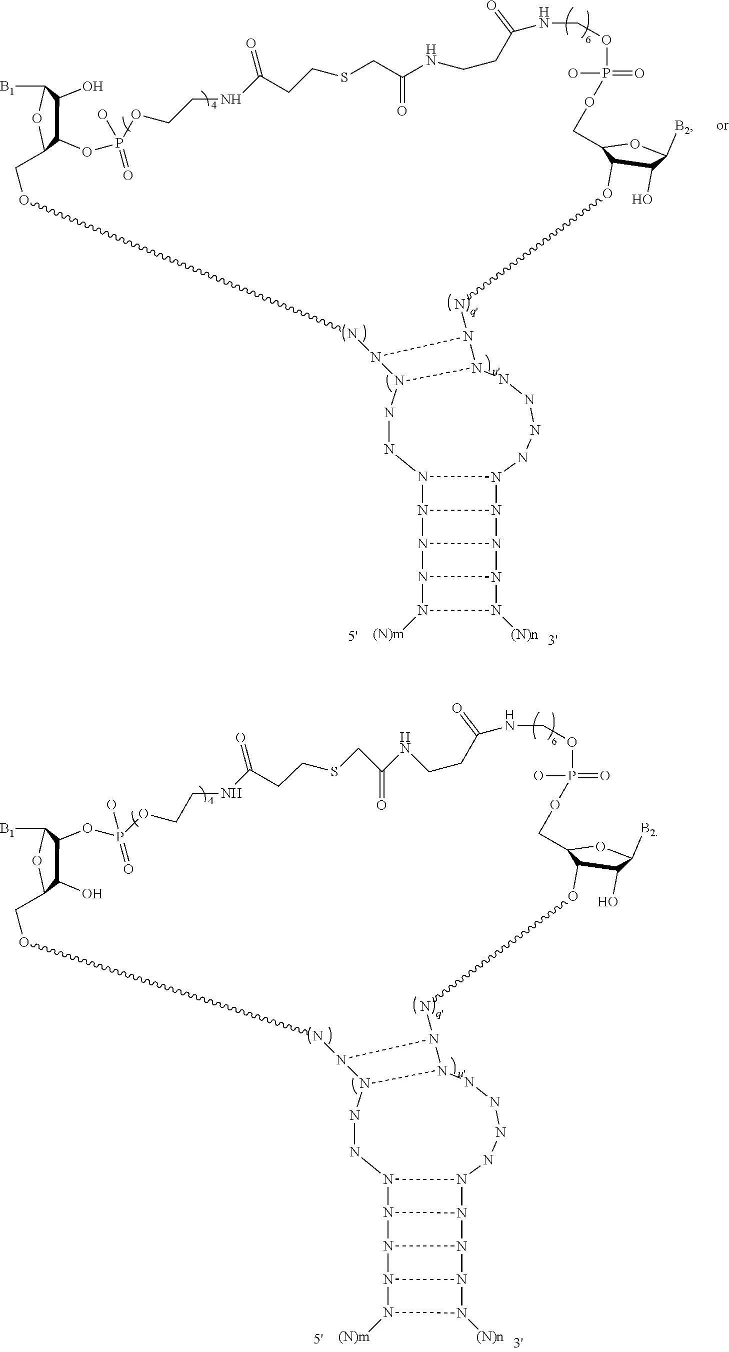

In some embodiments, the guide molecule comprising a urea is of sequence listed in Table 9 from the Examples section, wherein [L] is a thioether linkage. In some embodiments, [L] indicates the following linkage between two nucleotides with nucleobases B₁ and B₂:

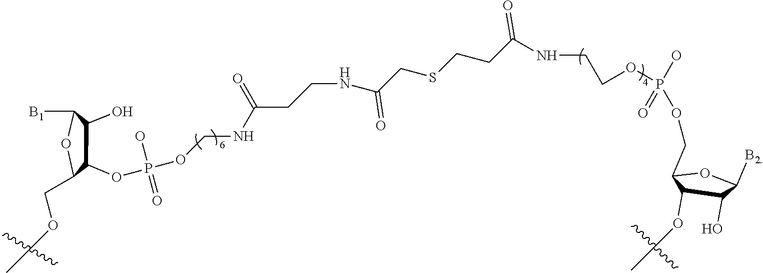

In some embodiments, in any formulae of this application, R_{2′} and R_{3′} are each independently H, OH, fluoro, chloro, bromo, NH₂, SH, S—R′, or O—R′ wherein each R′ is independently a protecting group or an optionally substituted alkyl group. In some embodiments, R_{2′} and R_{3′} are each independently H, OH, halogen, NH₂, or O—R′ wherein each R′ is independently a protecting group or an optionally substituted alkyl group. In some embodiments, R_{2′} and R_{3′} are each independently H, fluoro, and O—R′ wherein R′ is a protecting group or an optionally substituted alkyl group. In some embodiments, R_{2′} is H. In some embodiments, R_{3′} is H. In some embodiments, R_{2′} is halogen. In some embodiments, R_{3′} is halogen. In some embodiments, R_{2′} is fluorine. In some embodiments, R_{3′} is fluorine. In some embodiments, R_{2′} is O—R′. In some embodiments, R_{3′} is O—R′. In some embodiments, R_{2′} is O-Me. In some embodiments, R_{3′} is O-Me.

In some embodiments, in any formulae of this application, p and q are each independently 0, 1, 2, 3, 4, 5, or 6. In some embodiments, p and q are each independently 2. In some embodiments, p and q are each independently 0. In some embodiments, p′ and q′ are each independently 0, 1, 2, 3, or 4. In some embodiments, p′ and q′ are each independently 2. In some embodiments, p′ and q′ are each independently 0.

In some embodiments, in any formulae of this application, u is an integer between 2 and 22, inclusive. In some embodiments, u is an integer between 3 and 22, inclusive. In some embodiments, u is an integer between 4 and 22, inclusive. In some embodiments, u is an integer between 8 and 22, inclusive. In some embodiments, u is an integer between 12 and 22, inclusive. In some embodiments, u is an integer between 0 and 22, inclusive. In some embodiments, u is an integer between 2 and 14, inclusive. In some embodiments, u is an integer between 4 and 14, inclusive. In some embodiments, u is an integer between 8 and 14, inclusive. In some embodiments, u is an integer between 0 and 14, inclusive. In some embodiments, u is an integer between 0 and 4, inclusive. In some embodiments, in any formulae of this application, u′ is an integer between 2 and 22, inclusive. In some embodiments, u′ is an integer between 3 and 22, inclusive. In some embodiments, u′ is an integer between 4 and 22, inclusive. In some embodiments, u′ is an integer between 8 and 22, inclusive. In some embodiments, u′ is an integer between 12 and 22, inclusive. In some embodiments, u′ is an integer between 0 and 22, inclusive. In some embodiments, u′ is an integer between 2 and 14, inclusive. In some embodiments, u′ is an integer between 4 and 14, inclusive. In some embodiments, u′ is an integer between 8 and 14, inclusive. In some embodiments, u′ is an integer between 0 and 14, inclusive. In some embodiments, u′ is an integer between 0 and 4, inclusive.

In some embodiments, in any formulae of this application, N is independently a ribonucleotide, a deoxyribonucleotide, a modified ribonucleotide, or a modified deoxyribonucleotide. Nucleotide modifications are discussed below.

In some embodiments, in any formulae of this application, c is an integer 20 or greater. In some embodiments, c is an integer between 20 and 60, inclusive. In some embodiments, c is an integer between 20 and 40, inclusive. In some embodiments, c is an integer between 40 and 60, inclusive. In some embodiments, c is an integer between 30 and 60, inclusive. In some embodiments, c is an integer between 20 and 50, inclusive.

In some embodiments, in any formulae of this application, t is an integer 20 or greater. In some embodiments, t is an integer between 20 and 80, inclusive. In some embodiments, t is an integer between 20 and 50, inclusive. In some embodiments, t is an integer between 50 and 80, inclusive. In some embodiments, t is an integer between 20 and 70, inclusive. In some embodiments, t is an integer between 30 and 80, inclusive.

In some embodiments, in any formulae of this application, s is an integer between 1 and 10, inclusive. In some embodiments, s is an integer between 3 and 9, inclusive. In some embodiments, s is an integer between 1 and 8, inclusive. In some embodiments, s is an integer between 0 and 10, inclusive. In some embodiments, s is an integer between 2 and 6, inclusive.

In some embodiments, in any formulae of this application, x is an integer between 1 and 3, inclusive. In some embodiments, x is 1. In some embodiments, x is 2. In some embodiments, x is 3. In some embodiments, in any formulae of this application, y is greater than x. In some embodiments, y is an integer between 3 and 5, inclusive. In some embodiments, y is 3. In some embodiments, y is 4. In some embodiments, y is 5. In some embodiments, x is 1 and y is 3. In some embodiments, x is 2 and y is 4.

In some embodiments, in any formulae of this application, m is an integer 15 or greater. In some embodiments, m is an integer between 15 and 50, inclusive. In some embodiments, m is an integer 16 or greater. In some embodiments, m is an integer 17 or greater. In some embodiments, m is an integer 18 or greater. In some embodiments, m is an integer 19 or greater. In some embodiments, m is an integer 20 or greater. In some embodiments, m is an integer between 20 and 40, inclusive. In some embodiments, m is an integer between 30 and 50, inclusive. In some embodiments, m is an integer between 15 and 30, inclusive.

In some embodiments, in any formulae of this application, n is an integer 30 or greater. In some embodiments, n is an integer between 30 and 70, inclusive. In some embodiments, n is an integer between 30 and 60, inclusive. In some embodiments, n is an integer between 40 and 70, inclusive.

In some embodiments, in any formulae of this application, L, R, L₁ and R₁ are each independently a non-nucleotide linker. In some embodiments, L, R, L₁ and R₁ each independently comprise a moiety selected from the group consisting of polyethylene, polypropylene, polyethylene glycol, and polypropylene glycol. In some embodiments, L₁ and R₁ are each independently —(CH₂)_{w}—, —(CH₂)_{w}—NH—C(O)—(CH₂)_{w}—NH—, —(OCH₂CH₂)ᵥ—NH—C(O)—(CH₂)_{w}—, or —(CH₂CH₂O)ᵥ—, and each w is an integer between 1-20, inclusive and each v is an integer between 1-10, inclusive. In some embodiments, L₁ is —(CH₂)_{w}—. In some embodiments, L₁ is —(CH₂)_{w}—NH—C(O)—(CH₂)_{w}—NH—. In some embodiments, L₁ is —(OCH₂CH₂)ᵥ—NH—C(O)—(CH₂)_{w}—. In some embodiments, L₁ is —(CH₂)₆—. In some embodiments, L₁ is —(CH₂)₆—NH—C(O)—(CH₂)₁—NH—. In some embodiments, L₁ is —(OCH₂CH₂)₄—NH—C(O)—(CH₂)₂—. In some embodiments, R₁ is —(CH₂CH₂O)ᵥ—. In some embodiments, R₁ is —(CH₂)_{w}—NH—C(O)—(CH₂)_{w}—NH—. In some embodiments, R₁ is —(OCH₂CH₂)ᵥ—NH—C(O)—(CH₂)_{w}—. In some embodiments, R₁ is —(CH₂CH₂O)₄—. In some embodiments, L₁ is —(CH₂)₆—NH—C(O)—(CH₂)₁—NH—. In some embodiments, R₁ is —(OCH₂CH₂)₄—NH—C(O)—(CH₂)₂—. In some embodiments, L₁ is —(CH₂)₆— and R₁ is —(CH₂CH₂O)₄—. In some embodiments, L₁ is —(CH₂)₆—NH—C(O)—(CH₂)₁—NH— and R₁ is —(OCH₂CH₂)₄—NH—C(O)—(CH₂)₂—.

In some embodiments, in any formulae of this application, R₂ is O, and in some embodiments, R₂ is S. In some embodiments, R₃ is O⁻, and in some embodiments, R₃ is COO⁻. In some embodiments, R₂ is O and R₃ is O⁻. In some embodiments, R₂ is O and R₃ is COO⁻. In some embodiments, R₂ is S and R₃ is O⁻. In some embodiments, R₂ is S and R₃ is COO⁻. One skilled in the art will recognize that R₃ can also exist in a protonated form (OH and COOH). Throughout this application, we intend to encompass both the deprotonated and protonated forms of R₃.

In some embodiments, in any formulae of this application, each [symbol] independently represents two complementary nucleotides, optionally two complementary nucleotides that are hydrogen bonding base-paired. In some embodiments, all [symbol] represent two complementary nucleotides that are hydrogen bonding base-paired. In some embodiments, some [symbol] represent two complementary nucleotides and some [symbol] represent two complementary nucleotides that are hydrogen bonding base-paired.

In some embodiments, in any formulae of this application, B₁ and B₂ are each independently a nucleobase. In some embodiments, B₁ is guanine and B₂ is cytosine. In some embodiments, B₁ is cytosine and B₂ is guanine. In some embodiments, B₁ is adenine and B₂ is uracil. In some embodiments, B₁ is uracil and B₂ is adenine. In some embodiments, B₁ and B₂ are complementary. In some embodiments, B₁ and B₂ are complementary and base-paired through hydrogen bonding. In some embodiments, B₁ and B₂ are complementary and not base-paired through hydrogen bonding. In some embodiments, B₁ and B₂ are not complementary.

Synthesis of Guide Molecules

Another aspect of the invention is a method of synthesizing a unimolecular guide molecule, the method comprising the steps of:
annealing a first oligonucleotide and a second oligonucleotide to form a duplex between a 3′ region of the first oligonucleotide and a 5′ region of the second oligonucleotide, wherein the first oligonucleotide comprises a first reactive group which is at least one of a 2′ reactive group and a 3′ reactive group, and wherein the second oligonucleotide comprises a second reactive group which is a 5′ reactive group; and
conjugating the annealed first and second oligonucleotides via the first and second reactive groups to form a unimolecular guide molecule that includes a covalent bond linking the first and second oligonucleotides.

In some embodiments, the first reactive group and the second reactive group are selected from the functional groups listed above under “Overview.” In some embodiments, the first reactive group and the second reactive group are each independently an amine moiety, a sulfhydryl moiety, a bromoacetyl moiety, a hydroxyl moiety, or a phosphate moiety. In some embodiments, the first reactive group and the second reactive group are both amine moieties. In some embodiments, the first reactive group is a sulfhydryl moiety, and the second reactive group is a bromoacetyl moiety. In some embodiments, the first reactive group is a bromoacetyl moiety, and the second reactive group is a sulfhydryl moiety. In some embodiments, the first reactive group is a hydroxyl moiety and the second reactive group is a phosphate moiety. In some embodiments, the first reactive group is a phosphate moiety, and the second reactive group is a hydroxyl moiety.

In some embodiments, the step of conjugating comprises a concentration of first nucleotide in the range of 10 μM to 1 mM. In some embodiments, the step of conjugating comprises a concentration of second nucleotide in the range of 10 μM to 1 mM. In some embodiments, the concentration of either the first or second nucleotide is 10 μM, 50 μM, 100 μM, 200 μM, 400 μM, 600 μM, 800 μM, or 1 mM.

In some embodiments, the step of conjugating comprises a pH in the range of 5.0 to 9.0. In some embodiments, the pH is 5.0, 5.5, 6.0, 6.5, 7.0, 7.5, 8.0, 8.5, or 9.0. In some embodiments, the pH is 6.0. In some embodiments, the pH is 8.0. In some embodiments, the pH is 8.5.

In some embodiments, the step of conjugating is performed under argon. In some embodiments, the step of conjugating is performed under ambient atmosphere.

In some embodiments, the step of conjugating is performed in water. In some embodiments, the step of conjugating is performed in water with a cosolvent. In some embodiments, the cosolvent is DMSO, DMF, NMP, DMA, morpholine, pyridine, or MeCN. In some embodiments, the cosolvent is DMSO. In some embodiments, the cosolvent is DMF.

In some embodiments, the step of conjugating is performed at a temperature in the range of 0° C. to 40° C. In some embodiments, the temperature is 0° C., 4° C., 10° C., 20° C., 25° C., 30° C., 37° C., or 40° C. In some embodiments, the temperature is 25° C. In some embodiments, the temperature is 4° C.

In some embodiments, the step of conjugating is performed in the presence of a divalent metal cation. In some embodiments, the divalent metal cation is Mg²⁺, Ca²⁺, Sr²⁺, Ba²⁺, Cr²⁺, Mn²⁺, Fe²⁺, Co²⁺, Ni²⁺, Cu²⁺, or Zn²⁺. In some embodiments, the divalent metal cation is Mg²⁺.

In some embodiments, the step of conjugating comprises a cross-linking reagent or a cross-linker (see “Overview” above). In some embodiments, the cross-linker is multifunctional, and in some embodiments the cross-linker is bifunctional. In some embodiments, the multifunctional cross-linker is heterofunctional or homofunctional. In some embodiments, the cross-linker contains a carbonate. In some embodiments, the carbonate-containing cross-linker is disuccinimidyl carbonate, diimidazole carbonate, or bis-(p-nitrophenyl) carbonate. In some embodiments, the carbonate-containing cross-linker is disuccinimidyl carbonate.

In some embodiments, the step of conjugating comprises a concentration of bifunctional crosslinking reagent in the range of 1 mM to 100 mM. In some embodiments, the concentration of bifunctional crosslinking reagent is 1 mM, 10 mM, 20 mM, 40 mM, 60 mM, 80 mM, or 100 mM. In some embodiments, the concentration of bifunctional crosslinking reagent is 100 to 1000 times greater than the concentration of each of the first and second oligonucleotides. In some embodiments, the concentration of bifunctional crosslinking reagent is 100, 200, 400, 600, 800, or 1000 times greater than the concentration of the first oligonucleotide. In some embodiments, the concentration of bifunctional crosslinking reagent is 100, 200, 400, 600, 800, or 1000 times greater than the concentration of the second oligonucleotide.

In some embodiments, the step of conjugating is performed in the presence of a chelating reagent. In some embodiments, the chelating reagent is ethylenediaminetetraacetic acid (EDTA), or a salt thereof.

In some embodiments, the step of conjugating is performed in the presence of an activating agent. In some embodiments, the activating agent is a carbodiimide, or salt thereof. In some embodiments, the carbodiimide is 1-ethyl-3-(3-dimethylaminopropyl)carbodiimide (EDC), N,N′-dicyclohexylcarbodiimide (DCC) or N,N′-diisopropylcarbodiimide (DIC), or a salt thereof. In some embodiments, the carbodiimide is 1-ethyl-3-(3-dimethylaminopropyl)carbodiimide (EDC), or a salt thereof.

In some embodiments, the step of conjugating comprises a concentration of activating agent that is in the range of 1 mM to 100 mM. In some embodiments, the concentration of activating agent is 1 mM, 10 mM, 20 mM, 40 mM, 60 mM, 80 mM, or 100 mM. In some embodiments, the concentration of activating agent is 100 to 1000 times greater than the concentration of each of the first and second oligonucleotides. In some embodiments, the concentration of activating agent is 100, 200, 400, 600, 800, or 1000 times greater than the concentration of the first oligonucleotide. In some embodiments, the concentration of activating agent is 100, 200, 400, 600, 800, or 1000 times greater than the concentration of the second oligonucleotide.

In some embodiments, the step of conjugating is performed in the presence of a stabilizing agent. In some embodiments, the stabilizing agent is imidazole, cyanoimidazole, pyridine, or dimethylaminopyridine, or a salt thereof. In some embodiments, the stabilizing agent is imidazole. In some embodiments, the step of conjugating is performed in the presence of both an activating agent and a stabilizing agent. In some embodiments, the step of conjugating is performed in the presence of 1-ethyl-3-(3-dimethylaminopropyl)carbodiimide (EDC) and imidazole, or salts thereof.

In some embodiments, the method of synthesizing a unimolecular guide molecule generates a guide molecule of any formula disclosed above.

In some embodiments, the method of synthesizing a unimolecular guide molecule results in a guide molecule with a urea linker. In some embodiments, first reactive group and the second reactive group are both amines, and the first and second reactive groups are cross-linked with a carbonate-containing bifunctional crosslinking reagent to form a urea linker. In some embodiments, the carbonate-containing bifunctional crosslinking reagent is disuccinimidyl carbonate. In some embodiments, the method comprises a first oligonucleotide of formula:

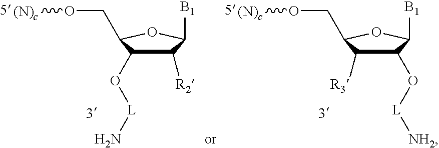

or a salt thereof. In some embodiments, the method comprises a second oligonucleotide of formula:

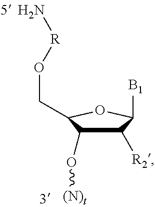

or a salt thereof.

In some embodiments, the method of synthesizing a unimolecular guide molecule results in a guide molecule with a thioether linker. In some embodiments, first reactive group is a sulfhydryl group and the second reactive group is a bromoacetyl group, or the first reactive group is a bromoacetyl group and the second reactive group is a sulfhydryl group. In some embodiments, the first reactive group and the second reactive group react in the presence of a chelating agent to form a thioether linkage. In some embodiments, the first reactive group and the second reactive group undergo a substitution reaction to form a thioether linkage. In some embodiments, the method comprises a first oligonucleotide of formula:

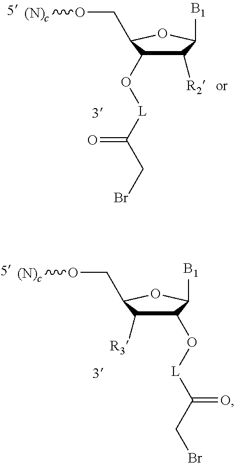

or a salt thereof, and
the second oligonucleotide is of formula:

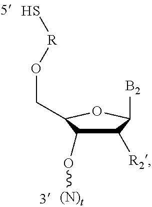

or a salt thereof; or the method comprises a first oligonucleotide of formula:

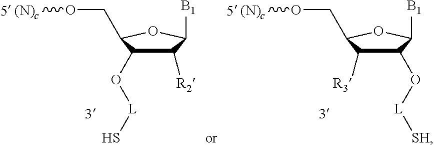

or a salt thereof, and
the second oligonucleotide is of formula:

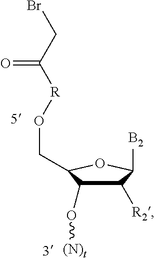

or a salt thereof.

In some embodiments, the method of synthesizing a unimolecular guide molecule results in a guide molecule with a phosphodiester linker. In some embodiments, first reactive group comprises a 2′ or 3′ hydroxyl group and the second reactive group comprises a 5′ phosphate moiety. In some embodiments, the first and second reactive groups are conjugated in the presence of an activating agent to form a phosphodiester linker. In some embodiments, the activating agent is EDC. In some embodiments, the method comprises a first oligonucleotide of formula:

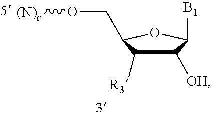

or a salt thereof; and
the second oligonucleotide is of formula:

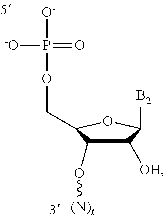

or a salt thereof.

In some embodiments, the method of synthesizing a unimolecular guide molecule generates a unimolecular guide molecule with at least one 2′-5′ phosphodiester linkage in a duplex region.

Oligonucleotide Intermediates

Certain embodiments of this disclosure are related to oligonucleotide intermediates that are useful for the synthesis of cross-linked synthetic guide molecules. In some embodiments, the oligonucleotide intermediates are useful for the synthesis of guide molecules comprising a urea linkage, a thioether linkage or a phosphodiester linkage. In some embodiments, the oligonucleotide intermediates comprise an annealed duplex.

In certain embodiments, the oligonucleotide intermediates are useful in the synthesis of guide molecules comprising a urea linkage. In some embodiments, the oligonucleotide intermediates are of

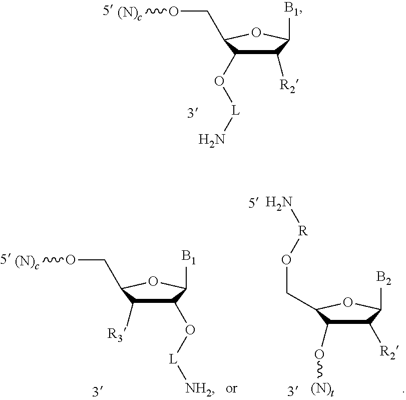

In some embodiments, the oligonucleotide intermediates are of formula:

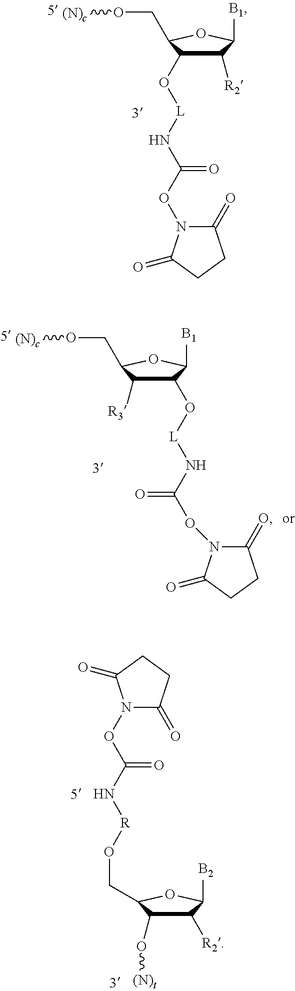

In some embodiments, the oligonucleotide intermediates are of formula:

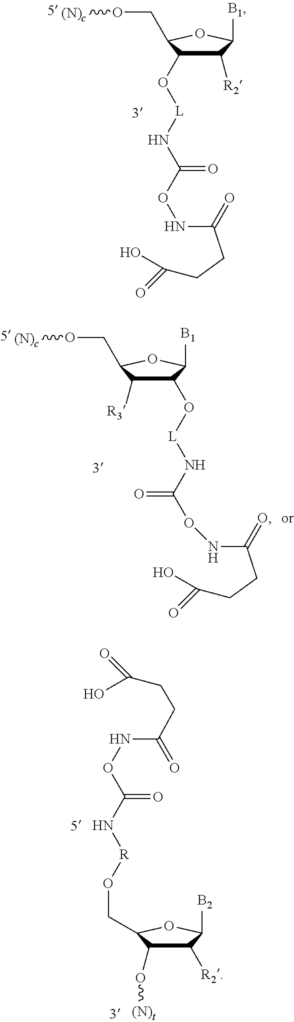

In some embodiments, the oligonucleotide intermediates are of formula:

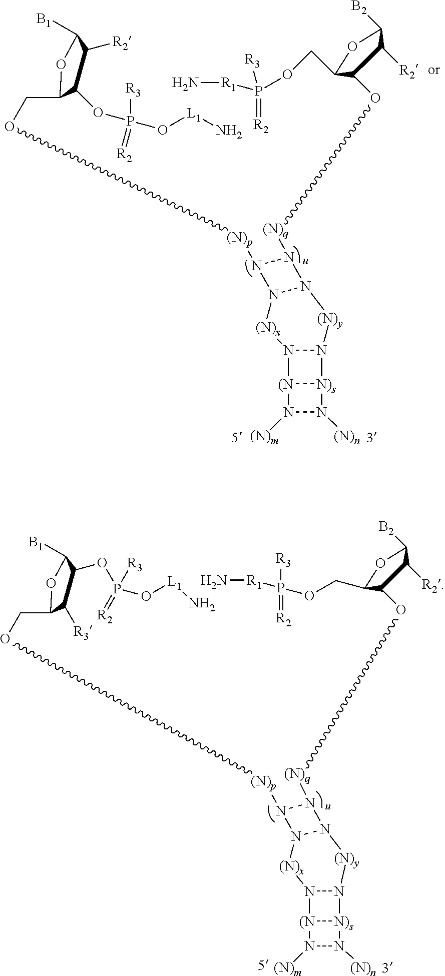

In some embodiments, the oligonucleotide intermediates are of formula:

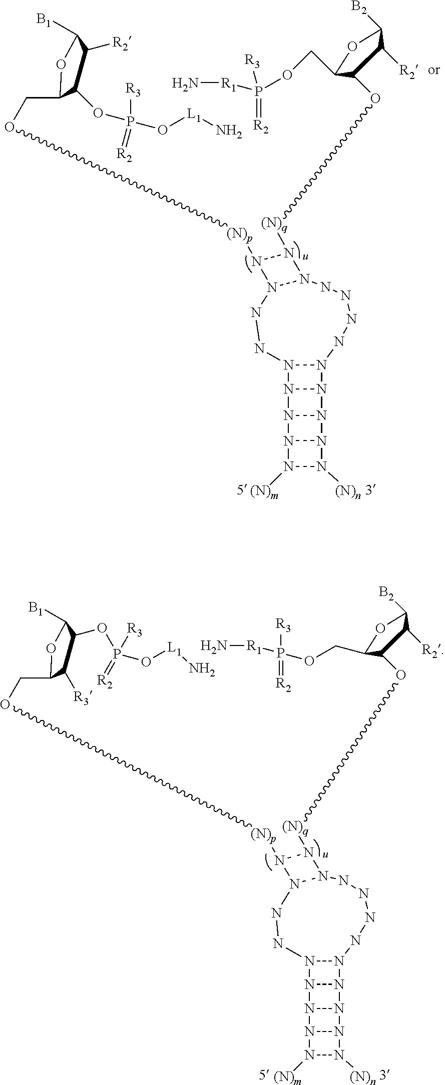

In some embodiments, the oligonucleotide intermediates are of formula:

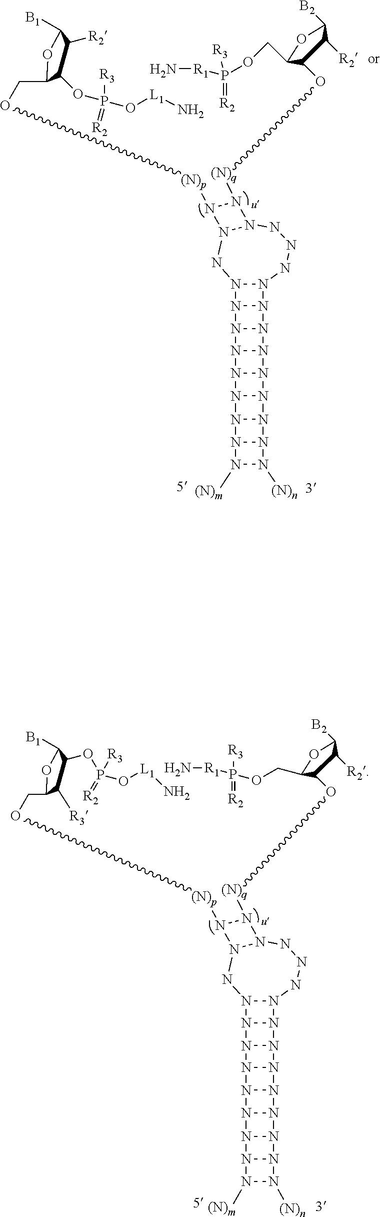

In certain embodiments, the oligonucleotide intermediates are useful in the synthesis of guide molecules comprising a thioether linkage. In some embodiments, the oligonucleotide intermediates are of formula:

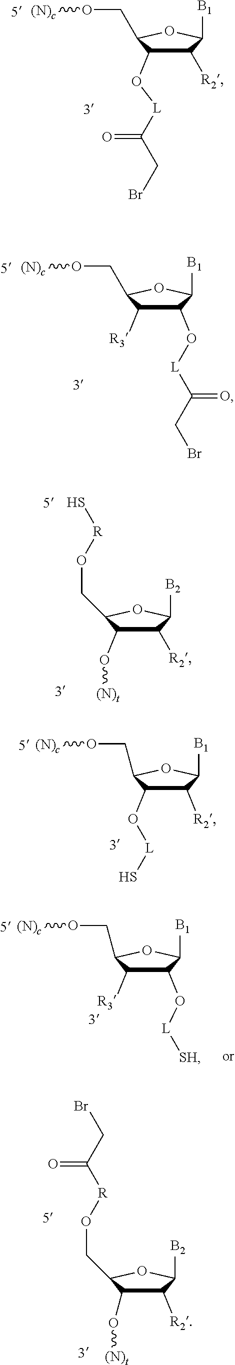

In some embodiments, the oligonucleotide intermediates are of formula:

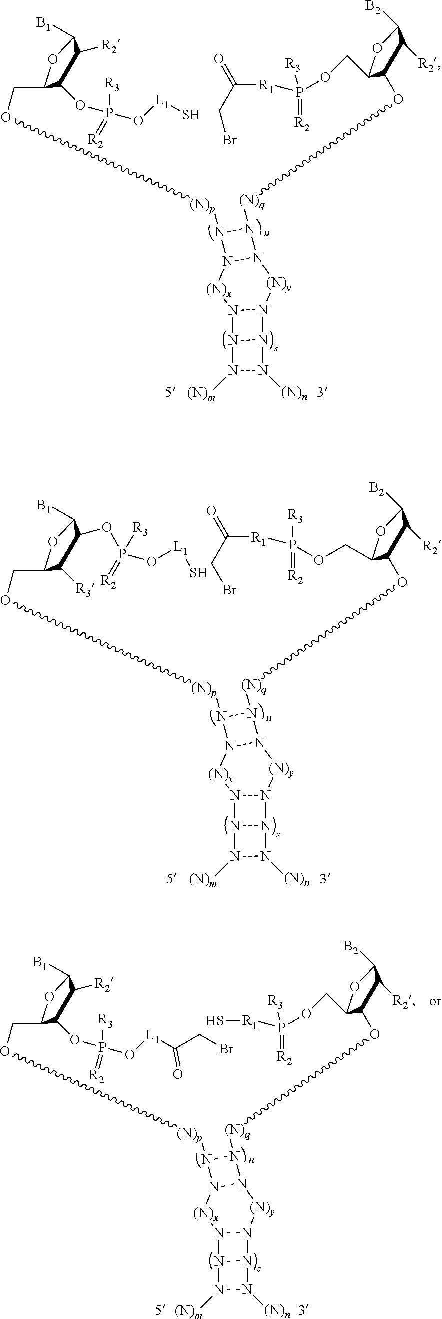

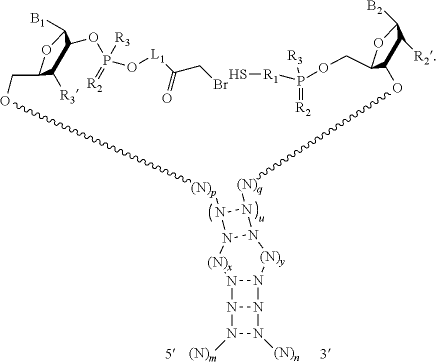

In some embodiments, the oligonucleotide intermediates are of formula:

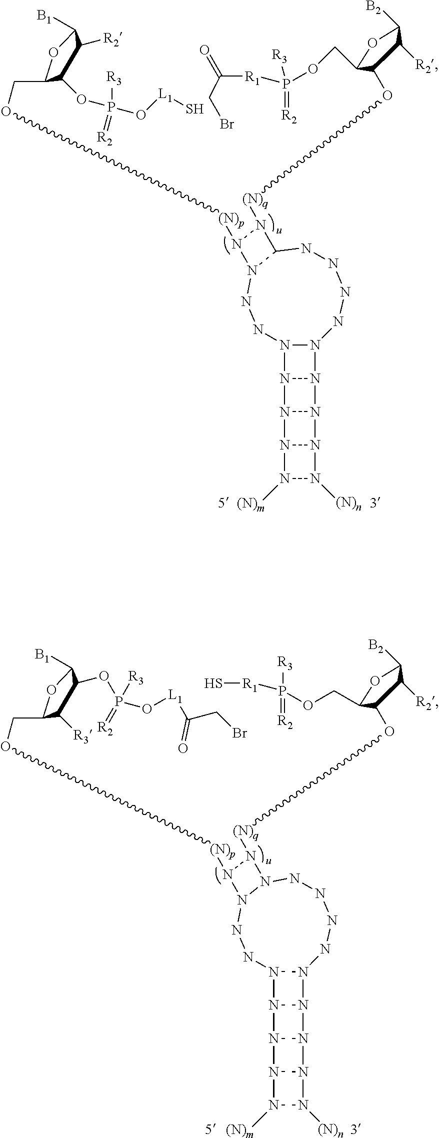

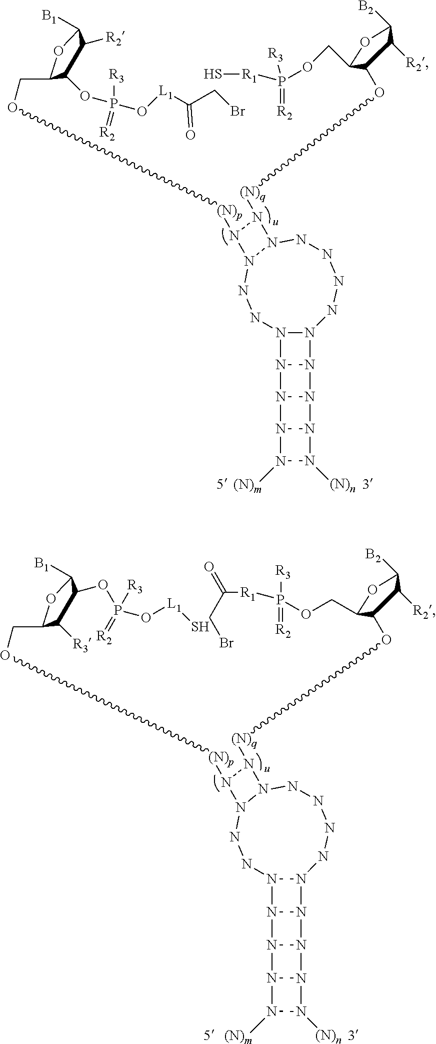

In some embodiments, the oligonucleotide intermediates are of formula:

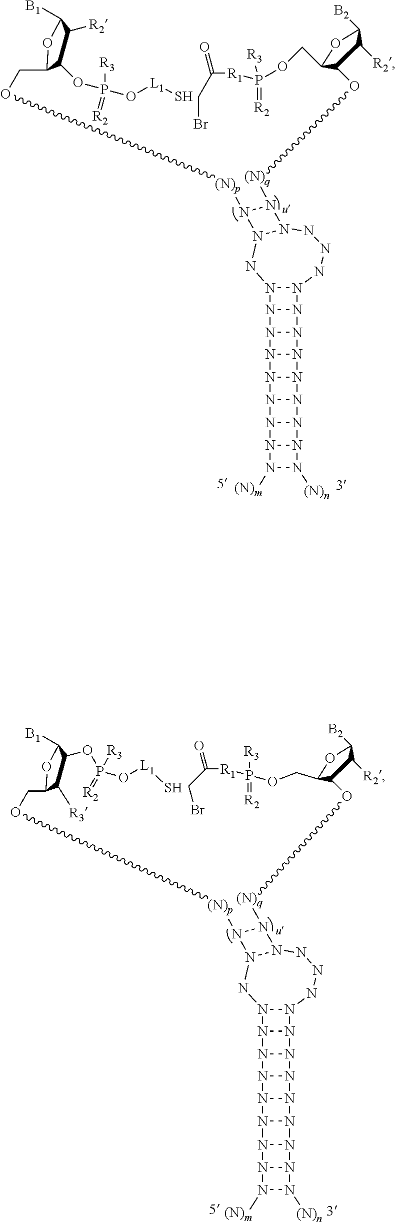

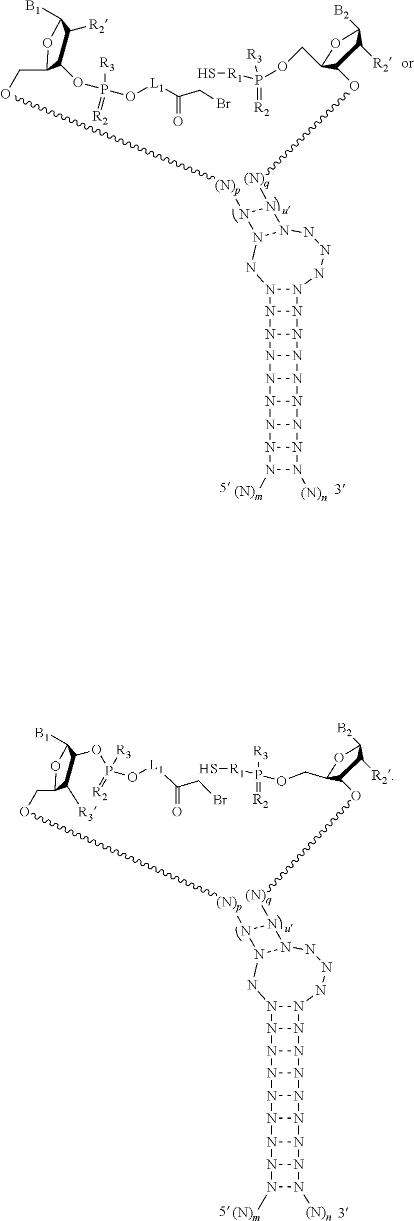

In certain embodiments, the oligonucleotide intermediates are useful in the synthesis of guide molecules comprising a phosphodiester linkage. In some embodiments, the oligonucleotide intermediates are of formula:

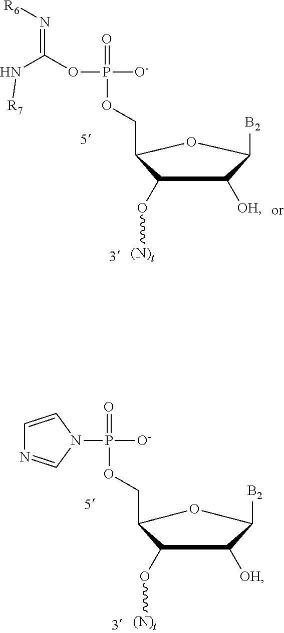

wherein R₆ and R₇ are each independently substituted or unsubstituted alkyl, or substituted or unsubstituted carbocyclyl. In some embodiments, the oligonucleotide intermediates are of formula:

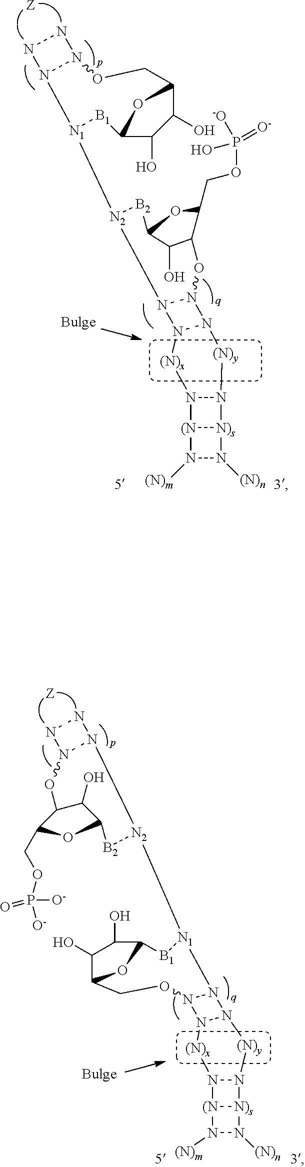

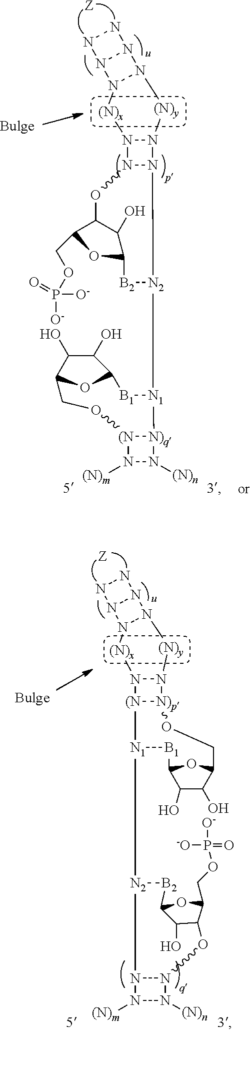

wherein Z represents a nucleotide loop which is 4-6 nucleotides long, optionally 4 or 6 nucleotides long.

Certain embodiments of this disclosure relate to oligonucleotide compounds that are formed as side products in a cross linking reaction. These oligonucleotide compounds may or may not be useful as guide molecules. In some embodiments, the oligonucleotide compound is of formula:

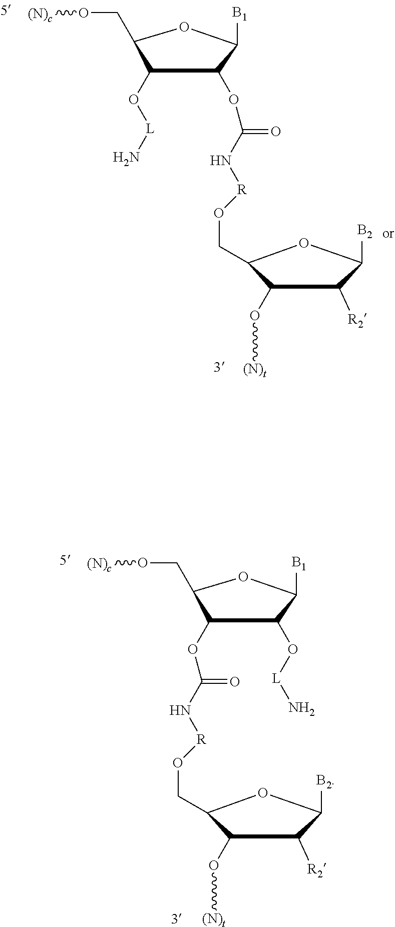

Compositions of Chemically Conjugated Guide Molecules

Certain embodiments of this disclosure are related to compositions comprising synthetic guide molecules described above and to compositions generated by the methods described above. In some embodiments, the composition is characterized in that greater than 90% of guide molecules in the composition are full length guide molecules. In some embodiments, the composition is characterized in that greater than 85% of guide molecules in the composition comprise an identical targeting domain sequence.

In some embodiments, the composition has not been subjected to a purification step. In some embodiments, the composition of guide molecules for a CRISPR system consists essentially of guide molecules of formula:

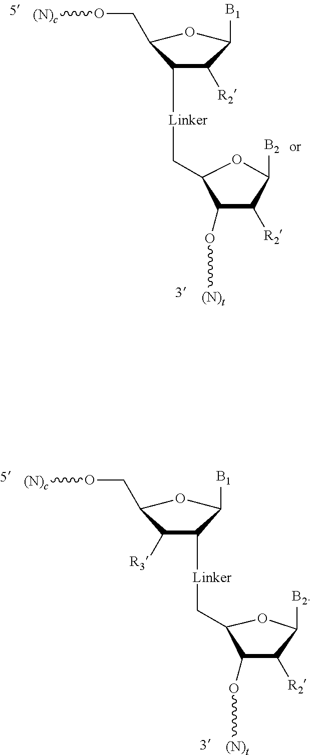

In some embodiments, the composition consists essentially of guide molecules of formula:

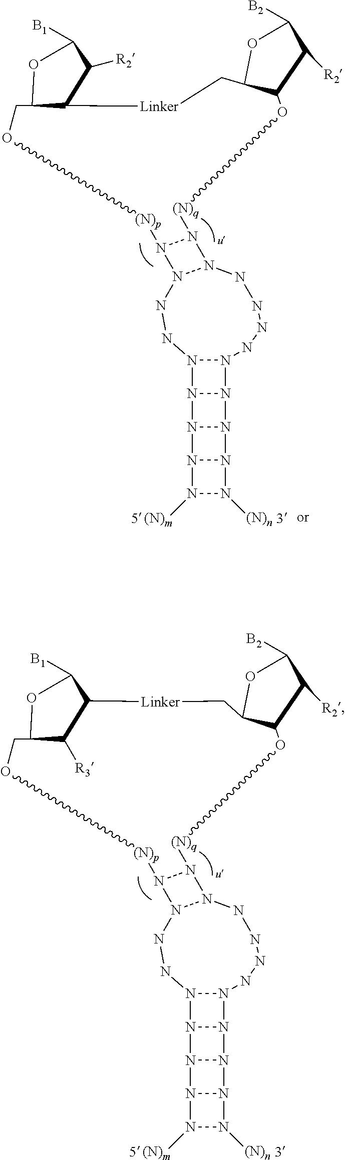

or a salt thereof. In some embodiments, the composition consists essentially of guide molecules of formula:

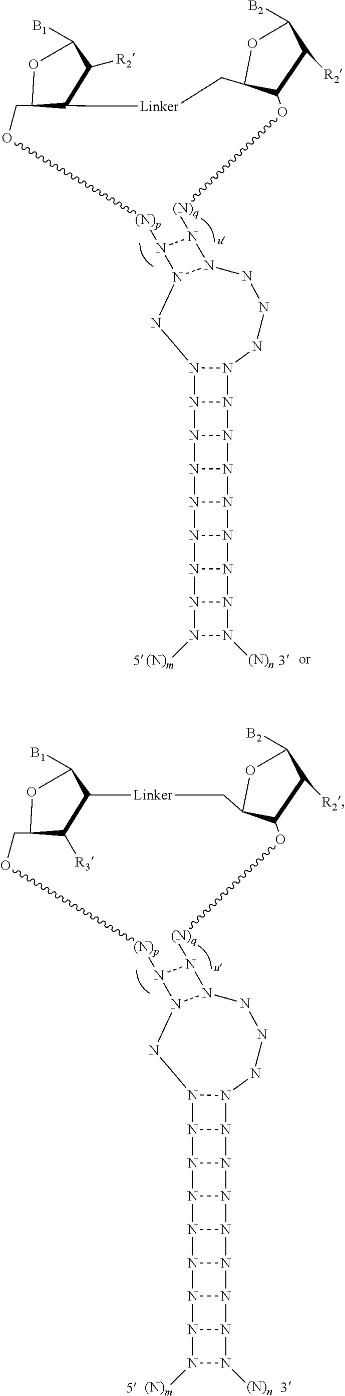

or a salt thereof. In some embodiments, the composition consists essentially of guide molecules of formula:

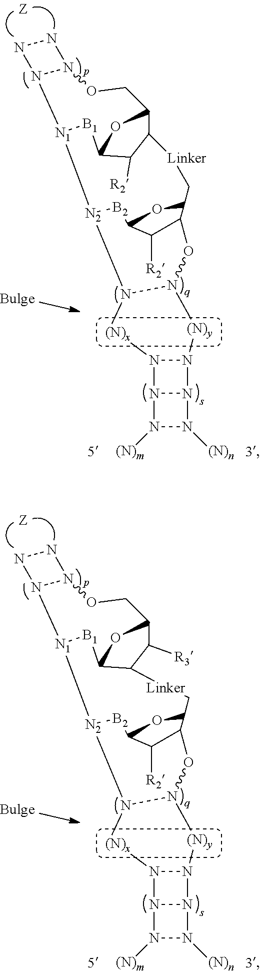

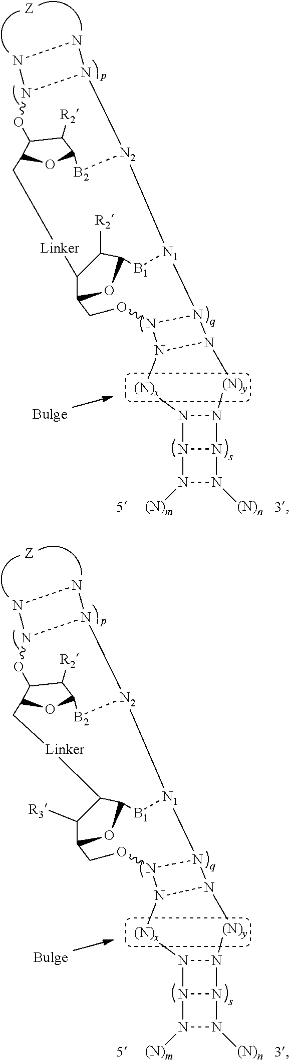

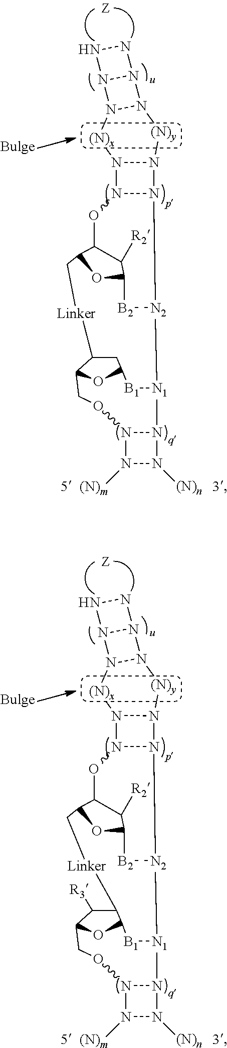

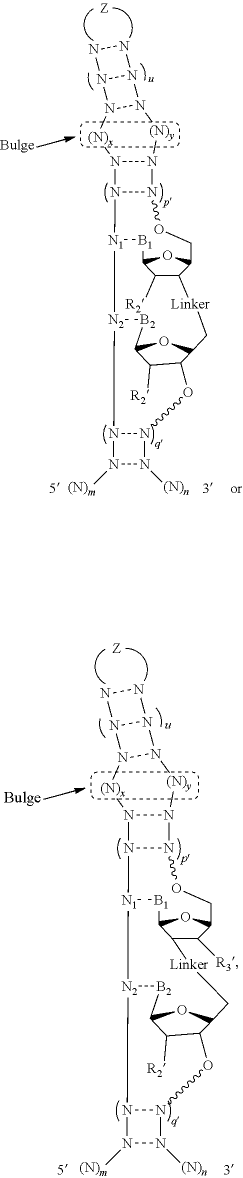

or a salt thereof.

In some embodiments, the composition comprises oligonucleotide intermediates (described above) in the presence or absence of a synthetic guide molecule. In some embodiments, the oligonucleotide intermediates of the composition are of formula:

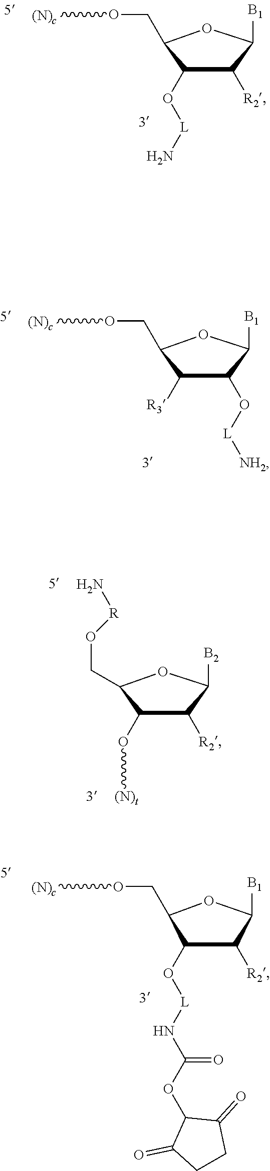

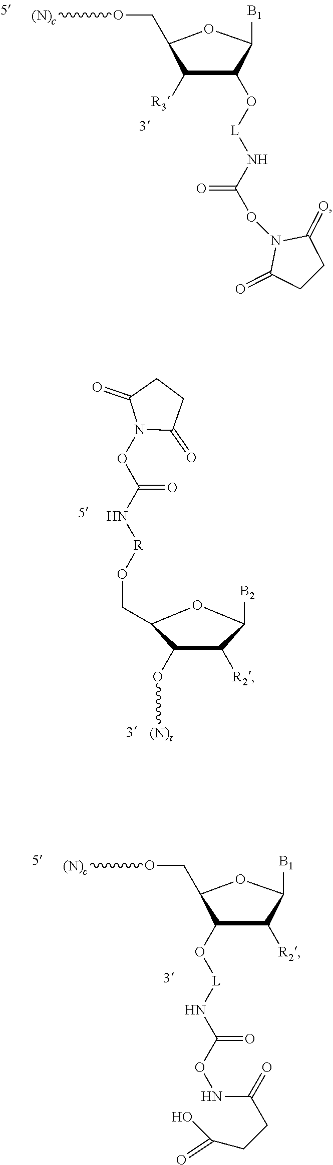

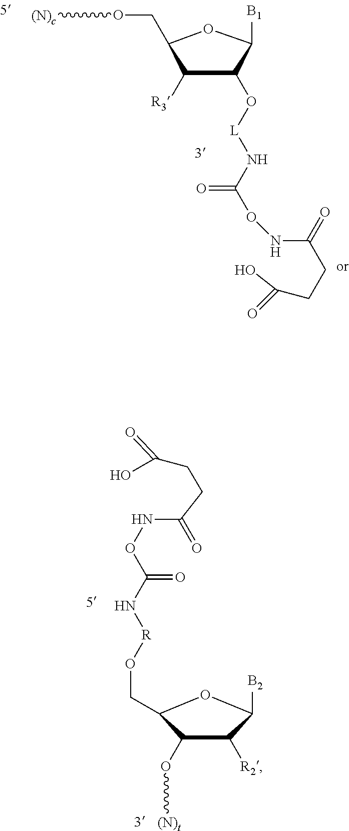

and the synthetic guide molecule is of formula:

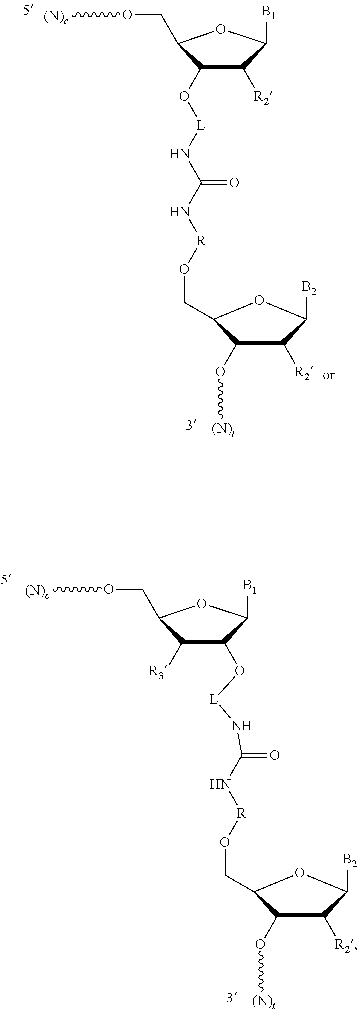

or a pharmaceutically acceptable salt thereof. In some embodiments, the composition comprises oligonucleotide intermediates with an annealed duplex of formula:

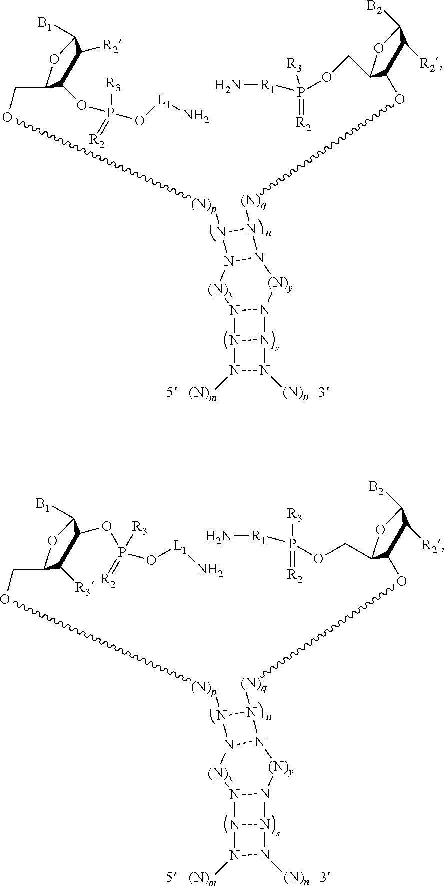

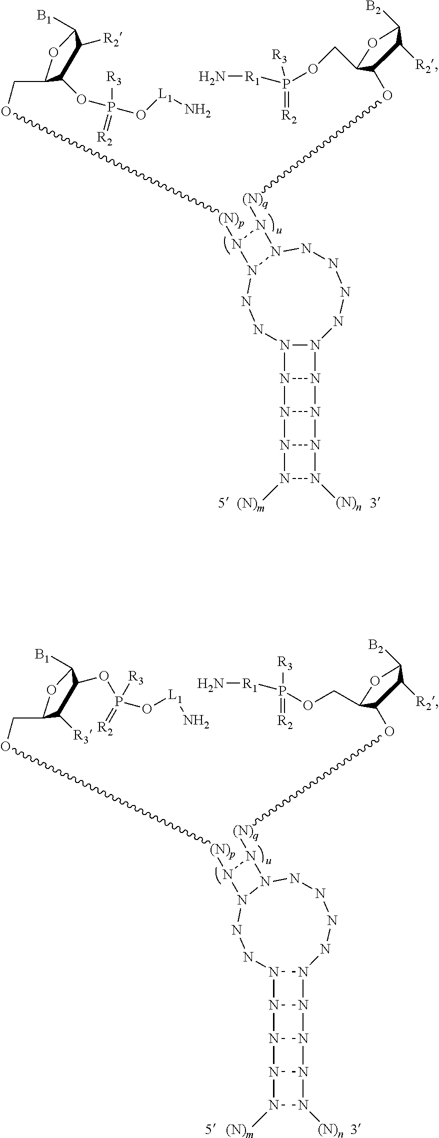

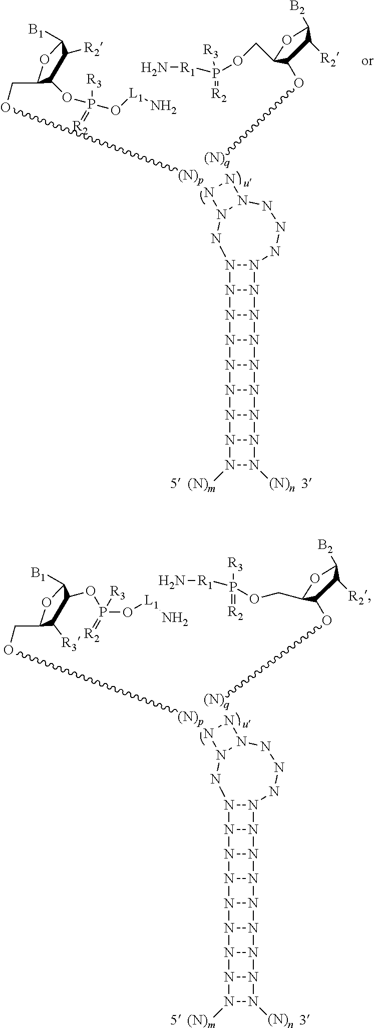

or a salt thereof, in the presence or absence of a synthetic guide molecule of formula:

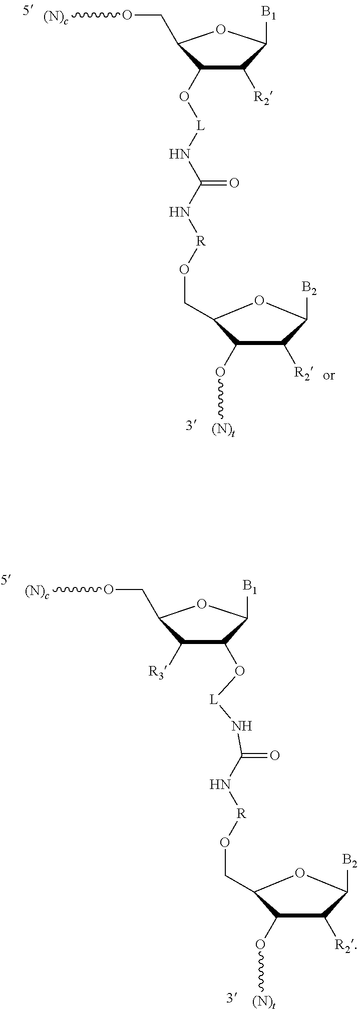

In some embodiments, the oligonucleotide intermediates in the composition are of formula:

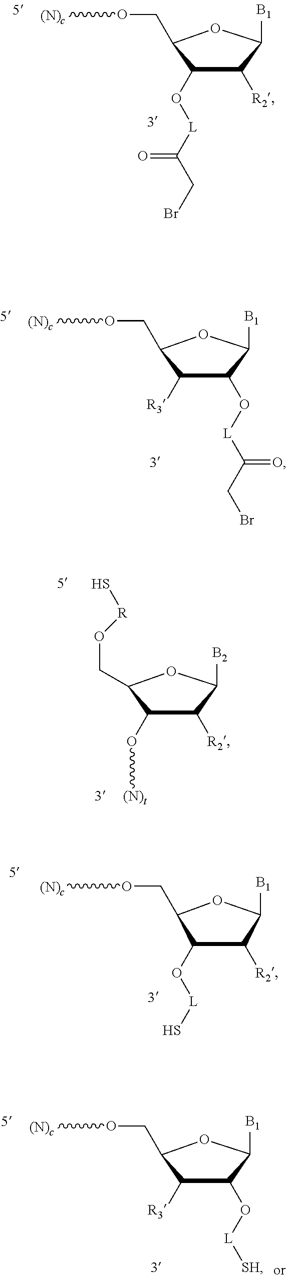

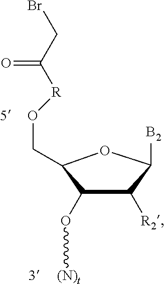

or a salt thereof, and the synthetic guide molecule is of formula:

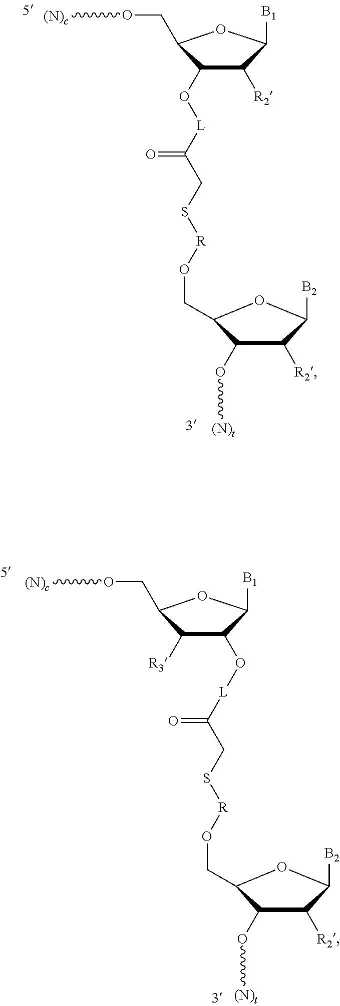

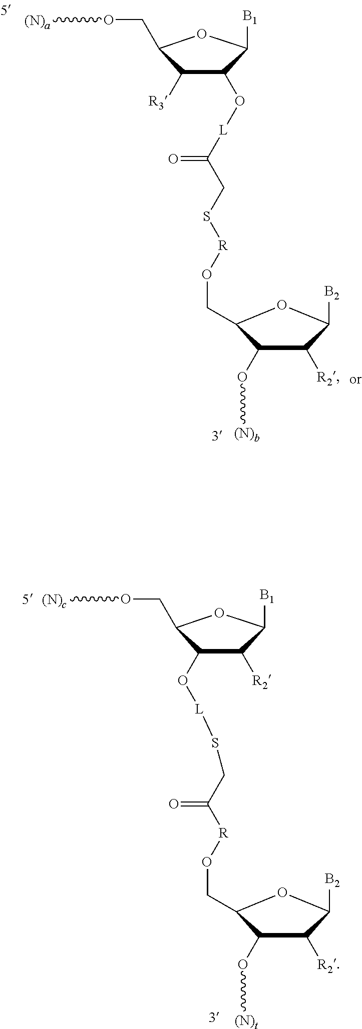

In some embodiments, the composition comprises oligonucleotide intermediates with an annealed duplex of formula:

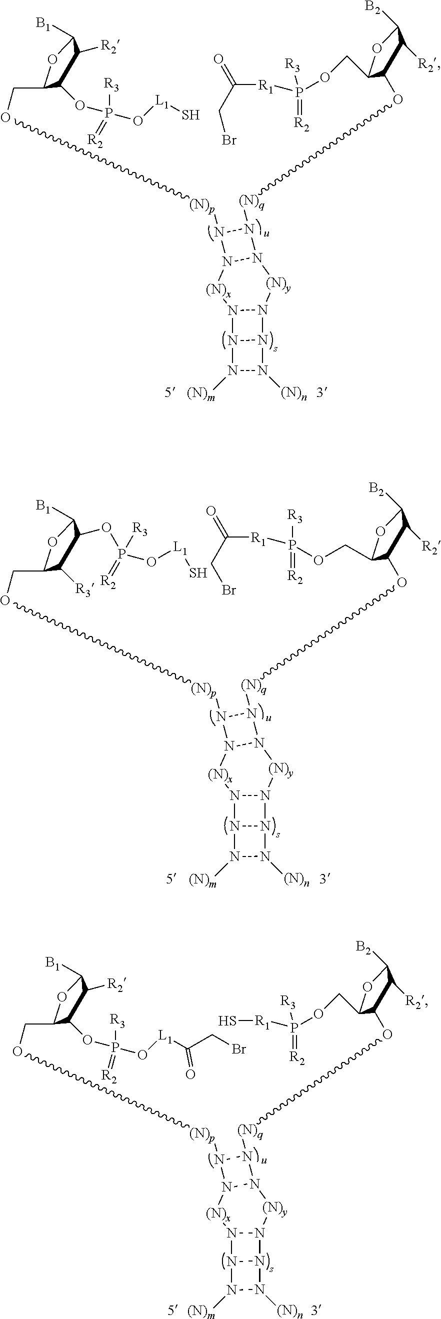

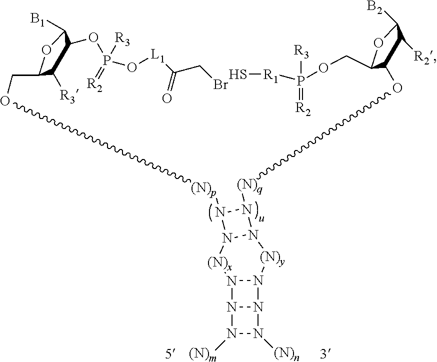

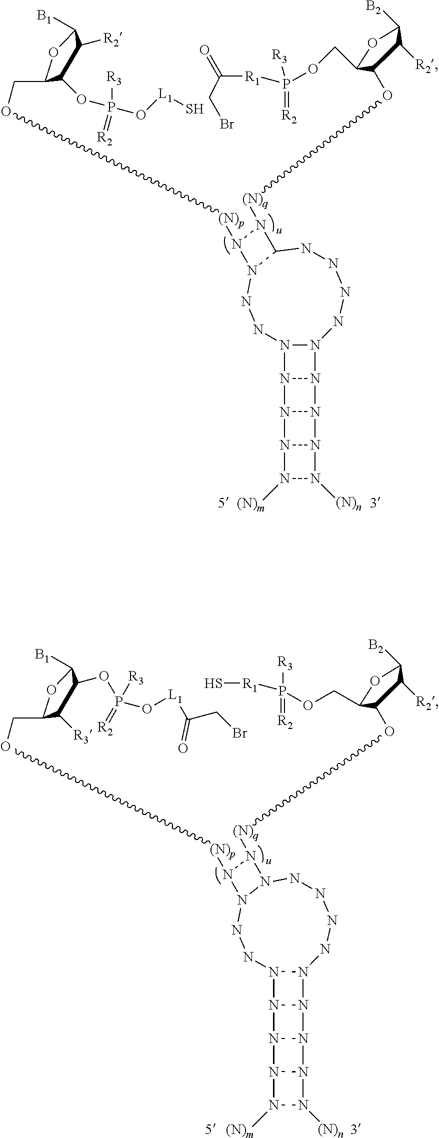

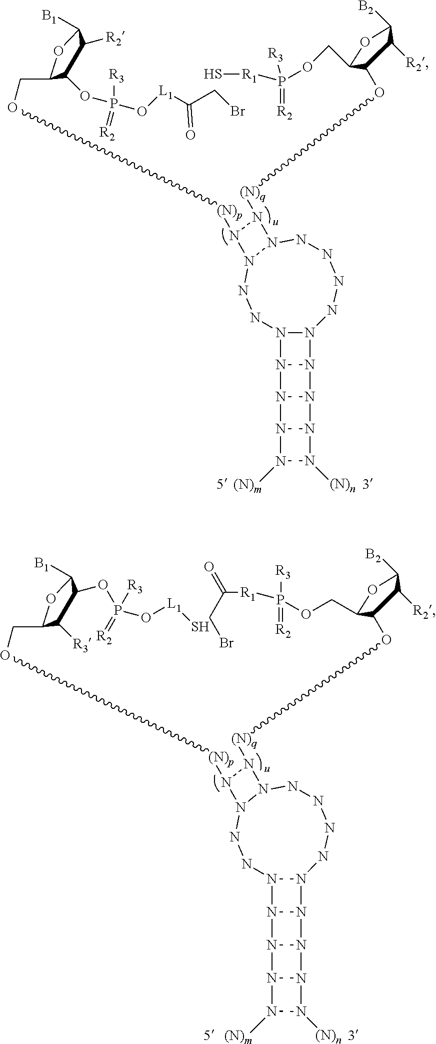

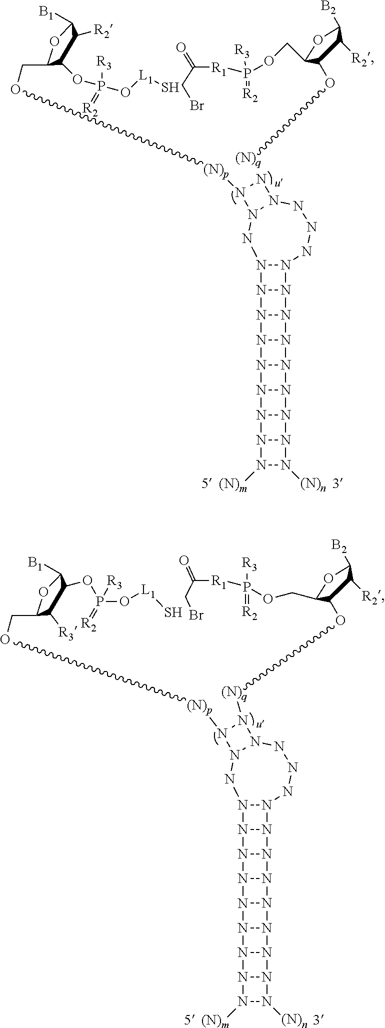

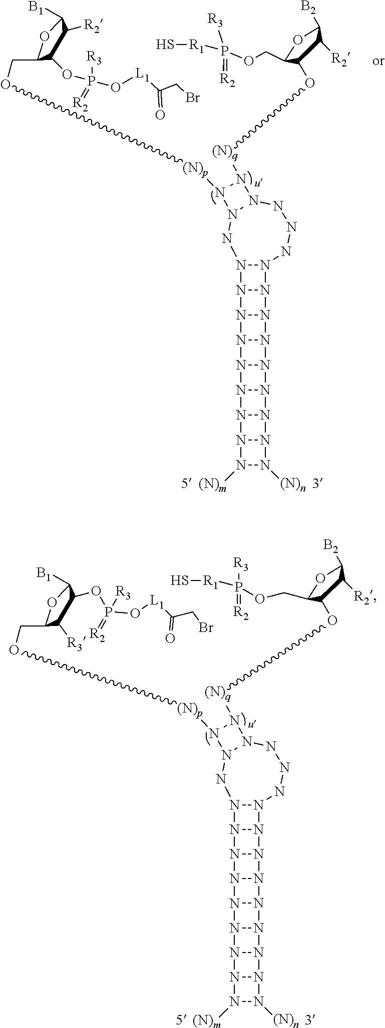

in the presence or absence of a synthetic guide molecule of formula:

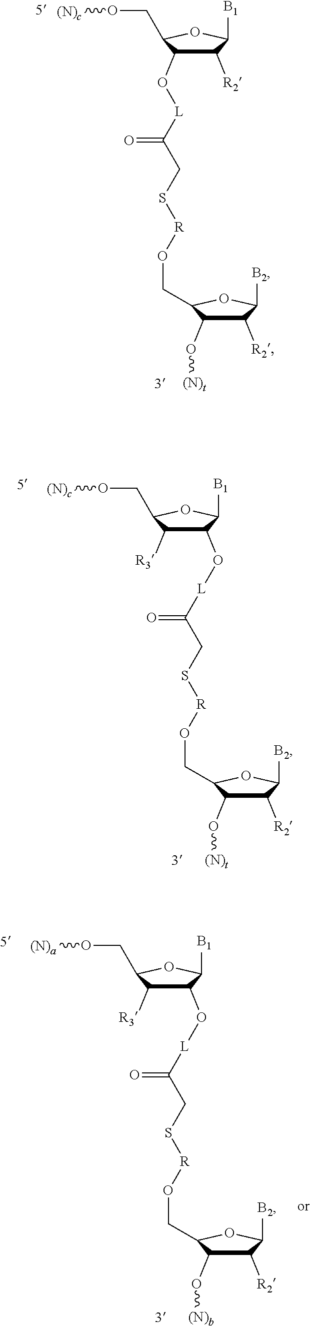

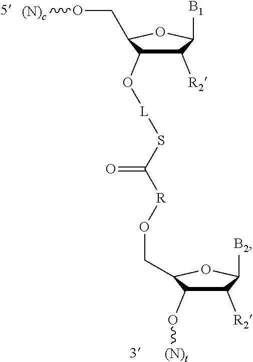

or a salt thereof.

In some embodiments, the oligonucleotide intermediates of the composition are of formula:

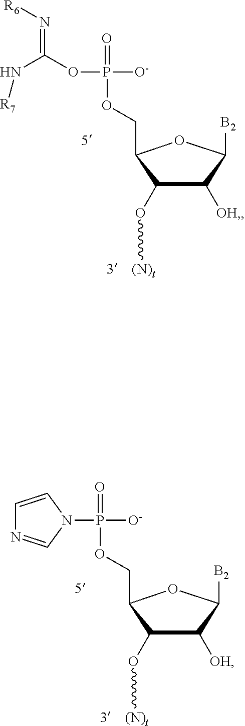

or, and the synthetic guide molecule is of formula:

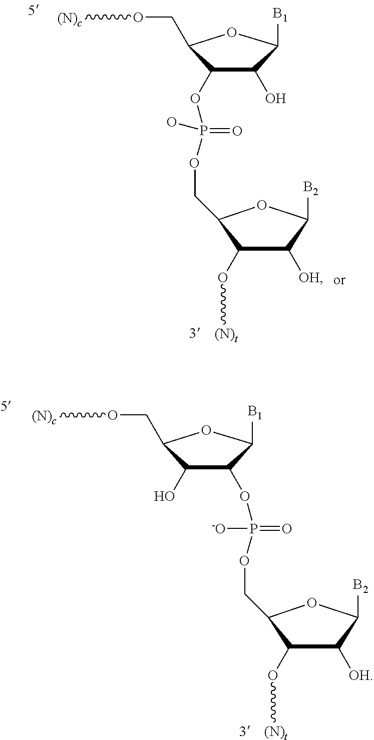

In some embodiments, the composition comprises oligonucleotide intermediates with an annealed duplex of formula:

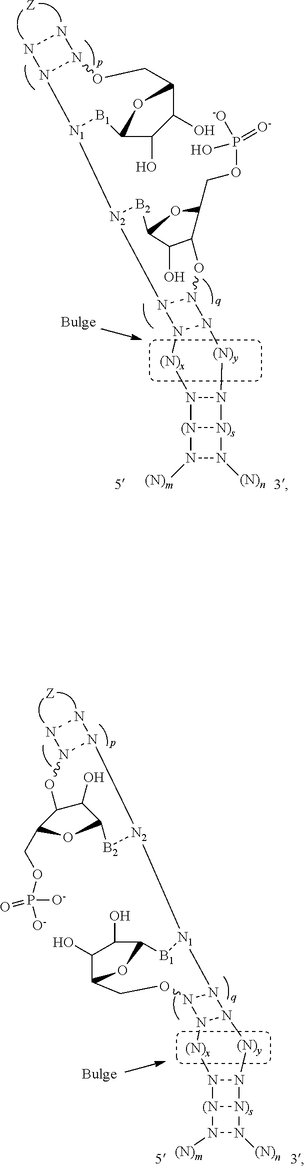

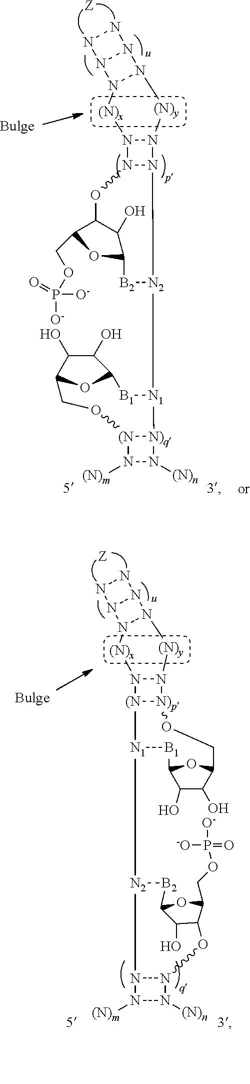

or a salt thereof.

In some embodiments, the composition is substantially free of homodimers. In some embodiments, the composition that is substantially free of homodimers and/or byproducts comprises a guide molecule that was synthesized using a method comprising a homobifunctional cross linking reagent. In some embodiments, the composition that is substantially free of homodimers and/or byproducts comprises a guide molecule with a urea linkage. In some embodiments, the guide molecule is of formula:

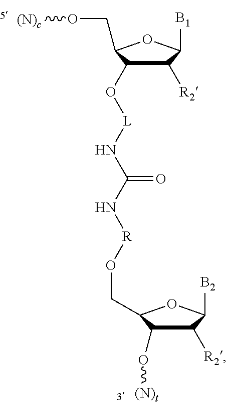

or a pharmaceutically acceptable salt thereof, wherein the composition is substantially free of molecules of formula:

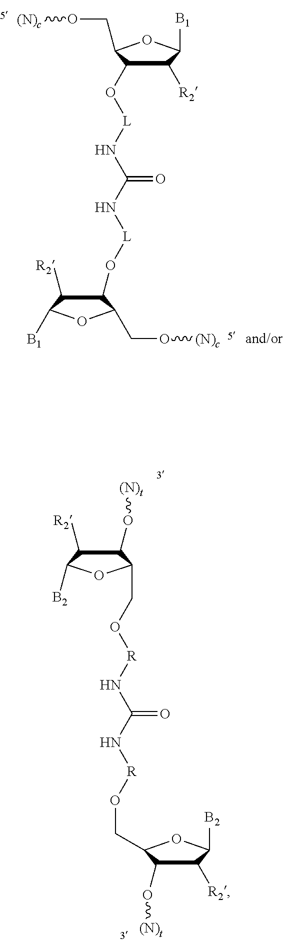

or a pharmaceutically acceptable salt thereof. In some embodiments, the guide molecule is of formula:

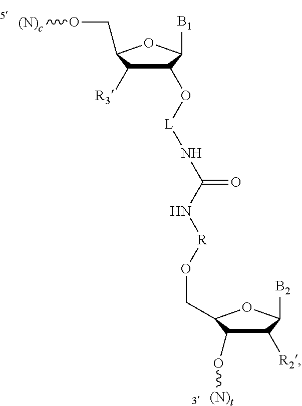

or a pharmaceutically acceptable salt thereof, wherein the composition is substantially free of molecules of formula:

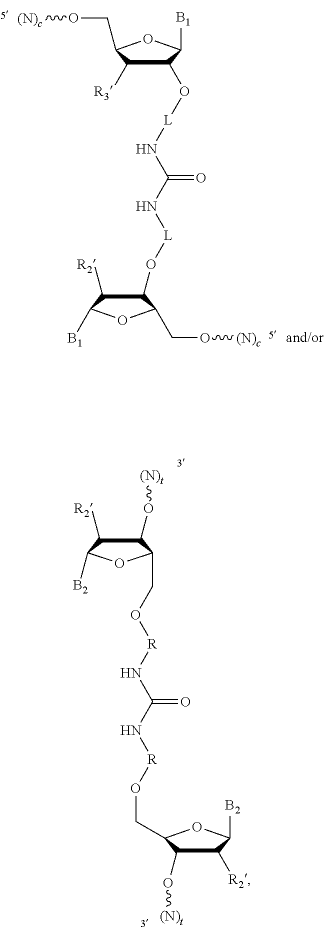

or a pharmaceutically acceptable salt thereof.

In some embodiments, the composition is substantially free of byproducts. In some embodiments, the composition comprises a guide molecule comprising a urea linkage. In some embodiments, the composition comprises a guide molecule of formula:

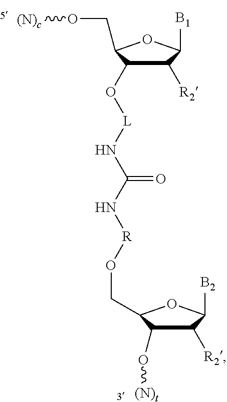

or a pharmaceutically acceptable salt thereof, wherein the composition is substantially free of molecules of formula:

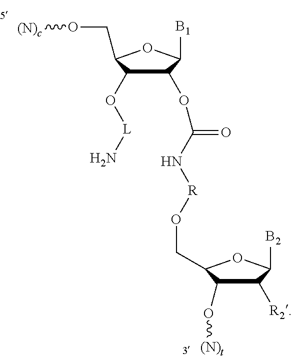

In some embodiments, the composition comprises a guide molecule of formula:

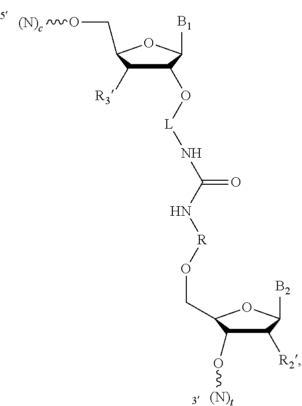

or a pharmaceutically acceptable salt thereof, wherein the composition is substantially free of molecules of formula:

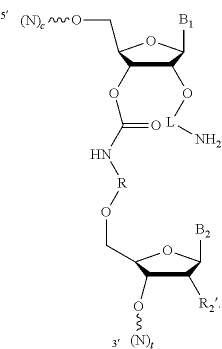

In some embodiments, the composition is not substantially free of byproducts. In some embodiments, the composition comprises (a) a synthetic unimolecular guide molecule for a CRISPR system, wherein the guide molecule is of formula:

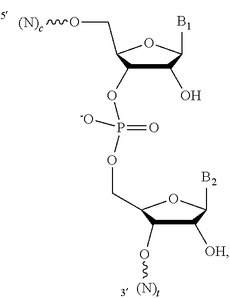

or a pharmaceutically acceptable salt thereof; and (b) one or more of: (i) a carbodiimide, or a salt thereof; (ii) imidazole, cyanoimidazole, pyridine, and dimethylaminopyridine, or a salt thereof; and (iii) a compound of formula:

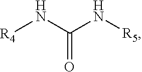

or a salt thereof, wherein R₄ and R₅ are each independently substituted or unsubstituted alkyl, or substituted or unsubstituted carbocyclyl. In some embodiments, the carbodiimide is EDC, DCC, or DIC. In some embodiments, the composition comprises EDC. In some embodiments, the composition comprises imidazole.

In some embodiments, the composition is substantially free of n+1 and/or n−1 species. In some embodiments, the composition comprises less than about 10%, 5%, 2%, 1%, or 0.1% of guide molecules comprising a truncation relative to a reference guide molecule sequence. In some embodiments, at least about 85%, 90%, 95%, 98%, or 99% of the guide molecules comprise a 5′ sequence comprising nucleotides 1-20 of the guide molecule that is 100% identical to a corresponding 5′ sequence of the reference guide molecule sequence.

In some embodiments, the composition comprises essentially a guide molecule of formula:

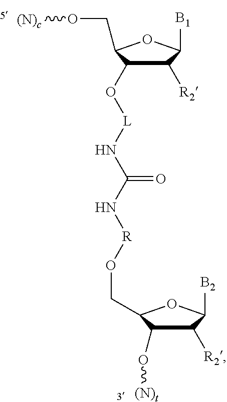

or a pharmaceutically acceptable salt thereof,
wherein the composition is substantially free of molecules of formula:

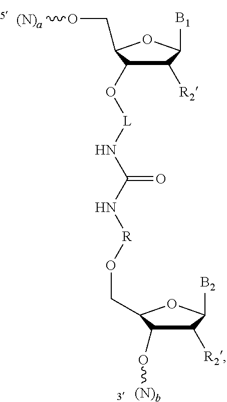

or a pharmaceutically acceptable salt thereof,
wherein a is not equal to c; and/or b is not equal to t. In some embodiments, the composition comprises essentially a guide molecule of formula:

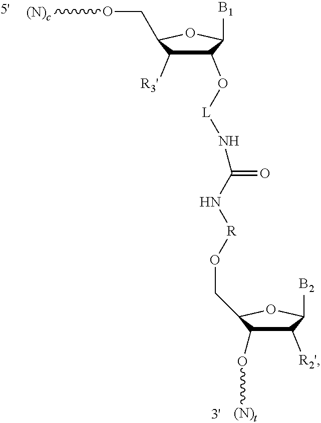

or a pharmaceutically acceptable salt thereof,
wherein the composition is substantially free of molecules of formula:

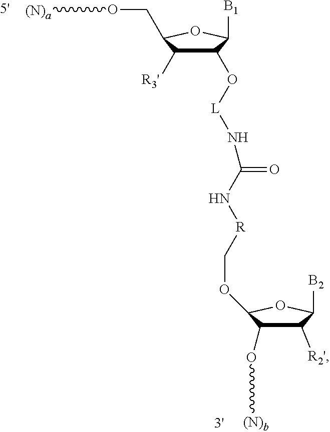

or a pharmaceutically acceptable salt thereof,
wherein a is not equal to c; and/or b is not equal to t.

In some embodiments, the composition comprises essentially guide molecules of formula:

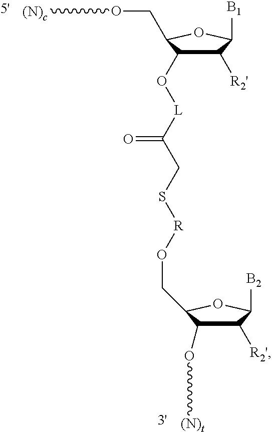

or a pharmaceutically acceptable salt thereof,
wherein the composition is substantially free of molecules of formula:

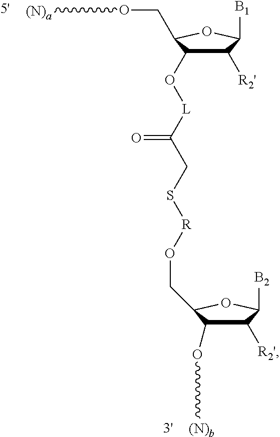

or a pharmaceutically acceptable salt thereof,
wherein a is not equal to c; and/or b is not equal to t. In some embodiments, the composition comprises essentially guide molecules of formula:

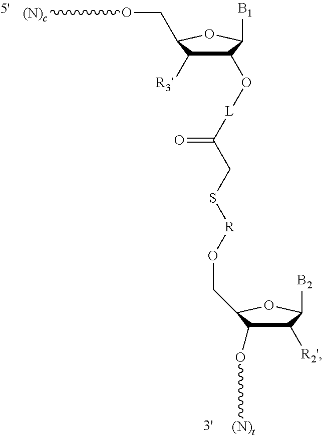

or a pharmaceutically acceptable salt thereof,
wherein the composition is substantially free of molecules of formula:

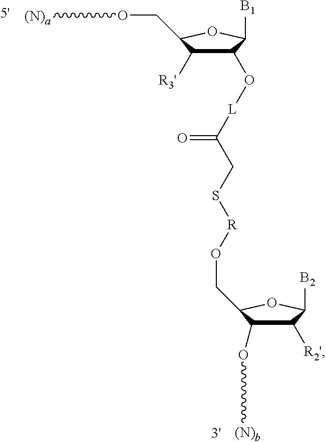

or a pharmaceutically acceptable salt thereof,
wherein a is not equal to c; and/or b is not equal to t. In some embodiments, the composition comprises essentially guide molecules of formula:

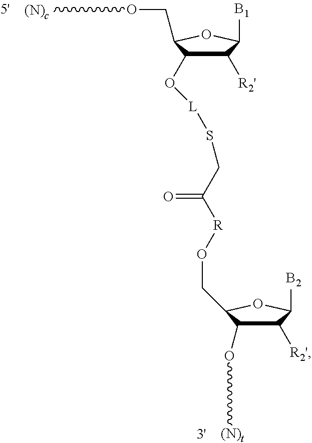

or a pharmaceutically acceptable salt thereof,
wherein the composition is substantially free of molecules of formula:

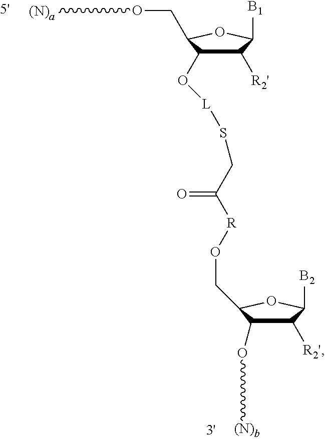

or a pharmaceutically acceptable salt thereof,
wherein a is not equal to c; and/or b is not equal to t. In some embodiments, the composition comprises essentially guide molecules of formula:

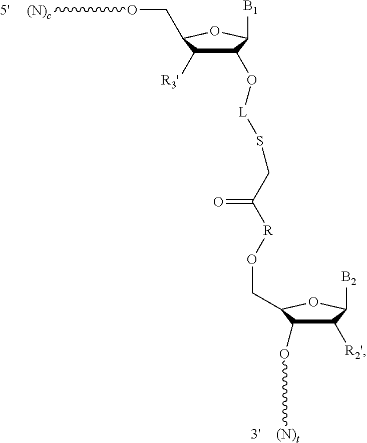

or a pharmaceutically acceptable salt thereof,
wherein the composition is substantially free of molecules of formula:

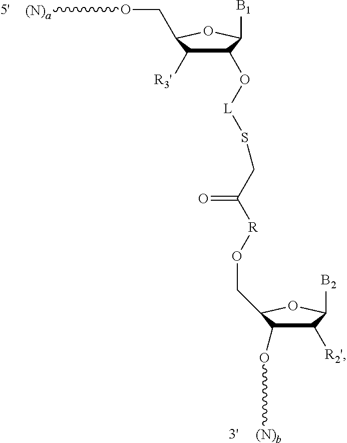

or a pharmaceutically acceptable salt thereof,
wherein a is not equal to c; and/or b is not equal to t. In some embodiments, a is less than c, and/or b is less than t.

In some embodiments, the composition comprises a guide molecule of formula:

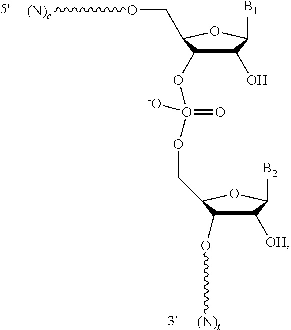

or a pharmaceutically acceptable salt thereof, wherein the composition is substantially free of molecules of formula:

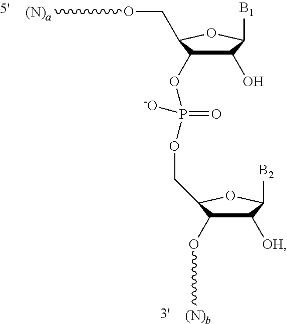

or a pharmaceutically acceptable salt thereof,
wherein a+b is c+t−k, wherein k is an integer between 1 and 10, inclusive.

In one embodiment, the composition comprises a synthetic unimolecular guide molecule for a CRISPR system, wherein the guide molecule is of formula:

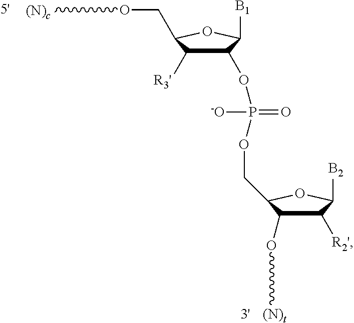

or a pharmaceutically acceptable salt thereof, wherein the 2′-5′ phosphodiester linkage depicted in the formula is between two nucleotides in the duplex. In some embodiments, the guide molecule is of formula:

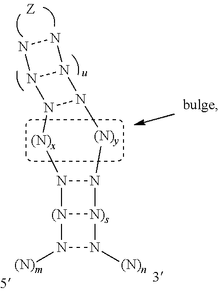

or a pharmaceutically acceptable salt thereof, wherein at least one phosphodiester linkage between two nucleotides in a duplex region depicted in the formula is a 2′-5′ phosphodiester linkage. In some embodiments, the 2′-5′ phosphodiester linkage is between two nucleotides that are located 5′ of the bulge. In some embodiments, the 2′-5′ phosphodiester linkage is between two nucleotides that are located 5′ of the nucleotide loop Z and 3′ of the bulge. In some embodiments, the 2′-5′ phosphodiester linkage is between two nucleotides that are located 3′ of the nucleotide loop Z and 5′ of the bulge. In some embodiments, the 2′-5′ phosphodiester linkage is between two nucleotides that are located 3′ of the bulge.

Guide Molecule Design

Methods for selection and validation of target sequences as well as off-target analyses have been described previously, e.g., in Mali; Hsu; Fu et al., 2014 Nat biotechnol 32(3): 279-84, Heigwer et al., 2014 Nat methods 11(2):122-3; Bae et al. (2014) Bioinformatics 30(10): 1473-5; and Xiao A et al. (2014) Bioinformatics 30(8): 1180-1182. Each of these references is incorporated by reference herein. As a non-limiting example, guide molecule design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

The stem loop structure and position of a chemical linkage in a synthetic unimolecular guide molecule may also be designed. The inventors recognized the value of using Gibbs free energy differences (ΔG) to predict the ligation efficiency of chemical conjugation reactions. Calculation of ΔG is performed using OligoAnalyzer (available at www.idtdna.com/calc/analyzer) or similar tools. Comparison of ΔG of heterodimerization to form the desired annealed duplex and ΔG of homodimerization of two identical oligonucleotides may predict the experimental outcome of chemical conjugation. When ΔG of heterodimerization is less than ΔG of homodimerization, ligation efficiency is predicted to be high. This prediction method is explained further in Example XX.

Guide Molecule Modifications

The activity, stability, or other characteristics of guide molecules can be altered through the incorporation of certain modifications. As one example, transiently expressed or delivered nucleic acids can be prone to degradation by, e.g., cellular nucleases. Accordingly, the guide molecules described herein can contain one or more modified nucleosides or nucleotides which introduce stability toward nucleases. While not wishing to be bound by theory it is also believed that certain modified guide molecules described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, may be reduced or eliminated altogether by the modifications presented herein.

Certain exemplary modifications discussed in this section can be included at any position within a guide molecule sequence including, without limitation at or near the 5′ end (e.g., within 1-10, 1-5, 1-3, or 1-2 nucleotides of the 5′ end) and/or at or near the 3′ end (e.g., within 1-10, 1-5, 1-3, or 1-2 nucleotides of the 3′ end). In some cases, modifications are positioned within functional motifs, such as the repeat-anti-repeat duplex of a Cas9 guide molecule, a stem loop structure of a Cas9 or Cpf1 guide molecule, and/or a targeting domain of a guide molecule.

As one example, the 5′ end of a guide molecule can include a eukaryotic mRNA cap structure or cap analog (e.g., a G(5)ppp(5)G cap analog, a m7G(5)ppp(5)G cap analog, or a 3′-O-Me-m7G(5)ppp(5)G anti reverse cap analog (ARCA)), as shown below:

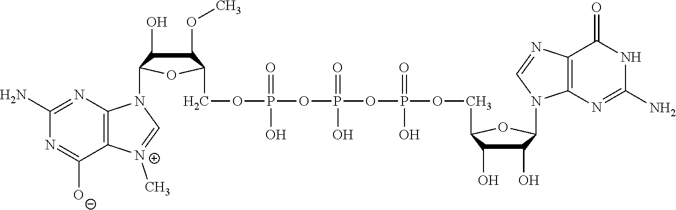

The cap or cap analog can be included during either chemical or enzymatic synthesis of the guide molecule.

Along similar lines, the 5′ end of the guide molecule can lack a 5′ triphosphate group. For instance, in vitro transcribed guide molecules can be phosphatase-treated (e.g., using calf intestinal alkaline phosphatase) to remove a 5′ triphosphate group.

Another common modification involves the addition, at the 3′ end of a guide molecule, of a plurality (e.g., 1-10, 10-20, or 25-200) of adenine (A) residues referred to as a polyA tract. The polyA tract can be added to a guide molecule during chemical or enzymatic synthesis, using a polyadenosine polymerase (e.g., E. coli Poly(A)Polymerase).

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

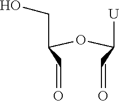

wherein “U” can be an unmodified or modified uridine.

The 3′ terminal U ribose can be modified with a 2′3′ cyclic phosphate as shown below:

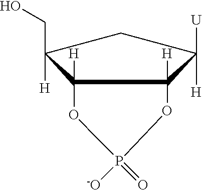

wherein “U” can be an unmodified or modified uridine.

Guide RNAs can contain 3′ nucleotides which can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the guide molecule, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphorothioate (PhTx) group. In certain embodiments, one or more of the nucleotides of the guide molecule can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In certain embodiments, a guide molecule can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, guide molecules include the sugar group ribose, which is a 5-membered ring having an oxygen. Exemplary modified guide molecules can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a guide molecule comprises a 4′-S, 4′-Se or a 4′-C-aminomethyl-2′-O-Me modification.

In certain embodiments, deaza nucleotides, e.g., 7-deaza-adenosine, can be incorporated into the guide molecule. In certain embodiments, O- and N-alkylated nucleotides, e.g., N6-methyl adenosine, can be incorporated into the guide molecule. In certain embodiments, one or more or all of the nucleotides in a guide molecule are deoxynucleotides.

Nucleotides of a guide molecule may also be modified at the phosphodiester linkage. Such modification may include phosphonoacetate, phosphorothioate, thiophosphonoacetate, or phosphoroamidate linkages. In some embodiments, a nucleotide may be linked to its adjacent nucleotide via a phosphorothioate linkage. Furthermore, modifications to the phosphodiester linkage may be the sole modification to a nucleotide or may be combined with other nucleotide modifications described above. For example, a modified phosphodiester linkage can be combined with a modification to the sugar group of a nucleotide. In some embodiments, 5′ or 3′ nucleotides comprise a 2′-OMe modified ribonucleotide residue that is linked to its adjacent nucleotide(s) via a phosphorothioate linkage.

### RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g., complex with) a guide molecule (e.g., gRNA); and (b) together with the guide molecule (e.g., gRNA), associate with, and optionally cleave or modify, a target region of a DNA that includes (i) a sequence complementary to the targeting domain of the guide molecule (e.g., gRNA) and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g. Cas9 vs. Cpf1), species (e.g. S. pyogenes vs. S. aureus) or variation (e.g., full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to guide molecule targeting domains (or “spacers”). Together with protospacer sequences, PAM sequences define target regions or sequences for specific RNA-guided nuclease/guide molecule combinations.

Various RNA-guided nucleases may require different sequential relationships between PAMs and protospacers. In general, Cas9s recognize PAM sequences that are 3′ of the protospacer as visualized relative to the guide molecule.

Cpf1, on the other hand, generally recognizes PAM sequences that are 5′ of the protospacer as visualized relative to the guide molecule.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the guide molecule targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389, Sep. 12, 2013 (Ran), incorporated by reference herein), or that that do not cut at all.

Cas9

Crystal structures have been determined for S. pyogenes Cas9 (Jinek 2014), and for S. aureus Cas9 in complex with a unimolecular guide RNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g. a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in guide molecule:DNA recognition, while the REC domain is thought to interact with the repeat:anti-repeat duplex of the guide molecule and to mediate the formation of the Cas9/guide molecule complex.

The NUC lobe comprises a RuvC domain, an HNH domain, and a PAM-interacting (PI) domain. The RuvC domain shares structural similarity to retroviral integrase superfamily members and cleaves the non-complementary (i.e. bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to FINN endonuclease motifs, and cleaves the complementary (i.e. top) strand of the target nucleic acid. The PI domain, as its name suggests, contributes to PAM specificity.

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the guide molecule falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

Cpf1

The crystal structure of Acidaminococcus sp. Cpf1 in complex with crRNA and a double-stranded (ds) DNA target including a TTTN PAM sequence has been solved by Yamano et al. (Cell. 2016 May 5; 165(4): 949-962 (Yamano), incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 guide molecule (the handle) adopts a pseudonot structure, rather than a stem loop structure formed by the repeat:antirepeat duplex in Cas9 guide molecules.

Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran and Yamano, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain of a Cas9 will result in a nickase that cleaves the complementary or top strand.

On the other hand, inactivation of a Cas9 HNH domain results in a nickase that cleaves the bottom or non-complementary strand.

Modifications of PAM specificity relative to naturally occurring Cas9 reference molecules has been described by Kleinstiver et al. for both S. pyogenes (Kleinstiver et al., Nature. 2015 Jul. 23; 523(7561):481-5 (Kleinstiver I) and S. aureus (Kleinstiver et al., Nat Biotechnol. 2015 December; 33(12): 1293-1298 (Klienstiver II)). Kleinstiver et al. have also described modifications that improve the targeting fidelity of Cas9 (Nature, 2016 Jan. 28; 529, 490-495 (Kleinstiver III)). Each of these references is incorporated by reference herein.

RNA-guided nucleases have been split into two or more parts, as described by Zetsche et al. (Nat Biotechnol. 2015 February; 33(2):139-42 (Zetsche II), incorporated by reference), and by Fine et al. (Sci Rep. 2015 Jul. 1; 5:10777 (Fine), incorporated by reference).

RNA-guided nucleases can be, in certain embodiments, size-optimized or truncated, for instance via one or more deletions that reduce the size of the nuclease while still retaining guide molecule association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotechnology 32, 577-582 (2014), which is incorporated by reference for all purposes herein.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

### Nucleic Acids Encoding RNA-Guided Nucleases

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease may comprise a nuclear localization sequence (NLS). Nuclear localization sequences are known in the art.

### Functional Analysis of Candidate Molecules

Candidate RNA-guided nucleases, guide molecules, and complexes thereof, can be evaluated by standard methods known in the art. See, e.g. Cotta-Ramusino. The stability of RNP complexes may be evaluated by differential scanning fluorimetry, as described below.

Differential Scanning Fluorimetry (DSF)

The thermostability of ribonucleoprotein (RNP) complexes comprising guide molecules and RNA-guided nucleases can be measured via DSF. The DSF technique measures the thermostability of a protein, which can increase under favorable conditions such as the addition of a binding RNA molecule, e.g., a guide molecule.

A DSF assay can be performed according to any suitable protocol, and can be employed in any suitable setting, including without limitation (a) testing different conditions (e.g., different stoichiometric ratios of guide molecule: RNA-guided nuclease protein, different buffer solutions, etc.) to identify optimal conditions for RNP formation; and (b) testing modifications (e.g. chemical modifications, alterations of sequence, etc.) of an RNA-guided nuclease and/or a guide molecule to identify those modifications that improve RNP formation or stability. One readout of a DSF assay is a shift in melting temperature of the RNP complex; a relatively high shift suggests that the RNP complex is more stable (and may thus have greater activity or more favorable kinetics of formation, kinetics of degradation, or another functional characteristic) relative to a reference RNP complex characterized by a lower shift. When the DSF assay is deployed as a screening tool, a threshold melting temperature shift may be specified, so that the output is one or more RNPs having a melting temperature shift at or above the threshold. For instance, the threshold can be 5-10° C. (e.g. 5°, 6°, 7°, 8°, 9°, 10°) or more, and the output may be one or more RNPs characterized by a melting temperature shift greater than or equal to the threshold.

Two non-limiting examples of DSF assay conditions are set forth below:

To determine the best solution to form RNP complexes, a fixed concentration (e.g. 2 μM) of Cas9 in water+10× SYPRO Orange® (Life Technologies cat #S-6650) is dispensed into a 384 well plate. An equimolar amount of guide molecule diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of guide molecule with fixed concentration (e.g. 2 μM) Cas9 in optimal buffer from assay 1 above and incubating (e.g. at RT for 10′) in a 384 well plate. An equal volume of optimal buffer+10× SYPRO Orange® (Life Technologies cat #S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

### Genome Editing Strategies

The genome editing systems described above are used, in various embodiments of the present disclosure, to generate edits in (i.e. to alter) targeted regions of DNA within or obtained from a cell. Various strategies are described herein to generate particular edits, and these strategies are generally described in terms of the desired repair outcome, the number and positioning of individual edits (e.g. SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region; or (c) interruption of all or part of a targeted region. This grouping is not intended to be limiting, or to be binding to any particular theory or model, and is offered solely for economy of presentation. Skilled artisans will appreciate that the listed outcomes are not mutually exclusive and that some repairs may result in other outcomes. The description of a particular editing strategy or method should not be understood to require a particular repair outcome unless otherwise specified.

Replacement of a targeted region generally involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double stranded, as described in greater detail below. Single or double stranded templates can be exogenous, in which case they will promote gene correction, or they can be endogenous (e.g. a homologous sequence within the cellular genome), to promote gene conversion. Exogenous templates can have asymmetric overhangs (i.e. the portion of the template that is complementary to the site of the DSB may be offset in a 3′ or 5′ direction, rather than being centered within the donor template), for instance as described by Richardson et al. (Nature Biotechnology 34, 339-344 (2016), (Richardson), incorporated by reference). In instances where the template is single stranded, it can correspond to either the complementary (top) or non-complementary (bottom) strand of the targeted region.

Gene conversion and gene correction are facilitated, in some cases, by the formation of one or more nicks in or around the targeted region, as described in Ran and Cotta-Ramusino. In some cases, a dual-nickase strategy is used to form two offset SSBs that, in turn, form a single DSB having an overhang (e.g. a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends may be stochastic in nature, indel mutations tend to be variable, occurring along a distribution, and can be influenced by a variety of factors, including the specific target site, the cell type used, the genome editing strategy used, etc. Even so, it is possible to draw limited generalizations about indel formation: deletions formed by repair of a single DSB are most commonly in the 1-50 bp range, but can reach greater than 100-200 bp. Insertions formed by repair of a single DSB tend to be shorter and often include short duplications of the sequence immediately surrounding the break site. However, it is possible to obtain large insertions, and in these cases, the inserted sequence has often been traced to other regions of the genome or to plasmid DNA present in the cells.

Indel mutations—and genome editing systems configured to produce indels—are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g. ±1, ±2, ±3, etc. As one example, in a lead-finding setting, multiple guide molecules can be screened to identify those guide molecules that most efficiently drive cutting at a target site based on an indel readout under controlled conditions. Guides that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the guide molecule constant and varying certain other reaction conditions or delivery methods.

Multiplex Strategies

While exemplary strategies discussed above have focused on repair outcomes mediated by single DSBs, genome editing systems according to this disclosure may also be employed to generate two or more DSBs, either in the same locus or in different loci. Strategies for editing that involve the formation of multiple DSBs, or SSBs, are described in, for instance, Cotta-Ramusino.

Donor Template Design

Donor template design is described in detail in the literature, for instance in Cotta-Ramusino. DNA oligomer donor templates (oligodeoxynucleotides or ODNs), which can be single stranded (ssODNs) or double-stranded (dsODNs), can be used to facilitate HDR-based repair of DSBs, and are particularly useful for introducing alterations into a target DNA sequence, inserting a new sequence into the target sequence, or replacing the target sequence altogether.

Whether single-stranded or double stranded, donor templates generally include regions that are homologous to regions of DNA within or near (e.g. flanking or adjoining) a target sequence to be cleaved. These homologous regions are referred to here as “homology arms,” and are illustrated schematically below:

[5′ homology arm]-[replacement sequence]-[3′ homology arm].

The homology arms can have any suitable length (including 0 nucleotides if only one homology arm is used), and 3′ and 5′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In some embodiments, both the 5′ and the 3′ homology arms can be shortened to avoid including certain sequence repeat elements. In addition, some homology arm designs can improve the efficiency of editing or increase the frequency of a desired repair outcome. For example, Richardson et al. Nature Biotechnology 34, 339-344 (2016) (Richardson), which is incorporated by reference, found that the relative asymmetry of 3′ and 5′ homology arms of single stranded donor templates influenced repair rates and/or outcomes.

Replacement sequences in donor templates have been described elsewhere, including in Cotta-Ramusino et al. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired. Another common sequence modification involves the alteration of one or more sequences that are complementary to, or code for, the PAM sequence of the RNA-guided nuclease or the targeting domain of the guide molecule(s) being used to generate an SSB or DSB, to reduce or eliminate repeated cleavage of the target site after the replacement sequence has been incorporated into the target site.

Where a linear ssODN is used, it can be configured to (i) anneal to the nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN may have any suitable length, e.g., about, at least, or no more than 150-200 nucleotides (e.g., 150, 160, 170, 180, 190, or 200 nucleotides).

It should be noted that a template nucleic acid can also be a nucleic acid vector, such as a viral genome or circular double stranded DNA, e.g., a plasmid. Nucleic acid vectors comprising donor templates can include other coding or non-coding elements. For example, a template nucleic acid can be delivered as part of a viral genome (e.g., in an AAV or lentiviral genome) that includes certain genomic backbone elements (e.g., inverted terminal repeats, in the case of an AAV genome) and optionally includes additional sequences coding for a guide molecule and/or an RNA-guided nuclease. In certain embodiments, the donor template can be adjacent to, or flanked by, target sites recognized by one or more guide molecules, to facilitate the formation of free DSBs on one or both ends of the donor template that can participate in repair of corresponding SSBs or DSBs formed in cellular DNA using the same guide molecules. Exemplary nucleic acid vectors suitable for use as donor templates are described in Cotta-Ramusino.

Whatever format is used, a template nucleic acid can be designed to avoid undesirable sequences. In certain embodiments, one or both homology arms can be shortened to avoid overlap with certain sequence repeat elements, e.g., Alu repeats, LINE elements, etc.

### Target Cells

Genome editing systems according to this disclosure can be used to manipulate or alter a cell, e.g., to edit or alter a target nucleic acid. The manipulating can occur, in various embodiments, in vivo or ex vivo.

A variety of cell types can be manipulated or altered according to the embodiments of this disclosure, and in some cases, such as in vivo applications, a plurality of cell types are altered or manipulated, for example by delivering genome editing systems according to this disclosure to a plurality of cell types. In other cases, however, it may be desirable to limit manipulation or alteration to a particular cell type or types. For instance, it can be desirable in some instances to edit a cell with limited differentiation potential or a terminally differentiated cell, such as a photoreceptor cell in the case of Maeder, in which modification of a genotype is expected to result in a change in cell phenotype. In other cases, however, it may be desirable to edit a less differentiated, multipotent or pluripotent, stem or progenitor cell. By way of example, the cell may be an embryonic stem cell, induced pluripotent stem cell (iPSC), hematopoietic stem/progenitor cell (HSPC), or other stem or progenitor cell type that differentiates into a cell type of relevance to a given application or indication.

As a corollary, the cell being altered or manipulated is, variously, a dividing cell or a non-dividing cell, depending on the cell type(s) being targeted and/or the desired editing outcome.

When cells are manipulated or altered ex vivo, the cells can be used (e.g. administered to a subject) immediately, or they can be maintained or stored for later use. Those of skill in the art will appreciate that cells can be maintained in culture or stored (e.g. frozen in liquid nitrogen) using any suitable method known in the art.

### Implementation of Genome Editing Systems: Delivery, Formulations, and Routes of Administration

As discussed above, the genome editing systems of this disclosure can be implemented in any suitable manner, meaning that the components of such systems, including without limitation the RNA-guided nuclease, guide molecule, and optional donor template nucleic acid, can be delivered, formulated, or administered in any suitable form or combination of forms that results in the transduction, expression or introduction of a genome editing system and/or causes a desired repair outcome in a cell, tissue or subject. Tables 5 and 6 set forth several, non-limiting examples of genome editing system implementations. Those of skill in the art will appreciate, however, that these listings are not comprehensive, and that other implementations are possible. With reference to Table 5 in particular, the table lists several exemplary implementations of a genome editing system comprising a single guide molecule and an optional donor template. However, genome editing systems according to this disclosure can incorporate multiple guide molecules, multiple RNA-guided nucleases, and other components such as proteins, and a variety of implementations will be evident to the skilled artisan based on the principles illustrated in the table. In the table, [N/A] indicates that the genome editing system does not include the indicated component.

TABLE 5
Genome Editing System Components

| RNA-guided | | Donor | |
|---|---|---|---|
| Nuclease | gRNA | Template | Comments |
| Protein | RNA | [N/A] | An RNA-guided nuclease protein |
| | | | complexed with a gRNA molecule |
| | | | (an RNP complex) |
| Protein | RNA | DNA | An RNP complex as described above |
| | | | plus a single-stranded or double |
| | | | stranded donor template. |
| Protein | DNA | [N/A] | An RNA-guided nuclease protein |
| | | | plus gRNA transcribed from DNA. |
| Protein | DNA | DNA | An RNA-guided nuclease protein |
| | | | plus gRNA-encoding DNA and a |
| | | | separate DNA donor template. |
| Protein | DNA | | An RNA-guided nuclease protein |
| | | | and a single DNA encoding both a |
| | | | gRNA and a donor template. |
| DNA | | | A DNA or DNA vector encoding an |
| | | | RNA-guided nuclease, a gRNA and a |
| | | | donor template. |
| DNA | DNA | [N/A] | Two separate DNAs, or two separate |
| | | | DNA vectors, encoding the RNA- |
| | | | guided nuclease and the gRNA, |
| | | | respectively. |
| DNA | DNA | DNA | Three separate DNAs, or three |
| | | | separate DNA vectors, encoding the |
| | | | RNA-guided nuclease, the gRNA and |
| | | | the donor template, respectively. |
| DNA | | [N/A] | A DNA or DNA vector encoding an |
| | | | RNA-guided nuclease and a gRNA |
| DNA | | DNA | A first DNA or DNA vector encoding |
| | | | an RNA-guided nuclease and a |
| | | | gRNA, and a second DNA or DNA |
| | | | vector encoding a donor template. |
| DNA | DNA | | A first DNA or DNA vector encoding |
| | | | an RNA-guided nuclease and second |
| | | | DNA or DNA vector encoding a |
| | | | gRNA and a donor template. |
| DNA | | | A first DNA or DNA vector encoding |
| | DNA | | an RNA-guided nuclease and a donor |
| | | | template, and a second DNA or DNA |
| | | | vector encoding a gRNA |
| DNA | | | A DNA or DNA vector encoding an |
| | RNA | | RNA-guided nuclease and a donor |
| | | | template, and a gRNA |

| | | |
|---|---|---|
| RNA | [N/A] | An RNA or RNA vector encoding an |
| | | RNA-guided nuclease and comprising |
| | | a gRNA |
| RNA | DNA | An RNA or RNA vector encoding an |
| | | RNA-guided nuclease and comprising |
| | | a gRNA, and a DNA or DNA vector |
| | | encoding a donor template. |

Table 6 summarizes various delivery methods for the components of genome editing systems, as described herein. Again, the listing is intended to be exemplary rather than limiting.

TABLE 6

| | | Delivery | | | |
|---|---|---|---|---|---|
| | | into Non- | | | Type of |
| | | Dividing | Duration of | Genome | Molecule |
| Delivery Vector/Mode | | Cells | Expression | Integration | Delivered |
| Physical (e.g., electroporation, | | YES | Transient | NO | Nucleic Acids |
| particle gun, Calcium Phosphate | | | | | and Proteins |
| transfection, cell compression or | | | | | |
| squeezing) | | | | | |
| Viral | Retrovirus | NO | Stable | YES | RNA |
| | Lentivirus | YES | Stable | YES/NO with | RNA |
| | | | | modifications | |
| | Adenovirus | YES | Transient | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

Nucleic Acid-Based Delivery of Genome Editing Systems

Nucleic acids encoding the various elements of a genome editing system according to the present disclosure can be administered to subjects or delivered into cells by art-known methods or as described herein. For example, RNA-guided nuclease-encoding and/or guide molecule-encoding DNA, as well as donor template nucleic acids can be delivered by, e.g., vectors (e.g., viral or non-viral vectors), non-vector based methods (e.g., using naked DNA or DNA complexes), or a combination thereof.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes, HSCs). Nucleic acid vectors, such as the vectors summarized in Table 6, can also be used.

Nucleic acid vectors can comprise one or more sequences encoding genome editing system components, such as an RNA-guided nuclease, a guide molecule and/or a donor template. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cas9 coding sequence that includes one or more nuclear localization sequences (e.g., a nuclear localization sequence from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art, and are described in Cotta-Ramusino.

Nucleic acid vectors according to this disclosure include recombinant viral vectors. Exemplary viral vectors are set forth in Table 6, and additional suitable viral vectors and their use and production are described in Cotta-Ramusino. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art, and are summarized in Cotta-Ramusino. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure. Exemplary lipids for use in nanoparticle formulations, and/or gene transfer are shown in Table 7, and Table 8 lists exemplary polymers for use in gene transfer and/or nanoparticle formulations.

TABLE 71
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)propyl]N,N,N-trimethylammonium chloride | DOTMA | Cationic |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N-dimethyl-1- | DOSPA | Cationic |
| propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]-dimethylammonium | CLIP-1 | Cationic |
| chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] imidazolinium | DOTIM | Cationic |
| chloride | | |
| N1-Cholesteryloxycarbonyl-3,7-diazanonane-1,9-diamine | CDAN | Cationic |
| 2-(3-[Bis(3-arnino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2-DMA | Cationic |
| dilinoleyl-methyl-4-dimethylaminobutyrate | DLin-MC3-DMA | Cationic |

TABLE 8
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the guide molecule component described herein, are delivered. In certain embodiments, the nucleic acid molecule is delivered at the same time as one or more of the components of the Genome editing system. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Genome editing system are delivered. In certain embodiments, the nucleic acid molecule is delivered by a different means than one or more of the components of the genome editing system, e.g., the RNA-guided nuclease component and/or the guide molecule component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the guide molecule component can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

Delivery of RNPs and/or RNA Encoding Genome Editing System Components

RNPs (complexes of guide molecules and RNA-guided nucleases) and/or RNAs encoding RNA-guided nucleases and/or guide molecules, can be delivered into cells or administered to subjects by art-known methods, some of which are described in Cotta-Ramusino. In vitro, RNA-guided nuclease-encoding and/or guide molecule-encoding RNA can be delivered, e.g., by microinjection, electroporation, transient cell compression or squeezing (see, e.g., Lee 2012). Lipid-mediated transfection, peptide-mediated delivery, GalNAc- or other conjugate-mediated delivery, and combinations thereof, can also be used for delivery in vitro and in vivo.

In vitro, delivery via electroporation comprises mixing the cells with the RNA encoding RNA-guided nucleases and/or guide molecules, with or without donor template nucleic acid molecules, in a cartridge, chamber or cuvette and applying one or more electrical impulses of defined duration and amplitude. Systems and protocols for electroporation are known in the art, and any suitable electroporation tool and/or protocol can be used in connection with the various embodiments of this disclosure.

Route of Administration

Genome editing systems, or cells altered or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In addition, components can be formulated to permit release over a prolonged period of time. A release system can include a matrix of a biodegradable material or a material which releases the incorporated components by diffusion. The components can be homogeneously or heterogeneously distributed within the release system. A variety of release systems can be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein.

Multi-Modal or Differential Delivery of Components

Skilled artisans will appreciate, in view of the instant disclosure, that different components of genome editing systems disclosed herein can be delivered together or separately and simultaneously or nonsimultaneously. Separate and/or asynchronous delivery of genome editing system components can be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., a RNA-guided nuclease molecule, guide molecule, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

By way of example, the components of a genome editing system, e.g., a RNA-guided nuclease and a guide molecule, can be delivered by modes that differ in terms of resulting half-life or persistent of the delivered component the body, or in a particular compartment, tissue or organ. In certain embodiments, a guide molecule can be delivered by such modes. The RNA-guided nuclease molecule component can be delivered by a mode which results in less persistence or less exposure to the body or a particular compartment or tissue or organ.

More generally, in certain embodiments, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In certain embodiments, the second mode of delivery comprises a relatively transient element, e.g., an RNA or protein.

In certain embodiments, the first component comprises a guide molecule, and the delivery mode is relatively persistent, e.g., the guide molecule is transcribed from a plasmid or viral vector, e.g., an AAV or lentivirus. Transcription of these genes would be of little physiological consequence because the genes do not encode for a protein product, and the guide molecules are incapable of acting in isolation. The second component, a RNA-guided nuclease molecule, is delivered in a transient manner, for example as mRNA or as protein, ensuring that the full RNA-guided nuclease molecule/guide molecule complex is only present and active for a short period of time.

Furthermore, the components can be delivered in different molecular form or with different delivery vectors that complement one another to enhance safety and tissue specificity.

Use of differential delivery modes can enhance performance, safety, and/or efficacy, e.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation active complex is minimized outside the overlap of the target regions. Thus, in certain embodiments, a first component, e.g., a guide molecule is delivered by a first delivery mode that results in a first spatial, e.g., tissue, distribution. A second component, e.g., a RNA-guided nuclease molecule is delivered by a second delivery mode that results in a second spatial, e.g., tissue, distribution. In certain embodiments, the first mode comprises a first element selected from a liposome, nanoparticle, e.g., polymeric nanoparticle, and a nucleic acid, e.g., viral vector. The second mode comprises a second element selected from the group. In certain embodiments, the first mode of delivery comprises a first targeting element, e.g., a cell specific receptor or an antibody, and the second mode of delivery does not include that element. In certain embodiments, the second mode of delivery comprises a second targeting element, e.g., a second cell specific receptor or second antibody.

When the RNA-guided nuclease molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the guide molecule and the RNA-guided nuclease molecule are packaged in separated delivery vehicles with distinct but overlapping tissue tropism, the fully functional complex is only be formed in the tissue that is targeted by both vectors.

### Examples

Certain principles of the present disclosure are illustrated by the non-limiting examples that follow.

### Example 1: Exemplary Process for Conjugation of Amine-Functionalized Guide Molecule Fragments with Disuccinimidyl Carbonate

As illustrated in FIG. 1A, a first 5′ guide molecule fragment (e.g., a 34mer) is synthesized with a (C₆)—NH₂ linker at the 3′ end, and a second 3′ guide molecule fragment (e.g., a 66mer) is synthesized with a TEG-NH₂ linker at the 5′ end. The two guide molecule fragments are mixed at a molar ratio of 1:1 in a pH 8.5 buffer comprising 10 mM sodium borate, 150 mM NaCl, and 5 mM MgCl₂. The resulting guide molecule concentration is about 50 to 100 μM. The two guide molecule fragments are annealed, followed by addition of disuccinimidyl carbonate (DSC) in DMF (2.5 mM final concentration). The reaction mixture is vortexed briefly and then mixed at room temperature for 1 hour, followed by removal of excess disuccinimidyl carbonate, and anion-exchange HPLC purification.

### Example 2: Exemplary Process for Conjugation of Thiol-Functionalized Guide Molecule Fragment to Bromoacetyl-Functionalized Guide Molecule Fragment

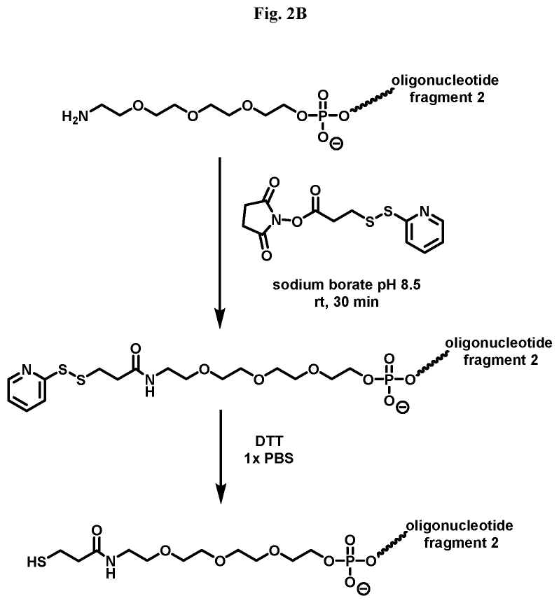

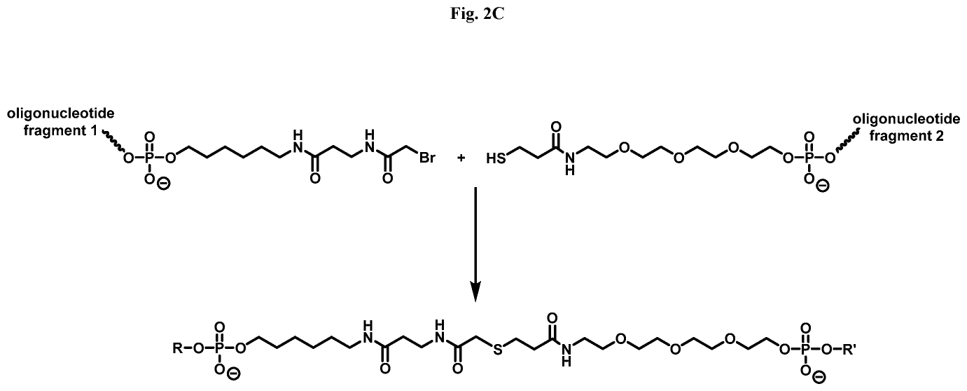

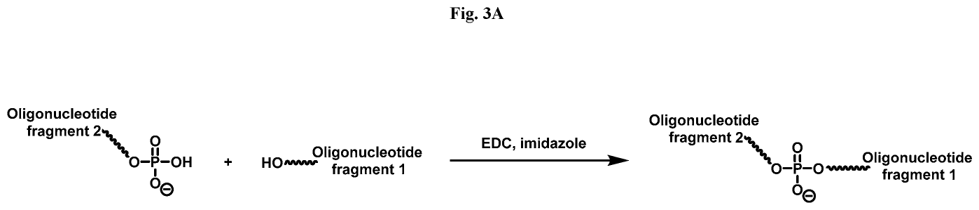

As illustrated in FIG. 2A, a first 5′ guide molecule fragment (e.g., a 34mer) is synthesized with a (C₆)—NH₂ linker at the 3′ end. It is suspended in 100 mM borate buffer at pH 8.5. The guide molecule concentration is about 100 μM to 1 mM. 0.2 volumes of succinimidyl-3-(bromoacetamido)propionate (SBAP) in DMSO (50 equivalents) are added to the guide molecule solution. After mixing for 30 minutes at room temperature, 10 volumes of 100 mM phosphate buffer at pH 7.0 is added. The mixture is concentrated 10× or more on 10,000 MW Amicon. The mixture is further processed by (a) adding 10 volumes of water, and (b) concentrating 10× or more on 10,000 MW Amicon. Steps (a) and (b) are repeated 3 times to afford a first 5′ guide molecule fragment (e.g., 34mer) with a bromoacetyl moiety at the 3′ end.

As illustrated in FIG. 2B, a second 3′ guide molecule fragment (e.g., a 66mer) is synthesized with a TEG-NH₂ linker at the 5′ end. It is suspended in 100 mM borate buffer at pH 8.5 comprising 1 mM EDTA. The guide molecule concentration is about 100 μM to 1 mM. 0.2 volumes of succinimidyl-3-(2-pyridyldithio)propionate (SPDP) in DMSO (50 equivalents) are added to the guide molecule solution. After mixing for 1 hour at room temperature, 1 M dithiothreitol (DTT) is added in 1× PBS. The final concentration of DTT in the mixture is 20 mM. After mixing for 30 minutes at room temperature, 5 M NaCl is added to result a final concentration of 0.3 M NaCl in the mixture followed by addition of 3 volumes of ethanol. The mixture is further processed by: (a) cooling to −20° C. for 15 minutes; (b) centrifuging at 17,000 g (preferably at 4° C.) for 5 minutes; (c) removing the supernatant; (d) suspending the residue in 0.3 M NaCl (sparged with argon); and (e) adding 3 volumes of ethanol. Steps (a)-(e) are repeated 3 times. The resulting pellet (i.e., second 3′ guide molecule fragment with a thiol at the 5′ end) is dried under vacuum.

As illustrated in FIG. 2C, the second 3′ guide molecule fragment (e.g., 66mer) with a thiol at the 5′ end is suspended in 100 mM phosphate buffer at pH 8 comprising 2 mM EDTA (sparged with argon). The guide molecule concentration is about 100 μM to 1 mM. The first 5′ guide molecule fragment (e.g., 34mer) with a bromoacetyl moiety at the 3′ end is suspended in water (about 0.1 volumes relative to the volume of the second 3′ guide molecule fragment mixture). The guide molecule concentration is about 100 μM to 1 mM. The first 5′ guide molecule fragment mixture is added to the second 3′ guide molecule fragment mixture (sparged with argon). The reaction mixture is mixed overnight at room temperature, followed by an anion-exchange HPLC purification.

### Example 3: Exemplary Process for Conjugation of Phosphate Guide Molecule Fragments to 3′ Hydroxyl Guide Molecule Fragments with Carbodiimide

As illustrated in FIGS. 3A and 3B, a first 5′ guide molecule fragment (e.g., a 34mer) is synthesized using standard phosphoramidite chemistry. A second 3′ guide molecule fragment (e.g., a 66mer) comprising a 5′-phosphate is also synthesized. The first and second guide molecule fragments are mixed at a molar ratio of 1:1 in a coupling buffer (100 mM 2-(N-morpholino)ethanesulfonic acid (MES), pH 6, 150 mM NaCl, 5 mM MgCl₂, and 10 mM ZnCl₂). The two guide molecule fragments are annealed, followed by addition of 100 mM 1-ethyl-3-(3-dimethylaminopropyl)carbodiimide (EDC) and 90 mM imidazole. The reaction mixture is mixed at 4° C. for 1-5 days, followed by desalting and anion-exchange HPLC purification.

### Example 4: Assessment of Guide Molecule Activity in HEK293T Cells

The activity of guide molecules conjugated in accordance with the process of Example 2 was assessed in HEK293T cells via a T7E1 cutting assay. For clarity, all guide molecules used in this Example contained identical targeting domain sequences, and substantially similar RNA backbone sequences, as shown in Table 9, below. In the table, targeting domain sequences are denoted as degenerate sequences by “N”s, while the position of a cross-link between two guide molecule fragments is denoted by an [L].

TABLE 9

| Guide molecule or | | |
|---|---|---|
| guide molecule | SEQ ID | |
| fragment | NO. | Sequence |
| 100mer gRNA | 32 | NNNNNNNNNNNNNNNNNNNNGUUUUAGAGCUAGAAAUA |
| | | GCAAGUUAAAAUAAGGCUAGUCCGUUAUCAACUUGAAA |
| | | AAGUGGCACCGAGUCGGUGCUUUU |
| 34mer 5′ gRNA | 33 | NNNNNNNNNNNNNNNNNNNNGUUUUAGAGCUAGA |
| fragment | | |
| 66mer 3′ gRNA | 34 | AAUAGCAAGUUAAAAUAAGGCUAGUCCGUUAUCAACUU |
| fragment | | GAAAAAGUGGCACCGAGUCGGUGCUUUU |
| 100mer conjugated | 35 | NNNNNNNNNNNNNNNNNNNNGUUUUAGAGCUAGA[L]A |
| gRNA | | AUAGCAAGUUAAAAUAAGGCUAGUCCGUUAUCAACUUG |
| | | AAAAAGUGGCACCGAGUCGGUGCUUUU |

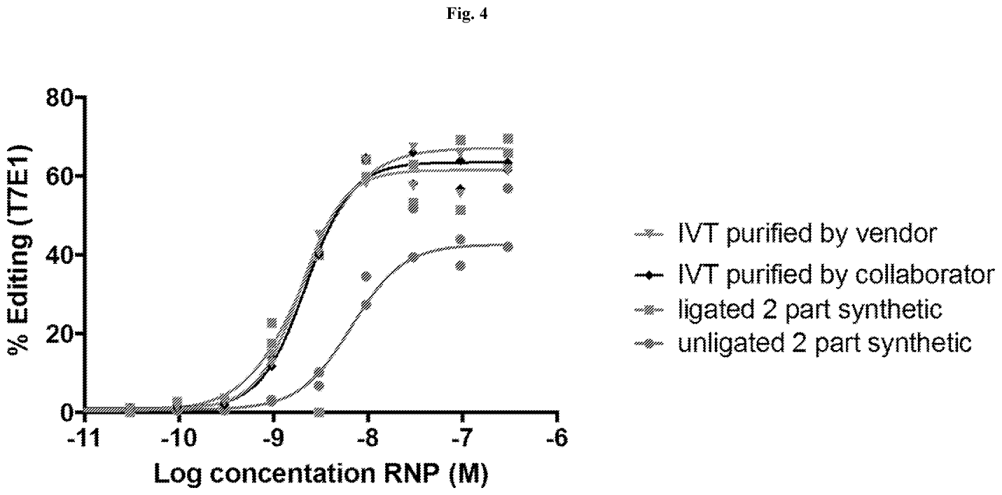

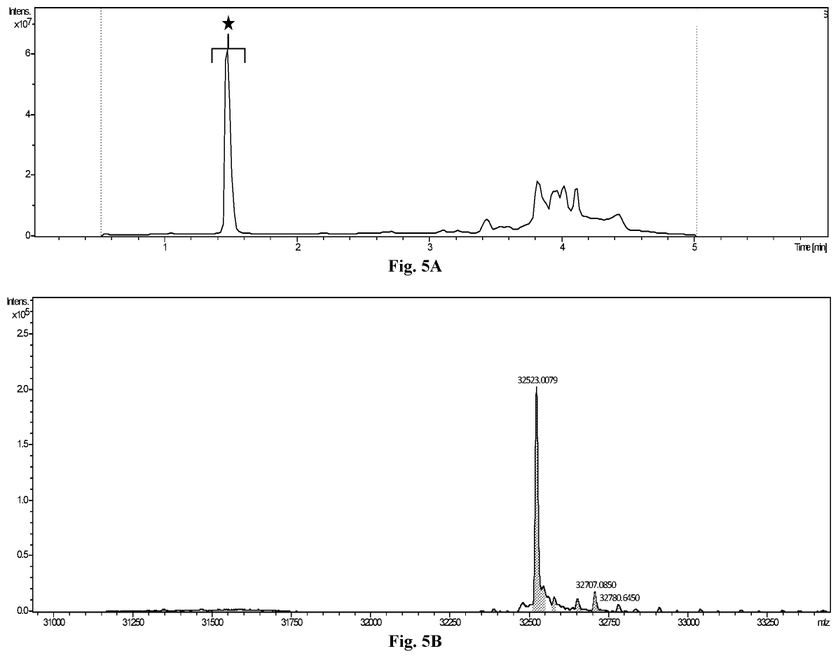

Varying concentrations of ribonucleoprotein complexes comprising, variously, a unimolecular guide molecule generated by IVT, a synthetic unimolecular guide molecule (i.e., prepared without conjugation), or a synthetic unimolecular guide molecule conjugated by the bromoacetyl-thiol process of Example 2 were introduced into HEK293T cells by lipofection (CRISPR-Max, Thermo Fisher Scientific, Waltham, Mass.), and genomic DNA was harvested later. Cleavage was assessed using a standard T7E1 cutting assay, using a commercial kit (Surveyor™ commercially available from Integrated DNA Systems, Coralville, Iowa). Results are presented in FIG. 4.

As the results show, the conjugated guide molecule supported cleavage in HEK293 cells in a dose-dependent manner that was consistent with that observed with the unimolecular guide molecule generated by IVT or the synthetic unimolecular guide molecule. It should be noted that unconjugated annealed guide molecule fragments supported a lower level of cleavage, though in a similar dose-dependent manner. These results suggest that guide molecules conjugated according to the methods of this disclosure support high levels of DNA cleavage in substantially the same manner as unimolecular guide molecules generated by IVT or synthetic unimolecular guide molecules.

### Example 5: Evaluation of Guide Molecule Purity by Gel Electrophoresis and Mass Spectrometry

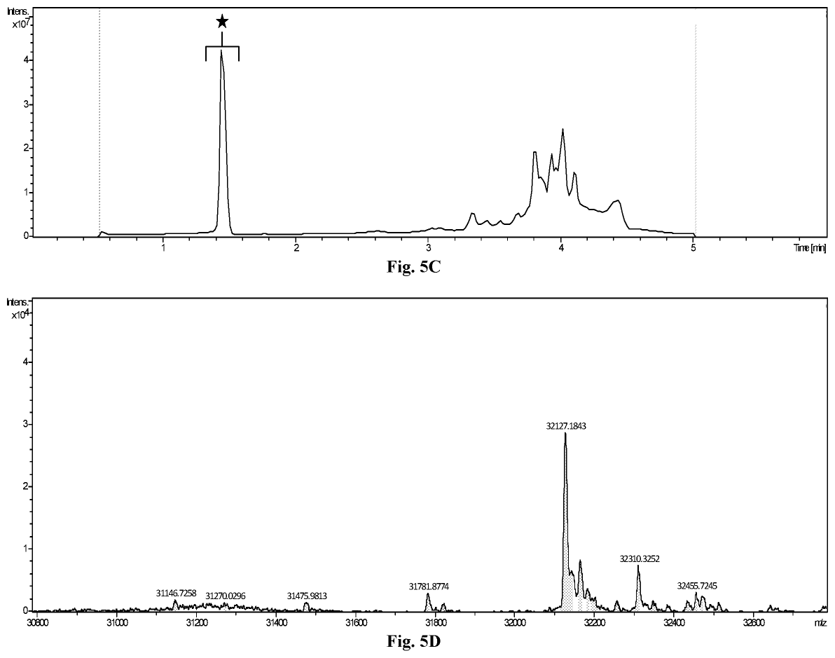

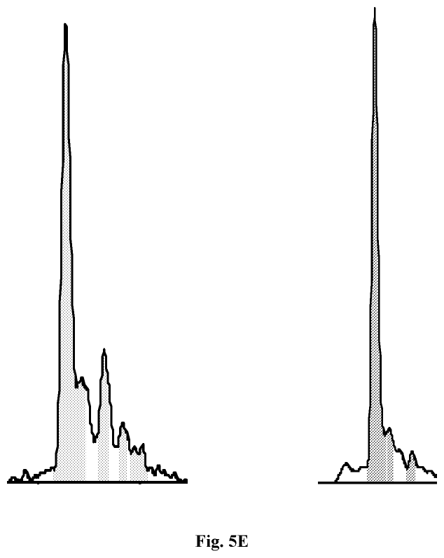

The purity of a composition of guide molecules conjugated with a urea linker according to the process of Example 1 was compared by total ion current chromatography and mass spectrometry with the purity of a composition of commercially prepared synthetic unimolecular guide molecules (i.e., prepared without conjugation). 100 pmol of an analyte was injected for mass analysis. The analysis was achieved by LC-MS on a Bruker microTOF-QII mass spectrometer equipped with a Waters ACQUITY UPLC system. A ThermoDNAPac C18 column was used for separation. Results are shown in FIG. 5.

FIG. 5A shows a representative ion chromatograph and FIG. 5B shows a deconvoluted mass spectrum of an ion-exchange purified guide molecule conjugated with a urea linker according to the process of Example 1. FIG. 5C shows a representative ion chromatograph and FIG. 5D shows a deconvoluted mass spectrum of a commercially prepared synthetic unimolecular guide molecule. Mass spectra were assessed for the highlighted peaks in the ion chromatographs. FIG. 5E shows expanded versions of the mass spectra. The mass spectrum for the commercially prepared synthetic unimolecular guide molecule is on the left side (34% purity by total mass) while the mass spectrum for the guide molecule conjugated with a urea linker according to the process of Example 1 is on the right side (72% purity by total mass).

### Example 6: Evaluation of Guide Molecule Purity by Sequence Analysis

The purity of a composition of guide molecules conjugated with a urea linkage, as described in Example 1, was compared with the purity of a composition of commercially prepared synthetic unimolecular guide molecules (i.e., prepared without conjugation) and a composition of guide molecules conjugated with a thioether linkage, as described in Example 2. All compositions of guide molecules were based on the same predetermined guide molecule sequence.

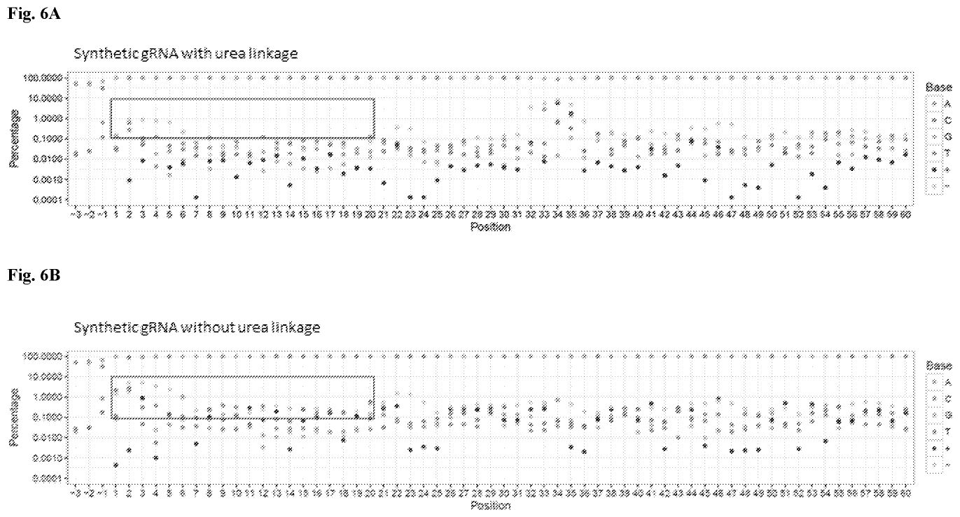

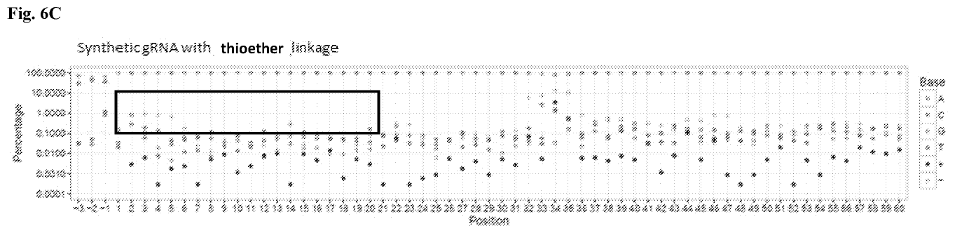

FIG. 6A shows a plot depicting the frequency with which individual bases and length variances occurred at each position from the 5′ end of complementary DNAs (cDNAs) generated from synthetic unimolecular guide molecules that included a urea linkage, and FIG. 6B shows a plot depicting the frequency with which individual bases and length variances occurred at each position from the 5′ end of cDNAs generated from commercially prepared synthetic unimolecular guide molecules (i.e., prepared without conjugation). Boxes surround the 20 bp targeting domain of the guide molecule. In this example, guide molecules that included the urea linkage resulted in greater sequence fidelity in the targeting domain (i.e., less than 1% of guide molecules included a deletion at any given position, and less than 1% of guide molecules included a substitution at any given position) compared to the guide molecules from the commercially prepared synthetic unimolecular guide molecules (in which less than 10% of guide molecules included a deletion at any given position, and less than 5% included a substitution at any given position).

FIG. 6C shows a plot depicting the frequency with which individual bases and length variances occurred at each position from the 5′ end of cDNAs generated from synthetic unimolecular guide molecules that included the thioether linkage. As shown in FIG. 6C, high levels of 5′ sequence fidelity were seen, demonstrating production of compositions of guide molecules with a high level of sequence fidelity and purity. The alignments in FIG. 6A (urea linkage) and FIG. 6C (thioether linkage) also showed a region of relatively high frequency of mismatches/indels at the linkage site (position 34). These data suggest that guide molecules synthesized by the methods of this disclosure demonstrate decreased frequency of deletions and substitutions as compared to commercially available guide molecules.

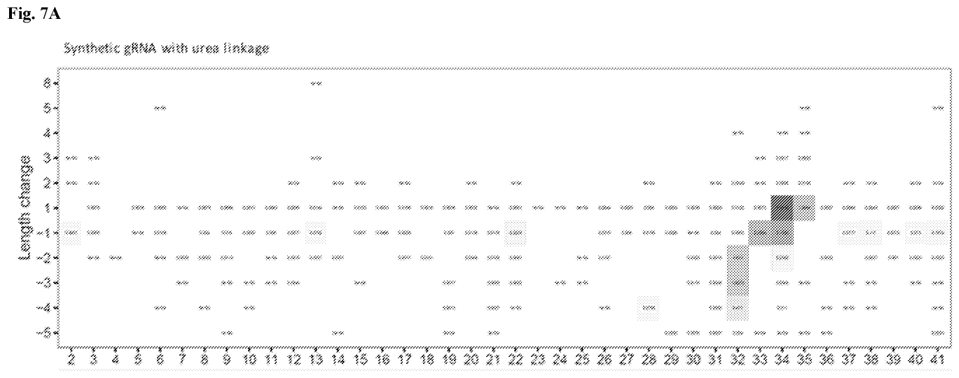

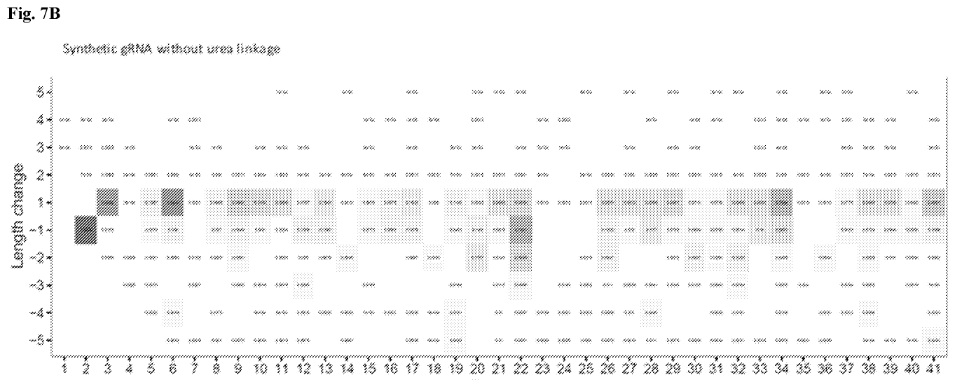

FIGS. 7A and 7B are graphs depicting internal sequence length variances (+5 to −5) at the first 41 positions from the 5′ ends of cDNAs generated from synthetic unimolecular guide molecules that included the urea linkage (FIG. 7A), and from commercially prepared synthetic unimolecular guide molecules (i.e., prepared without conjugation) (FIG. 7B). As shown, guide molecules that included the urea linkage had a reduction in the frequency and length of insertions/deletions, relative to the commercially prepared synthetic unimolecular guide molecules (i.e., prepared without conjugation).

### Example 7: Assessment of Guide Molecule Activity in CD34+ Cells

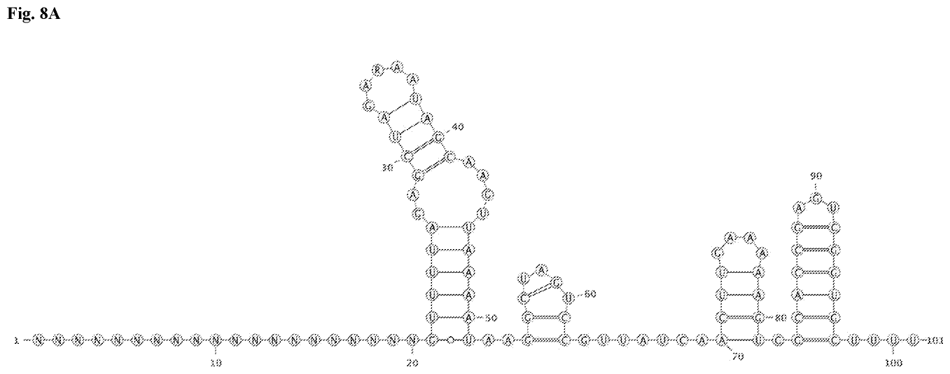

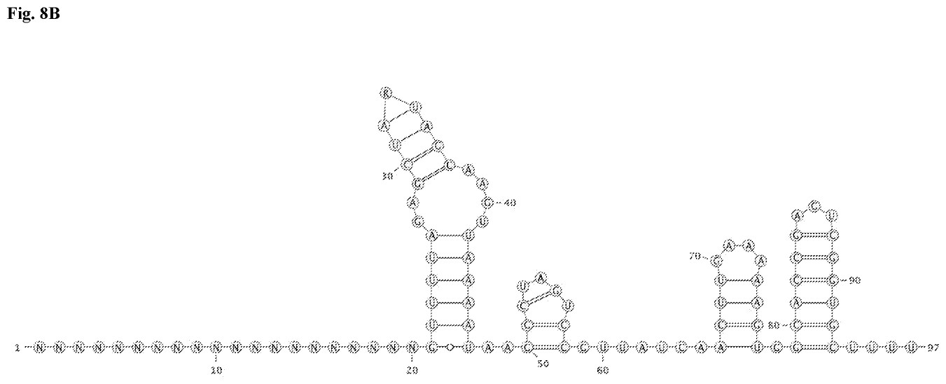

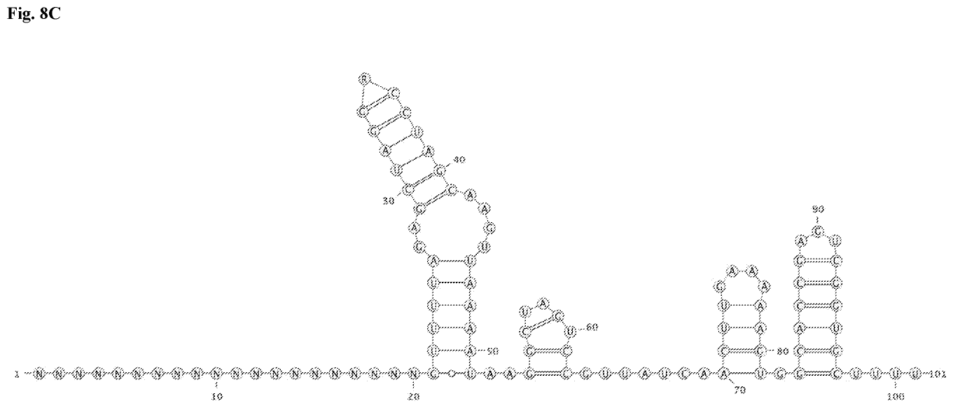

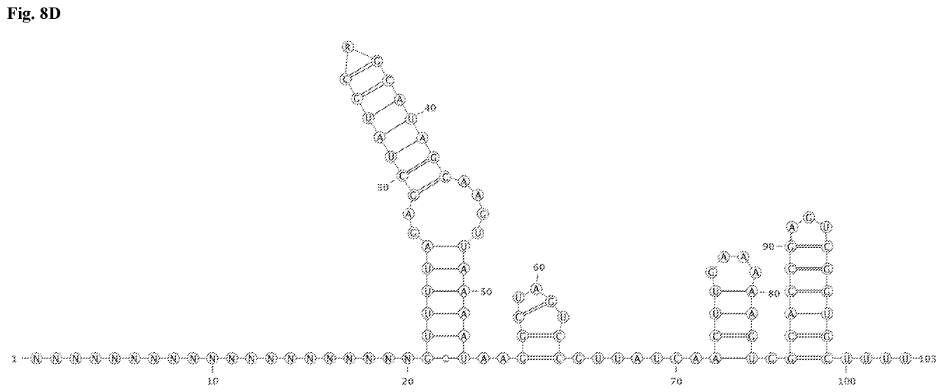

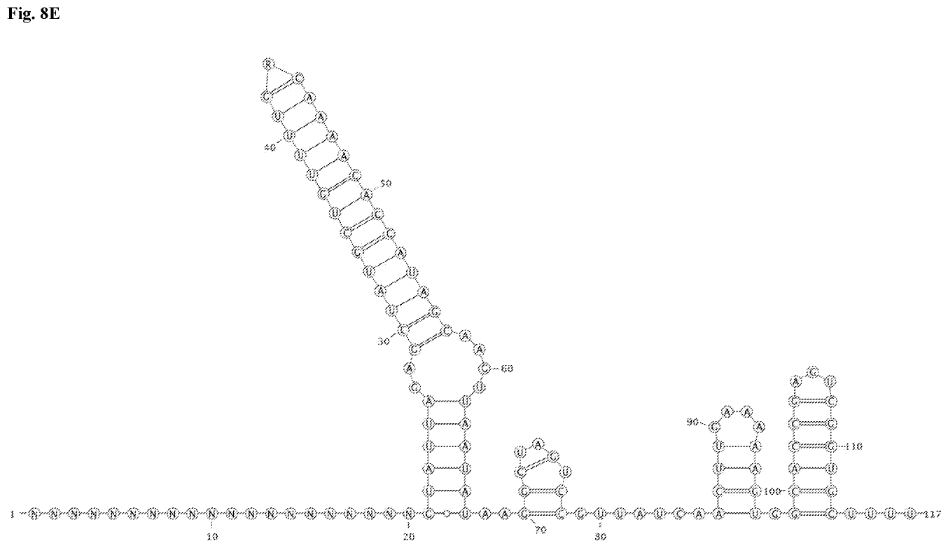

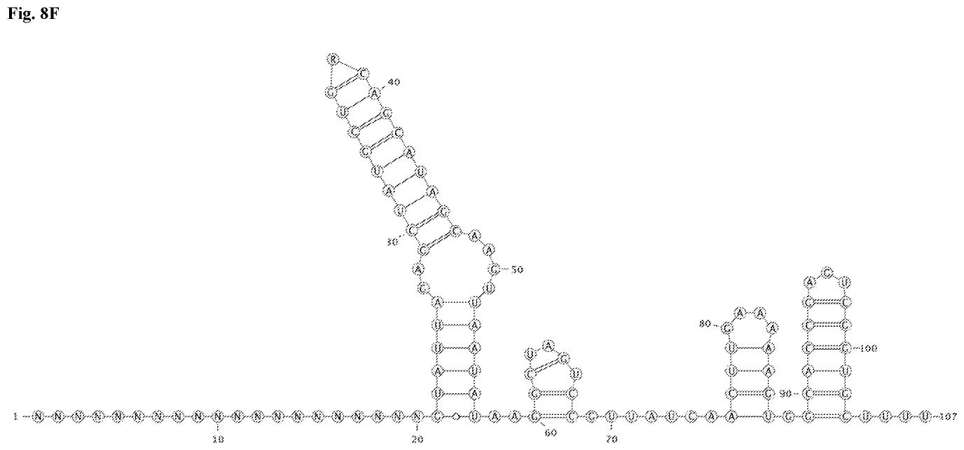

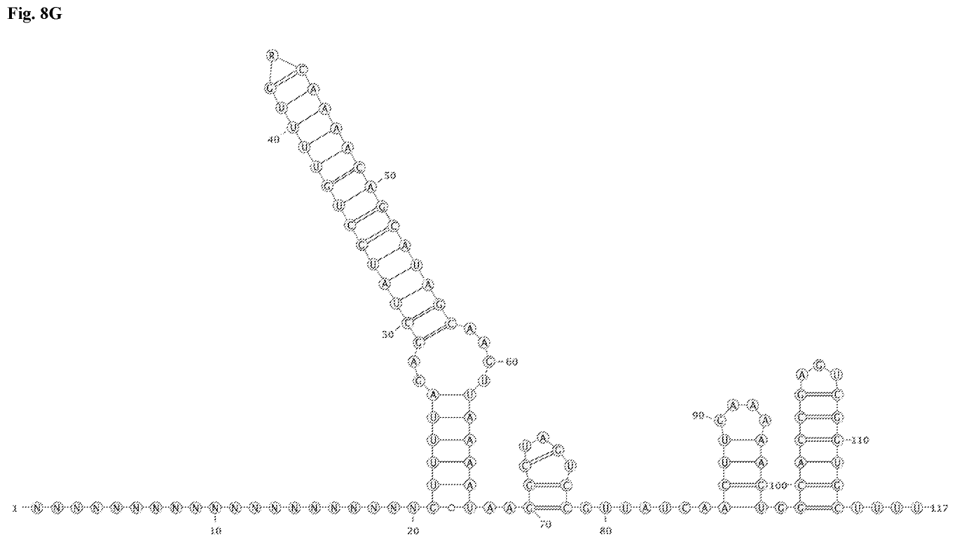

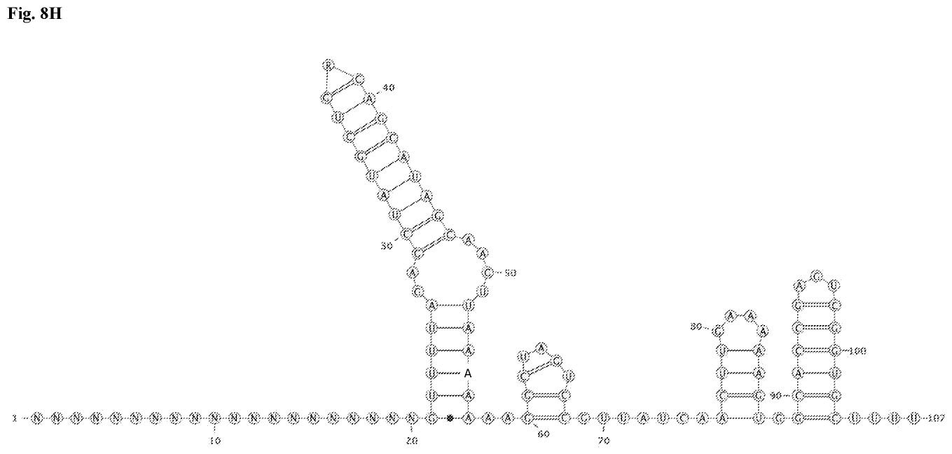

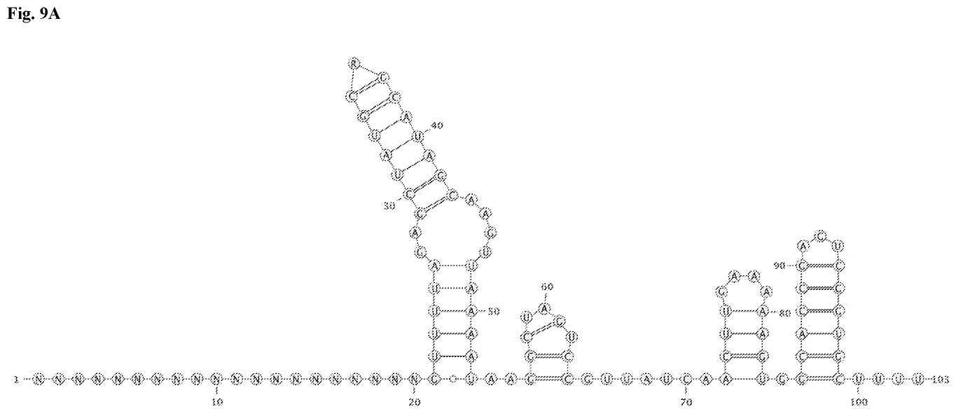

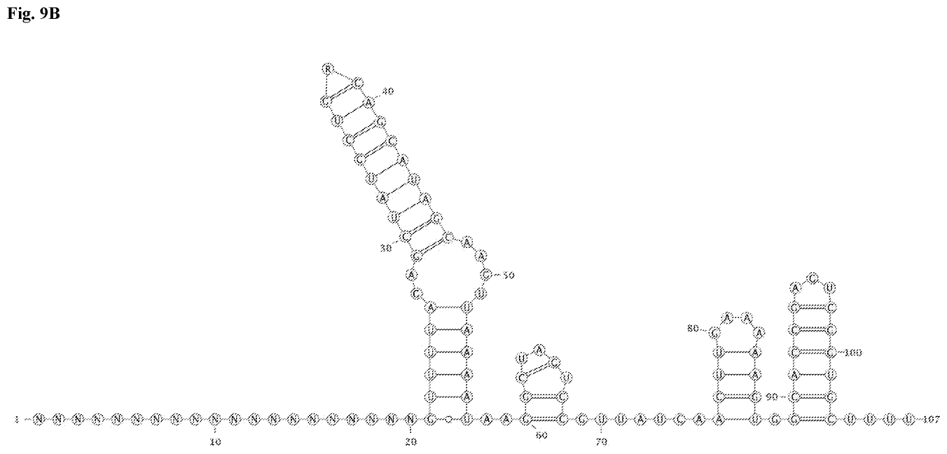

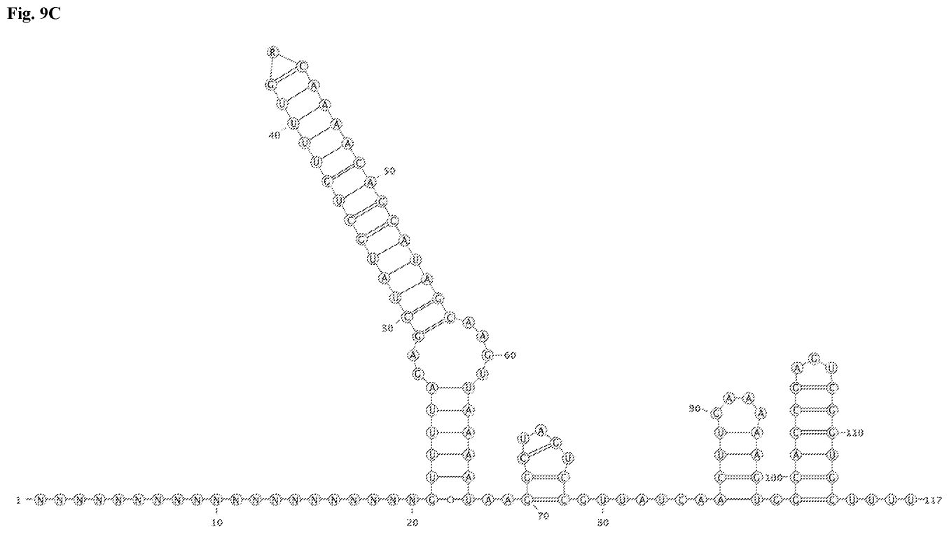

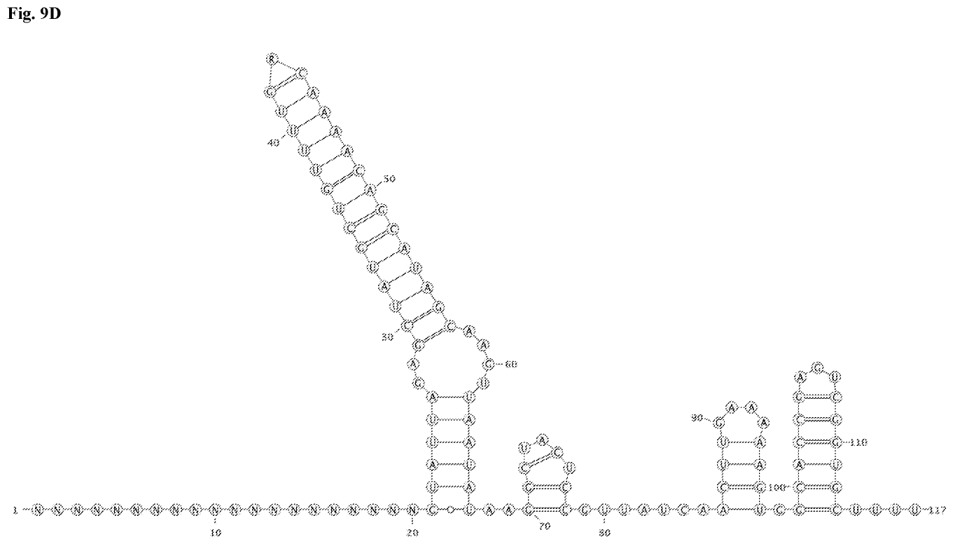

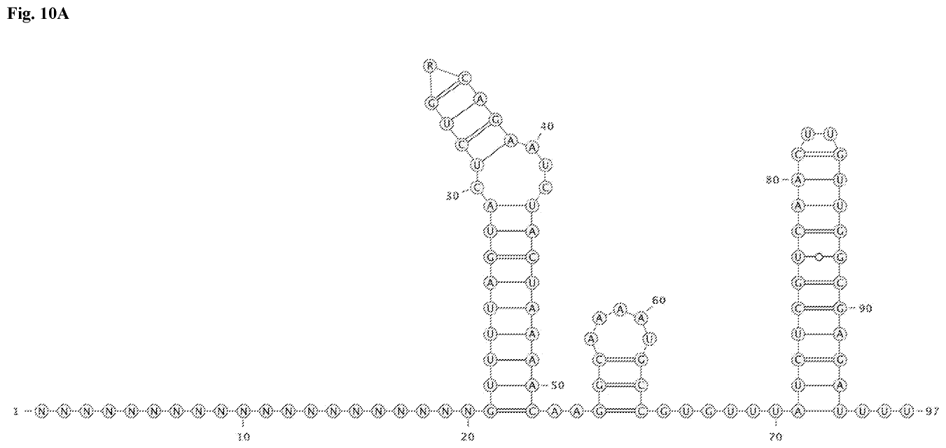

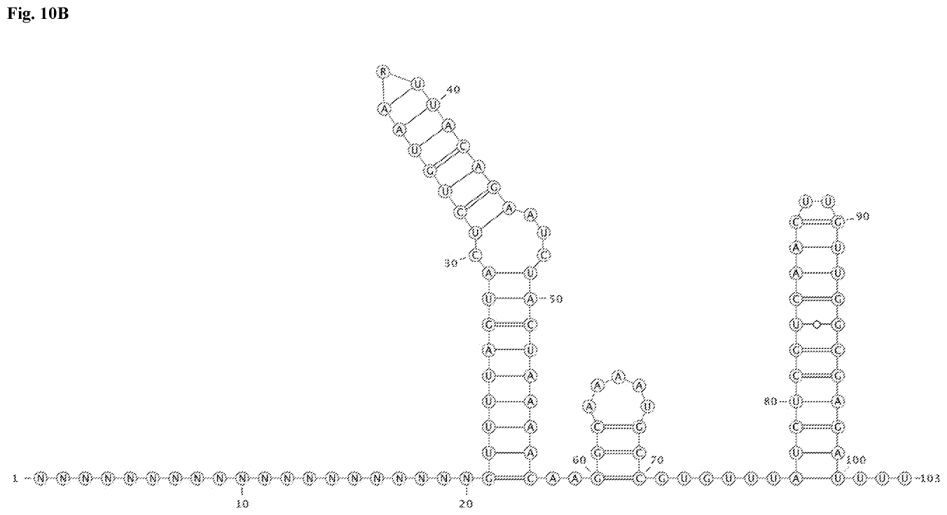

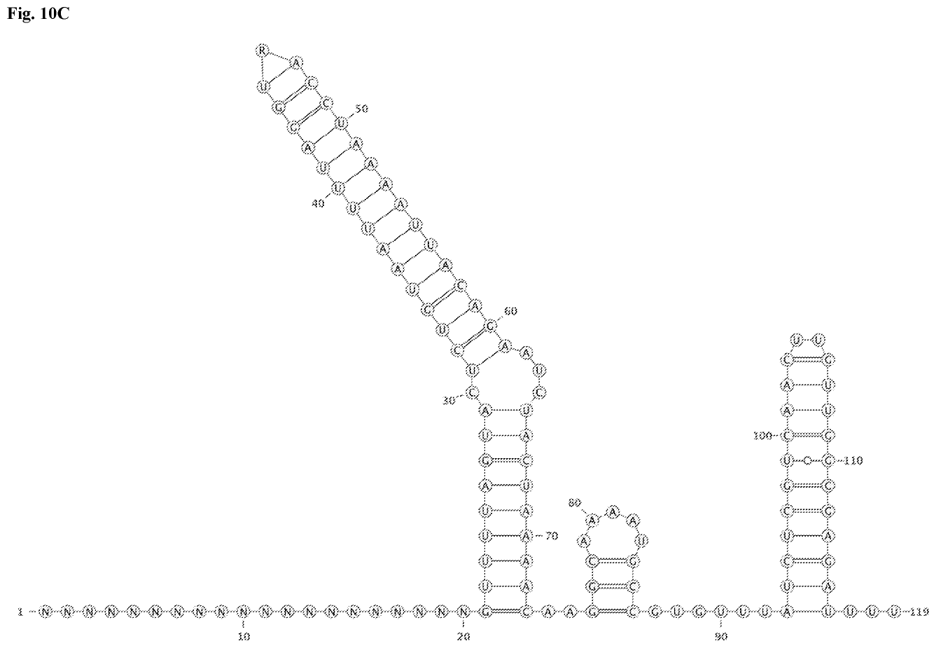

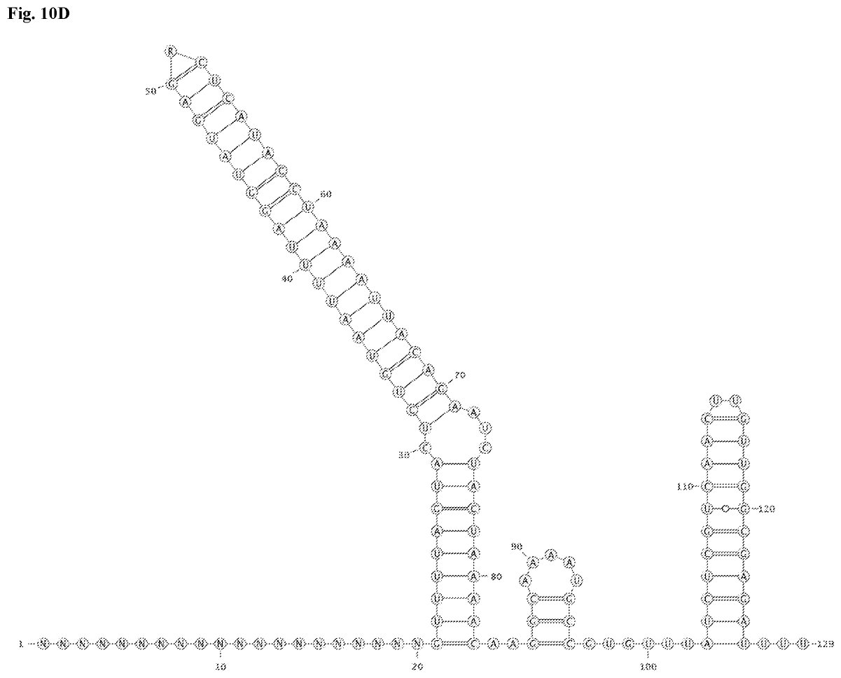

The activity of guide molecules with urea linkages conjugated in accordance with the process of Example 1 was assessed in CD34+ cells via next generation sequencing techniques. Guide molecules discussed in this Example contained one of three targeting domain sequences and various guide molecule backbone sequences, as shown in Table 10, below and FIGS. 8A-L, 9A-E, and 10A-D. The position of the urea linkage between two guide molecule fragments is denoted by [UR] in Table 10 and ® in FIGS. 8A-L, 9A-E, and 10A-D. The guide molecules with the first two targeting domain sequences (denoted gRNA 1 followed by a letter or gRNA 2 followed by a letter) were based on a S. pyogenes gRNA backbone while the guide molecules with the third targeting domain sequence (denoted gRNA 3 followed by a letter) were based on a S. aureus gRNA backbone.

The conjugated guide molecules were resuspended in pH 7.5 buffer, melted and reannealed, and then added to a suspension of S. pyogenes Cas9 to yield a solution with 55 μM fully-complexed ribonucleoprotein.

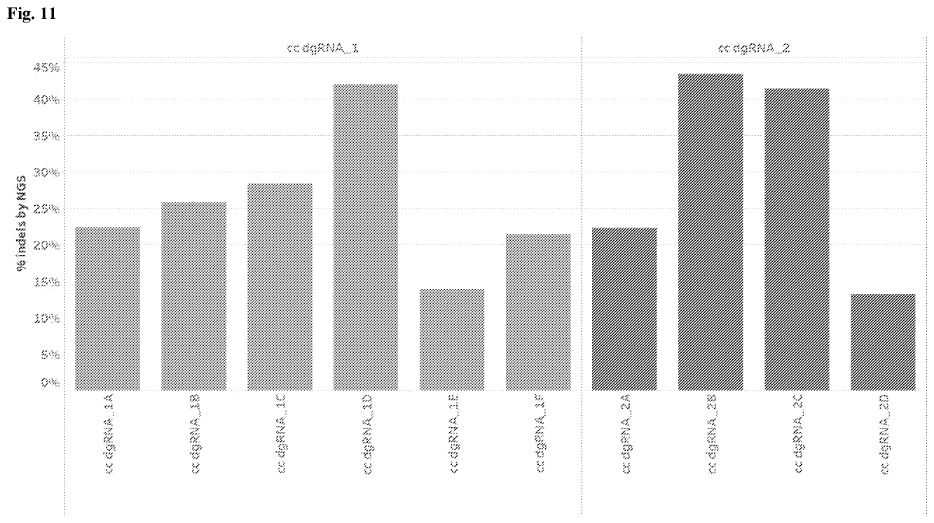

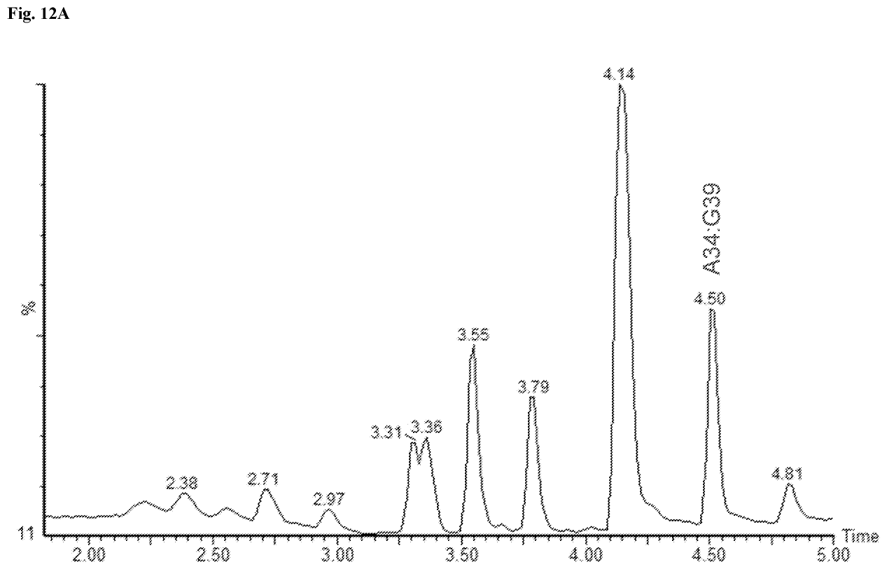

Human CD34+ cells were counted, centrifuged to a pellet and resuspended in P3 Nucleofection Buffer, then dispensed to each well of a 96-well Nucleocuvette Plate that was pre-filled with human HSC media (StemSpan™ Serum-Free Expansion Medium, StemCell Technologies, Vancouver, British Columbia, Canada) to yield 50,000 cells/well. A fully-complexed ribonucleoprotein solution as described above was added to each well in the Nucleocuvette Plate, followed by gentle mixing. Nucleofection was performed on an Amaxa Nucleofector System (Lonza, Basel, Switzerland). Nucleofected cells were incubated for 72 h at 37° C. and 5% CO₂ to allow editing to plateau. Genomic DNA was then extracted from nucleofected cells using the DNAdvance DNA isolation Kit according to manufacturer's instructions. Cleavage was assessed using next generation sequencing techniques to quantify % insertions and deletions (indels) relative to the wild-type human reference sequence. Results for gRNAs in Table 10 that were tested in CD34+ cells are presented in FIG. 11.

As the results in FIG. 11 show, ligated guide molecules generated according to Example 1 support DNA cleavage in CD34+ cells. % indels were found to increase with increasing stemloop length, but incorporation of a U-A swap adjacent to the stemloop sequence (see gRNA 1E, gRNA IF, and gRNA 2D) mitigates the effect. These data suggest chemically conjugated synthetic unimolecular guide molecules with a longer stemloop feature result in higher levels of DNA cleavage in cells. In addition, DNA cleavage activity is independent of ligation efficiency and must be determined empirically.

TABLE 10

| | SEQ | |
|---|---|---|
| Guide | ID | |
| molecule | NO. | Sequence |
| gRNA | 36 | GUAACGGCAGACUUCUCCUCGUUUUAGAGCUAGA[UR]AAUAGCAAGUUAAAAUAA |
| 1A | | GGCUAGUCCGUUAUCAACUUGAAAAAGUGGCACCGAGUCGGUGCUUUU |
| gRNA | 37 | GUAACGGCAGACUUCUCCUCGUUUUAGAGCUA[UR]UAGCAAGUUAAAAUAAGGCU |
| 1B | | AGUCCGUUAUCAACUUGAAAAAGUGGCACCGAGUCGGUGCUUUU |
| gRNA | 38 | GUAACGGCAGACUUCUCCUCGUUUUAGAGCUAGG[UR]CCUAGCAAGUUAAAAUAA |
| 1C | | GGCUAGUCCGUUAUCAACUUGAAAAAGUGGCACCGAGUCGGUGCUUUU |
| gRNA | 39 | GUAACGGCAGACUUCUCCUCGUUUUAGAGCUAUGC[UR]GCAUAGCAAGUUAAAAU |
| 1D | | AAGGCUAGUCCGUUAUCAACUUGAAAAAGUGGCACCGAGUCGGUGCUUUU |
| gRNA | 40 | GUAACGGCAGACUUCUCCUCGUAUUAGAGCUAUGCUGUUUUG[UR]CAAAACAGCA |
| 1E | | UAGCAAGUUAAUAUAAGGCUAGUCCGUUAUCAACUUGAAAAAGUGGCACCGAGUCG |
| | | GUGCUUUU |
| gRNA | 41 | GUAACGGCAGACUUCUCCUCGUAUUAGAGCUAUGCUG[UR]CAGCAUAGCAAGUUA |
| 1F | | AUAUAAGGCUAGUCCGUUAUCAACUUGAAAAAGUGGCACCGAGUCGGUGCUUUU |
| gRNA | 42 | GUAACGGCAGACUUCUCCUCGUUUUAGAGCUAUGCUGUUUUG[UR]CAAAACAGCA |
| 1G | | UAGCAAGUUAAAAUAAGGCUAGUCCGUUAUCAACUUGAAAAAGUGGCACCGAGUCG |
| | | GUGCUUUU |
| gRNA | 43 | GUAACGGCAGACUUCUCCUCGUUUUAGAGCUAUGCUG[UR]CAGCAUAGCAAGUUA |
| 1H | | AAAAAAGGCUAGUCCGUUAUCAACUUGAAAAAGUGGCACCGAGUCGGUGCUUUU |
| gRNA | 44 | GUAACGGCAGACUUCUCCUCGUUUUAGAGCUAAAGA[UR]AAUUUAGCAAGUUAAA |
| 1I | | AUAAGGCUAGUCCGUUAUCAACUUGAAAAAGUGGCACCGAGUCGGUGCUUUU |
| gRNA | 45 | GUAACGGCAGACUUCUCCUCGUUUUAGAGCUAAA[UR]UUUAGCAAGUUAAAAUAA |
| 1J | | GGCUAGUCCGUUAUCAACUUGAAAAAGUGGCACCGAGUCGGUGCUUUU |
| gRNA | 46 | GUAACGGCAGACUUCUCCUCGUUUUAGAGCUAAAGGGA[UR]AACCUUUAGCAAGU |
| 1K | | UAAAAUAAGGCUAGUCCGUUAUCAACUUGAAAAAGUGGCACCGAGUCGGUGCUUUU |
| gRNA | 47 | GUAACGGCAGACUUCUCCUCGUUUUAGAGCUAGdA[UR]AAUAGCAAGUUAAAAUA |
| 1L | | AGGCUAGUCCGUUAUCAACUUGAAAAAGUGGCACCGAGUCGGUGCUUUU |
| gRNA | 48 | CUAACAGUUGCUUUUAUCACGUUUUAGAGCUAUGC[UR]GCAUAGCAAGUUAAAAU |
| 2A | | AAGGCUAGUCCGUUAUCAACUUGAAAAAGUGGCACCGAGUCGGUGCUUUU |
| gRNA | 49 | CUAACAGUUGCUUUUAUCACGUUUUAGAGCUAUGCUG[UR]CAGCAUAGCAAGUUA |
| 2B | | AAAUAAGGCUAGUCCGUUAUCAACUUGAAAAAGUGGCACCGAGUCGGUGCUUUU |
| gRNA | 50 | CUAACAGUUGCUUUUAUCACGUUUUAGAGCUAUGCUGUUUUG[UR]CAAAACAGCA |
| 2C | | UAGCAAGUUAAAAUAAGGCUAGUCCGUUAUCAACUUGAAAAAGUGGCACCGAGUCG |
| | | GUGCUUUU |
| gRNA | 51 | CUAACAGUUGCUUUUAUCACGUAUUAGAGCUAUGCUGUUUUG[UR]CAAAACAGCA |
| 2D | | UAGCAAGUUAAUAUAAGGCUAGUCCGUUAUCAACUUGAAAAAGUGGCACCGAGUCG |
| | | GUGCUUUU |
| gRNA | 52 | CUAACAGUUGCUUUUAUCACGUUUUAGAGCUAGA[UR]AAUAGCAAGUUAAAAUAA |
| 2E | | GGCUAGUCCGUUAUCAACUUGAAAAAGUGGCACCGAGUCGGUGCUUUU |
| gRNA | 53 | GUAACGGCAGACUUCUCCUCGUUUUAGUACUCUG[UR]CAGAAUCUACUAAAACAA |
| 3A | | GGCAAAAUGCCGUGUUUAUCUCGUCAACUUGUUGGCGAGAUUUU |
| gRNA | 54 | GUAACGGCAGACUUCUCCUCGUUUUAGUACUCUGUAA[UR]UUACAGAAUCUACUA |
| 3B | | AAACAAGGCAAAAUGCCGUGUUUAUCUCGUCAACUUGUUGGCGAGAUUUU |
| gRNA | 55 | GUAACGGCAGACUUCUCCUCGUUUUAGUACUCUGUAAUUUUAGGU[UR]ACCUAAA |
| 3C | | AUUACAGAAUCUACUAAAACAAGGCAAAAUGCCGUGUUUAUCUCGUCAACUUGUUG |
| | | GCGAGAUUUU |
| gRNA | 56 | GUAACGGCAGACUUCUCCUCGUUUUAGUACUCUGUAAUUUUAGGUAUGAG[UR]CU |
| 3D | | CAUACCUAAAAUUACAGAAUCUACUAAAACAAGGCAAAAUGCCGUGUUUAUCUCGU |
| | | CAACUUGUUGGCGAGAUUUU |

### Example 8: Evaluation of Computational Model of Ligation Efficiency

The ligation efficiency of the reaction described in Example 1 is one measure of the suitability of a particular guide molecule structure. Since the reactive functional group of the first and second guide molecule fragments in Example 1 is the same (an amine), competitive homo-coupling is a potential side product. This Example evaluated whether ligation efficiency (i.e., the % of hetero-coupled product in the reaction product) can be predicted through computational modeling of the free energy difference of the homo-coupling reaction (ΔG₁), compared to the free energy difference of the hetero-coupling reaction (ΔG₂) using the OligoAnalyzer 3.1 tool available at http://www.idtdna.com/calc/analyzer. Results of this analysis are shown in Table 11.

TABLE 11

| Guide | SEQ | Ligation | ΔG₁ | ΔG₂ | ΔG₂ − ΔG₁ |
|---|---|---|---|---|---|
| molecule | ID NO. | efficiency | (kcal/mol) | (kcal/mol) | (kcal/mol) |
| gRNA 1A | 36 | −55% | −6.90 | −10.93 | −4.03 |
| gRNA 1C | 38 | 18% | −6.90 | −10.93 | −4.03 |
| gRNA 1D | 39 | 50% | −6.90 | −12.27 | −5.37 |
| gRNA 1E | 40 | 50% | −6.34 | −24.95 | −18.61 |
| gRNA 1F | 41 | 31% | −6.34 | −15.82 | −9.48 |
| gRNA 1G | 42 | 12% | −6.90 | −24.95 | −18.05 |
| gRNA 1H | 43 | 60% | −6.90 | −15.82 | −8.92 |
| gRNA 1I | 44 | −50% | −6.90 | −10.93 | −4.03 |
| gRNA 1J | 45 | −50% | −6.90 | −10.93 | −4.03 |
| gRNA 1K | 46 | −55% | −6.90 | −10.93 | −4.03 |
| gRNA 2A | 48 | 18% | −6.34 | −12.27 | −5.93 |
| gRNA 2C | 50 | 48% | −6.84 | −24.95 | −18.11 |
| gRNA 2D | 51 | 45% | −6.84 | −24.95 | −18.11 |
| gRNA 2E | 52 | 5% | −6.34 | −8.64 | −2.30 |

As shown in Table 11, ligation efficiency (as measured by densitometry following gel analysis) was well predicted for most sequences with a more negative ΔG₂−ΔG₁ value corresponding to a more favorable ligation efficiency (e.g., compare gRNAs 2A and 2C). However, the ligation efficiency to form certain guide molecules was not always correlated with the ΔG2−ΔG₁ value (e.g., see gRNA 1G where a more negative ΔG₂−ΔG₁ value did not lead to higher ligation efficiency), indicating that modifications and experimentation may be required for conjugating certain guide molecule fragments. For example, ligation efficiency of gRNA 1G was improved by implementing a U-A swap in the sequence of the lower stem (compare ligation efficiency of gRNA 1G with gRNA 1E), where the U-A swap was designed to prevent staggered annealing of two guide molecule fragments before ligation.

### Example 9: Characterization of Urea Linkage by Mass Spectrometry

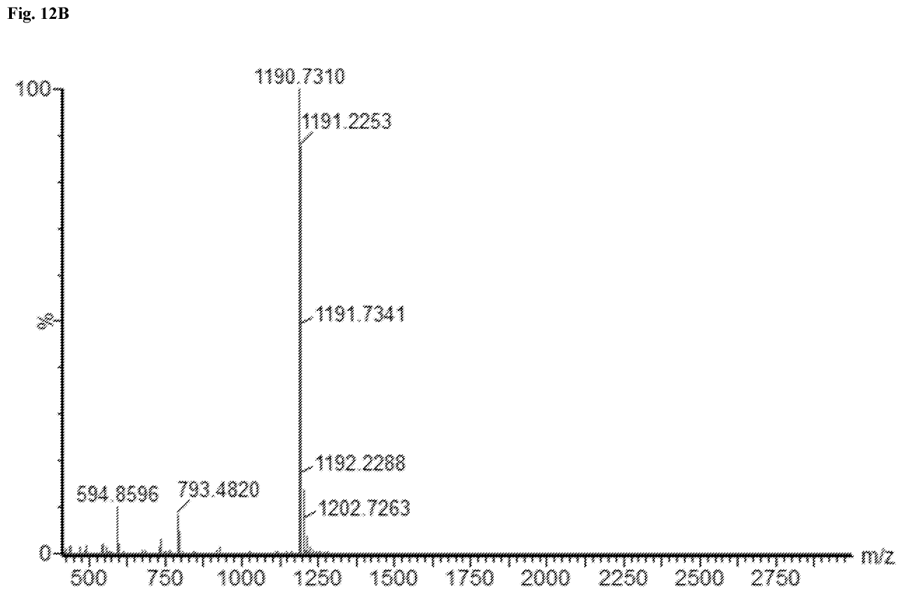

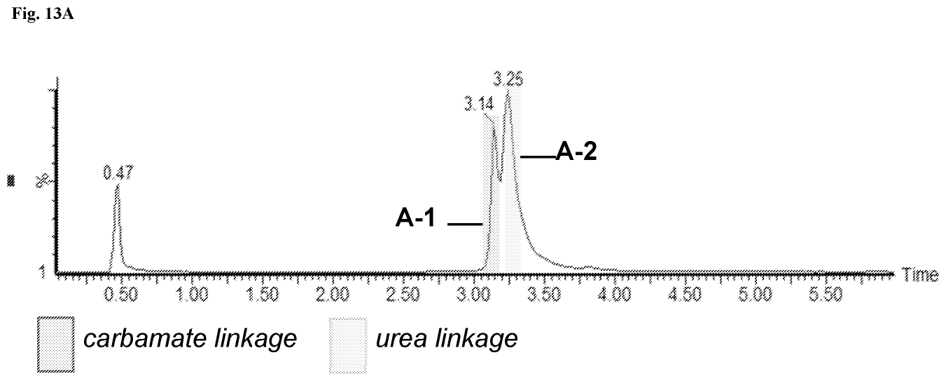

A chemically conjugated guide molecule, containing a urea linkage and synthesized as described in Example 1, was characterized by mass spectrometry. After synthesis, chemical ligation, and purification, gRNA 1A (see Table 10) was cleaved into fragments at the 3′-end of each G nucleotide in the primary sequence using the T1 endonuclease. These fragments were analyzed using LC-MS. In particular, the fragment containing the urea linkage, A-[UR]-AAUAG (A34:G39), was detected at a retention time of 4.50 min with m/z=1190.7 (FIG. 12A and FIG. 12B). LC/MS-MS analysis of this precursor ion revealed collision-induced dissociation fragment ions consistent with a urea linkage in gRNA 1A.

### Example 10: Characterization of a Carbamate Side Product

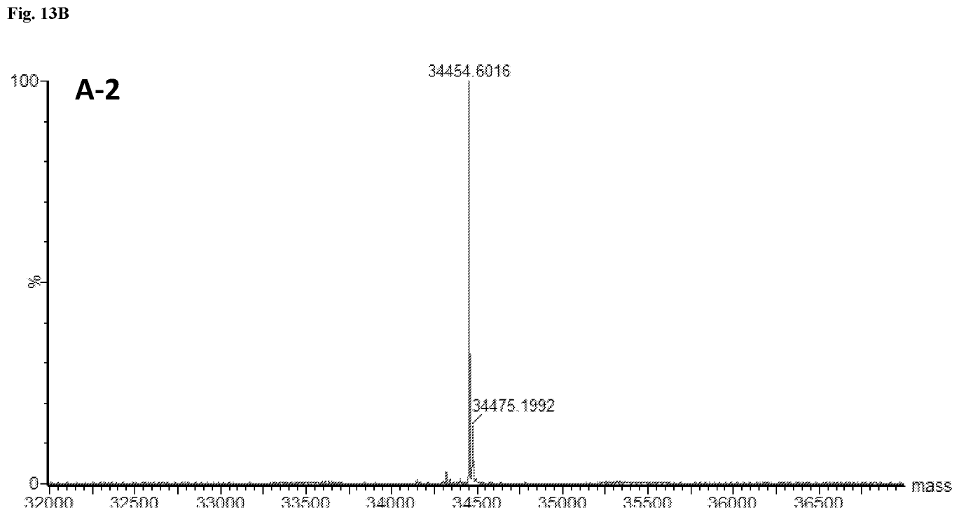

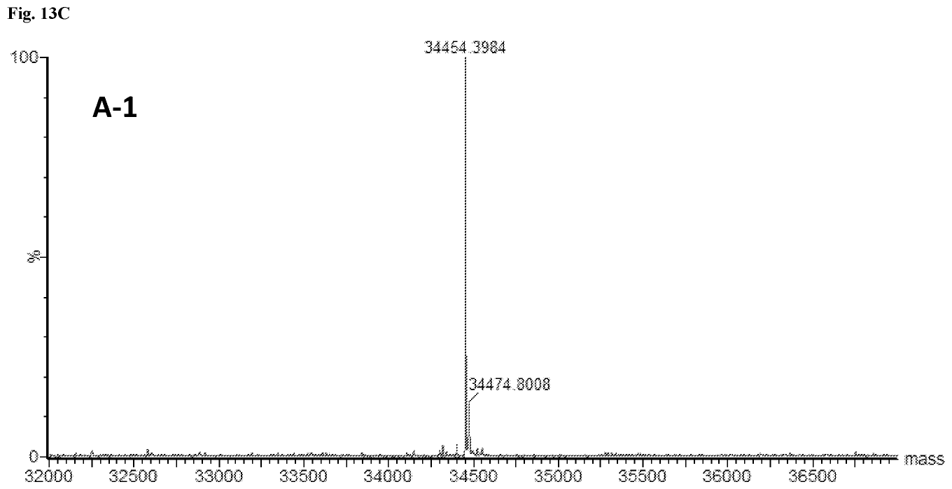

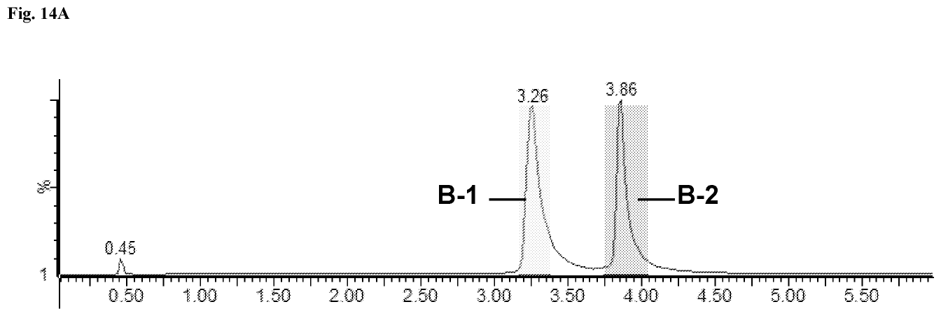

FIG. 13A shows LC-MS data for an unpurified composition of urea-linked guide molecules with both a major product (A-2, retention time of 3.25 min) and a minor product (A-1, retention time of 3.14 min) present. We note that the minor product (A-1) in FIG. 13A was enriched by combining fractions from the anion exchange purification that contained a higher percentage of carbamate minor product for purposes of illustration. The side product is typically detected in up to 10% yield in the synthesis of guide molecules in accordance with the process of Example 1. Analysis of each peak by mass spectrometry indicated that both products have the same molecular weight (see FIG. 13B and FIG. 13C).

In light of this, we hypothesized that the minor product was a carbamate side product resulting from a reaction between the 5′-NH₂ on the 5′ end of the 3′ guide molecule fragment and the 2′-OH on the 3′ end of the 5′ guide molecule fragment, as follows:

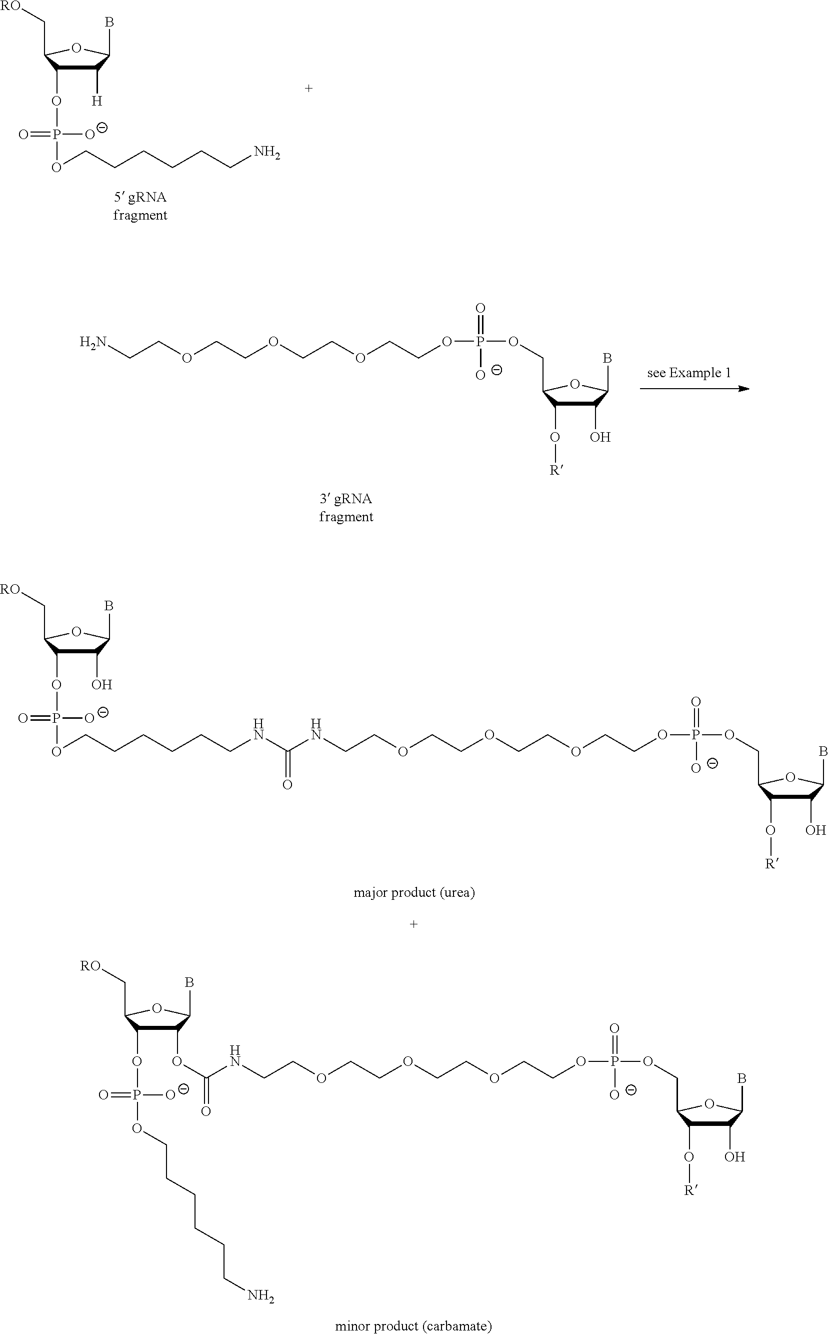

To further confirm the assignment of the carbamate side product, chemical modification with phenoxyacetic acid N-hydroxysuccinimide ester was performed. Basic chemical principles predict that only the minor product (carbamate) has a reactive nucleophilic center (free amine), and therefore only the minor product will be chemically functionalized. Addition of phenoxyacetic acid N-hydroxysuccinimide ester to the crude composition of urea-linked guide molecules should therefore result in a mixture of the major product (urea) and a chemically modified minor product (carbamate):

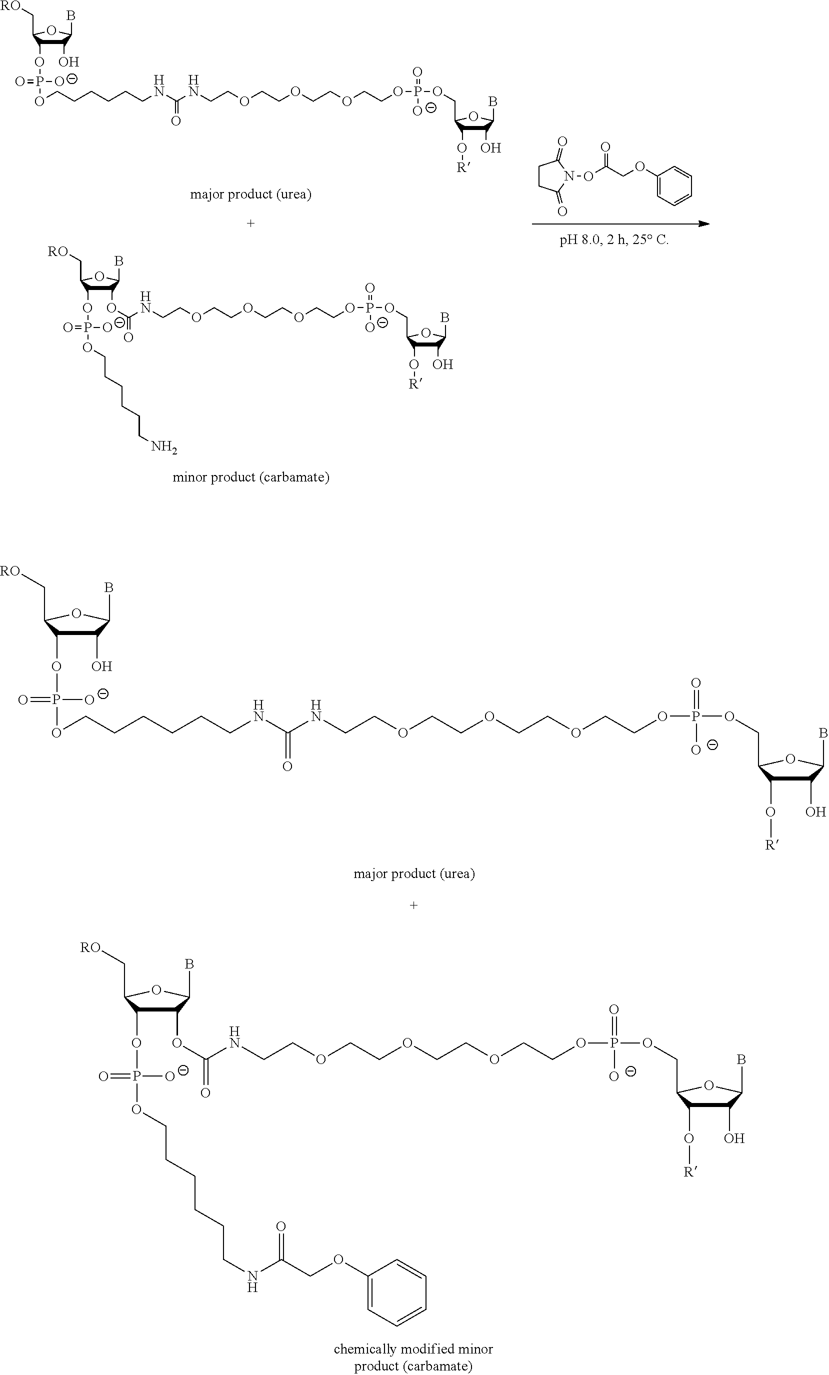

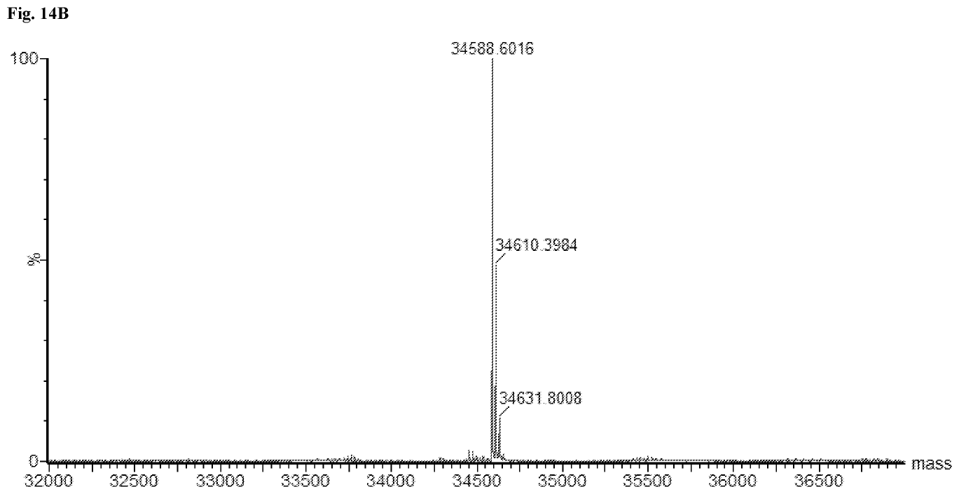

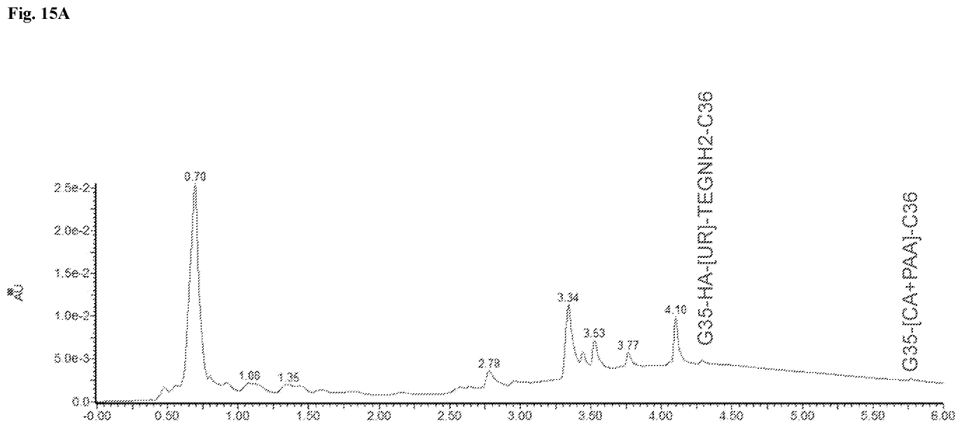

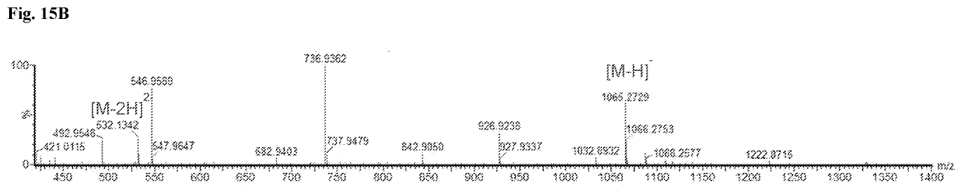

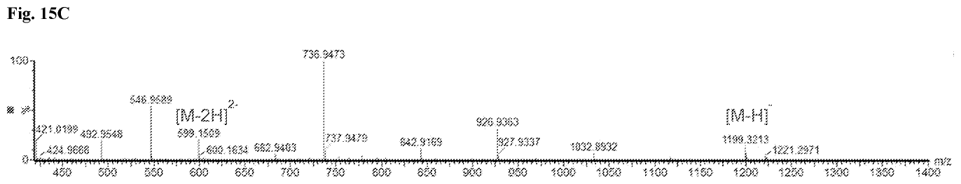

FIG. 14A shows LC-MS data for the guide molecule composition after chemical modification. The major product (B-1, urea) has the same retention time as in the original analysis (3.26 min, FIG. 13A), while the retention time of minor product (B-1, carbamate) has shifted to 3.86 min, consistent with chemical functionalization of the free amine moiety. Furthermore, mass spectrometric analysis of the peak at 3.86 min (M+134) indicates the predicted functionalization has occurred (see FIG. 14B). These results suggest the minor product is indeed a carbamate side product.

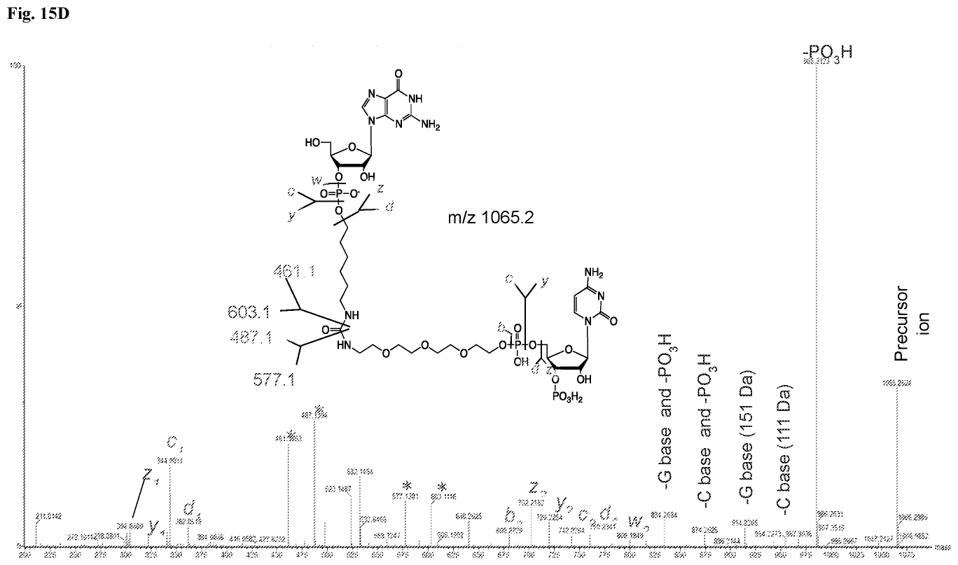

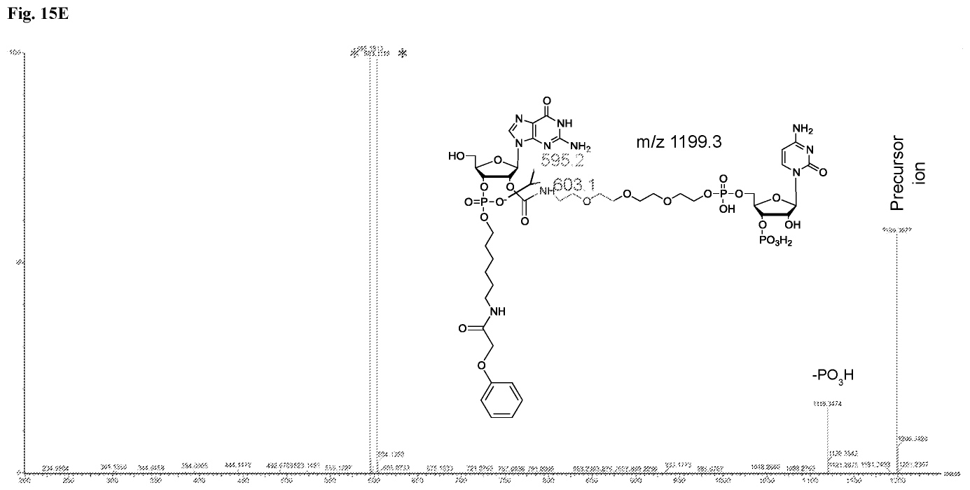

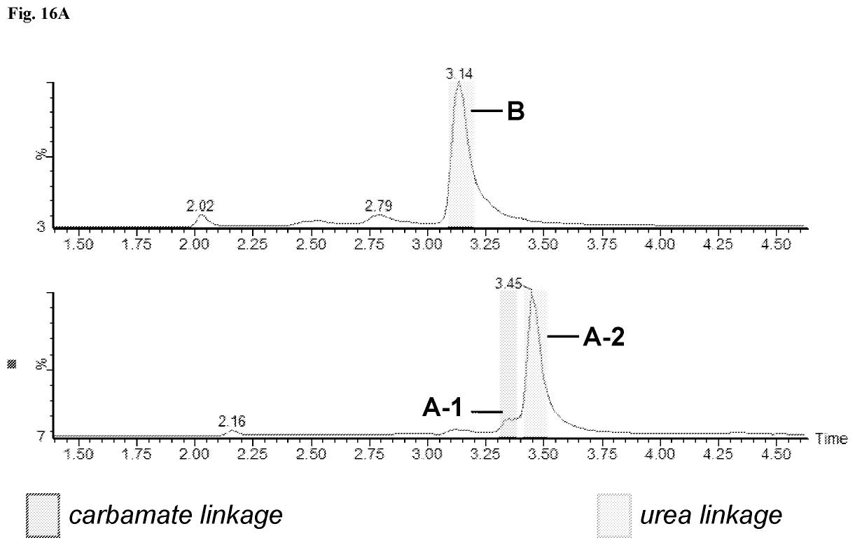

To further confirm the identity of the carbamate side product, the mixture of major product (urea) and chemically modified minor product (carbamate) were subjected to digestion with ribonuclease A (see Example 9), which cleaved the guide molecules at the 3′-end of each G nucleotide in the primary sequence. The fragments were then analyzed by LC-MS, and both the urea linkage (G35-[UR]-C36) and the chemically modified carbamate linkage (G35-[CA+PAA]-C36) were detected. FIG. 15A shows the LC-MS trace of the fragment mixture with the urea linkage at a retention time of 4.31 min and the chemically modified carbamate linkage at a retention time of 5.77 min. FIG. 15B shows the mass spectrum of the peak at 4.31 min, where m/z=532.1 is assigned to [M−2H]²⁻, and FIG. 15C shows the mass spectrum of the peak at 5.77 min, where m/z=599.1 is assigned to [M−2H]²⁻. The mass spectra were further analyzed using LC-MS/MS techniques. The LC-MS/MS spectrum (FIG. 15D) of the urea linked product at m/z 532.1, [M−2H]²⁻, contains the typical a-d and x-z ions that are observed in oligonucleotide collision-induced dissociation (CID) experiments. In addition, MS/MS fragment ions on either side of the UR linkage from the 5′-end (m/z=487.1 and 461.1) and the 3′-end (m/z=603.1 and 577.1) were observed. In contrast, only two product ions were observed in the LC-MS/MS spectrum (FIG. 15E) of the chemically modified carbamate linked product at m/z 599.1, [M−2H]²⁻, including a MS/MS fragment ion from the 5′-end of the carbamate linkage (m/z=595.2) and the 3′-end of the carbamate linkage (m/z=603.1).

### Example 11: Nucleotide Modifications for Single Product Formation

We hypothesized that formation of the carbamate side product as described in Example 10 could be prevented through strategic 2′-modifications in the nucleotide at the 3′ end of the 5′ guide molecule fragment. For example, replacing the 2′-OH in the nucleotide at the 3′ end of the 5′ guide molecule fragment with a 2′-H, synthesis of a urea-linked guide molecule in accordance with the process of Example 1 was hypothesized to yield a single urea-linked product with no carbamate side product:

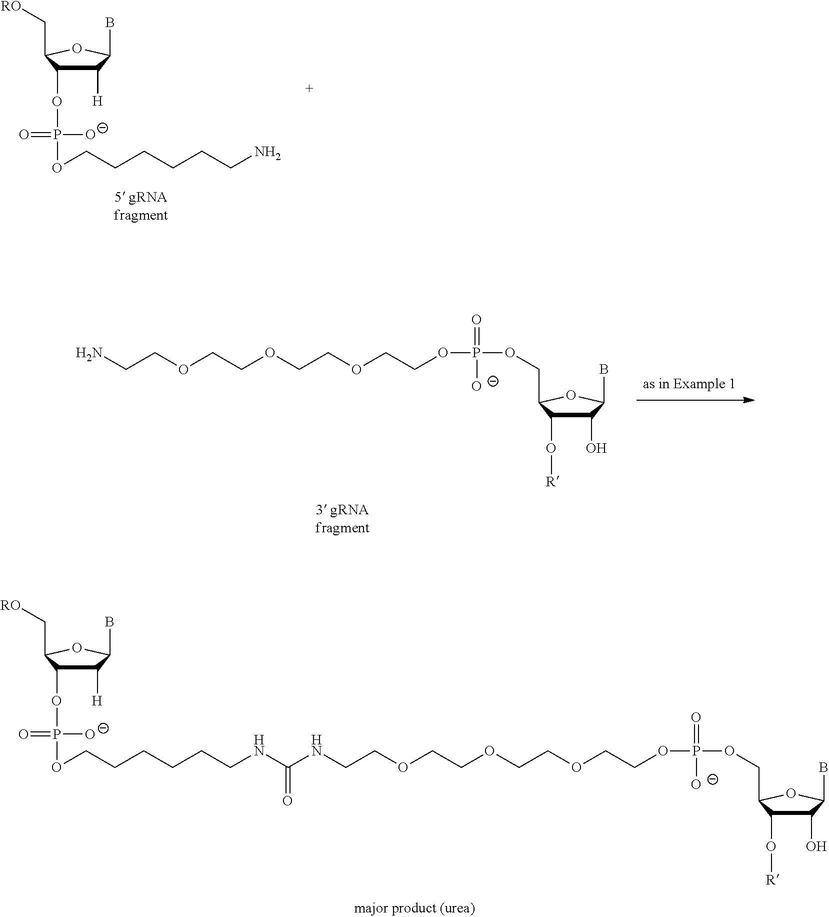

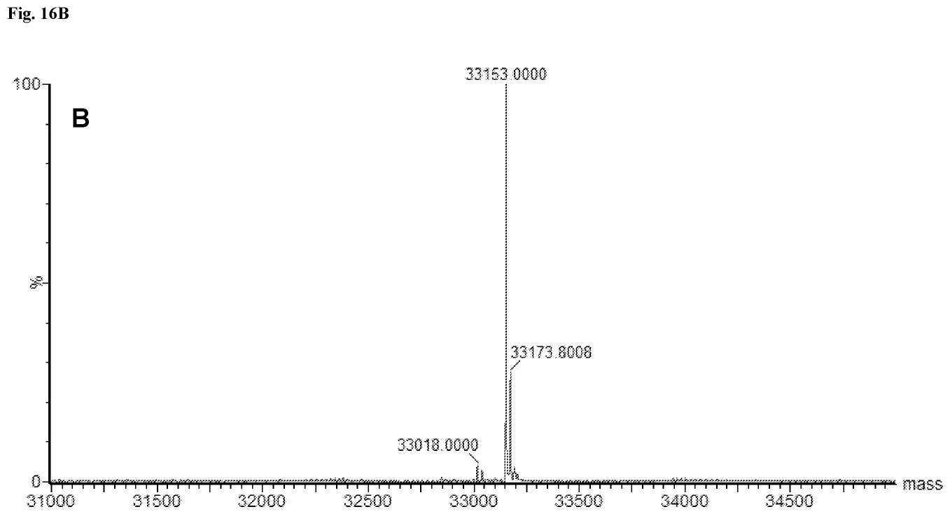

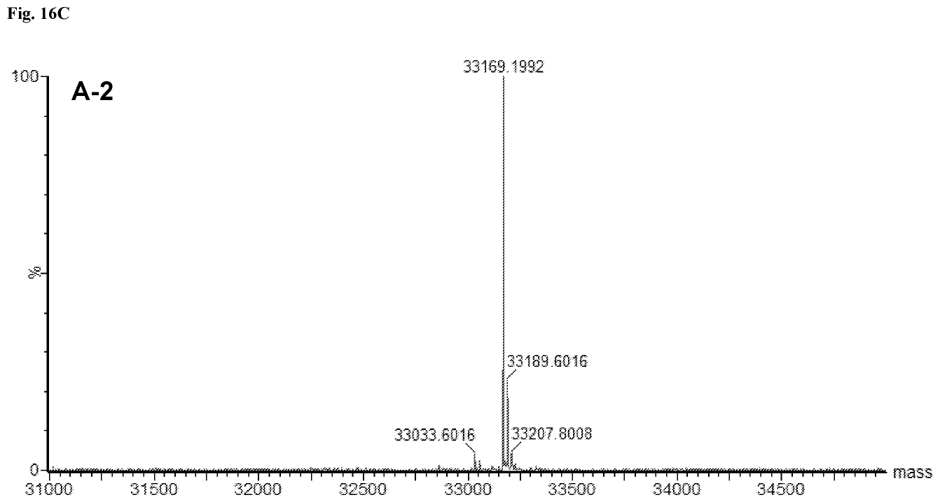

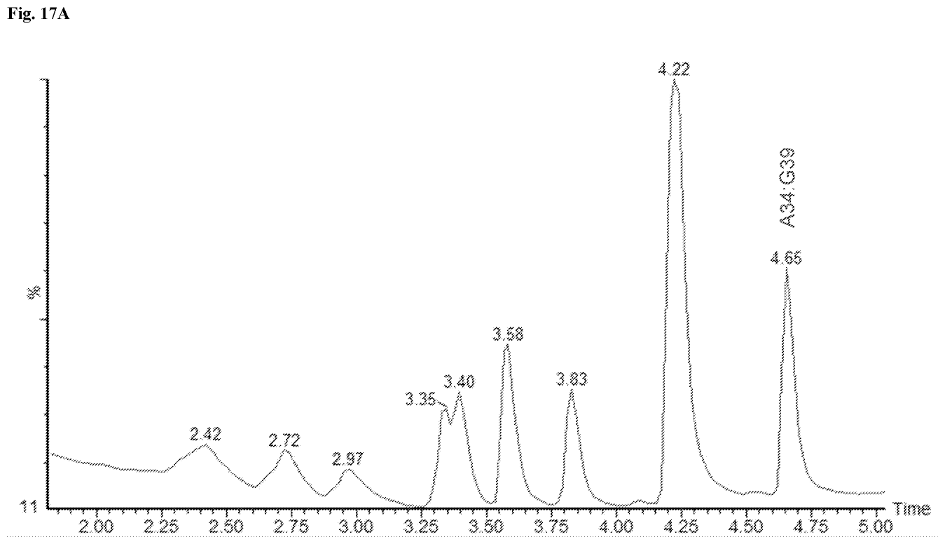

FIG. 16A shows LC-MS data of the crude reaction mixture for a reaction with a 2′-H modified 5′ guide molecule fragment (upper spectrum), compared to a crude reaction mixture for a reaction with an unmodified version of the same 5′ guide molecule (lower spectrum). There is no carbamate side product formation observed with the 2′-H modified 5′ guide molecule fragment (upper spectrum). In contrast, the crude reaction mixture for a reaction with an unmodified version of the same 5′ guide molecule fragment (lower spectrum) included a mixture of the major urea-linked product (A-2) and the minor carbamate side product (A-1). We note that, unlike in Example 10, the carbamate side product was not enriched and was therefore detected at much lower levels than in FIG. 13A of Example 10. Furthermore, mass spectrometric analysis of the product of the reaction with the 2′-H modified 5′ guide molecule fragment (B) gave M-16 (compared to A-2, the major unmodified urea-linked product), as expected for a molecule where a 2′-OH has been replaced with a 2′-H (see FIG. 16B and FIG. 16C).

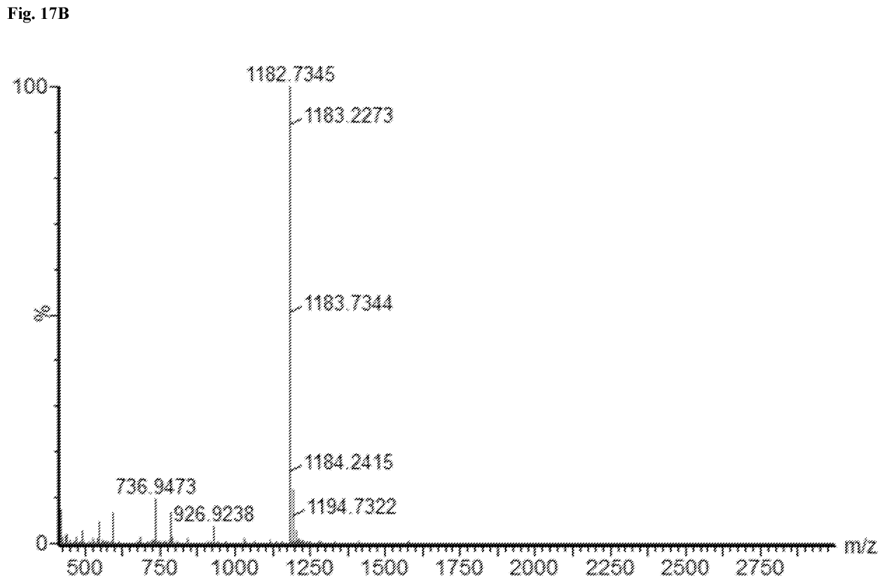

An analogous experiment was performed using gRNA 1L of Table 10, which contains the same 2′-H modification. The formation of a 2′-H modified, urea-linked guide molecule was confirmed by T1 endonuclease digestion, followed by mass spectrometric analysis (see Example 9). The fragment containing the urea linkage, (2′-H-A)-[UR]-AAUAG (A34:G39), was detected at a retention time of 4.65 min (FIG. 17A) with m/z=1182.7 (FIG. 17B). LC-MS/MS analysis of this precursor ion revealed fragment ions consistent with a urea linkage in the reaction with the 2′-H modified nucleotide.

These results suggest that through 2′-OH modifications in the nucleotide at the 3′ end of the 5′ guide molecule fragment, the formation of the carbamate side product can be avoided. Consequently, the urea-ligated guide molecule is synthesized in high purity, which streamlines the overall process of producing a conjugated guide molecule.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

1-249. (canceled)

250. A unimolecular guide molecule for a CRISPR system comprising a non-nucleotide linkage comprising a urea, wherein the guide molecule is of formula: 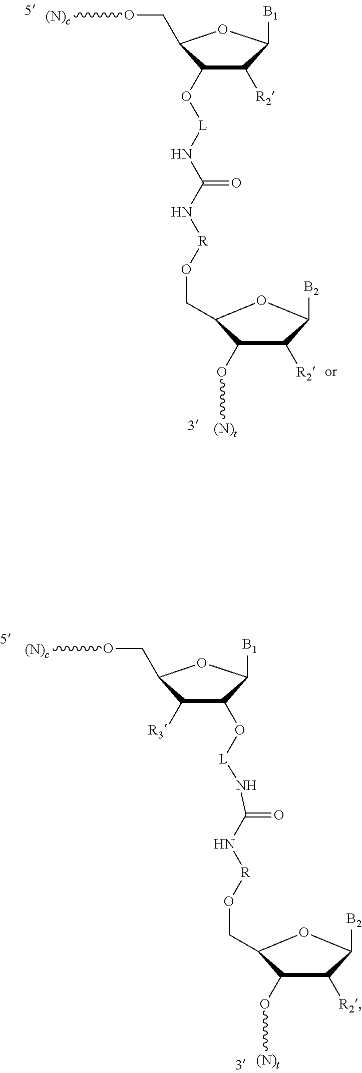
wherein:
each N in (N)_{c} and (N)ₜ is independently a nucleotide residue, each independently linked to its adjacent nucleotide(s) via a phosphodiester linkage, a phosphorothioate linkage, a phosphonoacetate linkage, a thiophosphonoacetate linkage, or a phosphoroamidate linkage;
(N)_{c} includes a 3′ region that is complementary or partially complementary to, and forms a duplex with, a 5′ region of (N)ₜ;
c is an integer 20 or greater;
t is an integer 20 or greater;
each [symbol] represents independently a phosphodiester linkage, a phosphorothioate linkage, a phosphonoacetate linkage, a thiophosphonoacetate linkage, or a phosphoroamidate linkage;
each of R₂′ and R₃′ is independently H, OH, fluoro, chloro, bromo, NH₂, SH, S—R′, or O—R′ wherein each R′ is independently a protection group or an alkyl group, wherein the alkyl group may be optionally substituted;
L and R are each independently a non-nucleotide linker; and
B₁ and B₂ are each independently a nucleobase.

251. The guide molecule of claim 250, wherein the guide molecule is of formula: 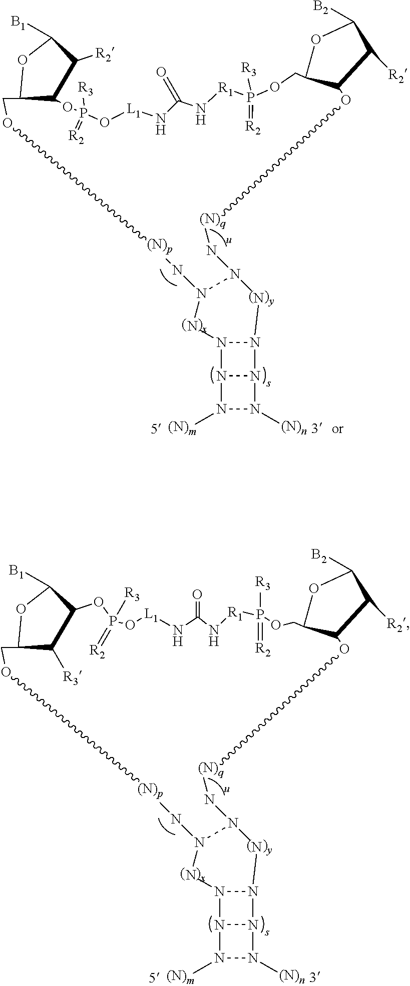
wherein:
L₁ and R₁ are each independently a non-nucleotide linker;
each R₂ is independently O or S;
each R₃ is independently O⁻ or COO⁻;
p and q are each independently an integer between 0 and 6, inclusive, and p+q is an integer between 0 and 6, inclusive;
u is an integer between 2 and 22, inclusive;
s is an integer between 1 and 10, inclusive;
x is an integer between 1 and 3, inclusive;
y is >x and an integer between 3 and 5, inclusive;
m is an integer 15 or greater;
n is an integer 30 or greater;
each N is independently a nucleotide residue, optionally a modified nucleotide residue, each independently linked to its adjacent nucleotide(s) via a phosphodiester linkage, a phosphorothioate linkage, a phosphonoacetate linkage, a thiophosphonoacetate linkage, or a phosphoroamidate linkage; and
each [symbol] independently represents two complementary nucleotides, optionally two complementary nucleotides that are hydrogen bonding base-paired.

252. The guide molecule of claim 251, wherein p and q are each 0.

253. The guide molecule of claim 251, wherein u is an integer between 4 and 14, inclusive.

254. The guide molecule of claim 251, wherein the guide molecule is of formula: 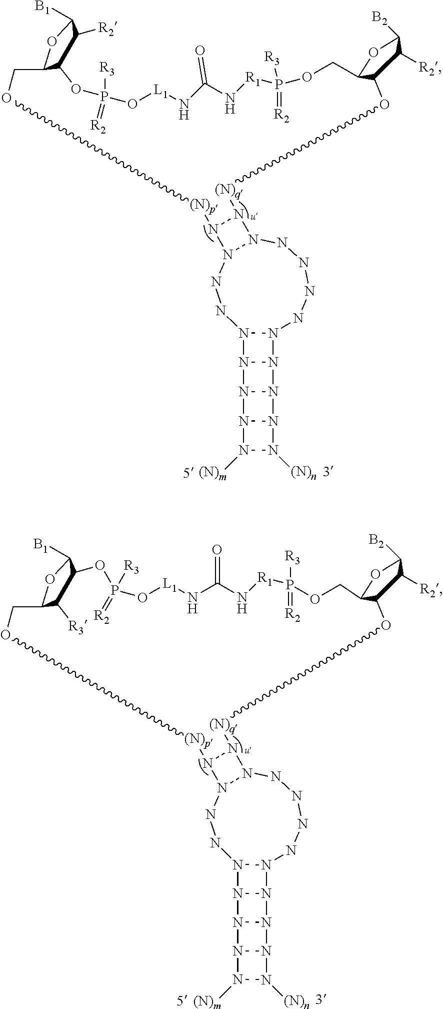 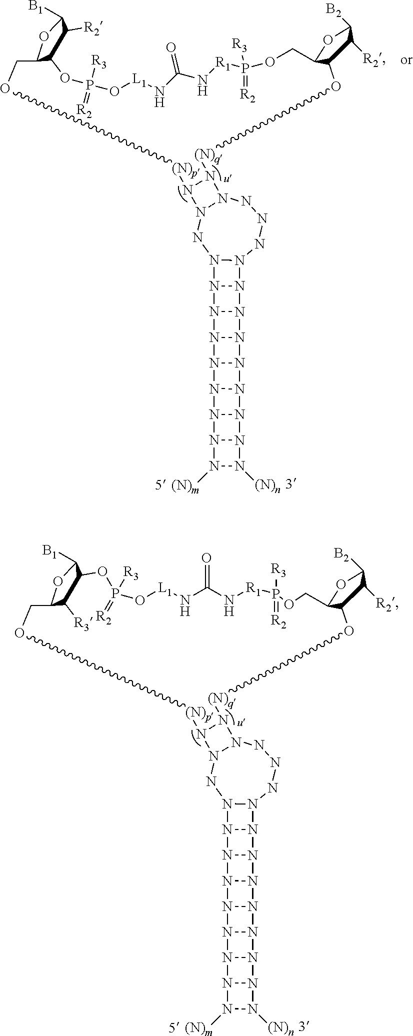
wherein:
u′ is an integer between 2 and 22, inclusive; and
p′ and q′ are each independently an integer between 0 and 4, inclusive, and p′+q′ is an integer between 0 and 4, inclusive.

255. The guide molecule of claim 254, wherein p′ and q′ are each 0.

256. The guide molecule of claim 251, wherein L₁ and R₁ are each independently selected from —(CH₂)_{w}—, —(CH₂)_{w}—NH—C(O)—(CH₂)_{w}—NH—, —(OCH₂CH₂)ᵥ—NH—C(O)—(CH₂)_{w}—, and —(CH₂CH₂O)ᵥ—, wherein each w is independently an integer between 1 and 20 inclusive, and each v is an integer between 1 and 10 inclusive.

257. The guide molecule of claim 256, wherein w is 6 and v is 4.

258. The guide molecule of claim 250, wherein the guide molecule is selected from:
SEQ ID NO. 36-[UR]-SEQ ID NO. 58;
SEQ ID NO. 37-[UR]-SEQ ID NO. 59;
SEQ ID NO. 38-[UR]-SEQ ID NO. 60;
SEQ ID NO. 39-[UR]-SEQ ID NO. 61;
SEQ ID NO. 40-[UR]-SEQ ID NO. 62;
SEQ ID NO. 41-[UR]-SEQ ID NO. 63;
SEQ ID NO. 42-[UR]-SEQ ID NO. 64;
SEQ ID NO. 43-[UR]-SEQ ID NO. 65;
SEQ ID NO. 44-[UR]-SEQ ID NO. 66;
SEQ ID NO. 45-[UR]-SEQ ID NO. 67;
SEQ ID NO. 46-[UR]-SEQ ID NO. 68;
SEQ ID NO. 47-[UR]-SEQ ID NO. 69;
SEQ ID NO. 48-[UR]-SEQ ID NO. 70;
SEQ ID NO. 49-[UR]-SEQ ID NO. 71;
SEQ ID NO. 50-[UR]-SEQ ID NO. 72;
SEQ ID NO. 51-[UR]-SEQ ID NO. 73;
SEQ ID NO. 52-[UR]-SEQ ID NO. 74;
SEQ ID NO. 53-[UR]-SEQ ID NO. 75;
SEQ ID NO. 54-[UR]-SEQ ID NO. 76;
SEQ ID NO. 55-[UR]-SEQ ID NO. 77; and
SEQ ID NO. 56-[UR]-SEQ ID NO. 78,
wherein [UR] is the non-nucleotide linkage comprising a urea.

259. A composition comprising a guide molecule of claim 250, or a pharmaceutically acceptable salt thereof.

260. The composition of claim 259, wherein the guide molecule is suspended in solution or in a pharmaceutically acceptable carrier.

261. The composition of claim 259, further comprising a Cas9 protein, wherein the guide molecule and the Cas9 protein form a complex capable of interacting with a target nucleic acid comprising (i) a sequence complementary to the targeting domain sequence; and (ii) a protospacer adjacent motif (PAM) sequence that is recognized by the Cas9 protein.

262. The composition of claim 259, wherein:
(i) the guide molecule is of formula: 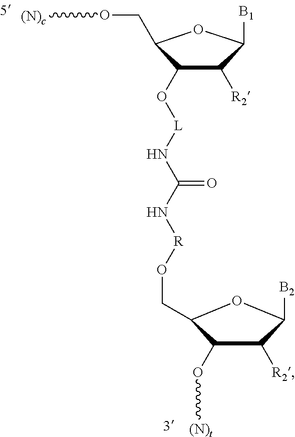
wherein the composition is substantially free of molecules of formula: 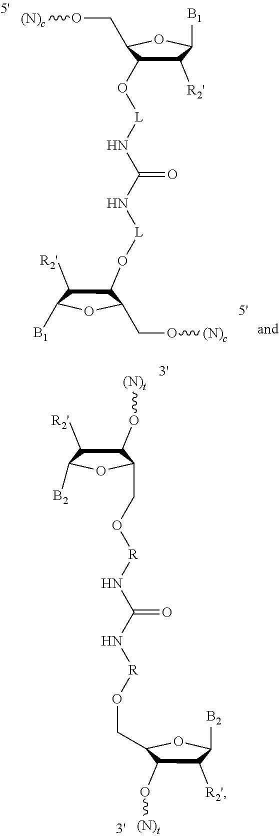
or a pharmaceutically acceptable salt thereof; or
(ii) the guide molecule is of formula: 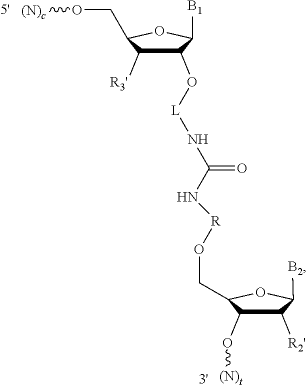
wherein the composition is substantially free of molecules of formula: 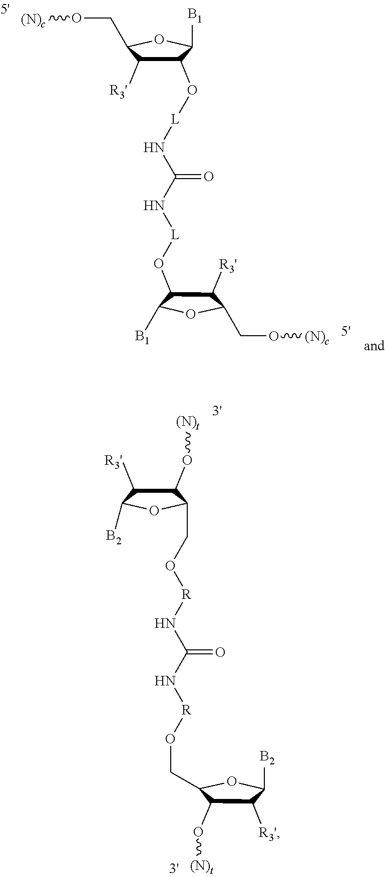
or a pharmaceutically acceptable salt thereof.

263. The composition of claim 259, wherein:
(i) the guide molecule is of formula: 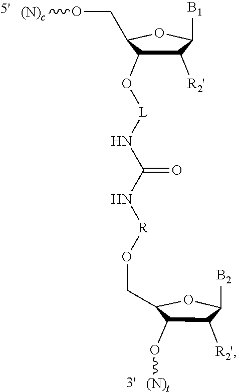
wherein the composition is substantially free of molecules of formula: 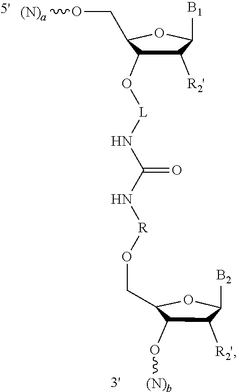
or a pharmaceutically acceptable salt thereof, wherein:
a is not equal to c; and/or
b is not equal to t; or
(ii) the guide molecule is of formula: 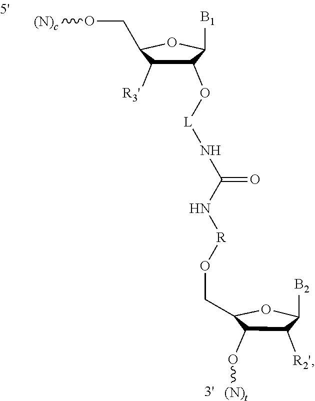
wherein the composition is substantially free of molecules of formula: 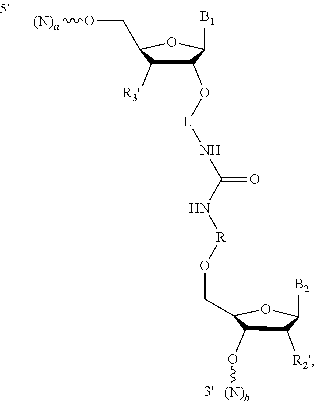
or a pharmaceutically acceptable salt thereof, wherein:
a is not equal to c; and/or
b is not equal to t.

264. The composition of claim 259, wherein:
(i) the guide molecule is of formula: 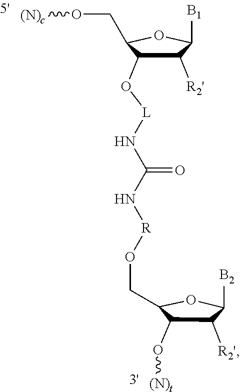
wherein the composition is substantially free of molecules of formula: 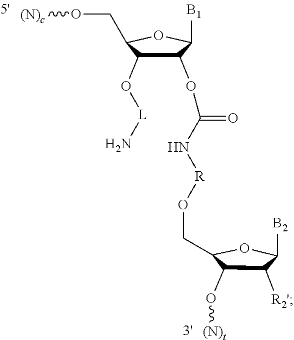
or
(ii) the guide molecule is of formula: 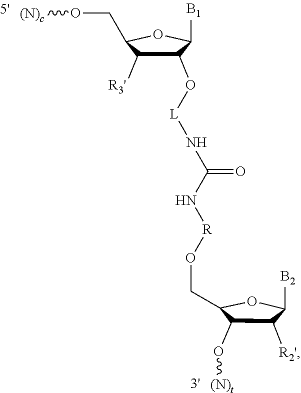
wherein the composition is substantially free of molecules of formula:

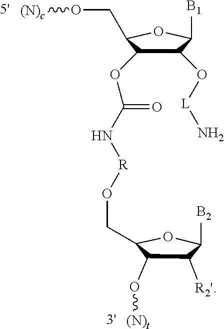

265. A method of synthesizing the guide molecule of claim 250, the method comprising steps of:
annealing a first oligonucleotide and a second oligonucleotide to form a duplex between a 3′ region of the first oligonucleotide and a 5′ region of the second oligonucleotide,
wherein the first oligonucleotide comprises a first reactive group, wherein the first reactive group comprises an amine moiety and is a 2′ reactive group or a 3′ reactive group, and
wherein the second oligonucleotide comprises a second reactive group, wherein the second reactive group comprises an amine moiety and is a 5′ reactive group; and
conjugating the annealed first and second oligonucleotides via the first and second reactive groups to form the guide molecule that includes a covalent bond linking the first and second oligonucleotides.

266. A method of altering a nucleic acid in a cell or subject comprising administering to the subject the guide molecule of claim 250.

267. A unimolecular guide molecule for a CRISPR system, wherein the guide molecule is of formula: 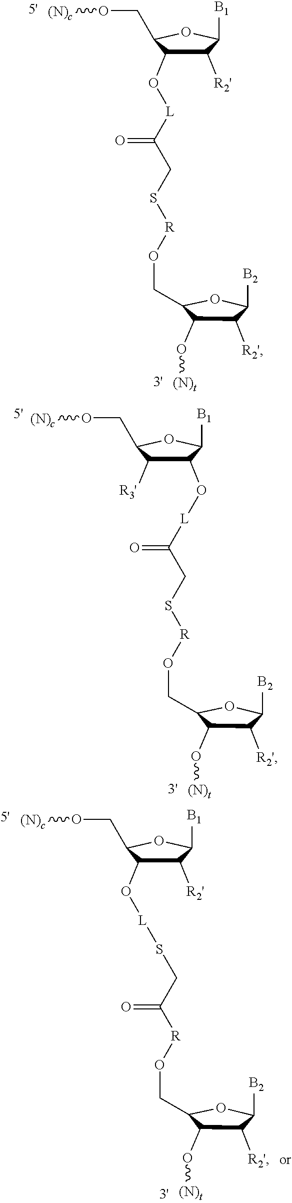 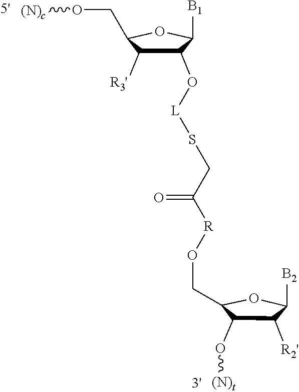
wherein:
each N in (N)_{c} and (N)ₜ is independently a nucleotide residue, each independently linked to its adjacent nucleotide(s) via a phosphodiester linkage, a phosphorothioate linkage, a phosphonoacetate linkage, a thiophosphonoacetate linkage, or a phosphoroamidate linkage;
(N)_{c} includes a 3′ region that is complementary or partially complementary to, and forms a duplex with, a 5′ region of (N)ₜ;
c is an integer 20 or greater;
t is an integer 20 or greater;
each [symbol] represents independently a phosphodiester linkage, a phosphorothioate linkage, a phosphonoacetate linkage, a thiophosphonoacetate linkage, or a phosphoroamidate linkage;
each of R₂′ and R₃′ is independently H, OH, fluoro, chloro, bromo, NH₂, SH, S—R′, or O—R′ wherein each R′ is independently a protection group or an alkyl group, wherein the alkyl group may be optionally substituted;
L and R are each independently a non-nucleotide linker; and
B₁ and B₂ are each independently a nucleobase.

268. A composition comprising a guide molecule of claim 267, or a pharmaceutically acceptable salt thereof.

269. A method of synthesizing the guide molecule of claim 267, the method comprising steps of:
annealing a first oligonucleotide and a second oligonucleotide to form a duplex between a 3′ region of the first oligonucleotide and a 5′ region of the second oligonucleotide,
wherein:
(i) the first oligonucleotide comprises a first reactive group, wherein the first reactive group comprises a bromoacetyl moiety and is a 2′ reactive group or a 3′ reactive group, and the second oligonucleotide comprises a second reactive group, wherein the second reactive group comprises a sulfhydryl moiety and is a 5′ reactive group; or
(ii) the first oligonucleotide comprises a first reactive group, wherein the first reactive group comprises a sulfhydryl moiety and is a 2′ reactive group or a 3′ reactive group, and the second oligonucleotide comprises a second reactive group, wherein the second reactive group comprises a bromoacetyl moiety and is a 5′ reactive group; and
conjugating the annealed first and second oligonucleotides via the first and second reactive groups to form the guide molecule that includes a covalent bond linking the first and second oligonucleotides.

270. A method of altering a nucleic acid in a cell or subject comprising administering to the subject the guide molecule of claim 267.

271. A composition comprising a unimolecular guide molecule for a CRISPR system, wherein the guide molecule is of formula: 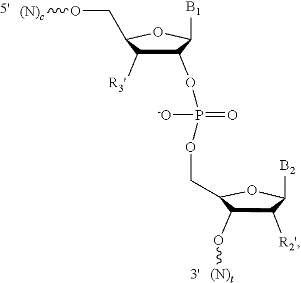
or a pharmaceutically acceptable salt thereof, wherein:
each N in (N)_{c} and (N)ₜ is independently a nucleotide residue, each independently linked to its adjacent nucleotide(s) via a phosphodiester linkage, a phosphorothioate linkage, a phosphonoacetate linkage, a thiophosphonoacetate linkage, or a phosphoroamidate linkage;
(N)_{c} includes a 3′ region that is complementary or partially complementary to, and forms a duplex with, a 5′ region of (N)ₜ;
the 2′-5′ phosphodiester linkage depicted in the formula is between two nucleotides in said duplex;
c is an integer 20 or greater;
t is an integer 20 or greater;
B₁ and B₂ are each independently a nucleobase;
each of R_{2′} and R₃′ is independently H, OH, fluoro, chloro, bromo, NH₂, SH, S—R′, or O—R′ wherein each R′ is independently a protection group or an alkyl group, wherein the alkyl group may be optionally substituted; and
each [symbol] represents independently a phosphodiester linkage, a phosphorothioate linkage, a phosphonoacetate linkage, a thiophosphonoacetate linkage, or a phosphoroamidate linkage.

272. A method of preparing the composition of claim 271, the method comprising steps of:
annealing a first oligonucleotide and a second oligonucleotide to form a duplex between a 3′ region of the first oligonucleotide and a 5′ region of the second oligonucleotide,
wherein the first oligonucleotide comprises a first reactive group, wherein the first reactive group comprises a hydroxyl moiety and is a 2′ reactive group or a 3′ reactive group, and
wherein the second oligonucleotide comprises a second reactive group, wherein the second reactive group comprises a phosphate moiety and is a 5′ reactive group; and
conjugating the annealed first and second oligonucleotides via the first and second reactive groups to provide a composition comprising a guide molecule that includes a covalent bond linking the first and second oligonucleotides.

273. A method of altering a nucleic acid in a cell or subject comprising administering to the subject the composition of claim 271.
